(19) United States
(12) Patent Application Publication
(10) Pub. No.: US 2007/0054282 A1
(43) Pub. Date: Mar. 8, 2007

(54) Method for the detection of gene transcripts in blood and uses thereof

(75) Inventor: Choong-Chin Liew, Toronto (CA)

Correspondence Address: PALMER & DODGE, LLP;KATHLEEN M. WILLIAMS, 111 HUNTINGTON AVENUE, BOSTON, MA, 02199, US

(73) Assignee: Chondrogene Limited

(21) Appl. No.: 11/313,302

(22) Filed: Dec. 20, 2005

Related U.S. Application Data
(63) Continuation of application No. PCT/US04/20836, filed on Jun. 21, 2004.
Application No. PCT/US04/20836 is a continuation-in-part of application No. 10/809,675, filed on Mar. 25, 2004.
Application No. 10/809,675 is a continuation-in-part of application No. 10/601,518, filed on Jun. 20, 2003.

(51) Int. Cl. C12Q 1/68

(52) U.S. Cl. 435/6

(57) ABSTRACT

The present invention relates generally to the identification of biomarkers of conditions including disease and non disease conditions as well as identifying compositions of biomarkers. The invention further provides a method of diagnosing disease, monitoring disease progression, and differentially diagnosing disease. The invention further provides for kits useful in diagnosing, monitoring disease progression and differentially diagnosing disease.

### RELATED APPLICATIONS

This application is a continuation of Application Serial No. PCT/US04/020836, filed on Jun. 21, 2004, which claims priority to application Ser. No. 10/809,675 filed on Mar. 25, 2004, which claims priority to application Ser. No. 10/601,518 filed on Jun. 20, 2003. Theses applications are incorporated herein by reference in their entirety.

### FIELD OF THE INVENTION

This application relates to the identification of biomarkers in blood, the identified biomarkers and compositions thereof, as well as methods related to the use of the biomarkers to monitor an individual's condition.

Tables

This application includes a compact disc in duplicate (2 compact discs: Tables—Copy 1 and Tables—Copy 2), which are hereby incorporated by reference in their entirety. Each compact disc is identical and contains the following files:

| | TABLE | DESCRIPTION | SIZEKB | CREATED | Text File Name |
|---|---|---|---|---|---|
| 1 | 1A | Sequence Related Table | 19 | 2004-06-18 | TABLE1A.TXT |
| | | regarding Comorbid | | | |
| | | Hypertension | | | |
| 2 | 1B | Sequence Related Table | 20 | 2004-06-18 | TABLE1B.TXT |
| | | regarding Comorbid Obesity | | | |
| 3 | 1C | Sequence Related Table | 14 | 2004-06-18 | TABLE1C.TXT |
| | | regarding Comorbid Allergies | | | |
| 4 | 1D | Sequence Related Table | 13 | 2004-06-18 | TABLE1D.TXT |
| | | regarding Comorbid Systemic | | | |
| | | Steroids | | | |
| 5 | 1E | Sequence Related Table | 48 | 2004-06-18 | TABLE1E.TXT |
| | | regarding Hypertension | | | |
| | | (Chondro) | | | |
| 6 | 1F | Sequence Related Table | 54 | 2004-06-18 | TABLE1F.TXT |
| | | regarding Obesity (Chondro) | | | |
| 7 | 1G | Sequence Related Table | 13 | 2004-06-16 | TABLE1G.TXT |
| | | regarding Comorbid | | | |
| | | Hypertension Only | | | |
| 8 | 1H | Sequence Related Table | 5 | 2004-06-16 | TABLE1H.TXT |
| | | regarding Hypertension OA | | | |
| | | Shared | | | |
| 9 | 1I | Sequence Related Table | 12 | 2004-06-16 | TABLE1I.TXT |
| | | regarding Comorbid Obesity | | | |
| | | Only | | | |
| 10 | 1J | Sequence Related Table | 4 | 2004-06-16 | TABLE1J.TXT |
| | | regarding Obesity OA Shared | | | |
| 11 | 1K | Sequence Related Table | 6 | 2004-06-16 | TABLE1K.TXT |
| | | regarding Comorbid Allergy | | | |
| | | Only | | | |
| 12 | 1L | Sequence Related Table | 6 | 2004-06-16 | TABLE1L.TXT |
| | | regarding Allergy OA Shared | | | |
| 13 | 1M | Sequence Related Table | 8 | 2004-06-16 | TABLE1M.TXT |
| | | regarding Comorbid Steroid | | | |
| | | Shared | | | |
| 14 | 1N | Sequence Related Table | 5 | 2004-06-16 | TABLE1N.TXT |
| | | regarding Steroid OA Shared | | | |
| 15 | 1O | Sequence Related Table | 9 | 2004-06-16 | TABLE1O.TXT |
| | | regarding Differentiating | | | |
| | | Systemic Steroids ( | | | |
| 16 | 1P | Sequence Related Table | 28 | 2004-06-16 | TABLE1P.TXT |
| | | regarding Diabetes | | | |
| 17 | 1Q | Sequence Related Table | 34 | 2004-06-16 | TABLE1Q.TXT |
| | | regarding Hyperlipidemia | | | |
| 18 | 1R | Sequence Related Table | 21 | 2004-06-16 | TABLE1R.TXT |
| | | regarding Lung Disease | | | |
| 19 | 1S | Sequence Related Table | 146 | 2004-06-16 | TABLE1S.TXT |
| | | regarding Bladder Cancer | | | |
| 20 | 1T | Sequence Related Table | 83 | 2004-06-16 | TABLE1T.TXT |
| | | regarding Bladder Cancer | | | |
| | | Staging | | | |
| 21 | 1U | Sequence Related Table | 117 | 2004-06-16 | TABLE1U.TXT |
| | | regarding Coronary Artery | | | |
| | | Disease | | | |
| 22 | 1V | Sequence Related Table | 78 | 2004-06-16 | TABLE1V.TXT |
| | | regarding Rheumatoid | | | |
| | | Arthritis | | | |
| 23 | 1W | Sequence Related Table | 44 | 2004-06-16 | TABLE1W.TXT |
| | | regarding Rheumatoid | | | |
| | | Arthritis | | | |
| 24 | 1X | Sequence Related Table | 36 | 2004-06-16 | TABLE1X.TXT |
| | | regarding Depression | | | |
| 25 | 1Y | Sequence Related Table | 7 | 2004-06-16 | TABLE1Y.TXT |
| | | regarding OA Staging | | | |
| 26 | 1Z | Sequence Related Table | 109 | 2004-06-16 | TABLE1Z.TXT |
| | | regarding Liver Cancer | | | |
| 27 | 1AA | Sequence Related Table | 110 | 2004-06-16 | TABLE1AA.TXT |
| | | regarding Schizophrenia | | | |
| 28 | 1AB | Sequence Related Table | 34 | 2004-06-16 | TABLE1AB.TXT |
| | | regarding Chagas Disease | | | |
| 29 | 1AC | Sequence Related Table | 13 | 2004-06-18 | TABLE1AC.TXT |
| | | regarding Asthma (Chondro) | | | |
| 30 | 1AD | Sequence Related Table | 15 | 2004-06-16 | TABLE1AD.TXT |
| | | regarding Asthma (Affy) | | | |
| | 1AE | Sequence Related Table | 31 | 2004-06-16 | TABLE1AE.TXT |
| | | regarding Lung Cancer | | | |
| | 1AG | Sequence Related Table | 29 | 2004-06-16 | TABLE1AG.TXT |
| | | regarding Hypertension | | | |
| | | (Affymetrix) | | | |
| | 1AH | Sequence Related Table | 35 | 2004-06-16 | TABLE1AH.TXT |
| | | regarding Obesity | | | |
| | | (Affymetrix) | | | |
| | 1AI | Sequence Related Table | 65 | 2004-06-16 | TABLE1AI.TXT |
| | | regarding Ankylosing | | | |
| | | Spondylitis (Affy) | | | |
| 31 | 2 | Sequence Related Table | 4 | 2004-06-16 | TABLE2.TXT |
| | | regarding OA Only | | | |
| | | Subtraction | | | |
| 32 | 3A | Sequence Related Table | 51 | 2004-06-16 | TABLE3A.TXT |
| | | regarding Schizophrenia v. | | | |
| | | MDS | | | |
| 33 | 3B | Sequence Related Table | 96 | 2004-06-16 | TABLE3B.TXT |
| | | regarding Hepatitis v. Liver | | | |
| | | Cancer | | | |
| 34 | 3C | Sequence Related Table | 114 | 2004-06-16 | TABLE3C.TXT |
| | | regarding Bladder Cancer v. | | | |
| | | Kidney Cancer | | | |
| 35 | 3D | Sequence Related Table | 121 | 2004-06-16 | TABLE3D.TXT |
| | | regarding Bladder Cancer v. | | | |
| | | Testicular Cancer | | | |
| 36 | 3E | Sequence Related Table | 132 | 2004-06-16 | TABLE3E.TXT |
| | | regarding Testicular Cancer v. | | | |
| | | Kidney Cancer | | | |
| 37 | 3F | Sequence Related Table | 15 | 2004-06-16 | TABLE3F.TXT |
| | | regarding Liver Cancer v. | | | |
| | | Stomach Cancer | | | |
| 38 | 3G | Sequence Related Table | 27 | 2004-06-16 | TABLE3G.TXT |
| | | regarding Liver Cancer v. | | | |
| | | Colon Cancer | | | |
| 39 | 3H | Sequence Related Table | 30 | 2004-06-16 | TABLE3H.TXT |
| | | regarding Stomach Cancer v. | | | |
| | | Colon Cancer | | | |
| 40 | 3I | Sequence Related Table | 49 | 2004-06-16 | TABLE3I.TXT |
| | | regarding OA v. RA | | | |
| 42 | 3K | Sequence Related Table | 3 | 2004-06-16 | TABLE3K.TXT |
| | | regarding Chagas Disease | | | |
| | | v. Heart Failure | | | |
| 43 | 3L | Sequence Related Table | 4 | 2004-06-16 | TABLE3L.TXT |
| | | regarding Chagas Disease v. | | | |
| | | CAD | | | |
| 45 | 3N | Sequence Related Table | 3 | 2004-06-16 | TABLE3N.TXT |
| | | regarding CAD v. Heart | | | |
| | | Failure | | | |
| 47 | 3P | Sequence Related Table | 17 | 2004-06-16 | TABLE3P.TXT |
| | | regarding Asymptomatic | | | |
| | | Chagas v. Symptomatic | | | |
| | | Chagas | | | |
| 48 | 3Q | Sequence Related Table | 13 | 2004-06-16 | TABLE3Q.TXT |
| | | regarding Alzheimer's' v. | | | |
| | | Schizophrenia | | | |
| 49 | 3R | Sequence Related Table | 12 | 2004-06-16 | TABLE3R.TXT |
| | | regarding Alzheimer's' v. | | | |
| | | Manic Depression | | | |
| 50 | 4A | Sequence Related Table | 112 | 2004-06-16 | TABLE4A.TXT |
| | | regarding OA v. Control | | | |
| | | (ChondroChip) | | | |
| 51 | 4B | Sequence Related Table | 144 | 2004-06-16 | TABLE4B.TXT |
| | | regarding OA v. Control | | | |
| | | (Affy) | | | |
| 52 | 4C | Sequence Related Table | 67 | 2004-06-16 | TABLE4C.TXT |
| | | regarding OA mild v. Control | | | |
| | | (ChondroChip) | | | |
| 53 | 4D | Sequence Related Table | 153 | 2004-06-16 | TABLE 4D.TXT |
| | | regarding OA mild v. Control | | | |
| | | (Affy) | | | |
| 54 | 4E | Sequence Related Table | 44 | 2004-06-16 | TABLE4E.TXT |
| | | regarding OA moderate v. | | | |
| | | Control (ChondroChip) | | | |
| 55 | 4F | Sequence Related Table | 152 | 2004-06-16 | TABLE4F.TXT |
| | | regarding OA moderate v. | | | |
| | | Control (Affy) | | | |
| 56 | 4G | Sequence Related Table | 46 | 2004-06-16 | TABLE4G.TXT |
| | | regarding OA marked v. | | | |
| | | Control (ChondroChip) | | | |
| 57 | 4H | Sequence Related Table | 173 | 2004-06-16 | TABLE4H.TXT |
| | | regarding OA marked v. | | | |
| | | Control (Affy) | | | |
| 58 | 4I | Sequence Related Table | 61 | 2004-06-16 | TABLE4I.TXT |
| | | regarding OA severe v. | | | |
| | | Control (ChondroChip) | | | |
| 59 | 4J | Sequence Related Table | 160 | 2004-06-16 | TABLE4J.TXT |
| | | regarding OA severe v. | | | |
| | | Control (Affy) | | | |
| 60 | 4K | Sequence Related Table | 24 | 2004-06-16 | TABLE4K.TXT |
| | | regarding OA mild v. | | | |
| | | moderate (ChondroChip) | | | |
| 61 | 4L | Sequence Related Table | 127 | 2004-06-16 | TABLE4L.TXT |
| | | regarding OA mild v. | | | |
| | | moderate (Affy) | | | |
| 62 | 4M | Sequence Related Table | 21 | 2004-06-16 | TABLE4M.TXT |
| | | regarding OA mild v. marked | | | |
| | | (ChondroChip) | | | |
| 63 | 4N | Sequence Related Table | 101 | 2004-06-16 | TABLE4N.TXT |
| | | regarding OA mild v. marked | | | |
| | | (Affy) | | | |
| 64 | 4O | Sequence Related Table | 35 | 2004-06-16 | TABLE4O.TXT |
| | | regarding OA mild v. severe | | | |
| | | (ChondroChip) | | | |
| 65 | 4P | Sequence Related Table | 180 | 2004-06-16 | TABLE4P.TXT |
| | | regarding OA mild v. severe | | | |
| | | (Affy) | | | |
| 66 | 4Q | Sequence Related Table | 21 | 2004-06-16 | TABLE4Q.TXT |
| | | regarding OA moderate v. | | | |
| | | marked (ChondroChip) | | | |
| 67 | 4R | Sequence Related Table | 115 | 2004-06-16 | TABLE4R.TXT |
| | | regarding OA moderate v. | | | |
| | | marked (Affy) | | | |
| 68 | 4S | Sequence Related Table | 15 | 2004-06-16 | TABLE4S.TXT |
| | | regarding OA moderate v. | | | |
| | | severe (ChondroChip) | | | |
| 69 | 4T | Sequence Related Table | 173 | 2004-06-16 | TABLE4T.TXT |
| | | regarding OA moderate v. | | | |
| | | severe (Affy) | | | |
| 70 | 4U | Sequence Related Table | 13 | 2004-06-16 | TABLE4U.TXT |
| | | regarding OA marked v. | | | |
| | | severe (ChondroChip) | | | |
| 71 | 4V | Sequence Related Table | 193 | 2004-06-16 | TABLE4V.TXT |
| | | regarding OA marked v. | | | |
| | | severe (Affy) | | | |
| 72 | 5A | Sequence Related Table | 24 | 2004-06-16 | TABLE5A.TXT |
| | | regarding Psoriasis v. Control | | | |
| 73 | 5B | Sequence Related Table | 82 | 2004-06-16 | TABLE5B.TXT |
| | | regarding Thyroid Disorder v. | | | |
| | | Control | | | |
| 74 | 5C | Sequence Related Table | 24 | 2004-06-16 | TABLE5C.TXT |
| | | regarding Irritable Bowel | | | |
| | | Syndrome v. Control | | | |
| 75 | 5D | Sequence Related Table | 21 | 2004-06-16 | TABLE5D.TXT |
| | | regarding Osteoporosis v. | | | |
| | | Control | | | |
| 76 | 5E | Sequence Related Table | 50 | 2004-06-16 | TABLE5E.TXT |
| | | regarding Migraine | | | |
| | | Headaches v. Control | | | |
| 77 | 5F | Sequence Related Table | 15 | 2004-06-16 | TABLE5F.TXT |
| | | regarding Eczema v. Control | | | |
| 78 | 5G | Sequence Related Table | 83 | 2004-06-16 | TABLE5G.TXT |
| | | regarding NASH v. Control | | | |
| 79 | 5H | Sequence Related Table | 51 | 2004-06-16 | TABLE5H.TXT |
| | | regarding Alzheimer's' v. | | | |
| | | Control | | | |
| 80 | 5I | Sequence Related Table | 65 | 2004-06-16 | TABLE5I.TXT |
| | | regarding Manic Depression | | | |
| | | v. Control | | | |
| 81 | 5J | Sequence Related Table | 8 | 2004-06-16 | TABLE5J.TXT |
| | | regarding Crohns' Colitis v. | | | |
| | | Control | | | |
| 82 | 5K | Sequence Related Table | 16 | 2004-06-16 | TABLE5K.TXT |
| | | regarding Chronic | | | |
| | | Cholecystits v. Control | | | |
| 83 | 5L | Sequence Related Table | 38 | 2004-06-16 | TABLE5L.TXT |
| | | regarding Heart Failure v. | | | |
| | | Control | | | |
| 84 | 5M | Sequence Related Table | 69 | 2004-06-16 | TABLE5M.TXT |
| | | regarding Cervical Cancer v. | | | |
| | | Control | | | |
| 88 | 5N | Sequence Related Table | 53 | 2004-06-16 | TABLE5N.TXT |
| | | regarding Stomach Cancer v. | | | |
| | | Control | | | |
| 89 | 5O | Sequence Related Table | 81 | 2004-06-16 | TABLE5O.TXT |
| | | regarding Kidney Cancer v. | | | |
| | | Control | | | |
| 90 | 5P | Sequence Related Table | 12 | 2004-06-16 | TABLE5P.TXT |
| | | regarding Testicular Cancer v. | | | |
| | | Control | | | |
| 91 | 5Q | Sequence Related Table | 83 | 2004-06-16 | TABLE5Q.TXT |
| | | regarding Colon Cancer v. | | | |
| | | Control | | | |
| 92 | 5R | Sequence Related Table | 39 | 2004-06-16 | TABLE5R.TXT |
| | | regarding Hepatitis B v. | | | |
| | | Control | | | |
| 93 | 5S | Sequence Related Table | 46 | 2004-06-16 | TABLE5S.TXT |
| | | regarding Pancreatic Cancer | | | |
| | | v. Control | | | |
| 95 | 5T | Sequence Related Table | 18 | 2004-06-16 | TABLE5T.TXT |
| | | regarding Asymptomatic | | | |
| | | Chagas v. Control | | | |
| 96 | 5U | Sequence Related Table | 17 | 2004-06-16 | TABLE5U.TXT |
| | | regarding Symptomatic | | | |
| | | Chagas v. Control | | | |
| | 5V | Sequence Related Table | 66 | 2004-06-16 | TABLE5V.TXT |
| | | regarding Advanced Bladder | | | |
| | | Cancer v. Control | | | |
| 97 | 6A | Sequence Related Table | 42 | 2004-06-16 | TABLE6A.TXT |
| | | regarding Cancer (all types) v. | | | |
| | | Control | | | |
| | 6B | Sequence Related Table | 13 | 2004-06-16 | TABLE6B.TXT |
| | | regarding Cardiovascular | | | |
| | | Disease v. Control | | | |
| | 6C | Sequence Related Table | 69 | 2004-06-16 | TABLE6C.TXT |
| | | regarding Neurological | | | |
| | | Diseases v. Control | | | |
| | 7A | Sequence Related Table | 12 | 2004-06-16 | TABLE7A.TXT |
| | | regarding Celebrex ® v. all | | | |
| | | Cox inhibitors except | | | |
| | | Celebrex | | | |
| 98 | 7B | Sequence Related Table | 12 | 2004-06-16 | TABLE7B.TXT |
| | | regarding Celebrex ® v. | | | |
| | | Control | | | |
| 99 | 7C | Sequence Related Table | 12 | 2004-06-18 | TABLE7C.TXT |
| | | regarding Vioxx ® v. Control | | | |
| 100 | 7D | Sequence Related Table | 11 | 2004-06-16 | TABLE7D.TXT |
| | | regarding Vioxx ® v. All Cox | | | |
| | | Inhibitors except Vioxx ® | | | |
| 101 | 7E | Sequence Related Table | 15 | 2004-06-16 | TABLE7E.TXT |
| | | regarding NSAIDS v. Control | | | |
| 102 | 7F | Sequence Related Table | 51 | 2004-06-16 | TABLE7F.TXT |
| | | regarding Cortisone v. Control | | | |
| 103 | 7G | Sequence Related Table | 72 | 2004-06-16 | TABLE7G.TXT |
| | | regarding Visco Supplement | | | |
| | | v. Control | | | |
| 104 | 7H | Sequence Related Table | 32 | 2004-06-16 | TABLE7H.TXT |
| | | regarding Lipitor ® v. Control | | | |
| 105 | 7I | Sequence Related Table | 6 | 2004-06-16 | TABLE7I.TXT |
| | | regarding Smoker v. Non- | | | |
| | | Smoker | | | |
| | 8A | Affymetrix Annotation Master | 12,488 | 2004-06-17 | TABLE8A.TXT |
| | | Table to Identify Sequence | | | |
| | | Related Information | | | |
| | 8B | ChondroChip Annotation | 3,536 | 2004-06-17 | TABLE8B.TXT |
| | | Master Table to Identify | | | |
| | | Sequence Related Information | | | |
| | 11 | Patent-In listing of the 223 | 187 | 2004-06-21 | TABLE11.TXT |
| | | EST sequences of Tables 1-7 | | | |
| | | with “no significant match” to | | | |
| | | known gene sequence. | | | |

LENGTHY TABLES FILED ON CD
The patent application contains a lengthy table section. A copy of the table is available in electronic form from the USPTO web site (http://seqdata.uspto.gov/?pageRequest=docDetail&DocID=US20070054282A1). An electronic copy of the table will also be available from the USPTO upon request and payment of the fee set forth in 37 CFR 1.19(b)(3).

### BACKGROUND

The blood is a vital part of the human circulatory system for the human body. Numerous cell types make up the blood tissue including leukocytes consisting of granulocytes (neutrophils, eosinophils and basophils), and agranuloctyes (lymphocytes, and monocytes), erythrocytes, platelets, as well as possibly many other undiscovered cell types.

The turnover of cells in the hematopoietic system is enormous. It was reported that over one trillion cells, including 200 billion erythrocytes and 70 billion neutrophilic leukocytes, turn over each day in the human body (Ogawa 1993).

The prior art is deficient in simple non-invasive methods to diagnose, prognose, and monitor progression and regression of disease and to identify markers related to one or more conditions. Although there has been a recent use of expression array phenotyping for identification and/or classification of biomarkers of disease, the source of biomarkers has been limited to those which are differentially expressed in tissue, thus requiring invasive diagnostic procedures (e.g. see Alon U, Barkai N, Notterman D A, Gish K, Ybarra S, Mack D, Levine A J: Broad patterns of gene expression revealed by clustering analysis of tumor and normal colon tissues probed by oligonucleotide arrays. Proc Natl Acad Sci USA 1999, 96:6745-6750; Schummer M, Ng W V, Bumgarner R E, Nelson P S, Schummer B, Bednarski D W, Hassell L, Baldwin R L, Karlan B Y, Hood L Comparative hybridization of an array of 21500 ovarian cDNAs for the discovery of genes overexpressed in ovarian carcinomas. Gene 1999, 238:375-385; van't Veer L J, Dai H, van de Vijver M J, He Y D, Hart A A, Mao M, Peterse H L, van der Kooy K, Marton M J, Witteveen A T, et al.: Gene expression profiling predicts clinical outcome of breast cancer. Nature 2002, 415:530-536;

### SUMMARY OF THE INVENTION

The present invention provides minimally invasive methods to identify biomarkers useful for diagnosing a condition, and biomarkers and compositions thereof, wherein the biomarkers of the condition are identified from a simple blood sample. Also encompassed are methods and kits utilizing said biomarkers, especially to diagnose, prognose, and monitor conditions, which include disease and non disease conditions. Accordingly, methods of diagnosing disease, monitoring disease progression, and differentially diagnosing disease are provided, as well as kits useful in diagnosing, monitoring disease progression and differentially diagnosing disease.

The process described herein requires the use of a blood sample and is, therefore, minimally invasive as compared to conventional practices used to detect disease using tissue sample biomarkers.

Also disclosed are methods representative of means of identifying biomarkers which are differentially expressed as between two populations, a first population having a condition and a second population having a second condition, or not having a condition. The biomarkers thus identified can be used to diagnose an individual with a condition, or differentially diagnose an individual as having either a first or second condition.

Other and further aspects, features, and advantages of the representations of the methods and products presented herein will be apparent from the following description of the presently preferred embodiments. These embodiments are given for the purpose of disclosure.

### BRIEF DESCRIPTION OF THE DRAWINGS

The above-recited features, advantages and objects of the invention, as well as others which will become clear, are attained and can be understood in detail and more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows a diagrammatic representation of RNA expression profiles of Whole blood samples from individuals having both osteoarthritis and hypertension as compared with RNA expression profiles from individuals without either osteoarthritis or hypertension (“normal”).

FIG. 2 shows a diagrammatic representation of RNA expression profiles of Whole blood samples from individuals who were identified as having both osteoarthritis and who were obese as described herein as compared with RNA expression profiles from individuals without either obesity or osteoarthritis (“normal”).

FIG. 3 shows a diagrammatic representation of RNA expression profiles of Whole blood samples from individuals who were identified as having both osteoarthritis and allergies as described herein as compared with RNA expression profiles from individuals without either allergies or osteoarthritis (“normal”).

FIG. 4 shows a diagrammatic representation of RNA expression profiles of Whole blood samples from individuals having osteoarthritis and who were subject to systemic steroids as described herein as compared with RNA expression profiles from individuals not taking systemic steroids and without osteoarthritis (“normal”).

FIG. 5 shows a diagrammatic representation of RNA expression profiles of Whole blood samples from individuals having hypertension as compared with RNA expression profiles from samples of both non-hypertensive and normal individuals.

FIG. 6 shows a diagrammatic representation of RNA expression profiles of Whole blood samples from individuals who were identified as obese as described herein as compared with RNA expression profiles from normal and non-obese individuals.

FIG. 7 shows a venn diagram illustrating a summary of the analysis comparing hypertension and OA patients vs. individuals without hypertension or OA (Table 1A), hypertension and OA patients vs. OA patients (Table 1G), and the intersection between the two populations of genes (Table 1H).

FIG. 8 shows a venn diagram illustrating a summary of the analysis comparing obesity and OA patients vs. individuals without obesity or OATable 1B), obesity and OA patients vs. OA patients (Table 1I), and the intersection between the two populations of genes (Table 1J).

FIG. 9 shows a venn diagram illustrating a summary of the analysis comparing allergy and OA patients vs individuals without allergy or OA (Table 1C), allergy and OA patients vs. OA patients (Table 1K), and the intersection between the two populations of genes (Table 1L).

FIG. 10 shows a venn diagram illustrating a summary of the analysis comparing systemic steroids and OA patients vs. individuals without OA and not exposed to systemic steroids (Table 1D), systemic steroids and OA patients vs. OA patients (Table 1M), and the intersection between the two populations of genes (Table 1N).

FIG. 11 shows a diagrammatic representation of RNA expression profiles of Whole blood samples from individuals who were identified as having OA and being on of three types of systemic steroids, including hormone replacement therapy, birth control and prednisone.

FIG. 12 shows a diagrammatic representation of RNA expression profiles of Whole blood samples from individuals who were identified as having type 2 diabetes as described herein as compared with RNA expression profiles from normal and non-type 2 diabetes individuals.

FIG. 13 shows a diagrammatic representation of RNA expression profiles of Whole blood samples from individuals who were identified as having hyperlipidemia as described herein as compared with RNA expression profiles from normal and non-hyperlipidemia patients.

FIG. 14 shows a diagrammatic representation of RNA expression profiles of Whole blood samples from individuals who were identified as having lung disease as described herein as compared with RNA expression profiles from normal and non lung disease individuals.

FIG. 15 shows a diagrammatic representation of RNA expression profiles of Whole blood samples from individuals who were identified as having bladder cancer as described herein as compared with RNA expression profiles from non bladder cancer individuals.

FIG. 16 shows a diagrammatic representation of RNA expression profiles of Whole blood samples from individuals who were identified as having advanced stage bladder cancer or early stage bladder cancer as described herein as compared with RNA expression profiles from non bladder cancer individuals.

FIG. 17 shows a diagrammatic representation of RNA expression profiles of Whole blood samples from individuals who were identified as having coronary artery disease (CAD) as described herein as compared with RNA expression profiles from non-coronary artery disease individuals.

FIG. 18 shows a diagrammatic representation of RNA expression profiles of Whole blood samples from individuals who were identified as having rheumatoid arthritis as described herein as compared with RNA expression profiles from non-rheumatoid arthritis individuals.

FIG. 19 shows a diagrammatic representation of RNA expression profiles of Whole blood samples from individuals who were identified as having depression as described herein as compared with RNA expression profiles from non-depression individuals.

FIG. 20 shows a diagrammatic representation of RNA expression profiles of Whole blood samples from individuals who were identified as having various stages of osteoarthritis as described herein as compared with RNA expression profiles from individuals without osteoarthritis.

FIG. 21 shows a diagrammatic representation of RNA expression profiles of Whole blood samples from individuals who were identified as having liver cancer as described herein as compared with RNA expression profiles from individuals not having liver cancer.

FIG. 22 shows a diagrammatic representation of RNA expression profiles of Whole blood samples from individuals who were identified as having schizophrenia as described herein as compared with RNA expression profiles from individuals not having schizophrenia.

FIG. 23 shows a diagrammatic representation of RNA expression profiles of Whole blood samples from individuals who were identified as having symptomatic or asymptomatic Chagas' disease as described herein as compared with RNA expression profiles from individuals without Chagaas Disease.

FIG. 24 shows a diagrammatic representation of RNA expression profiles of Whole blood samples from individuals who were identified as having asthma and OA as compared with individuals having OA but not asthma.

FIG. 25 shows a diagrammatic representation of RNA expression profiles of Whole blood samples from individuals who were identified as having manic depression syndrome as compared with those individuals who have schizophrenia.

FIG. 26 shows a representation of the presentation of various stages of OA in patients of with respect to the age group of the patients.

FIG. 27 shows RT-PCR of overexpressed genes in CAD peripheral blood cells identified using microarray experiments, including PBP, PF4 and F13A.

FIG. 28 shows the the “Blood Chip”, a cDNA microarray slide with 10,368 PCR products derived from peripheral blood cell cDNA libraries. Colors represent hybridization to probes labeled mth Cy3 (green) or Cy5 (red). Yellow spots indicate common hybri dization between both probes. In slide A, normal blood cell RNA samples were labeled with Cy3 and CAD blood cell RNA samples were labeled with Cy5. In slide B, Cy3 and Cy5 were switched to label the RNA samples. (Cluster analysis revealed distinct gene expression profiles for normal and CAD samples.)

### DETAILED DESCRIPTION

Disclosed herein are methods that would be understood by a person skilled in the art as representing means of identifying biomarkers which correlate to one or more nucleic acid transcripts which are differentially expressed in blood, according to a condition of interest, wherein the condition of interest includes a disease, a stage of disease, as well as other non-disease conditions. Also disclosed herein is a composition comprising the biomarker(s) identified as such, the biomarker(s) themselves, as well as methods of using the biomarker(s). Such methods include using the biomarkers to diagnose an individual as having a condition of interest or a certain stage of a condition of interest, and to differentiate between two or more conditions. Products which are representative of kits useful in diagnosing an individual as having a a condition of interest are also disclosed.

In one embodiment of the invention, a blood sample is collected from one or more individuals having a condition of interest, and RNA is isolated from said blood sample. In a preferred embodiment the blood sample is whole blood without prior fractionation. In another preferred embodiment, the blood sample is peripheral blood leukocytes. In another preferred embodiment, the blood sample is peripheral blood mononuclear cells (PBMCs).

Biomarkers are identified by measuring the level of one or more species of RNA transcripts or a synthetic nucleic acid copy (cDNA, cRNA etc.) thereof, from one or more individuals who have a condition of interest or who do not have said condition of interest and/or who are healthy and normal. In one embodiment, the level of one or more species of RNA transcripts is determined by quantitating the level of an RNA species of the invention. In one embodiment for example, mass spectrometry may be used to quantify the level of one or more species of RNA transcripts (Koster et al., 1996; Fu et al., 1998). In a preferred embodiment, the level of one or more species of RNA transcripts is determined using microarray analysis. In another preferred embodiment, the level of one or more species of RNA transcripts is measured using quantitative RT-PCR. In accordance with the present invention, there may be employed other conventional molecular biology, microbiology, and recombinant DNA techniques within the skill of the art in order to quantitatively or semi-quantitatively measure one or more species of RNA transcripts. Such techniques are explained fully in the literature. See, e.g., Sambrook, Fritsch & Maniatis, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984). In a preferred embodiment quantitative RT-PCR may be used for the purpose of measuring/quantitating transcripts in blood.

In a preferred embodiment, expression levels of one or more species of RNA transcripts from a population of samples having a condition of interest are compared those levels from a population of samples not having the condition of interest so as to identify biomarkers which are able to differentiate between the two populations. In another preferred embodiment, expression levels of one or more species of RNA transcripts from a population of samples having a first condition of interest are compared with a those from a population of samples having a second condition of interest so as to identify biomarkers which can differentiate between said conditions. In another preferred embodiment, when comparing two populations of individuals to identify biomarkers of a condition of interest, the populations are chosen such that the populations share at least one phenotype which is not the condition of. More preferably the populations have two or more, three or more, four or more etc. phenotypes in common. By phenotype is meant any trait which is not the condition of interest, for example, in a preferred embodiment individuals within the populations being used to identify biomarkers of a condition are of a similar age, sex, body mass index (BMI).

The identified biomarkers can be used to determine whether an individual has a condition of interest. As would be understood to a person skilled in the art, one can utilize the biomarkers identified, or combinations of the biomarkers identified, to characterize an unknown sample in accordance with “class prediction” methods as would be understood by a person skilled in the art.

The following terms shall have the definitions set out below:

A “cDNA” is defined as copy-DNA or complementary-DNA, and is a product of a reverse transcription reaction from an mRNA transcript. “RT-PCR” refers to reverse transcription polymerase chain reaction and results in production of cDNAs that are complementary to the mRNA template(s). RT-PCR includes “QRT-PCR”, quantitative real time reverse transcription polymerase chain reaction which uses a labeling means to quantitate the level of mRNA transcription and can either be done using the one step or two step protocols for the making of cDNA and the amplification step. The labeling means can include SYBR® green intercolating dye; TaqMan® probes and Molecular Beacons® as well as others as would be understood by a person skilled in the art.

The term “oligonucleotide” is defined as a molecule comprised of two or more deoxyribonucleotides and/or ribonucleotides, preferably more than three. Its exact size will depend upon many factors which, in turn, depend upon the ultimate function and use of the oligonucleotide. The upper limit may be 15, 20, 25, 30, 40 or 50 nucleotides in length. The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 15-25 or more nucleotides, although it may contain fewer nucleotides. The factors involved in determining the appropriate length of primer are readily known to one of ordinary skill in the art.

As used herein, random sequence primers refer to a composition of primers of random sequence, i.e. not directed towards a specific sequence. These sequences possess sufficient nucleotides complementary to a polynucleotide to hybridize with said polynucleotide and the primer sequence need not reflect the exact sequence of the template.

“Restriction fragment length polymorphism” refers to variations in DNA sequence detected by variations in the length of DNA fragments generated by restriction endonuclease digestion.

A standard Northern blot assay can be used to ascertain the relative amounts of mRNA in a cell or tissue obtained from plant or other tissue, in accordance with conventional Northern hybridization techniques known to those persons of ordinary skill in the art. The Northern blot uses a hybridization probe, e.g. radiolabelled cDNA, either containing the full-length, single stranded DNA or a fragment of that DNA sequence at least 20 (preferably at least 30, more preferably at least 50, and most preferably at least 100 consecutive nucleotides in length). The DNA hybridization probe can be labeled by any of the many different methods known to those skilled in this art. The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

As defined herein, a “nucleic acid array” and “microarray” refers to a plurality of unique nucleic acids (or “nucleic acid members”) attached to a support where each of the nucleic acid members is attached to a support in a unique pre-selected region. In one embodiment, the nucleic acid probe attached to the surface of the support is DNA. In a preferred embodiment, the nucleic acid probe attached to the surface of the support is either cDNA or oligonucleotides. In another preferred embodiment, the nucleic acid probe attached to the surface of the support is cDNA synthesized by polymerase chain reaction (PCR). The term “nucleic acid”, as used herein, is interchangeable with the term “polynucleotide”. In another preferred embodiment, a “nucleic acid array” refers to a plurality of unique nucleic acids attached to nitrocellulose or other membranes used in Southern and/or Northern blotting techniques.

As used herein, “an individual” refers to a human subject as well as a non-human subject such as a mammal, an invertebrate, a vertebrate, a rat, a horse, a dog, a cat, a cow, a chicken, a bird, a mouse, a rodent, a primate, a fish, a frog and a deer. The examples herein are not meant to limit the methodology of the present invention to a human subject only, as the instant methodology is useful in the fields of veterinary medicine, animal sciences and such. The term “individual” refers to a human subject and a non-human subject who are condition free and also includes a human and a non-human subject diagnosed with one or more conditions, as defined herein. “Co-morbid individuals” or “comorbidity” or “individuals considered as co-morbid” are individuals who have more than one condition as defined herein. For example a patient diagnosed with both osteoarthritis and hypertension is considered to present with comorbidities.

As used herein, “detecting” refers to determining the presence of a one or more species of RNA transcripts, for example cDNA, RNA or EST, by any method known to those of skill in the art or taught in numerous texts and laboratory manuals (see for example, Ausubel et al. Short Protocols in Molecular Biology (1995) 3rd Ed. John Wiley & Sons, Inc.). For example, methods of detection include but are not limited to, RNA fingerprinting, Northern blotting, polymerase chain reaction, ligase chain reaction, Qbeta replicase, isothermal amplification method, strand displacement amplification, transcription based amplification systems, nuclease protection (SI nuclease or RNAse protection assays) as well as methods disclosed in WO88/10315, WO89/06700, PCT/US87/00880, PCT/US89/01025.

As used herein, a “condition” of the invention refers to a mode or state of being including a physical, emotional, psychological or pathological state. A condition can be as a result of both “genetic” and/or “environmental” factors. By “genetic factors” is meant genetically inherited factors or characteristics inherent as a result of the genetic make up of the individual. By “environmental factors” is meant those factors which are not genetically inherited, but which are the result of exposure to internal or external influences. In one embodiment of the invention, a condition is a disease as defined herein. In another embodiment of the invention, a condition is a stage of a disease as defined herein. In yet another embodiment of the invention, a condition is a mode or state of being which is not a disease. For example in one embodiment, a condition which is not a disease is a condition resulting from the progression of time. A condition resulting from progression of time can include, but is not limited to: memory loss, loss of skin elasticity, loss of muscle tone, and loss of sexual desire. In a further embodiment of the invention a condition which is not a disease is a treatment. A treatment can include, but is not limited to disease modifying treatments as well as treatments useful in mitigating the symptoms of disease. For example treatments can include drugs specific for a disease of the invention. In a preferred embodiment, treatments can include drugs specific for Alzheimer's, Cardiovascular disease, Manic Depression Syndrome, Schizophrenia, Diabetes and Osteoarthritis. For example, treatments can include but are not limited to VIOXX®, Celebrex®, NSAIDS, Cortisone, Visco supplement, Lipitor®, Adriamycin®, Cytoxan®, Herceptin®, Nolvadex® Avastin®, Erbitux®, Fluorouracil®, Largactil®, Sparine®, Vesprin®, Stelazine®, Fentazine®, Prolixin®, Compazine®, Tindal®, Modecate®, Moditen®, Mellarin, Serentil, Norvane, ®, Fluanxol®, Clopixol®, Taractan®, Depixol®, Clopixol®, Haldol®, Haldol® Decanoate, Orap®, Inapsine®, Imap®, Semap®, Loxitane®, Daxol®, lithium, anticonvulsants (for ex. carbamazepine) and antidepressants and Moban®. More generally and addition, a treatment can include any treatment or drug described in the Compendium of Pharmaceuticals and Specialties, Canadian Pharmaceutical Association; 26^{th} edition, June, 1991; Krogh, Compendium of Pharmaceuticals and Specialties, Canadian Pharmaceutical Association; 27^{th} edition, April, 1992. In a further embodiment, a condition of the invention which is not a disease is a response to environmental factors including but not limited to pollution, environmental toxins, lead poisoning, mercury posining, exposure to genetically modified organisms, exposure to radioactivity, pesticides, insecticides, and cigarette smoke, alcohol, or exercise. In a further embodiment, a condition is a state of health.

As used herein, a disease of the invention includes, but is not limited to, blood disorder, blood lipid disease, autoimmune disease, arthritis (including osteoarthritis, rheumatoid arthritis, lupus, allergies, juvenile rheumatoid arthritis and the like), bone or joint disorder, a cardiovascular disorder (including heart failure, congenital heart disease; rheumatic fever, valvular heart disease; corpulmonale, cardiomyopathy, myocarditis, pericardial disease; vascular diseases such as atherosclerosis, acute myocardial infarction, ischemic heart disease and the like), obesity, respiratory disease (including asthma, pneumonitis, pneumonia, pulmonary infections, lung disease, bronchiectasis, tuberculosis, cystic fibrosis, interstitial lung disease, chronic bronchitis emphysema, pulmonary hypertension, pulmonary thromboembolism, acute respiratory distress syndrome and the like), hyperlipidemias, endocrine disorder, immune disorder, infectious disease, muscle wasting and whole body wasting disorder, neurological disorders (including migraines, seizures, epilepsy, cerebrovascular diseases, alzheimers, dementia, Parkinson's, ataxic disorders, motor neuron diseases, cranial nerve disorders, spinal cord disorders, meningitis and the like) including neurodegenerative and/or neuropsychiatric diseases and mood disorders (including schizophrenia, anxiety, bipolar disorder; manic depression and the like, skin disorder, kidney disease, scleroderma, stroke, hereditary hemorrhage telangiectasia, diabetes, disorders associated with diabetes (e.g., PVD), hypertension, Gaucher's disease, cystic fibrosis, sickle cell anemia, liver disease, pancreatic disease, eye, ear, nose and/or throat disease, diseases affecting the reproductive organs, gastrointestinal diseases (including diseases of the colon, diseases of the spleen, appendix, gall bladder, and others) and the like. For further discussion of human diseases, see Mendelian Inheritance in Man: A Catalog of Human Genes and Genetic Disorders by Victor A. McKusick (12th Edition (3 volume set) June 1998, Johns Hopkins University Press, ISBN: 0801857422) and Harrison's Principles of Internal Medicine by Braunwald, Fauci, Kasper, Hauser, Longo, & Jameson (15th Edition 2001), the entirety of which is incorporated herein.

In another embodiment of the invention, a disease refers to an immune disorder, such as those associated with overexpression of a gene or expression of a mutant gene (e.g., autoimmune diseases, such as diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, automimmune thyroiditis, dermatitis (including atopic dermatitis and eczematous dermatitis), psoriasis, Sjogren's Syndrome, Crohn's disease, ulcerative colitis, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

In another embodiment, a disease of the invention is a cellular proliferative and/or differentiative disorder that includes, but is not limited to, cancer e.g., carcinoma, sarcoma or other metastatic disorders and the like. As used herein, the term “cancer” refers to cells having the capacity for autonomous growth, i.e., an abnormal state of condition characterized by rapidly proliferating cell growth. “Cancer” is meant to include all types of cancerous growths or oncogenic processes, metastatic tissues or malignantly transformed cells, tissues, or organs, irrespective of histopathologic type or stage of invasiveness. Examples of cancers include but are not limited to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumour, in situ, Krebs 2, Merkel cell, mucinous, non-small cell lung, oat cell, papillary, scirrhous, bronchiolar, bronchogenic, squamous cell, and transitional cell), histiocytic disorders, leukaemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumour, adeno-carcinoma, adenoma, cholangioma, cholesteatoma, cylindroma, cystadenocarcinoma, cystadenoma, granulosa cell tumour, gynandroblastoma, hepatoma, hidradenoma, islet cell tumour, Leydig cell tumour, papilloma, Sertoli cell tumour, theca cell tumour, leiomyoma, leiomyosarcoma, myoblastoma, mymoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic respiratory tract, and urogenital), neurofibromatosis, and cervical dysplasia, and other conditions in which cells have become immortalized or transformed.

“Cardiovascular Disease” is defined herein as any disease or disorder of the cardiovascular system and includes arteriosclerosis, heart valve disease, arrhythmia, and, orthostatic hypotension, shock, endocarditis, diseases of the aorta and its branches, disorders of the peripheral vascular system, and congenital heart disease as a disease affecting the heart or blood vessels. Cardiovascular diseases include coronary artery disease, hearart failure, and hypertension.

As used herein “Neurological Disease” is defined as a disorder of the nervous system, and include disorders that involve the central nervous system (brain, brainstem and cerebellum), the peripheral nervous system (including cranial nerves), and the autonomic nervous system (parts of which are located in both central and peripheral nervous system). In particular neurological disease includes alzheimers', schizophrenia, and manic depression syndrome.

As used herein, a “population” or a “population of individuals” of the invention refers to a population of two or more individuals wherein the individuals have at least a single condition of interest in common. A population of the invention can also have two or more conditions in common. A population of the invention can also be comprised of two or more individuals who do not have a condition of interest.

As used herein, “diagnosis” refers to the ability to demonstrate an increased likelihood that an individual has a specific condition or conditions. Diagnosis also refers to the ability to demonstrate an increased likelihood that an individual does not have a specific condition. More particularly “diagnosis” refers to the ability to demonstrate an increased likelihood that an individual has one condition as compared to a second condition. More particularly “diagnosis” refers to a process whereby there is an increased likelihood that an individual is properly characterized as having a condition (“true positive”) or is properly characterized as not having a condition (“true negative”) while minimizing the likelihood that the individual is improperly characterized with said condition (“false positive”) or improperly characterized as not being afflicted with said condition (“false negative”).

As used herein, “treatment” refers to the administration of a drug, pharmaceutical, nutraceutical, or other form of therapeutic regime which has the potential to reverse or ameliorate the pathology of a disease condition, produce a change in a condition as measured by either the lessening of the number or severity of symptoms or effects of the condition, as determined by a physician. In a preferred embodiment a treatment of the invention is a treatment for a disease. In another preferred embodiment, a treatment of the invention is a treatment of a disease selected from the group of: liver cancer, urinary bladder cancer, gallbladder cancer, brain cancer, prostate cancer, ovarian cancer, cervical cancer, kidney cancer, gastric cancer, colon cancer, lung cancer, breast cancer, nasopharyngeal cancer, pancreatic cancer, osteoarthritis, depression, hypertension, heart failure, obesity, rheumatoid arthritis, hyperlipidemia, lung disease, Chagas' disease, allergies, schizophrenia and asthma, manic depression syndrome, ankylosing spondylitis, guillain barre syndrome, fibromyalgia, multiple sclerosis, muscular dystrophy, septic joint arthroplasty, hepatitis, Crohn's disease or colitis, or malignant hyperthermia susceptibility, psoriasis, thyroid disorder, irritable bowel syndrome, osteoporosis, migraines, eczema, or a heart murmer.

As used herein, a “response to treatment” indicates a physiological change as a result of the “application of treatment” to a condition where “treatment” includes pharmaceuticals, neutraceuticals, and other drugs or treatment regimes. The relative success of a response to treatment is determined by a physician. As used herein, by the term “treatment regime” is meant a course of treatment ranging from a single application or dose to multiple applications of one or more doses over time.

As used herein, a “biomarker” is a molecule which corresponds to a species of a nucleic acid transcript that has a quantitatively differential concentration or level in blood with respect to an aspect of the condition of interest. As such, a biomarker includes a synthetic nucleic acid copolymer thereof, including cRNA, cDNA, and the like. A species of a nucleic acid transcript includes any nucleic acid transcript which is transcribed from any part of the individual's chromosomal and extrachromosomal genome including for example the mitochondrial genome. Preferably a species of a nucleic acid transcript is an RNA transcript, preferably the RNA transcript includes a primary transcript, a spliced transcript, an alternatively spliced transcript, or an mRNA. An aspect of the condition of interest includes the presence or absence of the condition in an individual or group of individuals for which the biomarker is identified or assayed, and also includes the stage of progression or regression of a condition including a disease condition. For example, a biomarker is a molecule which corresponds to a species of an RNA transcript which is present at an increased level s or a decreased level of in the blood of an individual or a population of individuals having at least one condition of interest, when compared to the level of said transcript in the blood from a population of individuals not having said condition of interest. Molecules encompassed by the term biomarker include ESTs, cDNAs, primers, etc. A biomarker can be used either solely or in conjunction with one or more other identified biomarkers, so as to allow diagnosis of a condition of interest as defined herein.

As used herein, the term “concentration or level” of a species of an RNA transcript refers to the measurable quantity of a given biomarker. The ““concentration or level”” of a species of an RNA transcript can be determined by measuring the level of RNA using semi-quantitative methods such as microarray hybridization or more quantitative measurements such as quantitative real-time RT-PCR which corresponds in direct proportion with the extent to which the gene is expressed. The “concentration or level” of a species of an RNA transcript is determined by methods well known in the art. As used herein the term “differential expression” refers to a difference in the level of expression of a species of an RNA nucleic acid transcript, as measured by the amount or level of RNA or can also include a measurement of the protein encoded by the gene corresponding to the nucleic acid transcript, in a sample or population of samples as compared with the amount or level of RNA or protein expression of the same nucleic acid transcript in a second sample or second population of samples. The term “differentially expressed” or “changes in the level of expression” refers to an increase or decrease in the measurable expression level of a given biomarker in a sample as compared with the measurable expression level of a given biomarkerin a second sample. The term “differentially expressed” or “changes in the level of expression” can also refer to an increase or decrease in the measurable expression level of a given biomarkerin a population of samples as compared with the measurable expression level of a biomarkerin a second population of samples. As used herein, “differentially expressed” when referring to a single sample can be measured using the ratio of the level of expression of a given nbiomarker in said sample as compared with the mean expression level of the given biomarker of a control population wherein the ratio is not equal to 1.0. Differentially expressed can also be used to include comparing a first population of samples as compared with a second population of samples or a single sample to a population of samples using either a ratio of the level of expression or using p-value. When using p-value, a nucleic acid transcript is identified as being differentially expressed as between a first and second population when the p-value is less than 0.1. More preferably the p-value is less than 0.05. Even more preferably the p-value is less than 0.01. More preferably still the p-value is less than 0.005. Most preferably the p-value is less than 0.001. When determining whether a nucleic acid transcript is differentially expressed on the basis of the ratio of the level of expression, a nucleic acid transcript is differentially expressed if the ratio of the level of expression of a nucleic acid transcript in a first sample as compared with a second sample is greater than or less than 1.0. For example, a ratio of greater than 1.2, 1.5, 1.7, 2, 3, 4, 10, 20 or a ratio less than 1, for example 0.8, 0.6, 0.4, 0.2, 0.1, 0.05. In another embodiment of the invention a nucleic acid transcript is differentially expressed if the ratio of the mean of the level of expression of a first population as compared with the mean level of expression of the second population is greater than or less than 1.0 For example, a ratio of greater than 1.2, 1.5, 1.7, 2, 3, 4, 10, 20 or a ratio less than 1, for example 0.8, 0.6, 0.4, 0.2, 0.1, 0.05 In another embodiment of the invention a nucleic acid transcript is differentially expressed if the ratio of its level of expression in a first sample as compared with the mean of the second population is greater than or less than 1.0 and includes for example, a ratio of greater than 1.2, 1.5, 1.7, 2, 3, 4, 10, 20, or a ratio less than 1, for example 0.8, 0.6, 0.4, 0.2, 0.1, 0.05. “ Differentially increased expression” refers to 1.1 fold, 1.2 fold, 1.4 fold, 1.6 fold, 1.8 fold, or more, relative to a standard, such as the mean of the expresion level of the second population. “Differentially decreased expression” refers to less than 1.0 fold, 0.8 fold, 0.6 fold, 0.4 fold, 0.2 fold, 0.1 fold or less, relative to a standard, such as the mean of the expresion level of the second population.

A nucleic acid transcript is also said to be differentially expressed in two samples if one of the two samples contains no detectable expression of the nucleic acid transcript. Absolute quantification of the level of expression of a nucleic acid transcript can be accomplished by including known concentration(s) of one or more control nucleic acid transcript, generating a standard curve based on the amount of the control s nucleic acid transcript and extrapolating the expression level of the “unknown” nucleic acid transcript, for example, from the real-time RT PCR hybridization intensities of the unknown with respect to the standard curve.

By a nucleic acid transcript that is “expressed in blood” is meant a nucleic acid transcript that is expressed in one or more cells of blood, wherein the cells of blood include monocytes, leukocytes, lymphocytes, erythrocytes, all other cells derived directly from haemopoietic or mesenchymal stem cells, or cells derived directly from a cell which typically makes up the blood.

The term “biomarker” further includes any molecule that correlates to, or is reflective of the transcript produced from any region of nucleic acid that can be transcribed, as the invention contemplates detection of RNA or equivalents thereof, i.e., cDNA or EST. A biomarker of the invention includes but is not limited to regions which are translated into proteins which are specific for or involved in a particular biological process, such as apoptosis, differentiation, stress response, aging, proliferation, etc.; cellular mechanism genes, e.g. cell-cycle, signal transduction, metabolism of toxic compounds, and the like; disease associated genes, e.g. genes involved in cancer, schizophrenia, diabetes, high blood pressure, atherosclerosis, viral-host interaction, infection and the like. A biomarker of the the invention includes, but is not limited to transcripts transcribed from immune response genes. A gene of the invention is a biomarker of a condition and can be a biomarker of disease, or a biomarker of a non disease condition as defined herein.

For example, a biomolecule can be reflective of or correlate to the transcript from any gene, including an oncogene (Hanahan, D. and R. A. Weinberg, Cell (2000) 100:57; and Yokota, J., Carcinogenesis (2000) 21(3):497-503) whose expression within a cell induces that cell to become converted from a normal cell into a tumor cell. Examples of genes which produce transcript(s) to which a biomarker is correlated to or reflective of, include, but are not limited to, include cytokine genes (Rubinstein, M., et al., Cytokine Growth Factor Rev. (1998) 9(2):175-81); idiotype (Id) protein genes (Benezra, R., et al., Oncogene (2001) 20(58):8334-41; Norton, J. D., J. Cell Sci. (2000) 113(22):3897-905); prion genes (Prusiner, S. B., et al., Cell (1998) 93(3):337-48; Safar, J., and S. B. Prusiner, Prog. Brain Res. (1998) 117:421-34); genes that express molecules that induce angiogenesis (Gould, V. E. and B. M. Wagner, Hum. Pathol. (2002) 33(11):1061-3); genes encoding adhesion molecules (Chothia, C. and E. Y. Jones, Annu. Rev. Biochem. (1997) 66:823-62; Parise, L. V., et al., Semin. Cancer Biol. (2000) 10(6):407-14); genes encoding cell surface receptors (Deller, M. C., and Y. E. Jones, Curr. Opin. Struct. Biol. (2000) 10(2):213-9); genes of proteins that are involved in metastasizing and/or invasive processes (Boyd, D., Cancer Metastasis Rev. (1996) 15(1):77-89; Yokota, J., Carcinogenesis (2000) 21(3):497-503); genes of proteases as well as of molecules that regulate apoptosis and the cell cycle (Matrisian, L. M., Curr. Biol. (1999) 9(20):R776-8; Krepela, E., Neoplasma (2001) 48(5):33249; Basbaum and Werb, Curr. Opin. Cell Biol. (1996) 8:731-738; Birkedal-Hansen, et al., Crit. Rev. Oral Biol. Med. (1993) 4:197-250; Mignatti and Rifkin, Physiol. Rev. (1993) 73:161-195; Stetler-Stevenson, et al., Annu. Rev. Cell Biol. (1993) 9:541-573; Brinkerhoff, E., and L. M. Matrisan, Nature Reviews (2002) 3:207-214; Strasser, A., et al., Annu. Rev. Biochem. (2000) 69:217-45; Chao, D. T. and S. J. Korsmeyer, Annu. Rev. Immunol. (1998) 16:395-419; Mullauer, L., et al., Mutat. Res. (2001) 488(3):211-31; Fotedar, R., et al., Prog. Cell Cycle Res. (1996) 2:147-63; Reed, J. C., Am. J. Pathol. (2000) 157(5):1415-30; D'Ari, R., Bioassays (2001) 23(7):563-5); or multi-drug resistance genes, suach as MDR1 gene (Childs, S., and V. Ling, Imp. Adv. Oncol. (1994) 21-36). In another embodiment, a gene which produces transcript(s) to which a biomarker is correlated to or reflective of, include, but are not limited to, an immune response gene or a non-immune response gene. By an immune response gene is meant a primary defense response gene located outside the major histocompatibility region (MHC) that is initially triggered in response to a foreign antigen to regulate immune responsiveness. All other genes expressed in blood are considered to be non-immune response genes. For example, an immune response gene would be understood by a person skilled in the art to include: cytokines including interleukins and interferons such as TNF-alpha, IL-10, IL-12, IL-2, IL-4, IL-10, IL-12, IL-13, TGF-Beta, IFN-gamma; immunoglobulins, complement and the like (see for example Bellardelli, F. Role of interferons and other cytokines in the regulation of the immune response APMIS. 1995 March; 103(3): 161-79;);.

Construction of a Nucleic Acid Array

A nucleic acid microarray (RNA, DNA, cDNA, PCR products or ESTs) according to the invention can be constructed as follows:

Nucleic acids (RNA, DNA, cDNA, PCR products or ESTs) (˜40 μl) are precipitated with 4 μl ( 1/10 volume) of 3M sodium acetate (pH 5.2) and 100 ul (2.5 volumes) of ethanol and stored overnight at −20° C. They are then centrifuged at 3,300 rpm at 4° C. for 1 hour. The obtained pellets are washed with 50 μl ice-cold 70% ethanol and centrifuged again for 30 minutes. The pellets are then air-dried and resuspended well in 50% dimethylsulfoxide (DMSO) or 20 μl 3×SSC overnight. The samples are then deposited either singly or in duplicate onto Gamma Amino Propyl Silane (Corning CMT-GAPS or CMT-GAP2, Catalog No. 40003, 40004) or polylysine-coated slides (Sigma Cat. No. P0425) using a robotic GMS 417 or 427 arrayer (Affymetrix, CA). The boundaries of the DNA spots on the microarray are marked with a diamond scriber. The invention provides for arrays where 10-20,000 different DNAs are spotted onto a solid support to prepare an array, and also may include duplicate or triplicate DNAs.

The arrays are rehydrated by suspending the slides over a dish of warm particle free ddH2O for approximately one minute (the spots will swell slightly but not run into each other) and snap-dried on a 70-80° C. inverted heating block for 3 seconds. DNA is then UV crosslinked to the slide (Stratagene, Stratalinker, 65 mJ—set display to “650” which is 650×100 μJ) or baked at 80° C. for two to four hours. The arrays are placed in a slide rack. An empty slide chamber is prepared and filled with the following solution: 3.0 grams of succinic anhydride (Aldrich) is dissolved in 189 ml of 1-methyl-2-pyrrolidinone (rapid addition of reagent is crucial); immediately after the last flake of succinic anhydride dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are then stored in the slide box at room temperature until use.

Nucleic Acid Arrays

A nucleic acid array comprises any combination of the nucleic acid sequences generated from, or complementary to nucleic acid transcripts, or regions thereof, including the species of nucleic acid transcripts present in blood. Preferably, for identifying biomarkers of a disease or condition of interest, one utilizes a microarray so as to minimize cost and time of the experiment. In one embodiment, the microarray is an EST microarray which includes ESTs complementary to genes expressed in blood. A microarray according to the invention preferably comprises between 10, 100, 500, 1000, 5000, 10,000 and 15,000 nucleic acid members, and more preferably comprises at least 5000 nucleic acid members. The nucleic acid members are known or novel nucleic acid sequences described herein, or any combination thereof. A microarray according to the invention is used to assay for differential levels of species of transcripts RNA expression profiles present in blood samples from healthy patients as compared to patients with a disease.

Microarrays

Microarrays include those arrays which encompass transcripts which are expressed in the individual. In one embodiment, a microarray which encompasses transcripts which are expressed in humans. In a preferred embodiment microarrays of the invention can be either cDNA based arrays or oligonucleotide based arrays.

Oligonucleotide Arrays

In a preferred embodiment, the oligonucleotide based microarrays of Affymetrix® are utilized. More particularly the Affymetrix® Human Genome U133 (HG-U133) Set, consisting of two GeneChip® arrays, contains almost 45,000 probe sets representing more than 39,000 transcripts derived from approximately 33,000 well-substantiated human genes. This set design uses sequences selected from GenBank®, dbEST, and RefSeq. More recently Affymetrix® has available the U133 Plus 2.0 GeneChip® which represents over 47,000 transcripts. It is expected as more genes and transcripts are identified as a result of the human genome sequencing project, additional generations of microarrays will be developed.

The sequence clusters were created from the UniGene database (Build 133, Apr. 20, 2001). They were then refined by analysis and comparison with a number of other publicly available databases including the Washington University EST trace repository and the University of California, Santa Cruz Golden Path human genome database (April 2001 release).

The HG-U133A Array includes representation of the RefSeq database sequences and probe sets related to sequences previously represented on the Human Genome U95Av2 Array. The HG-U133B Array contains primarily probe sets representing EST clusters.

The U133 Plus 2.0 Array includes all probe sets represented on the GeneChip Human Genome U133 Set (U133A and U133B). The U133 Plus 2.0 includes an additional 6,500 genes for analysis of over 47,000 transcripts.

cDNA Based Arrays

15 K ChondroChip™—The ChondroChip™ is an EST based microarray and includes approximately 15,000 ESTs complementy to genes also expressed in human chondrocytes. Various versions of the 15K ChondroChip™ were used, depending upon the experiment in an effort to utilize a microarray which reduced redundancy so as to increase the percentage of unique genes and thus encompass representation of as much of the entire genome as possible.

Controls on the ChondroChip™—There are two types of controls used on microarrays. First, positive controls are genes whose expression level is invariant between different stages of investigation and are used to monitor:

a) target DNA binding to the slide,
b) quality of the spotting and binding processes of the target DNA onto the slide,
c) quality of the RNA samples, and
d) efficiency of the reverse transcription and fluorescent labeling of the probes.

Second, negative controls are external controls derived from an organism unrelated to and therefore unlikely to cross-hybridize with the sample of interest. These are used to monitor for:
a) variation in background fluorescence on the slide, and
b) non-specific hybridization.

There are currently 63 control spots on the ChondroChip™ consisting of:

| Type | No. |
|---|---|
| Positive Controls: | 2 |
| Alien DNA | 12 |
| A. thaliana DNA | 10 |
| Spotting Buffer | 41 |

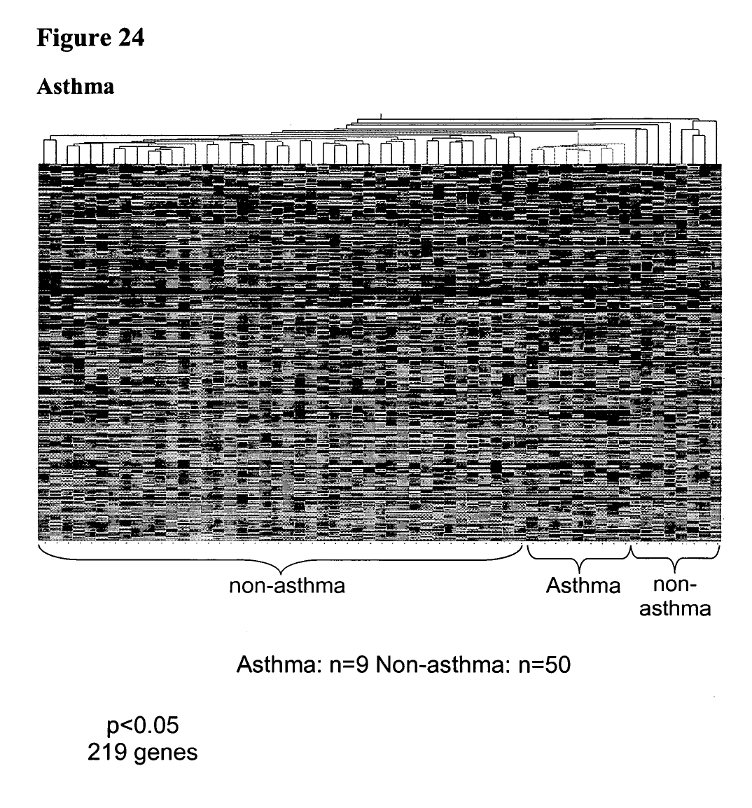

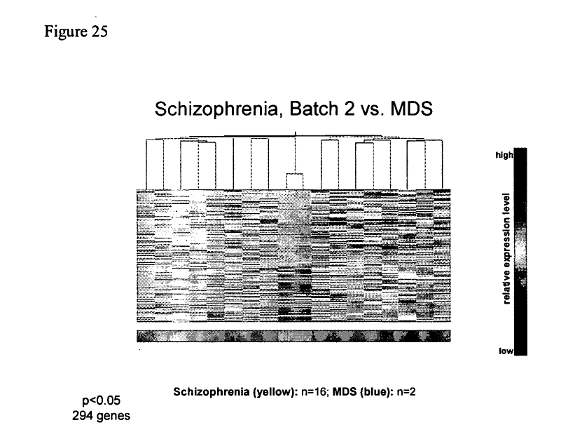

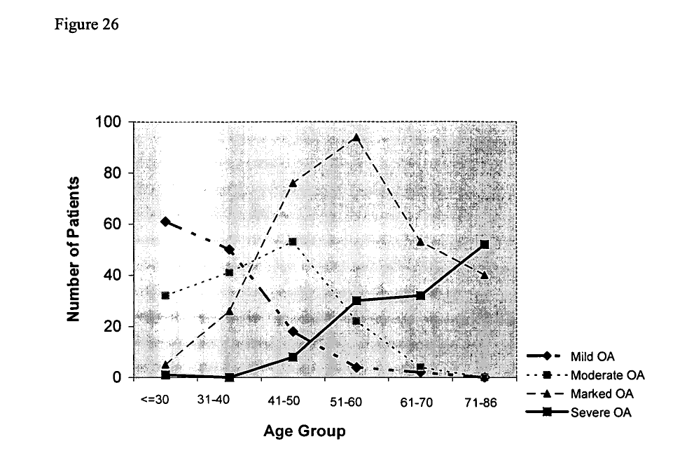

BloodChip™—The “BloodChip™” can also be used. The BloodChip is a cDNA microarray slide with 10,368 PCR products derived from peripheral blood cell cDNA libraries as shown in FIG. 24.
30K BodyChip™—The BodyChip™ is an EST based microarray which incorporates the unique cDNA clones from both the BloodChip™ and the ChondroChip™. The BodyChip™ includes coverage of over 30,000 genes.
Identifying Biomarkers Useful in Accordance with the Invention
Collection of Blood

Blood is drawn according to the methods of standard phlebotomy. A blood sample useful according to the invention is a blood sample ranging in volume from as little as a drop of blood to 100 ml, more preferably a blood sample is 10 ml to 60 ml, even more preferably a blood sample is between 25 ml to 40 ml. A blood sample that is useful according to the invention is in an amount that is sufficient for the detection of one or more genes according to the invention.

In one embodiment, 30 mls of blood is isolated and stored on ice within a K₃/EDTA tube. In another embodiment, one can utilize tubes for storing blood which contain stabilizing agents such as disclosed in U.S. Pat. No. 6,617,170. In another embodiment the PAXgene™ blood RNA system:provided by PreAnalytiX, a Qiagen/BD company may be used to collect blood. The PAXgene™ system is standardized on convenient BD Vacutainer™ technology. In yet another embodiment, the Tempus™ blood RNA collection tubes, offered by Applied Biosystems may be used. Tempus™ collection tubes provide a closed evacuated plastic tube containing RNA stabilizing reagent for whole blood collection, processing and subsequently RNA isolation.

In a preferred embodiment, RNA is isolated from said blood sample stored on ice within 24 hours, more preferably within 10 hours, even more preferably within 6 hours of collection most preferably immediately after drawing said blood. In another preferred embodiment, wherein stabilizers are utilized, such as with the PAXgene™ system, RNA is isolated from said blood sample can be isolated after storage at room temperature for 24 days, or isolated from a blood sample stored at 4° C. for a number of weeks.

Isolation and Preparation of RNA

Blood Samples

In another aspect of the invention, a blood sample, as used herein, refers to a sample of whole blood without prior fractionation, a sample of subsets of blood cells, and a sample of specific types of blood cells. Accordingly, a blood sample includes, but is not limited to, whole blood without prior fractionation, peripheral blood leukocytes (PBL's), granulocytes, agranulocytes, T lymphocytes, B lymphocytes, monocytes, macrophages, eosinophils, neutrophils, basophils, erythrocytes, and platelets separated from whole blood.

Whole Blood

In one embodiment, a blood sample of the invention is whole blood without prior fractionation. By whole blood is meant blood which is unfractionated. Whole blood includes a drop of blood, a pinprick of blood. Whole blood also includes blood in which the serum or plasma is removed. Whole blood without prior fractionation can be used directly, or one can remove the serum or plasma and isolate RNA or mRNA from the remaining blood sample in accordance with methods well known in the art. The use of whole blood without fractionation is preferred since it avoids the costly and time-consuming need to separate out the cell types within the blood (Kimoto Kimoto Y (1998) Mol. Gen. Genet 258:233-239; Chelly J et al. (1989). Proc. Nat. Acad. Sci. USA. 86:2617-2621; Chelly J et al. (1988). Nature 333:858-860). In a preferred embodiment, the whole blood sample can have the plasma or serum removed by centrifugation, using preferably gentle centrifugation at 300-800×g for five to ten minutes._In another preferred embodiment, lysis buffer is added to the whole blood sample without prior fractionation, prior to extraction of RNA. Lysis Buffer (1 L) 0.6 g EDTA; 1.0 g KHCO₂, 8.2 g NH₄Cl adjusted to pH 7.4 (using NaOH). Once mixed with lysing buffer, the sample can be centrifuged and the cell pellet containing the RNA or mRNA extracted in accordance with methods known in the art (see for example Sambrook et al.)

Peripheral Blood Leukocytes (PBLs)

In another embodiment, a blood sample of the invention is a sample of peripheral blood leukocytes (PBLs). Whole blood without prior fractionation is obtained from a normal patient or from an individual diagnosed with, or suspected of having a disease or condition, according to methods of phlebotomy well known in the art. PBLs are separated from the remainder of the blood using methods known in the art. For example, PBLs can be separated using a Ficoll® gradient.

In another embodiment, a blood sample of the invention is a sample of granulocytes. In another embodiment, a blood sample of the invention is a sample of neutrophils, eosinophils, basophils or any combination thereof. In another embodiment, a blood sample of the invention is a sample of agranulocytes. In another embodiment, a blood sample of the invention is a sample of lympocytes, monocytes or a combination thereof. In yet another embodiment, a blood sample of the invention is a sample of T lymphocytes, B lymphocytes or a combination thereof

In one aspect, a whole blood sample without prior fractionation is obtained from a normal patient or from an individual diagnosed with, or suspected of having, a disease or condition according to methods of phlebotomy well known in the art. A whole blood sample without prior fractionation that is useful according to the invention is in an amount that is sufficient for the detection of one or more nucleic acid sequences according to the invention. In a preferred embodiment, a whole blood sample without prior fractionation is in an amount ranging from 1 μl to 100 ml, more preferably 10 μl to 50 ml, even more preferably 10 μl to 25 ml and most preferably 10 μl to 1 ml.

Quantitation of RNA using Microarray Analysis

In one embodiment of the invention, the expression levels of transcripts from individuals or populations of individuals having a condition, or not having a condition are measured using an array. In a preferred embodiment either a cDNA based microarray or an oligonucleotide based microarray are used, for example, the ChondroChip™ or the Affymetrix GeneChip® U133A, U133B or U133 Plus version are utilized.

Microarray hybridization experiments utilizing the Affymetrix® GeneChip® platforms (U133A and U133 Plus 2.0) microarray's are preferably performed in accordance with the Affymetrix® instructions.

Microarray hybridization experiments utilizing the ChondroChip™ are preferably performed as described below.

Preparation of Fluorescent DNA Probe from mRNA

Fluorescently labeled target nucleic acid samples are prepared for analysis with an array of the invention.

In one embodiment of the invention, labeled cDNA is prepared for hybridization to the ChondroChip™ microarray using 2 μg Oligo-dT primers annealed to 2 μg of mRNA isolated from a blood sample of a patient in a total volume of 15 μl, by heating to 70° C. for 10 min, and cooled on ice.

In another embodiment of the invention, 20 ug of total RNA can be utilized for preparation of labeled cDNA for purposes of hybridization.

In another embodiment of the invention, RNA can be amplified (aRNA) from either total RNA or mRNA. In a preferred embodiment aRNA is made from total RNA. Total RNA is extracted with TRIzol as stated previously. 0.1˜0.5 ug total RNA from each sample is then subjected to RNA amplification using RNA Amplification Kit (Arcturus, Catalog #KIT0201) following the user guide. 2.5 ug amplified RNA was then used for probe labeling by reverse transcription with 1 mM Cy3 or Cy5 (Pharmacia). The protocol used for hybridization was based on that described previously (H. Zhang 2002).

The mRNA is reverse transcribed by incubating the sample at 42° C. for 1.5-2 hours in a 100 μl volume containing a final concentration of 50 mM Tris-HCl (pH 8.3), 75 mM KCl, 3 mM MgCl₂, 25 mM DTT, 25 mM unlabeled dNTPs, 400 units of Superscript II (200 U/μL, Gibco BRL), and 15 mM of Cy3 or Cy5 (Amersham). RNA is then degraded by addition of 15 μl of 0.1N NaOH, and incubation at 70° C. for 10 min. The reaction mixture is neutralized by addition of 15 μl of 0.1N HCl, and the volume is brought to 500 μl with TE (10 mM Tris, 1 mM EDTA), and 20 μg of Cot1 human DNA (Gibco-BRL) is added.

The labeled target nucleic acid sample is purified by centrifugation in a Centricon-30 micro-concentrator (Amicon). If two different target nucleic acid samples (e.g., two samples derived from a healthy patient vs. patient with a disease) are being analyzed and compared by hybridization to the same array, each target nucleic acid sample is labeled with a different fluorescent label (e.g., Cy3 and Cy5) and separately concentrated. The separately concentrated target nucleic acid samples (Cy3 and Cy5 labeled) are combined into a fresh centricon, washed with 500 μl TE, and concentrated again to a volume of less than 7 μl. 1 μL of 10 μg/μl polyA RNA (Sigma, #P9403) and 1 μl of 10 μg/μl tRNA (Gibco-BRL, #15401-011) is added and the volume is adjusted to 9.5 μl with distilled water. For final target nucleic acid preparation 2.1 μl 20×SSC (1.5M NaCl, 150 mM NaCitrate (pH8.0)) and 0.35 μl 10% SDS is added.

Hybridization

Labeled nucleic acid is denatured by heating for 2 min at 100° C., and incubated at 37° C. for 20-30 min before being placed on a nucleic acid array under a 22 mm×22 mm glass cover slip. Hybridization is carried out at 65° C. for 14 to 18 hours in a custom slide chamber with humidity maintained by a small reservoir of 3×SSC. The array is washed by submersion and agitation for 2-5 min in 2×SSC with 0.1% SDS, followed by 1×SSC, and 0.1×SSC. Finally, the array is dried by centrifugation for 2 m inn a slide rack in a Beckman GS-6 tabletop centrifuge in Microplus carriers at 650 RPM for 2 min.

Signal Detection and Data Generation

Following hybridization of an array with one or more labeled target nucleic acid samples, arrays are scanned immediately using a GMS Scanner 418 and Scanalyzer software (Michael Eisen, Stanford University), followed by GeneSpring™ software (Silicon Genetics, CA) analysis. Alternatively, a GMS Scanner 428 and Jaguar software may be used followed by GeneSpring™ software analysis.

If one target nucleic acid sample is analyzed, the sample is labeled with one fluorescent dye (e.g., Cy3 or Cy5).

After hybridization to a microarray as described herein, fluorescence intensities at the associated nucleic acid members on the microarray are determined from images taken with a custom confocal microscope equipped with laser excitation sources and interference filters appropriate for the Cy3 or Cy5 fluorescence.

The presence of Cy3 or Cy5 fluorescent dye on the microarray indicates hybridization of a target nucleic acid and a specific nucleic acid member on the microarray. The intensity of Cy3 or Cy5 fluorescence represents the amount of target nucleic acid which is hybridized to the nucleic acid member on the microarray, and is indicative of the expression level of the specific nucleic acid member sequence in the target sample.

After hybridization, fluorescence intensities at the associated nucleic acid members on the microarray are determined from images taken with a custom confocal microscope equipped with laser excitation sources and interference filters appropriate for the Cy3 and Cy5 fluors. Separate scans are taken for each fluor at a resolution of 225 μm2 per pixel and 65,536 gray levels. Normalization between the images is used to adjust for the different efficiencies in labeling and detection with the two different fluors. This is achieved by manual matching of the detection sensitivities to bring a set of internal control genes to nearly equal intensity followed by computational calculation of the residual scalar required for optimal intensity matching for this set of genes.

The presence of Cy3 or Cy5 fluorescent dye on the microarray indicates hybridization of a target nucleic acid and a specific nucleic acid member on the microarray. The intensities of Cy3 or Cy5 fluorescence represent the amount of target nucleic acid which is hybridized to the nucleic acid member on the microarray, and is indicative of the expression level of the specific nucleic acid member sequence in the target sample. If a nucleic acid member on the array shows no color, it indicates that the element is not expressed in sufficient levels to be detected in either sample. If a nucleic acid member on the array shows a single color, it indicates that a labeled gene is expressed only in that cell sample. The appearance of both colors indicates that the gene is expressed in both tissue samples. The ratios of Cy3 and Cy5 fluorescence intensities, after normalization, are indicative of differences of expression levels of the associated nucleic acid member sequence in the two samples for comparison. A ratio of expression not equal to 1.0 is used as an indication of differential gene expression.

The array is scanned in the Cy 3 and Cy5 channels and stored as separate 16-bit TIFF images. The images are incorporated and analyzed using Scanalyzer™ software which includes a gridding process to capture the hybridization intensity data from each spot on the array. The fluorescence intensity and background-subtracted hybridization intensity of each spot is collected and a ratio of measured mean intensities of Cy5 to Cy3 is calculated. A linear regression approach is used for normalization and assumes that a scatter plot of the measured Cy5 versus Cy3 intensities should have a slope of one. The average of the ratios is calculated and used to rescale the data and adjust the slope to one. A post-normalization cutoff of a ratio not equal to 1.0—is used to identify differentially expressed genes.

Annotation to Identify those RNA Transcripts which are Differentially Expressed in Blood

In one aspect of the invention, Affymetrix® GeneChip® platforms (U133A and U133 Plus 2.0) microarrays are used. As would be understood by a person skilled in the art, each “gene ID” on an Affymetrix® microarray represents a number of oligonucleotide probe pairs corresponding to a region of transcribed rna, each probe pair consists of a matched and a mismatched oligonucleotide, wherein the matched oligonucleotide is 100% complementary to a RNA which is transcribed in humans. The mismatched oligonucleotide is less than 100% complementary to a region of a gene or a region of RNA which is transcribed in humans. Microarrays of the invention useful for identifying biomarkers for human conditions include the U95 array, the U133A array, the U133B array or the U133 plus 2.0 array. As would be understood by a person skilled in the art, the term “gene ID” can also be termed “spot number” or “spot ID” or “probe set ID”. An example of a gene ID used by Affymetrix® is 160020_at; 1494_f_at,; or 200003_s_at.

Gene ID's are annotated by Affymetrix and the results of the annotation are available on the Affymetrix website at www.affymetrix.com. As used herein “annotation” when used in the context of the Affymetrix® microarray is the information which allows one to identify the expressed RNA and, if applicable, the resulting protein translated by the expressed RNA which is being measured as a result of the binding of RNA to the probe pairs of the microarray. The annotation master table for the Affymetrix human microarrays is disclosed in Table 8A. Details as to the annotation provided in Table 8A are shown below in Table 9.

TABLE 9

| Affymetrix | 15kChondroChip | |
|---|---|---|
| Probe Set ID | CloneID | Affymetrix ID for the probe or |
| | | ChondroGene's cDNA clone |
| | | ID |
| Target Description | Target | Description of the represented |
| | Description | gene |
| Representative | Accession | Genbank (or internal in the |
| Public ID | | case of some Affy IDs) |
| | | database identifier(s) for the |
| | | represented gene |
| Overlapping | | Details of overlapping |
| Transcripts | | transcripts found in a |
| | | chromosomal region that |
| | | aligns with a target sequence. |
| | Aliases | Gene name synonyms. |
| Gene Title | Gene Title | Name of represented gene. |
| Gene Symbol | Gene Symbol | Official symbol of represented |
| | | gene. |
| UniGene ID | UniGene ID | The identifier for the UniGene |
| | | cluster to which the |
| | | represented gene belongs. |
| Ensembl | | Ensembl database identifier |
| | | for the represented gene. |
| LocusLink | LocusLink | LocusLink database |
| | | identifier(s) for the |
| | | represented gene. |
| SwissProt | | SwissProt database |
| | | identifier(s) for the |
| | | represented gene. |
| RefSeq Protein ID | RefSeq | Protein Reference Sequence |
| | Protein ID | database identifier(s) for the |
| | | represented gene. |
| RefSeq | | Transcript Reference |
| Transcript ID | | Sequence database |
| | | identifier(s) for the |
| | | represented gene. |

In another aspect of the invention, cDNA based arrays such as the ChondroChip™ are used. Sequences corresponding to EST sequences are spotted onto the microarray. Sequences used include those previously identified using cartilage tissue library clones as outlined in H. Zhang et al. Osteoarthritis and Cartilage (2002) 10, 950-960. The differentially expressed EST sequences of the microarrays of the invention are annotated by searching against available databases, including the “nt”, “nr”, “est”, “gss” and “htg” data bases available through NCBI to determine putative identities for ESTs matching to known genes or other ESTs. Functional characterisation of ESTs with known gene matches are made according to any known method. Preferably, differentially expressed EST sequences are compared to the non-redundant Genbank/EMBL/DDBJ and dbEST databases using the BLAST algorithm (Altschul S F, Gish W, Miller W, Myers E W, Lipman D J. Basic local alignment search tool. J Mol Biol 1990;215:403-10). A minimum value of P=10⁻¹⁰ and nucleotide sequence identity >95%, where the sequence identity is non-contiguous or scattered, are required for assignments of putative identities for ESTs matching to known genes or to other ESTs. Construction of a non-redundant list of genes represented in the EST set is done with the help of Unigene, Entrez and PubMed at the National Center for Biotechnology Information (NCBI) web site at www.ncbi.nlm.nih.gov.

Genes are identified from ESTs according to known methods. To identify novel genes from an EST sequence, the EST should preferably be at least 100 nucleotides in length, and more preferably 150 nucleotides in length, for annotation. Preferably, the EST exhibits open reading frame characteristics (i.e., can encode a putative polypeptide).

Having identified an EST corresponding to a larger sequence, other portions of the larger sequence which comprises the EST can be used in assays to elucidate gene function, e.g., to isolate polypeptides encoded by the gene, to generate antibodies specifically reactive with these polypeptides, to identify binding partners of the polypeptides (receptors, ligands, agonists, antagonists and the like) and/or to detect the expression of the gene (or lack thereof) in healthy or diseased individuals.

In another aspect, the invention provides for nucleic acid sequences that do not demonstrate a “significant match” to any of the publicly known sequences in sequence databases at the time a query is done. Longer genomic segments comprising these types of novel EST sequences can be identified by probing genomic libraries, while longer expressed sequences can be identified in cDNA libraries and/or by performing polymerase extension reactions (e.g., RACE) using EST sequences to derive primer sequences as is known in the art. Longer fragments can be mapped to particular chromosomes by FISH and other techniques and their sequences compared to known sequences in genomic and/or expressed sequence databases.

The amino acid sequences encoded by the ESTs can also be used to search databases, such as GenBank, SWISS-PROT, EMBL database, PIR protein database, Vecbase, or GenPept for the amino acid sequences of the corresponding full-length genes according to procedures well known in the art.

Alternative methods for analysing ESTs are also available. For example, the ESTs may be assembled into contigs with sequence alignment, editing, and assembly programs such as PHRED and PHRAP (Ewing, et al., 1998, Genome Res. 3:175, incorporated herein; and the web site at bozeman.genome.washington.edu). Contig redundancy is reduced by clustering nonoverlapping sequence contigs using the EST clone identification number, which is common for the nonoverlapping 5 and 3 sequence reads for a single EST cDNA clone. In one aspect, the consensus sequence from each cluster is compared to the non-redundant Genbank/EMBL/DDBJ and dbEST databases using the BLAST algorithm with the help of unigene, Entrez and PubMed at the NCBI site.

EST clones used to spot onto the ChondroChip™ have been annotated using the methods described above. Results are reported by clone name and the annotation disclosed in the ChondroChip™ Master Annotation Table 8B. As used herein “annotation” when used in the context of the ChondroChip™ allows one to identify the expressed RNA and, if applicable, the resulting protein translated by the expressed RNA which is being measured as a result of the binding of RNA to ChondroChip™ microarray. The details of the annotation shown in Table 9 above.

Measure of Level of Species of Transcripts in Blood Using Quantitative Real Time RT-PCR

In another aspect of the invention, the level of one or more species of transcripts of the invention can be determined using quantitative methods including QRT-PCR, RNA from blood (either whole blood without prior fractionation, peripheral blood leukocytes, PBMCs or another subfraction of blood) using quantitative reverse transcription (RT) in combination with the polymerase chain reaction (PCR).

Total RNA, or mRNA from blood is used as a template and a primer specific to the transcribed portion of a gene of the invention is used to initiate reverse transcription. Primer design can be accomplished utilizing commercially available software (e.g. Primer Designer 1.0, Scientific Sofware etc.). The product of the reverse transcription is subsequently used as a template for PCR.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts.

The method of PCR is well known in the art. PCR, is performed as described in Mullis and Faloona, 1987, Methods Enzymol., 155: 335, herein incorporated by reference.

PCR is performed using template DNA or cDNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10□ PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

QRT-PCR which is quantitative in nature can also be performed, using either reverse transcription and PCR in a two step procedure, or reverse transcription combined with PCR in a single step protocol so as to provide a quantitative measure of the level of one or more species of RNA transcripts in blood. One of these techniques, for which there are commercially available kits such as Taqman® (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman® system has the additional advantage of not requiring gel electrophoresis and allows for quantification when used with a standard curve.

A second technique useful for detecting PCR products quantitatively without is to use an intercolating dye such as the commercially available QuantiTect™ SYBR® Green PCR (Qiagen, Valencia Calif.). RT-PCR is performed using SYBR® green as a fluorescent label which is incorporated into the PCR product during the PCR stage and produces a flourescense proportional to the amount of PCR product.

Both Taqman® and QuantiTect™ SYBR® systems can be used subsequent to reverse transcription of RNA.

Additionally, other systems to quantitatively measure the level of one or more species of transcripts are known including Molecular Beacons® which uses a probe having a fluorescent molecule and a quencher molecule, the probe capable of forming a hairpin structure such that when in the hairpin form, the fluorescence molecule is quenched, and when hybridized the flourescense increases giving a quantitative measurement of one or more species of RNA transcripts.

Several other techniques for detecting PCR products quantitatively without electrophoresis may also be used according to the invention (see for example PCR Protocols, A Guide to Methods and Applications, Innis et al., Academic Press, Inc. N.Y., (1990)).

Identification of Useful Biomarkers

Using techniques which allow comparison as to the levels of one or more species of RNA transcripts in blood as described herein, one can identify useful biomarkers of a condition. For example one can identify those biomarkers which identify differential levels of one or more species of transcripts as between, for example, an individual or a population of individuals having a condition and an individual or a population of individuals not having a condition.

When comparing two or more samples for differences, results are reported as statistically significant when there is only a small probability that similar results would have been observed if the tested hypothesis (i.e., the RNA transcripts are not expressed at different levels) were not true. A small probability can be defined as the accepted threshold level at which the results being compared are considered significantly different. The accepted lower threshold is set at, but not limited to, 0.05 (i.e., there is a 5% likelihood that the results would be observed between two or more identical populations) such that any values determined by statistical means at or below this threshold are considered significant.

When comparing two or more samples for similarities, results are reported as statistically significant when there is only a small probability that similar results would have been observed if the tested hypothesis (i.e., the genes are not expressed at different levels) were not true. A small probability can be defined as the accepted threshold level at which the results being compared are considered significantly different. The accepted lower threshold is set at, but not limited to, 0.05 (i.e., there is a 5% likelihood that the results would be observed between two or more identical populations) such that any values determined by statistical means above this threshold are not considered significantly different and thus similar.

Preferably the identification of biomarkers is done using statistical analysis. For example, the Wilcox Mann Whitney rank sum test or a standard modified t-test such as a permutation t-test can be used. Additionally multigroup comparisons can also be done when there are three or more reference populations. In this case one can use statistical tests such as ANOVA or Kruskal Wallis which can then be analyzed using a post-hoc pairwise test such as the t-test, the Tukey test, or the student-Newman-Keuls test. Other multiclass comparison tests can also be used as would be understood by a person skilled in the art. See for example (Sokal and Rohlf (1987) Introduction to Biostatistics 2^{nd} edition, WH Freeman, New York), Yeung and Bumgarner, Multiclass classification of microarray data with repeated measurements: application to cancer Genome Biology 2003, 4:R83; Breiman, L. (2001) Statistical Modeling, the two cultures Statistical Science 16(3) 199-231 which are incorporated herein in their entirety.

In order to facilitate ready access, e.g. for comparison, review, recovery and/or modification, the expression profiles of patients with a condition or without a condition can be recorded in a database, whether in a relational database accessible by a computational device or other format, or a manually accessible indexed file of profiles as photographs, analogue or digital imaging, readouts spreadsheets etc. Typically the database is compiled and maintained at a central facility, with access being available locally and/or remotely.

As would be understood by a person skilled in the art, comparison as between the the level of one or more species of transcripts in blood as illustrated by an expression profile of a test individual suspected of having a condition of interest, with that of individuals with the condition of interest, as well as an analogous comparison of expression profiles between individuals with a certain stage or degree of progression of a disease condition, without said condition, or a healthy (“normal”) individual, so as to diagnose or prognose said test individual can occur via expression profiles generated concurrently or non concurrently. It would be understood that a database would be useful to generate said comparison.

As additional test samples from test patients are obtained, through clinical trials, further investigation, or the like, additional data can be determined in accordance with the methods disclosed herein and can likewise be added to a database to provide better reference data for comparison of healthy and/or non-disease patients and/or certain stage or degree of progression of a disease as compared with the test patient sample.

The ability to combine biomarkers provides an even greater potential to help distinguish as between two populations so as to allow diagnosis of a disease or condition. In order to identify useful combitions of biomarkers, each potential combination or set of biomarkers are evaluated for their ability to diagnose an unknown as having or not having a specific condition.

The diagnosing or prognosing may thus be performed by detecting the expression level of one gene, two or more genes, three or more genes, four or more genes, five or more genes, six or more genes, seven or more genes, eight or more genes, nine or more genes, ten or more genes, fifteen or more genes, twenty or more genes thirty or more genes, fifty or more genes, one hundred or more genes, two hundred or more genes, three hundred or more genes, five hundred or more genes or all of the genes disclosed for the specific condition in question.

Use of Expression Profiles for Diagnostic Purposes

As would be understood to a person skilled in the art, one can utilize sets of biomarkers which have been identified as statistically significant as described above in order to characterize an unknown sample as having said disease or not having said disease. This is commonly termed “class prediction”.

Methods that can be used for class prediction analysis have been well described and generally involve a training phase using samples with known classification and a testing phase from which the algorithm generalizes from the training data so as to predict classification of unknown samples (see for Example Slonim, D. (2002), Nature Genetics Supp. Vol 32 502-8, Raychaudhuri et al. (2001) Trends Biotechnol 19: 189-193; Khan et al. (2001) Nature Med. 7 673-9.; Golub et al. (1999) Science 286: 531-7. Hastie et al. (2000) Genome Biol. 1(2) Research 0003.1-0003.21 all of which are incorporated herein by reference in their entirety).

Use of Expression Profiles to Predict Disease State

One can also utilize sets of genes which have been identified as producing differential levels of transcripts in blood which are statistically significant as described above in order to predict whether an asymptomatic individual will develop symptoms of said condition or whether an individual with an early stage of a disease condition will develop a later stage of a disease condition.

For example, as a result of analyzing over 780 individuals, we have surprisingly shown that almost all individuals in the 56 and over age group have either moderate, marked or severe OA, and furthermore that almost all individuals in the 61 and over age group have either marked or severe OA only (see FIG. 35 and Table 3AE) even though there remain approximately 50% of Canadians over the age of 65 who do not show symptoms of osteoarthritis (Statistics Canada, Canadian Community Health Survey, 2000/2001). This data indicates that individuals with mild OA have a significantly increased chance of progressing to marked or severe OA as compared with individuals who do not have mild OA.

| | | Age Distirbution | | | | | | | | | | | | |
|---|---|---|---|---|---|---|---|---|---|---|---|---|---|---|
| OA | Sex | <=20 | 21-25 | 26-30 | 31-35 | 36-40 | 41-45 | 46-50 | 51-55 | 56-60 | 61-65 | 66-70 | 71-86 | Total |
| Mild | F | 5 | 8 | 17 | 12 | 9 | 3 | 0 | 1 | 0 | 0 | 0 | 0 | 55 |
| | M | 5 | 12 | 14 | 16 | 13 | 11 | 4 | 3 | 0 | 2 | 0 | 0 | 80 |
| 1-6 | Total | 10 | 20 | 31 | 28 | 22 | 14 | 4 | 4 | 0 | 2 | 0 | 0 | 135 |
| Moderate | F | 1 | 2 | 12 | 5 | 8 | 9 | 11 | 7 | 2 | 1 | 2 | 0 | 60 |
| | M | 4 | 6 | 7 | 10 | 18 | 16 | 17 | 8 | 5 | 1 | 0 | 0 | 92 |
| 7-12 | Total | 5 | 8 | 19 | 15 | 26 | 25 | 28 | 15 | 7 | 2 | 2 | 0 | 152 |
| Marked | F | 0 | 0 | 1 | 4 | 4 | 18 | 21 | 26 | 26 | 21 | 14 | 22 | 157 |
| | M | 1 | 0 | 3 | 7 | 11 | 10 | 27 | 28 | 14 | 10 | 8 | 18 | 137 |
| 13-18 | Total | 1 | 0 | 4 | 11 | 15 | 28 | 48 | 54 | 40 | 31 | 22 | 40 | 294 |
| Severe | F | 0 | 0 | 1 | 0 | 0 | 1 | 4 | 9 | 10 | 6 | 10 | 25 | 66 |
| | M | 0 | 0 | 0 | 0 | 0 | 2 | 1 | 2 | 9 | 6 | 10 | 27 | 57 |
| over 19 | Total | 0 | 0 | 1 | 0 | 0 | 3 | 5 | 11 | 19 | 12 | 20 | 52 | 123 |
| | Total | 16 | 28 | 55 | 54 | 63 | 70 | 85 | 84 | 66 | 47 | 44 | 92 | 704 |
| Normal | F | 4 | 5 | 8 | 4 | 8 | 4 | 3 | 1 | 0 | 0 | 0 | 0 | 37 |
| | M | 0 | 9 | 8 | 8 | 3 | 5 | 3 | 6 | 0 | 0 | 0 | 0 | 42 |
| | Total | 4 | 14 | 16 | 12 | 11 | 9 | 6 | 7 | 0 | 0 | 0 | 0 | 79 |

As a result, one can utilize the methods of class prediction analysis described herein in order to determine whether an individual will develop late stage OA by identifying individuals with early stages of OA.

As additional samples are obtained, for example during clinical trials, their expression profiles can be determined and correlated with the relevant subject data in the database and likewise be recorded in said database. Algorithms as described above can be used to query additional samples against the existing database to further refine the predictive determination by allowing an even greater association between the prediction of OA and one or more species of RNA transcripts signature.

The prediction of late stage OA may thus be performed by detecting the level of transcripts expressed by two or more genes, three or more genes, four or more genes, five or more genes, six or more genes, seven or more genes, eight or more genes, nine or more genes, ten or more genes, fifteen or more genes, twenty or more genes thirty or more genes, fifty or more genes, one hundred or more genes, two hundred or more genes, three hundred or more genes, five hundred or more genes or all of the genes disclosed for identifying mild OA.

The following references were cited herein:
Alon U et al. Proc Natl Acad Sci USA (1999), 96:6745-6750
Claudio J O et al. (1998). Genomics 50:44-52.
Chelly J et al. (1989). Proc. Nat. Acad. Sci. USA. 86:2617-2621.
Chelly J et al. (1988). Nature 333:858-860.
Drews J & Ryser S (1997). Nature Biotech. 15:1318-9.
Ferrie R M et al. (1992). Am. J. Hum. Genet. 51:251-62.
Fu D-J et al. (1998). Nat. Biotech 16: 381-4.
Gala J L et al. (1998). Clin. Chem. 44(3):472-81.
Geisterfer-Lowrance A A T et al. (1990). Cell 62:999-1006.
Groden J et al. (1991). Cell 66:589-600.
Hwang D M et al. (1997). Circulation 96:4146-4203.
Jandreski M A & Liew C C (1987). Hum. Genet. 76:47-53.
Jin O et al. (1990). Circulation 82:8-16
Kimoto Y (1998). Mol. Gen. Genet 258:233-239.
Koster M et al. (1996). Nat. Biotech 14: 1123-8.
Liew & Jandreski (1986). Proc. Nat. Acad. Sci. USA. 83:3175-3179
Liew C C et al. (1990). Nucleic Acids Res. 18:3647-3651.
Liew C C (1993). J Mol. Cell. Cardiol. 25:891-894
Liew C C et al. (1994). Proc. Natl. Acad. Sci. USA. 91:10645-10649.
Liew et al. (1997). Mol. and Cell. Biochem. 172:81-87.
Niimura H et al. (1998). New Eng. J. Med. 338:1248-1257.
Ogawa M (1993). Blood 81:2844-2853.
Santoro I M & Groden J (1997). Cancer Res. 57:488-494.
Schummer M et al. (1999), Gene 238:375-385
van't Veer L J et al. (2002) Nature 415:530-536;
Yeung and Bumgarner, (2003) Genome Biology 4:R83
Yuasa T et al. (1998). Japanese J Cancer Res. 89:879-882.
   Description of Tables:
   Table 1 shows genes that are differentially expressed in blood samples from patients with a disease or patients who are co-morbid as compared to blood samples from healthy patients or patients without said disease, or with only one of said co-morbid diseases
   Table 1A shows the identity of those genes that are differentially expressed in blood samples from patients with osteoarthritis and hypertension as compared with normal patients using the ChondroChip™ platform.
   Table 1B shows the identity of those genes that are differentially expressed in blood samples from patients with osteoarthritis and obesity as compared with normal patients using the ChondroChip™ platform.
   Table 1C shows the identity of those genes that are differentially expressed in blood samples from patients with osteoarthritis and allergies as compared with normal patients using the ChondroChip™ platform.
   Table 1D shows the identity of those genes that are differentially expressed in blood samples from patients with osteoarthritis and subject to systemic steroids as compared with normal patients using the ChondroChip™ platform.
   Table 1E shows the identity of those genes that are differentially expressed in blood samples from patients with hypertension as compared to non hypertension patients using the ChondroChip™ platform.
   Table 1F shows the identity of those genes that are differentially expressed in blood samples from patients obesity as compared to non obese patients using the ChondroChip™ platform.
   Table 1G shows the identity of those genes that are differentially expressed in blood samples from patients with hypertension and OA when compared with patients who have OA only wherein genes identified in Table 1A have been removed so as to identify genes which are unique to hypertension.
   Table 1H shows the identity of those genes which were identified in Table 1A which are shared with those genes differentially expressed in blood samples from patients with hypertension and OA when compared with patients who have OA only.
   Table 1I shows the identity of those genes that are differentially expressed in blood samples from patients who are obese and have OA when compared with patients who have OA only and wherein genes identified in Table 1B have been removed so as to identify genes which are unique to obesity.
   Table 1J shows the identify of those genes identified in Table 1B which are shared with those genes differentially expressed in blood samples from patients who are obese and have OA when compared with patients who have OA.
   Table 1K shows the identity of those genes that are differentially expressed in blood samples from patients with allergies and OA when compared with patients who have OA only wherein genes identified in Table 1C have been removed so as to identify genes which are unique to allergies.
   Table 1L shows the identify of those genes identified in Table 3C which are shared with those genes differentially expressed in blood samples from patients with allergies and OA when compared with patients who have OA only.
   Table 1M shows the identity of those genes that are differentially expressed in blood samples from patients who are on systemic steroids and have OA when compared with patients who have OA only wherein genes identified in Table 1D have been removed so as to identify genes which are unique to patients on systemic steroids.
   Table 1N shows the identify of those genes identified in Table 1D which are shared with those genes differentially expressed in blood samples from patients who are on systemic steroids and have OA when compared with patients who have OA only.
   Table 1O shows the identity of those genes that are differentially expressed in blood from patients taking either birth control, prednisone or hormone replacement therapy and presenting with OA using the ChondroChip™ platform.
   Table 1P shows the identity of those genes that are differentially expressed in blood samples from patients with type II diabetes as compared to patients without type II diabetes using the ChondroChip™ platform.
   Table 1Q shows the identity of those genes that are differentially expressed in blood samples from patients with Hyperlipidemia as compared to patients without Hyperlipidemia using the ChondroChip™ platform.
   Table 1R shows the identity of those genes that are differentially expressed in blood samples from patients with lung disease as compared to patients without lung disease using the ChondroChip™ platform.
   Table 1S shows the identity of those genes that are differentially expressed in blood samples from patients with bladder cancer as compared to patients without bladder cancer using the ChondroChip™ platform.
   Table 1T shows the identity of those genes that are differentially expressed in blood samples from patients with early stage bladder cancer, late stage bladder cancer or non-bladder cancer using the ChondroChip™ platform.
   Table 1U shows the identity of those genes that are differentially expressed in blood samples from patients with coronary artery disease (CAD) as compared to patients not having CAD using the ChondroChip™ platform.
   Table 1V shows the identity of those genes that are differentially expressed in blood samples from patients with rheumatoid arthritis as compared to patients not having rheumatoid arthritis using the ChondroChip™ platform.
   Table 1W shows the identity of those genes that are differentially expressed in blood samples from patients with rheumatoid arthritis as compared to patients not having rheumatoid arthritis using the Affymetrix® platform.
   Table 1X shows the identity of those genes that are differentially expressed in blood samples from patients with depression as compared with patients not having depression using the ChondroChip™ platform.
   Table 1Y shows the identity of those genes that are differentially expressed in blood samples from patients with various stages of osteoarthritis using the ChondroChip™ platform.
   Table 1Z shows the identity of those genes that are differentially expressed in blood samples from patients with liver cancer as compared with patients not having liver cancer using the Affymetrix® platform.
   Table 1AA shows the identity of those genes that are differentially expressed in blood samples from patients with schizophrenia as compared with patients not having schizophrenia using the Affymetrix® platform.
   Table 1AB shows the identity of those genes that are differentially expressed in blood samples from patients with Chagas disease as compared with patients not having Chagas disease using the Affymetrix® platform.
   Table 1AC shows the identity of those genes that are differentially expressed in blood samples from patients with asthma as compared with patients not having asthma using the ChondroChip™.
   Table 1AD shows the identity of those genes that are differentially expressed in blood samples from patients with asthma as compared with patients not having asthma using the Affymetrix® platform.
   Table 1AE shows the identity of those genes that are differentially expressed in blood samples from patients with lung cancer as compared with patients not having lung cancer using the Affymetrix® platform.
   Table 1AG shows the identity of those genes that are differentially expressed in blood samples from patients with hypertension as compared with patients not having hypertension using the Affymetrix® platform.
   Table 1AH shows the identity of those genes that are differentially expressed in blood samples from patients with obesity as compared with patients not having obesity using the Affymetrix® platform.
   Table 1AI shows the identity of those genes that are differentially expressed in blood samples from patients with ankylosing spondylitis using the Affymetrix® platform.
   Table 2 shows the identity of those genes that are differentially expressed in blood from patients with either mild or severe OA, but for which genes relevant to asthma, obesity, hypertension, systemic steroids and allergies have been removed.
   Table 3 shows those genes that are differentially expressed in blood samples from patients with a first disease as compared to blood samples from patients with a second disease so as to allow differential diagnosis as between said first and second disease.
   Table 3A shows the identity of those genes that are differentially expressed in blood from patients with schizophrenia as compared with manic depression syndrome (MDS) using the Affymetrix® platform.
   Table 3B shows the identity of those genes that are differentially expressed in blood from patients with hepatitis as compared with liver cancer using the Affymetrix® platform.
   Table 3C shows the identity of those genes that are differentially expressed in blood from patients with bladder cancer as compared with liver cancer using the Affymetrix® platform.
   Table 3D shows the identity of those genes that are differentially expressed in blood from patients with bladder cancer as compared with testicular cancer using the Affymetrix® platform.
   Table 3E shows the identity of those genes that are differentially expressed in blood from patients with testicular cancer as compared with kidney cancer using the Affymetrix® platform.
   Table 3F shows the identity of those genes that are differentially expressed in blood from patients with liver cancer as compared with stomach cancer using the Affymetrix® platform.
   Table 3G shows the identity of those genes that are differentially expressed in blood from patients with liver cancer as compared with colon cancer using the Affymetrix® platform.
   Table 3H shows the identity of those genes that are differentially expressed in blood from patients with stomach cancer as compared with colon cancer using the Affymetrix® platform.
   Table 3I shows the identity of those genes that are differentially expressed in blood from patients with Rheumatoid Arthritis as compared with Osteoarthritis using the Affymetrix® platform.
   Table 3K shows the identity of those genes that are differentially expressed in blood from patients with Chagas Disease as compared with Heart Failure using the Affymetrix® platform.
   Table 3L shows the identity of those genes that are differentially expressed in blood from patients with Chagas Disease as compared with Coronary Artery Disease using the Affymetrix® platform.
   Table 3N shows the identity of those genes that are differentially expressed in blood from patients with Coronary Artery Disease as compared with Heart Failure using the Affymetrix® platform.
   Table 3P shows the identity of those genes that are differentially expressed in blood from patients with Asymptomatic Chagas Disease as compared with Symptomatic Chagas Disease using the Affymetrix® platform.
   Table 3Q shows the identity of those genes that are differentially expressed in blood from patients with Alzheimer's' as compared with Schizophrenia using the Affymetrix® platform.
   Table 3R shows the identity of those genes that are differentially expressed in blood from patients with Alzheimer's' as compared with Manic Depression Syndrome using the Affymetrix® platform.
   Table 4 shows those genes that are differentially expressed in blood samples from patients with a stage of Osteoarthritis as compared to blood samples from patients with a second stage of Osteoarthritis so as to allow monitoring of progression and/or regression of disease.
   Table 4A shows the identity of those genes that are differentially expressed in blood from patients with Osteoarthritis as compared with patients without Osteoarthritis using the ChondroChip™ platform.
   Table 4B shows the identity of those genes that are differentially expressed in blood from patients with Osteoarthritis as compared with patients without Osteoarthritis using the Affymetrix® platform.
   Table 4C shows the identity of those genes that are differentially expressed in blood from patients with mold Osteoarthritis as compared with patients without mild Osteoarthritis using the ChondroChip™ platform.
   Table 4D shows the identity of those genes that are differentially expressed in blood from patients with mild Osteoarthritis as compared with patients without Osteoarthritis using the Affymetrix® platform.
   Table 4E shows the identity of those genes that are differentially expressed in blood from patients with moderate Osteoarthritis as compared with patients without Osteoarthritis using the ChondroChip™ platform.
   Table 4F shows the identity of those genes that are differentially expressed in blood from patients with moderate Osteoarthritis as compared with patients without Osteoarthritis using the Affymetrix® platform.
   Table 4G shows the identity of those genes that are differentially expressed in blood from patients with marked Osteoarthritis as compared with patients without Osteoarthritis using the ChondroChip™ platform.
   Table 4H shows the identity of those genes that are differentially expressed in blood from patients with marked Osteoarthritis as compared with patients without Osteoarthritis using the Affymetrix® platform.
   Table 4I shows the identity of those genes that are differentially expressed in blood from patients with severe Osteoarthritis as compared with patients without Osteoarthritis using the ChondroChip™ platform.
   Table 4J shows the identity of those genes that are differentially expressed in blood from patients with severe Osteoarthritis as compared with patients without Osteoarthritis using the Affymetrix® platform.
   Table 4K shows the identity of those genes that are differentially expressed in blood from patients with mild Osteoarthritis as compared with patients with moderate Osteoarthritis using the ChondroChip™ platform.
   Table 4L shows the identity of those genes that are differentially expressed in blood from patients with mild Osteoarthritis as compared with patients with moderate Osteoarthritis using the Affymetrix® platform.
   Table 4M shows the identity of those genes that are differentially expressed in blood from patients with mild Osteoarthritis as compared with patients with marked Osteoarthritis using the ChondroChip™ platform.
   Table 4N shows the identity of those genes that are differentially expressed in blood from patients with mild Osteoarthritis as compared with patients with marked Osteoarthritis using the Affymetrix® platform.
   Table 4O shows the identity of those genes that are differentially expressed in blood from patients with mild Osteoarthritis as compared with patients with severe Osteoarthritis using the ChondroChip™ platform.
   Table 4P shows the identity of those genes that are differentially expressed in blood from patients with mild Osteoarthritis as compared with patients with severe Osteoarthritis using the Affymetrix® platform.
   Table 4Q shows the identity of those genes that are differentially expressed in blood from patients with moderate Osteoarthritis as compared with patients with marked Osteoarthritis using the ChondroChip™ platform.
   Table 4R shows the identity of those genes that are differentially expressed in blood from patients with moderate Osteoarthritis as compared with patients with marked Osteoarthritis using the Affymetrix® platform.
   Table 4S shows the identity of those genes that are differentially expressed in blood from patients with moderate Osteoarthritis as compared with patients with severe Osteoarthritis using the ChondroChip™ platform.
   Table 4T shows the identity of those genes that are differentially expressed in blood from patients with moderate Osteoarthritis as compared with patients with severe Osteoarthritis using the Affymetrix® platform.
   Table 4U shows the identity of those genes that are differentially expressed in blood from patients with marked Osteoarthritis as compared with patients with severe Osteoarthritis using the ChondroChip™ platform.
   Table 4V shows the identity of those genes that are differentially expressed in blood from patients with marked Osteoarthritis as compared with patients with severe Osteoarthritis using the Affymetrix® platform.
   Table 5 shows those genes that are differentially expressed in blood samples from patients with a disease or condition of interest as compared to blood samples from patients without said disease or condition.
   Table 5A shows the identity of those genes that are differentially expressed in blood samples from patients with psoriasis as compared with patients not having hypertension using the Affymetrix® platform.
   Table 5B shows the identity of those genes that are differentially expressed in blood samples from patients with thyroid disorder as compared with patients not having thyroid disorder using the Affymetrix® platform.
   Table 5C shows the identity of those genes that are differentially expressed in blood samples from patients with irritable bowel syndrome as compared with patients not having irritable bowel syndrome using the Affymetrix® platform.
   Table 5D shows the identity of those genes that are differentially expressed in blood samples from patients with osteoporosis as compared with patients not having osteoporosis using the Affymetrix® platform.
   Table 5E shows the identity of those genes that are differentially expressed in blood samples from patients with migraine headaches as compared with patients not having migraine headaches using the Affymetrix® platform.
   Table 5F shows the identity of those genes that are differentially expressed in blood samples from patients with eczema as compared with patients not having eczema using the Affymetrix® platform.
   Table 5G shows the identity of those genes that are differentially expressed in blood samples from patients with NASH as compared with patients not having NASH using the Affymetrix® platform.
   Table 5H shows the identity of those genes that are differentially expressed in blood samples from patients with alzheimers' disease as compared with patients not having alzheimer's disease using the Affymetrix® platform.
   Table 5I shows the identity of those genes that are differentially expressed in blood samples from patients with Manic Depression Syndrome as compared with patients not having Manic Depression Syndrome using the Affymetrix® platform.
   Table 5J shows the identity of those genes that are differentially expressed in blood samples from patients with Crohn's Colitis as compared with patients not having Crohn's Colitis using the Affymetrix® platform.
   Table 5K shows the identity of those genes that are differentially expressed in blood samples from patients with Chronis Cholecystits as compared with patients not having Chronis Cholecystits using the Affymetrix® platform.
   Table 5L shows the identity of those genes that are differentially expressed in blood samples from patients with Heart Failure as compared with patients not having Heart Failure using the Affymetrix® platform.
   Table 5M shows the identity of those genes that are differentially expressed in blood samples from patients with Cervical Cancer as compared with patients not having Cervical Cancer using the Affymetrix® platform.
   Table 5N shows the identity of those genes that are differentially expressed in blood samples from patients with Stomach Cancer as compared with patients not having Stomach Cancer using the Affymetrix® platform.
   Table 5O shows the identity of those genes that are differentially expressed in blood samples from patients with Kidney Cancer as compared with patients not having Kidney Cancer using the Affymetrix® platform.
   Table 5P shows the identity of those genes that are differentially expressed in blood samples from patients with Testicular Cancer as compared with patients not having Testicular Cancer using the Affymetrix® platform.
   Table 5Q shows the identity of those genes that are differentially expressed in blood samples from patients with Colon Cancer as compared with patients not having Colon Cancer using the Affymetrix® platform.
   Table 5R shows the identity of those genes that are differentially expressed in blood samples from patients with Hepatitis B as compared with patients not having Hepatitis B using the Affymetrix® platform.
   Table 5S shows the identity of those genes that are differentially expressed in blood samples from patients with Pancreatic Cancer as compared with patients not having Pancreatic Cancer using the Affymetrix® platform.
   Table 5T shows the identity of those genes that are differentially expressed in blood samples from patients with Asymptomatic Chagas as compared with patients not having Chagas using the Affymetrix® platform.
   Table 5U shows the identity of those genes that are differentially expressed in blood samples from patients with Symptomatic Chagas as compared with patients not having Chagas using the Affymetrix® platform.
   Table 5V shows the identity of those genes that are differentially expressed in blood samples from patients with Bladder Cancer as compared with patients not having Bladder Cancer using the Affymetrix® platform.
   Table 6 shows those genes that are differentially expressed in blood samples from patients with any one of a series of related conditions as compared to blood samples from patients without said related conditions.
   Table 6A shows the identity of those genes that are differentially expressed in blood samples from patients with Cancer as compared with patients not having Cancer using the Affymetrix® platform.
   Table 6B shows the identity of those genes that are differentially expressed in blood samples from patients with Cardiovascular Disease as compared with patients not having a Cardiovascular Disease using the Affymetrix® platform.
   Table 6C shows the identity of those genes that are differentially expressed in blood samples from patients with a Neurological Disease as compared with patients not having a Neurological Disease using the Affymetrix® platform.
   Table 7 shows those genes that are differentially expressed in blood samples from with a condition wherein said condition is a treatment as compared to blood samples from patients without said condition.
   Table 7A shows the identity of those genes that are differentially expressed in blood samples from patients taking Celebrex® as compared with patients on a Cox Inhibitor which was not Celebrex® using the ChondroChip™ platform.
   Table 7B shows the identity of those genes that are differentially expressed in blood samples from patients taking Celebrex® as compared with patients not on Celebrex® using the ChondroChip™ platform.
   Table 7C shows the identity of those genes that are differentially expressed in blood samples from patients taking Vioxx® as compared with patients not on Vioxx® using the ChondroChip™ platform.
   Table 7D shows the identity of those genes that are differentially expressed in blood samples from patients taking Vioxx® as compared with patients on a Cox inhibitor but not on Vioxx® using the ChondroChip™ platform.
   Table 7E shows the identity of those genes that are differentially expressed in blood samples from patients taking NSAIDS as compared with patients not on NSAIDS using the ChondroChip™ platform.
   Table 7F shows the identity of those genes that are differentially expressed in blood samples from patients taking Cortisone as compared with patients not on Cortisone using the ChondroChip™ platform.
   Table 7G shows the identity of those genes that are differentially expressed in blood samples from patients taking Visco Supplement as compared with patients not on Visco Supplement using the ChondroChip™ platform.
   Table 7H shows the identity of those genes that are differentially expressed in blood samples from patients taking Lipitor® as compared with patients not on Lipitor® using the ChondroChip™ platform.
   Table 7I shows the identity of those genes that are differentially expressed in blood samples from patients who are smokers as compared with patients who are not smokers using the ChondroChip™ platform
   Table 8A is an annotation table showing the relationship between the gene ID identified in Tables 1-7 wherein the data was generated using the Affymetrix®platform and gene identified by the Affymetrix probe.
   Table 8B is an annotation table showing the relationship between the clone ID identified in Tables 1-7 wherein the data was generated using the ChondroChip™ platform and the gene identified by the EST clones.
   Table 9 shows the descriptions as to the various annotations provided for both the ChondroChip™ and the Affymetrix® microarray results.
   Table 10 shows how the incidence of different stages of OA varies with respect to age in males and females
   Table 11 shows 223 EST sequences of Tables 1A-7I with “no-significant match” to known gene sequence in Patent-In Format.
   Table 12 shows a list of genes showing greater than two fold differential expression in CAD peripheral blood cells relative to that of normal blood cells.

The following examples are given for the purpose of illustrating various embodiments of the invention and are not meant to limit the present invention in any fashion.

### EXAMPLE 1

Blood cDNA chip Microarray Data Analysis of RNA expression profiles of blood samples from individuals having coronary artery disease as compared with RNA expression profiles from normal individuals.

A microarray was constructed using cDNA clones from a human peripheral blood cell cDNA library, as described herein. A total of 10,368 polymerase chain reaction (PCR) products of the clones from the human peripheral blood cell cDNA library described herein were arrayed using GNS 417 arrayer (Affymetrix). RNA for microarray analysis was isolated from whole blood samples without prior fractionation, obtained from three male and one female patients with coronary heart disease (80-90% stenosis) receiving vascular extension drugs and awaiting bypass surgery, and three healthy male controls.

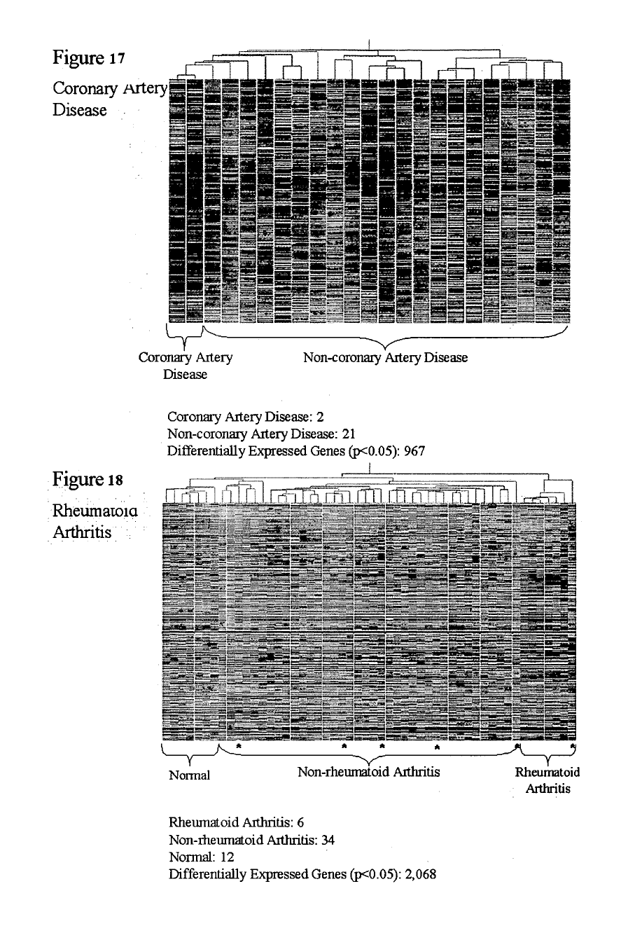

A method of high-fidelity mRNA amplification from 1 pg of total RNA sample was used. Cy5- or Cy3-dUTP was incorporated into cDNA probes by reverse transcription of anti-sense RNA, primed by oligo-dT. Labeled probes were purified and concentrated to the desired volume. Pre-hybridization and hybridization were performed following Hegde's protocol (Hegde P et al., A concise guide to cDNA microarray analysis. Biotechniques 2000;29: 548-56). After overnight hybridization and washing, hybridization signals were detected with a GMS 418 scanner at 635-nm (Cy5) and 532-nm (Cy3) wave lengths (see FIG. 17). Two RNA pools were labeled alternatively with Cy5- and Cy3-dUTP, and each experiment was repeated twice. Cluster analysis using GeneSpring™ 4.1.5 (Silicon Genetics) revealed two distinct groups consisting of four CAD and three normal control samples. Two images scanned at different wavelengths were super-imposed. Individual spots were identified on a customized grid. Of 10,368 spots, 10,012 (96.6%) were selected after the removal of spots with irregular shapes. Data quality was assessed with values of Ch1GTB2 and Ch2GTB2 provided by ScanAlyze. Only spots with Ch1GTB2 and Ch2GTB2 over 0.50 were selected. After evaluation of signal intensities, 8750 (84.4%) spots were left. Signal intensities were normalized using a scatter-plot of the signal intensities of the two channels. After normalization, the expression ratios of β-actin were 1.00+021 1 11+0.22, 1.14+0.20 and 1.30+0.18 (24 samples of β-actin were spotted on this slide as the positive control) in the four images. Differential expression of RNA was assessed as the ratio of two wave-length signal intensities. Spots showing a differential expression more than twofold relative to normal in all four experiments were identified as peripheral blood cell, differentially expressed candidate genes in CAD. 108 genes are differentially expressed in CAD peripheral blood cells. 43 genes are downregulated in CAD blood cells and 65 are upregulated (see Table 12). Functional characterization of these genes from which the differentially expressed RNA transcripts were transcribed shows that differential expression at the level of RNA transcription takes place in every gene functional category, indicating that profound changes occur in peripheral blood cells from patients with CAD.

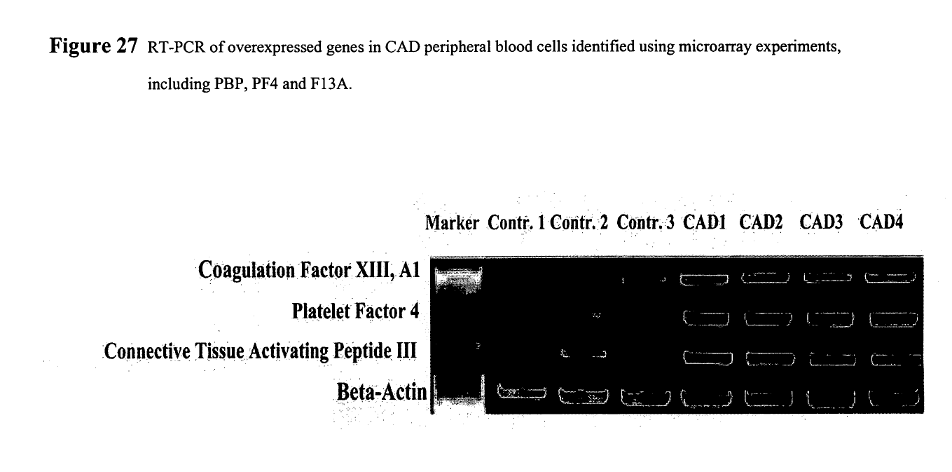

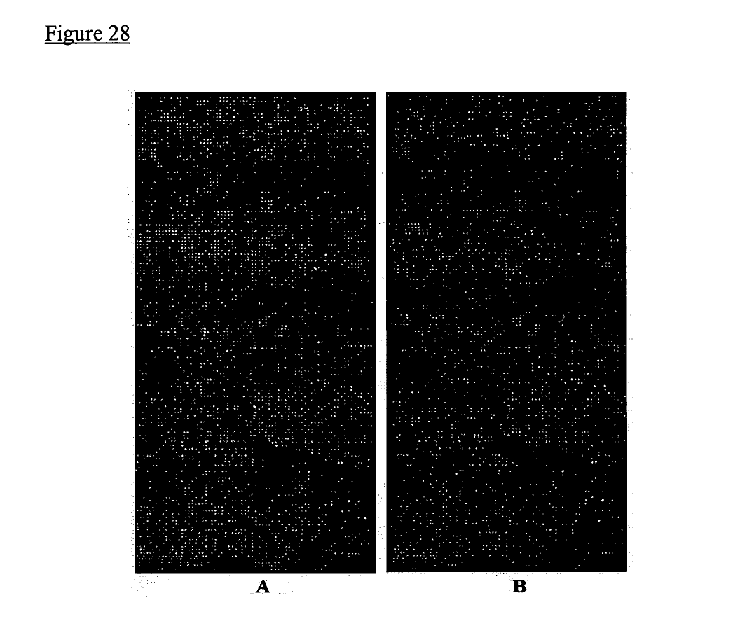

The differential expression of RNA transcribed from three genes, pro-platelet basic protein (PBP), platelet factor 4 (PF4) and coagulation factor XIII A1 (F13A), initially identified in the microarray data analysis, was further examined by reverse transcriptase-PCR (RT-PCR) using the Titan One-tube RT-PCR kit (Boehringer Mannheim). Reaction solution contains 0.2 mM each dNTP, 5 mM DTT, 1.5 mM MgCl 0.1 pg of total RNA from each sample and 20 pmol each of left and right primers of PBP (5′-GGTGCTGCTGCTTCTGTCAT-3′ (SEQ ID NO: 224) and 5′-GGCAGATTTT CCTCCCATCC-3′), (SEQ ID NO:225), F13A (5′-AGTCCACCGTGCTAACCATC-3′ (SEQ ID NO:226), and 5′-AGGGAGTCACTGCTCATGCT-3′) (SEQ ID NO:227), and PF4 (5′ GTTGCTGCTCCTGCCACTT 3′ (SEQ ID NO:228), and 5′ GTGGCTATCAGTTGGGCAGT-3′)(SEQ ID NO:229). RT-PCR steps are as follows: 1. reverse-transcription: 30 min at 60° C.; 2. PCR: 2 min at 94° C., followed by 30-35 cycles (as optimized for each gene) for 30 s at 94° C., 30 s at optimized annealing temperature and 2 min at 68° C.; 3. final extension: 7 min at 68° C. PCR products were electrophoresed on 1.5% agarose gels. Human (□β-actin primers (5′-GCGAGAAGATGACCCAGATCAT-3′ (SEQ ID NO:230) and 5′-GCTCAGGAGGAGCAATGATCTT-3 (SEQ ID NO:231) were used as the internal control. The RT-PCR analysis confirmed that the expression of the three secreted proteins: PBP, PF4 and F13A were all upregulated in CAD blood cells (see FIGS. 27 and 17)

TABLE 12

| | | | | Protein |
|---|---|---|---|---|
| | Accession | Fold | Functional | Accession |
| | number | (average) | category | Number |
| Upregulated gene in CAD | | | | |
| REV3-like, catalytic subunit | AF035537 | 2.3 | Cell cycle | NP_002903 |
| of DNA polymerase zeta | | | | |
| TGFB1-induced anti- | D86970 | 2.2 | Cell cycle | NP_510880 |
| apoptotic factor 1 | | | | |
| A disintegrin and | AA044656 | 2.7 | Cell signaling | NP_001101 |
| metalloproteinase domain 10 | | | | |
| Centaurin, delta 2 | AA351412 | 2 | Cell signaling | NP_631920 |
| Chloride intracellular | AA411940 | 2.2 | Cell signaling | NP_039234 |
| channel 4 | | | | |
| Endothelin receptor typeA | D90348 | 2.1 | Cell signaling | NP_001948 |
| Glutamate receptor, | N33821 | 2.4 | Cell signaling | NP_777567 |
| ionotropic | | | | |
| Mitogen-activated protein | L38486 | 3.7 | Cell signaling | NP_002395 |
| kinase 7 | | | | |
| Mitogen-activated protein | AB009356 | 4.5 | Cell signaling | NP_663306 |
| kinase kinase kinase 7 | | | | |
| Myristoylated alanine-rich | D10522 | 2.5 | Cell signaling | NP_002347 |
| protein kinase C substrate | | | | |
| NIMA-related kinase 7 | AA093324 | 3.5 | Cell signaling | NP_598001 |
| PAK2 | AA262968 | 3.5 | Cell signaling | Q13177 |
| Phospholipid scramblase 1 | AA054476 | 3.3 | Cell signaling | NP_066928 |
| Serum deprivation response | Z30112 | 4.5 | Cell signaling | NP_004648 |
| Adducin 3 | AA029158 | 2.9 | Cell structure | NP_063968 |
| Desmin | AF167579 | 4.4 | Cell structure | NP_001918 |
| Fibromodulin | W23613 | 2.9 | Cell structure | NP_002014 |
| Laminin, beta 2 | S77512 | 2.2 | Cell structure | NP_002283 |
| Laminin, beta 3 | L25541 | 2.4 | Cell structure | NP_000219 |
| Osteonectin | Y00755 | 3.1 | Cell structure | NP_003109 |
| CD59 antigen p18-20 | W01111 | 2.4 | Cell/organism | NP_000602 |
| | | | defense | |
| Clusterin | M64722 | 3.5 | Cell/organism | NP_001822 |
| | | | defense | |
| F13A | M14539 | 2.1 | Cell/organism | NP_000120 |
| | | | defense | |
| Defensin, alpha 1 | M26602 | 4.2 | Cell/organism | NP_004075 |
| | | | defense | |
| PF4 | M25897 | 2.1 | Cell/organism | NP_002610 |
| | | | defense | |
| PBP | M54995 | 5.5 | Cell/organism | NP_002695 |
| | | | defense | |
| E2F transcription factor 3 | D38550 | 2.1 | Gene expression | NP_001940 |
| Early growth response 1 | M62829 | 2.7 | Gene expression | NP_001955 |
| Eukaryotic translation | N86030 | 2.3 | Gene expression | NP_001393 |
| elongation factor 1 alpha 1 | | | | |
| Eukaryotic translation | M15353 | 2.1 | Gene expression | NP_001959 |
| initiation factor 4E | | | | |
| F-box and WD-40 domain | AB014596 | 2.7 | Gene expression | NP_387449 |
| protein 1B | | | | |
| Makorin, ring finger protein, 2 | AA331966 | 2.1 | Gene expression | NP_054879 |
| Non-canonical ubiquitin- | N92776 | 2.5 | Gene expression | NP_057420 |
| conjugating enzyme 1 | | | | |
| Nuclear receptor subfamily | Z30425 | 4.7 | Gene expression | NP_005113 |
| 1, group I, member 3 | | | | |
| Ring finger protein 11 | T08927 | 3 | Gene expression | NP_055187 |
| Transducin-like enhancer of | M99435 | 3.3 | Gene expression | NP_005068 |
| split 1 | | | | |
| Alkaline phosphatase, | AB011406 | 2.2 | Metabolism | NP_000469 |
| liver/bone/kidney | | | | |
| Annexin A3 | M63310 | 3.4 | Metabolism | NP_005130 |
| Branched chain | AA336265 | 4.8 | Metabolism | NP_005495.1 |
| aminotransferase 1, cytosolic | | | | |
| Cytochrome b | AF042500 | 2.5 | Metabolism | |
| Glutaminase | D30931 | 2.6 | Metabolism | NP_055720 |
| Lysophospholipase I | AF035293 | 2.8 | Metabolism | NP_006321 |
| NADH dehydrogenase 1, | AA056111 | 2.5 | Metabolism | NP_002485 |
| subcomplex unknown 1, 6 kDa | | | | |
| Phosphofructokinase | M26066 | 2.2 | Metabolism | NP_000280 |
| Ubiquinol-cytochrome c | M22348 | 2.5 | Metabolism | NP_006285 |
| reductase binding protein | | | | |
| CGI-110 protein | AA341061 | 2.4 | Unclassified | NP_057131 |
| Dactylidin | H95397 | 2.7 | Unclassified | NP_112225 |
| Deleted in split-hand/split- | T24503 | 2.4 | Unclassified | NP_006295 |
| foot 1 region | | | | |
| Follistatin-like 1 | R14219 | 2.7 | Unclassified | NP_009016 |
| FUS-interacting protein 1 | W37945 | 2.8 | Unclassified | NP_473357 |
| Hypothetical protein | W47233 | 7 | Unclassified | NP_112201 |
| FLJ12619 | | | | |
| Hypothetical protein from | N68247 | 2.7 | Unclassified | |
| EUROIMAGE 588495 | | | | |
| Hypothetical protein | AA251423 | 2.2 | Unclassified | NP_057702 |
| LOC51315 | | | | |
| KIAA1705 protein | T80569 | 2.7 | Unclassified | NP_009121.1 |
| Mesoderm induction early | AI650409 | 2.2 | Unclassified | NP_065999 |
| response 1 | | | | |
| Phosphodiesterase 4D- | AA740661 | 2.5 | Unclassified | NP_055459 |
| interacting protein | | | | |
| Preimplantation protein 3 | D59087 | 2.5 | Unclassified | NP_056202 |
| Putative nuclear protein | W33098 | 2.8 | Unclassified | NP_115788 |
| ORF1-FL49 | | | | |
| Similar to rat nuclear | H09434 | 2.2 | Unclassified | Q9H1E3 |
| ubiquitous casein kinase 2 | | | | |
| Similar to RIKEN | AA297412 | 2.5 | Unclassified | T02670 |
| Spectrin, beta | AI334431 | 2.5 | Unclassified | Q01082 |
| Stromal cell-derived factor | H71558 | 4.1 | Unclassified | NP_816929 |
| receptor 1 | | | | |
| Thioredoxin-related protein | AA421549 | 2.8 | Unclassified | NP_110437 |
| Transmembrane 4 | D29808 | 2.4 | Unclassified | NP_004606 |
| superfamily member 2 | | | | |
| Tumor endothelial marker 8 | D79964 | 2.5 | Unclassified | NP_444262 |
| Downregulated gene in CAD | | | | |
| CASP8 and FADD-like | AF015450 | 0.45 | Cell cycle | NP_003870 |
| apoptosis regulator | | | | |
| CD81 antigen | M33680 | 0.41 | Cell cycle | NP_004347 |
| Cell division cycle 25B | M81934 | 0.4 | Cell cycle | NP_068660 |
| DEAD/H (Asp-Glu-Ala- | AA985699 | 0.42 | Cell cycle | NP_694705 |
| Asp/His) box polypeptide 27 | | | | |
| F-box and leucine-rich repeat | R98291 | 0.27 | Cell cycle | NP_036440 |
| protein 11 | | | | |
| Minichromosome | H10286 | 0.43 | Cell cycle | NP_003897 |
| maintenance deficient 3 | | | | |
| associated protein | | | | |
| Protein phosphatase 2, | J02902 | 0.48 | Cell cycle | NP_055040 |
| regulatory subunit A, alpha | | | | |
| isoform | | | | |
| Thyroid autoantigen 70 kDa | J04607 | 0.25 | Cell cycle | NP_001460 |
| A disintegrin and | R32760 | 0.37 | Cell signaling | |
| metalloproteinase domain 17 | | | | |
| A kinase anchor protein 13 | M90360 | 0.31 | Cell signaling | NP_658913 |
| Calpastatin | AF037194 | 0.39 | Cell signaling | NP_006471 |
| Diacylglycerol kinase, alpha | AF064770 | 0.44 | Cell signaling | NP_001336 |
| 80 kDa | | | | |
| gamma-aminobutyric acid B | AJ012187 | 0.42 | Cell signaling | NP_068705 |
| receptor, 1 | | | | |
| Inositol polyphosphate-5- | U84400 | 0.41 | Cell signaling | NP_005532 |
| phosphatase, 145 kDa | | | | |
| Lymphocyte-specific protein | X05027 | 0.45 | Cell signaling | NP_005347 |
| tyrosine kinase | | | | |
| RAP1B, member of RAS | P09526 | 0.4 | Cell signaling | P09526 |
| oncogene family | | | | |
| Ras association | AF061836 | 0.43 | Cell signaling | NP_733835 |
| (RalGDS/AF-6) domain | | | | |
| family 1 | | | | |
| CDC42-effector protein 3 | AF104857 | 0.28 | Cell signaling | NP_006440 |
| Leupaxin | AF062075 | 0.31 | Cell signaling | NP_004802 |
| Annexin A6 | D00510 | 0.45 | Cell structure | NP_004024 |
| RAN-binding protein 9 | AB008515 | 0.41 | Cell structure | NP_005484 |
| Thymosin, beta 10 | M20259 | 0.26 | Cell structure | NP_066926 |
| GranzymeA | M18737 | 0.17 | Cell/organism | NP_006135 |
| | | | defense | |
| ThromboxaneA synthase 1 | M80646 | 0.44 | Cell/organism | NP_112246 |
| | | | defense | |
| Coatomer protein complex, | AA357332 | 0.39 | Gene expression | NP_057535 |
| subunit beta | | | | |
| Cold-inducible RNA-binding | H39820 | 0.27 | Gene expression | NP_001271 |
| protein | | | | |
| Leucine-rich repeat | U69609 | 0.44 | Gene expression | NP_004726 |
| interacting protein 1 | | | | |
| Proteasome subunit, alpha | D00762 | 0.31 | Gene expression | NP_687033 |
| type, 3 | | | | |
| Proteasome subunit, alpha | AF022815 | 0.35 | Gene expression | NP_689468 |
| type, 7 | | | | |
| Protein phosphatase 1G, | AI417405 | 0.5 | Gene expression | NP_817092 |
| gamma isoform | | | | |
| Ribonuclease/angiogenin | M36717 | 0.44 | Gene expression | NP_002930 |
| inhibitor | | | | |
| RNA-binding protein- | AF021819 | 0.3 | Gene expression | NP_009193 |
| regulatory subunit | | | | |
| Signal transducer and | U16031 | 0.45 | Gene expression | NP_003144 |
| activator of transcription 6 | | | | |
| Transcription factor A, | M62810 | 0.41 | Gene expression | NP_036383 |
| mitochondrial | | | | |
| Ubiquitin-specific protease 4 | AF017306 | 0.31 | Gene expression | NP_003354 |
| Dehydrogenase/reductase | AA100046 | 0.46 | Metabolism | NP_612461 |
| SDR family member 1 | | | | |
| Solute carrier family 25, | J03592 | 0.3 | Metabolism | NP_001627 |
| member 6 | | | | |
| Amplified in osteosarcoma | U41635 | 0.45 | Unclassified | NP_006803 |
| Expressed in activated | C00577 | 0.45 | Unclassified | NP_009198 |
| T/LAK lymphocytes | | | | |
| Integral inner nuclear | W00460 | 0.4 | Unclassified | NP_055134 |
| membrane protein | | | | |
| Phosphodiesterase 4D- | T95969 | 0.45 | Unclassified | NP_055459 |
| interacting protein | | | | |
| Tumor endothelial marker 7 | N93789 | 0.45 | Unclassified | NP_065138 |
| precursor | | | | |
| Wiskott-Aldrich syndrome | AF031588 | 0.22 | Unclassified | NP_003378 |
| protein interacting protein | | | | |

### EXAMPLE 2

This example demonstrates the use of the claimed invention to identify biomarkers of hyperlipidimea and use of same. As used herein, a “biomarker” is any nucleic acid based substance that corresponds to, and can specifically identify a RNA transcript.

As used herein, “hyperlipidemia” is defined as an elevation of lipid protein profiles and includes the elevation of chylomicrons, very low-density lipoproteins (VLDL), intermediate-density lipoproteins (IDL), low-density lipoproteins (LDL), and/or high-density lipoproteins (HDL) as compared with the general population. Hyperlipidemia includes hypercholesterolemia and/or hypertriglyceridemia. By hypercholesterolemia, it is meant elevated fasting plasma total cholesterol level of >200 mg/dL, and/or LDL-cholesterol levels of >130 mg/dL. A desirable level of HDL-cholesterol is >60 mg/dL. By hypertriglyceridemia it is meant plasma triglyceride (TG) concentrations of greater than the 90^{th} or 95^{th} percentile for age and sex and can include, for example, TG>160 mg/dL as determined after an overnight fast.

The level of one or more RNA transcripts expressed in blood obtained from one or more individuals with hyperlipidemia was determined as follows. Whole blood samples were taken from patients who were diagnosed with hyperlipidemia as defined herein. In each case, the diagnosis of hyperlipidemia was corroborated by a skilled Board certified physician. Total mRNA from lysed blood was isolated using TRIzol® reagent (GIBCO). Fluorescently labeled probes for each blood sample were generated as described above. Each probe was denatured and hybridized to a 15K Chondrogene Microarray Chip (ChondroChip™) and/or an Affymetrix GeneChip® microarray as described herein. The presence of a fluorescent dye on the microarray indicates hybridization of a target nucleic acid and a specific nucleic acid member on the microarray. The intensities of fluorescence dye represent the amount of target nucleic acid which is hybridized to the nucleic acid member on the microarray, and is indicative of the expression level of the specific nucleic acid member sequence in the target sample.

Those transcripts which display differing levels with respect to the levels of those from patients unaffected by hyperlipidemia were identified as being biomarkers for said disease of interest. Identification of genes differentially expressed in whole blood samples from patients with hyperlipidemia as compared to healthy patients was determined by statistical analysis using the Wilcox Mann Whitney rank sum test.

Classification or class prediction of a test sample as either having hypertension and OA or being normal can be done using the differentially expressed genes as shown in Table 1A in combination with well known statistical algorithms for class prediction as would be understood by a person skilled in the art and described herein. Commercially available programs such as those provided by Silicon Genetics (e.g. GeneSpring™) for Class Predication are also available.

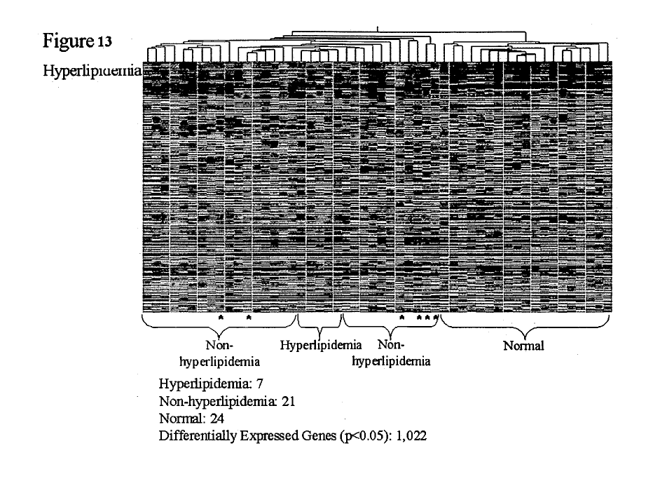

FIG. 13 shows a diagrammatic representation of RNA expression profiles of whole blood samples from individuals who were identified as having hyperlipidemia as described herein as compared with RNA expression profiles from normal and non-hyperlipidemia patients. Expression profiles were generated using GeneSpring™ software analysis as described herein. Each column represents the hybridization pattern resulting from a single individual. Normal individuals have no known medical conditions and were not taking any known medication. Non hyperlipidemia individuals presented without elevated cholesterol or elevated triglycerides but may have presented with other medical conditions and may be under various treatment regimes.

A dendogram analysis is shown above. Samples are clustered and marked as representing patients who have elevated lipids and/or cholesterol, are normal or do not have elevated lipids or cholesterol. The “*” indicates those patients who abnormally clustered as having either hyperlipidemia, normal or non-hyperlipidemia despite actual presentation. The number of hybridizations profiles determined for hyperlipidemia patients, non-hyperlipidemia patients and normal individuals are shown. Various experiments were performed as outlined above, and analyzed using either the Wilcox Mann Whitney rank sum test, or other statistical analysis as described herein and those genes identified with a p value of <0.05 as between the patients with hyperlipidemia as compared with patients without hyperlipidemia are shown in Table

Classification or class prediction of a test sample from an unknown patient in order to diagnose said individual with hyperlipidemia can be done using the differentially expressed genes as shown in Table 1D in combination with well known statistical algorithms for class prediction as would be understood by a person skilled in the art and is described herein. Commercially available programs such as those provided by Silicon Genetics (e.g. GeneSpring™) for Class Predication are also available.

In addition to Hyperlipidemia, biomarkers for the following diseases were identified using the above method steps to identify one or more genetic markers for the following diseases; Type II Diabetes, Hypertension, Obesity, Lung Disease, Bladder Cancer, Coronary Artery Disease, Rheumatoid Arthritis, Depression, Osteoarthritis, Liver Cancer, Schizophrenia, Chagas Disease, Asthma, Lung Cancer, Heart Failure, Psoriasis, Thyroid Disorder, Irritable Bowel Syndrome, Osteoporosis, Migraine Headaches, Eczema, NASH, Alzheimer's Disease, Manic Depression Syndrom, Crohn's Colitis, Chronic Cholecystits, Cervical Cancer, Stomach Cancer, Kidney Cancer, Testicular Cancer, Colon Cancer, Hepatitis B, and Pancreatic Cancer.

Diabetes

This example demonstrates the use of the claimed invention to identify biomarkers of diabetes and use of same.

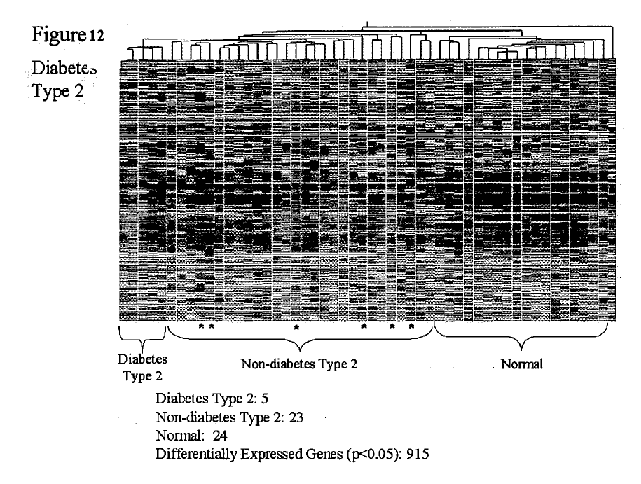

As used herein, “diabetes”, or “diabetes mellitus” includes both “type 1 diabetes” (insulin-dependent diabetes (IDDM)) and “type 2 diabetes” (insulin-independent diabetes (NIDDM). Both type 1 and type 2 diabetes characterized in accordance with Harrison's Principles of Internal Medicine 14th edition, as a person having a venous plasma glucose concentration≧140 mg/dL on at least two separate occasions after overnight fasting and venous plasma glucose concentration≧200 mg/dL at 2 h and on at least one other occasion during the 2-h test following ingestion of 75 g of glucose. Patients identified as having type 2 diabetes as described herein are those demonstrating insulin-independent diabetes as determined by the methods described above. Whole blood samples were taken from patients who were diagnosed with type 2 diabetes as defined herein. In each case, the diagnosis of type 2 diabetes was corroborated by a skilled Board certified physician. FIG. 12 shows a diagrammatic representation of RNA expression profilesRNA expression profiles of Whole blood samples from individuals who were identified as having type 2 diabetes as described herein as compared RNA expression profilesRNA expression profiles from individuals not having type 2 diabetes. RNA expression profilesRNA expression profiles were generated using GeneSpring™ software analysis as described herein. Hybridizations to create said RNA expression profilesRNA expression profiles were done using the 15K Chondrogene Microarray Chip (ChondroChip™) as described herein. Samples are clustered and marked as representing patients who have type 2 diabetes or control individuals. The number of hybridizations profiles determined for patients with type 2 diabetes or who are controls are shown. Various experiments were performed as outlined above, and analyzed using either the Wilcox Mann Whitney rank sum test, or other statistical tests as described herein, and those genes identified with a p value of <0.05 as between the patients with type 2 diabetes as compared with patients without type 2 diabetes are shown in Table 1P.

Classification or class prediction of a test sample from an unknown patient in order to diagnose said individual with type 2 diabetes can be done using the differentially expressed genes as shown in Table 1P in combination with well known statistical algorithms for class prediction as would be understood by a person skilled in the art and is described herein. Commercially available programs such as those provided by Silicon Genetics (e.g. GeneSpring™) for Class Predication are also available.

RNA Expression Profiles RNA Expression Profiles Lung Disease

This example demonstrates the use of the claimed invention to identify biomarkers of Lung Disease and use of same.

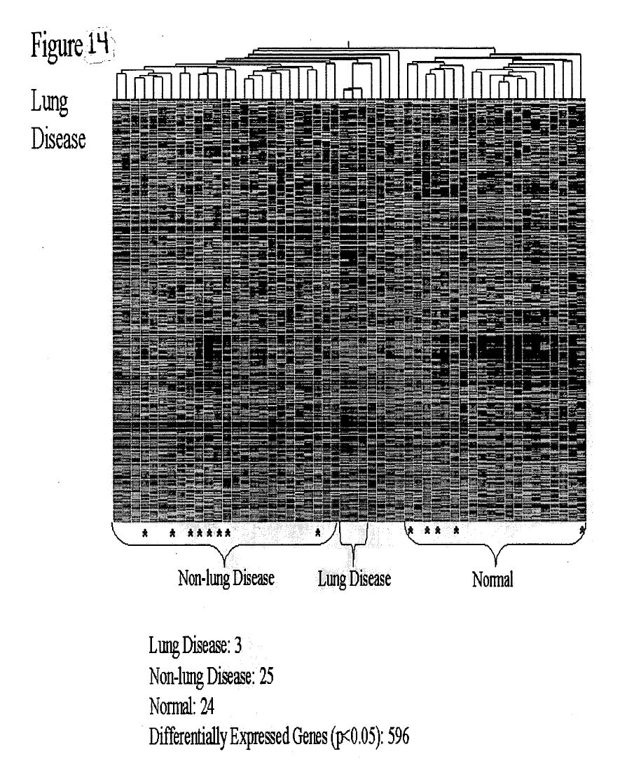

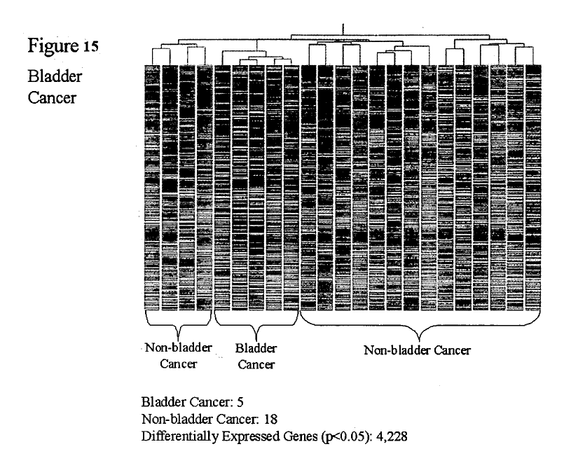

As used herein, “lung disease” encompasses any disease that affects the respiratory system and includes bronchitis, chronic obstructive lung disease, emphysema, asthma, and lung cancer. Patients identified as having lung disease includes patients having one or more of the above noted conditions. In each case, the diagnosis of lung disease was corroborated by a skilled Board certified physician. FIG. 14 shows a diagrammatic representation of RNA expression profilesRNA expression profiles of Whole blood samples from individuals who were identified as having lung disease as described herein as compared with RNA expression profilesRNA expression profiles from normal and non lung disease individuals. Samples are clustered and marked as representing patients who have lung disease, are normal or do not have lung disease. The “*” indicates those patients who abnormally clustered despite actual presentation. The number of hybridizations profiles determined for either the lung disease patients, non-lung disease patients and normal individuals are show. Various experiments were performed as outlined above, and analyzed using the Wilcox Mann Whitney rank sum test, or other statistical analysis as described herein and those genes identified with a p value of <0.05 as between the patients with lung disease as compared with patients without lung disease are shown in Table 1R.

Classification or class prediction of a test sample from an unknown patient in order to diagnose said individual with lung disease can be done using the differentially expressed genes as shown in Table 1R in combination with well known statistical algorithms for class prediction as would be understood by a person skilled in the art and is described herein. Commercially available programs such as those provided by Silicon Genetics (e.g. GeneSpring™) for Class Predication are also available.

Bladder Cancer

This example demonstrates the use of the claimed invention to identify biomarkers of bladder cancer and use of same.

As used herein, “bladder cancer” includes carcinomas that occur in the transitional epithelium lining the urinary tract, starting at the renal pelvis and extending through the ureter, the urinary bladder, and the proximal two-thirds of the urethra. As used herein, patients diagnosed with bladder cancer include patients diagnosed utilizing any of the following methods or a combination thereof: urinary cytologic evaluation, endoscopic evaluation for the presence of malignant cells, CT (computed tomography), MRI (magnetic resonance imaging) for metastasis status. In each case, the diagnosis of bladder cancer was corroborated by a skilled Board certified physician. FIG. 15 shows a diagrammatic representation of RNA expression profilesRNA expression profiles of Whole blood samples from individuals who were identified as having bladder cancer as described herein as compared with RNA expression profilesRNA expression profiles from non bladder cancer individuals. Expression profiles were generated using GeneSpring™ software analysis as described herein. Each column represents the hybridization pattern resulting from a single individual. Non bladder cancer individuals presented without bladder cancer, but may have presented with other medical conditions and may be under various treatment regimes. Hybridizations to create said RNA expression profilesRNA expression profiles were done using the Affymetrix U133A chip. A dendogram analysis is shown above. Samples are clustered and marked as representing patients who have bladder cancer, or do not have bladder cancer. The “*” indicates those patients who abnormally clustered as either bladder cancer, or non bladder cancer despite actual presentation. The number of hybridizations profiles determined for patients with bladder cancer and without bladder cancer to create said Figure are shown. Various experiments were performed as outlined above, and analyzed using either the Wilcox Mann Whitney rank sum test, or other statistical tests as described herein and those genes identified with a p value of <0.05 as between the patients with bladder cancer as compared with patients without bladder cancer are shown in Tables 1S. Classification or class prediction of a test sample from an unknown patient in order to diagnose said individual with bladder cancer can be done using the differentially expressed genes as shown in Table 1S in combination with well known statistical algorithms for class prediction as would be understood by a person skilled in the art and is described herein. Commercially available programs such as those provided by Silicon Genetics (e.g. GeneSpring™) for Class Predication are also available.

Coronary Artery Disease

This example demonstrates the use of the claimed invention to identify biomarkers of coronary artery disease and use of same.

As used herein, “Coronary artery disease” (CAD) is defined as a condition wherein at least one coronary artery has >50% luminal diameter stenosis, as diagnosed by coronary angiography and includes conditions in which there is atheromatous narrowing and subsequent occlusion of the vessel. CAD includes those conditions which manifest as angina, silent ischaemia, unstable angina, myocardial infarction, arrhythmias, heart failure, and sudden death. Patients identified as having CAD includes patients having one or more of the above noted conditions. In each case, the diagnosis of Coronary artery disease was corroborated by a skilled Board certified physician. FIG. 17 shows a diagrammatic representation of RNA expression profilesRNA expression profiles of Whole blood samples from individuals who were identified as having coronary artery disease (CAD) as described herein as compared with RNA expression profilesRNA expression profiles from non-coronary artery disease individuals. Expression profiles were generated using GeneSpring™ software analysis as described herein. Each column represents the hybridization pattern resulting from a single individual. Non coronary artery disease individuals presented without coronary artery disease, but may have presented with other medical conditions and may be under various treatment regimes. Hybridizations to create said RNA expression profilesRNA expression profiles were done using the Affymetrix U133A chip. A dendogram analysis is shown above. Samples are clustered and marked as representing patients who have coronary artery disease or do not have coronary artery disease. The “*” indicates those patients who abnormally clustered despite actual presentation. The number of hybridizations profiles determined for patients with CAD or without CAD are shown. Various experiments were performed as outlined above, and analyzed using either the Wilcox Mann Whitney rank sum test, or other statistical analysis as described herein and those genes identified with a p value of <0.05 as between the patients with coronary artery disease as compared with patients without coronary artery disease are shown in Table 1U.

Classification or class prediction of a test sample from an unknown patient in order to diagnose said individual with CAD can be done using the differentially expressed genes as shown in Table 1U in combination with well known statistical algorithms for class prediction as would be understood by a person skilled in the art and is described herein. Commercially available programs such as those provided by Silicon Genetics (e.g. GeneSpring™) for Class Predication are also available.

Rheumatoid Arthritis

This example demonstrates the use of the claimed invention to identify biomarkers of rheumatoid arthritis and use of same.

As used herein “Rheumatoid Arthritis” (RA) is defined as a chronic, multisystem disease of unknown etiology with the characteristic feature of persistent inflammatory synovitis. Said inflammatory synovitis usually involves peripheral joints in a systemic distribution. Patients having RA as defined herein were identified as having one or more of the following; (i) cartilage destruction, (ii) bone erosions and/or (iii) joint deformities. Whole blood samples were taken from patients who were diagnosed Rheumatoid arthritis as defined herein. In each case, the diagnosis of Rheumatoid arthritis was corroborated by a skilled Board certified physician. FIG. 18 shows a diagrammatic representation of RNA expression profilesRNA expression profiles of Whole blood samples from individuals who were identified as having rheumatoid arthritis as described herein as compared with RNA expression profilesRNA expression profiles from non-rheumatoid arthritis individuals. Expression profiles were generated using GeneSpring™ software analysis as described herein. Each column represents the hybridization pattern resulting from a single individual. Normal individuals have no known medical conditions and were not taking any known medication. Non rheumatoid arthritis individuals presented without rheumatoid arthritis, but may have presented with other medical conditions and may be under various treatment regimes. Hybridizations to create said RNA expression profilesRNA expression profiles were done using ChondroChip™ and Affymetrix U133A Chip. A dendogram analysis using the ChondroChip is shown above. Samples are clustered and marked as representing patients who have rheumatoid arthritis or do not have rheumatoid arthritis. The “*” indicates those patients who abnormally clustered despite actual presentation. The number of hybridizations profiles determined for patients with rheumatoid arthritis and without rheumatoid arthritis are shown. Various experiments were performed as outlined above and analyzed using either the Wilcox Mann Whitney rank sum test, or other statistical tests as described herein and those genes identified with a p value of <0.05 as between the patients with rheumatoid arthritis as compared with patients without rheumatoid arthritis are shown. Data generated using the ChondroChip™ array is shown in Table 1V whereas data generated using the Affymetrix U133A Chip is shown in Table 1W.

Classification or class prediction of a test sample from an unknown patient in order to diagnose said individual with rheumatoide arthritis can be done using the differentially expressed genes as shown in Table 1V and 1W in combination with well known statistical algorithms for class prediction as would be understood by a person skilled in the art and is described herein. Commercially available programs such as those provided by Silicon Genetics (e.g. GeneSpring™) for Class Predication are also available.

Depression

This example demonstrates the use of the claimed invention to identify biomarkers of depression and use of same.

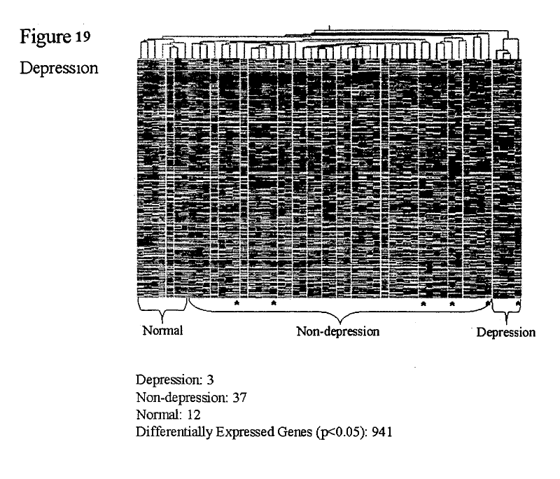

As used herein “depression” includes depressive disorders or depression in association with medical illness or substance abuse in addition to depression as a result of sociological situations. Patients defined as having depression were diagnosed mainly on the basis of clinical symptoms including a depressed mood episode wherein a person displays a depressed mood on a daily basis for a period of greater than 2 weeks. A depressed mood episode may be characterized by sadness, indifference, apathy, or irritability and is usually associated with changes in a number of neurovegetative functions, including sleep patterns, appetite and weight, fatigue, impairment in concentration and decision making. Whole blood samples were taken from patients who were diagnosed with depression as defined herein. In each case, the diagnosis of depression was corroborated by a skilled Board certified physician. FIG. 19 shows a diagrammatic representation of RNA expression profilesRNA expression profiles of Whole blood samples from individuals who were identified as having depression as described herein as compared with RNA expression profilesRNA expression profiles from non-depression individuals. Expression profiles were generated using GeneSpring™ software analysis as described herein. Each column represents the hybridization pattern resulting from a single individual. Normal individuals have no known medical conditions and were not taking any known medication. Non depression individuals presented without depression, but may have presented with other medical conditions and may be under various treatment regimes. Hybridizations to create said RNA expression profilesRNA expression profiles were done using ChondroChip™. A dendogram analysis is shown above. Samples are clustered and marked as representing patients who have depression, having non-depression or normal. The “*” indicates those patients who abnormally clustered despite actual presentation. The number of hybridizations profiles determined for patients with depression, non-depression and normal are shown. Various experiments were performed as outlined above, and analyzed using either the Wilcox Mann Whitney rank sum test, or other statistical tests as described herein and those genes identified with a p value of <0.05 as between the patients with depression as compared with patients without depression are shown in Table 1X.

Classification or class prediction of a test sample from an unknown patient in order to diagnose said individual with depression can be done using the differentially expressed genes as shown in Table 1X in combination with well known statistical algorithms for class prediction as would be understood by a person skilled in the art and is described herein. Commercially available programs such as those provided by Silicon Genetics (e.g. GeneSpring™) for Class Predication are also available.

Osteoarthritis

This example demonstrates the use of the claimed invention to identify biomarkers which differentiate various stages of Osteoarthritis and use of same.

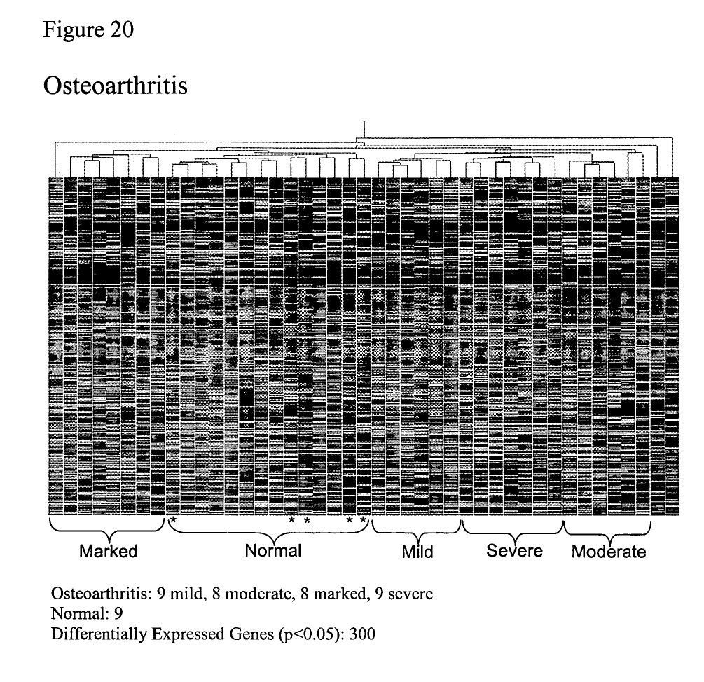

“Osteoarthritis” (OA), as used herein also known as “degenerative joint disease”, represents failure of a diarthrodial (movable, synovial-lined) joint. It is a condition, which affects joint cartilage, and or subsequently underlying bone and supporting tissues leading to pain, stiffness, movement problems and activity limitations. It most often affects the hip, knee, foot, and hand, but can affect other joints as well. OA severity can be graded according to the system described by Marshall (Marshall K W. J Rheumatol, 1996:23(4) 582-85). Briefly, each of the six knee articular surfaces was assigned a cartilage grade with points based on the worst lesion seen on each particular surface. Grade 0 is normal (0 points), Grade I cartilage is soft or swollen but the articular surface is intact (1 point). In Grade II lesions, the cartilage surface is not intact but the lesion does not extend down to subchondral bone (2 points). Grade III damage extends to subchondral bone but the bone is neither eroded nor eburnated (3 points). In Grade IV lesions, there is eburnation of or erosion into bone (4 points). A global OA score is calculated by summing the points from all six cartilage surfaces. If there is any associated pathology, such as meniscus tear, an extra point will be added to the global score. Based on the total score, each patient is then categorized into one of four OA groups: mild (1-6), moderate (7-12), marked (13-18), and severe (>18). As used herein, patients identified with OA may be categorized in any of the four OA groupings as described above. Whole blood samples were taken from patients who were diagnosed with osteoarthritis and a specific stage of osteoarthritis as defined herein. In each case, the diagnosis of osteoarthritis and the stage of osteoarthritis was corroborated by a skilled Board certified physician. FIG. 20 shows a diagrammatic representation of RNA expression profilesRNA expression profiles of Whole blood samples from individuals having various stages of osteoarthritis as compared with RNA expression profilesRNA expression profiles from normal individuals. Expression profiles were generated using GeneSpring™ software analysis as described herein. Each column represents the hybridization pattern resulting from a single individual. Normal individuals have no known medical conditions and were not taking any known medication. Hybridizations to create said RNA expression profilesRNA expression profiles were done using the ChondroChip™. A dendogram analysis is shown above. Samples are clustered and marked as representing patients who presented with different stages of osteoarthritis or normal. The “*” indicates those patients who abnormally clustered despite actual presentation. The number of hybridizations profiles determined for either osteoarthritis patients or normal individuals are shown in FIG. 20. Statistical analysis was done using an ANOVA test and those genes identified with a p value of <0.05 in pairwise comparisons between patients with mild, moderate, marked, severe or no osteoarthritis as shown in Table 1Y.

Liver Cancer

This example demonstrates the use of the claimed invention to identify biomarkers of liver cancer and use of same.

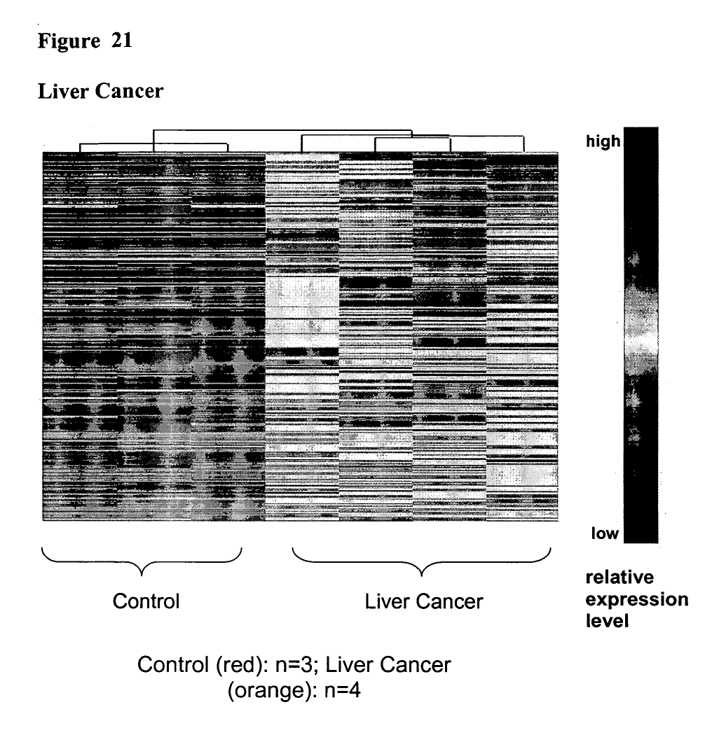

As used herein, “liver cancer” means primary liver cancer wherein the cancer initiates in the liver. Primary liver cancer includes both hepatomas or hepatocellular carcinomas (HCC) which start in the liver and chonalgiomas where cancers develop in the bile ducts of the liver. Whole blood samples were taken from patients who were diagnosed with liver cancer as defined herein. In each case, the diagnosis of liver cancer was corroborated by a skilled Board certified physician. FIG. 21 shows a diagrammatic representation of RNA expression profilesRNA expression profiles of Whole blood samples from individuals who were identified as having liver cancer as described herein as compared with RNA expression profilesRNA expression profiles from non-liver cancer disease individuals. Expression profiles were generated using GeneSpring™ software analysis as described herein. Each column represents the hybridization pattern resulting from a single individual. Control samples presented without liver cancer but may have presented with other medical conditions and may be under various treatment regimes. Hybridizations to create said RNA expression profilesRNA expression profiles were done using the Affymetrix® U133A chip. A dendogram analysis is shown above. Samples are clustered and marked as representing patients who have liver cancer or control. The number of hybridizations profiles determined for patients with liver cancer or who are controls are shown. Various experiments were performed as outlined above, and analyzed using either the Wilcox Mann Whitney rank sum test, or other statistical tests as described herein, and those genes identified with a p value of <0.05 as between the patients with liver cancer as compared with patients without liver cancer are shown in Table 1Z.

Classification or class prediction of a test sample from an unknown patient in order to diagnose said individual with liver cancer can be done using the differentially expressed genes as shown in Table 1Z in combination with well known statistical algorithms for class prediction as would be understood by a person skilled in the art and is described herein. Commercially available programs such as those provided by Silicon Genetics (e.g. GeneSpring™) for Class Prediction are also available.

Schizophrenia

This example demonstrates the use of the claimed invention to identify biomarkers of diabetes and use of same.

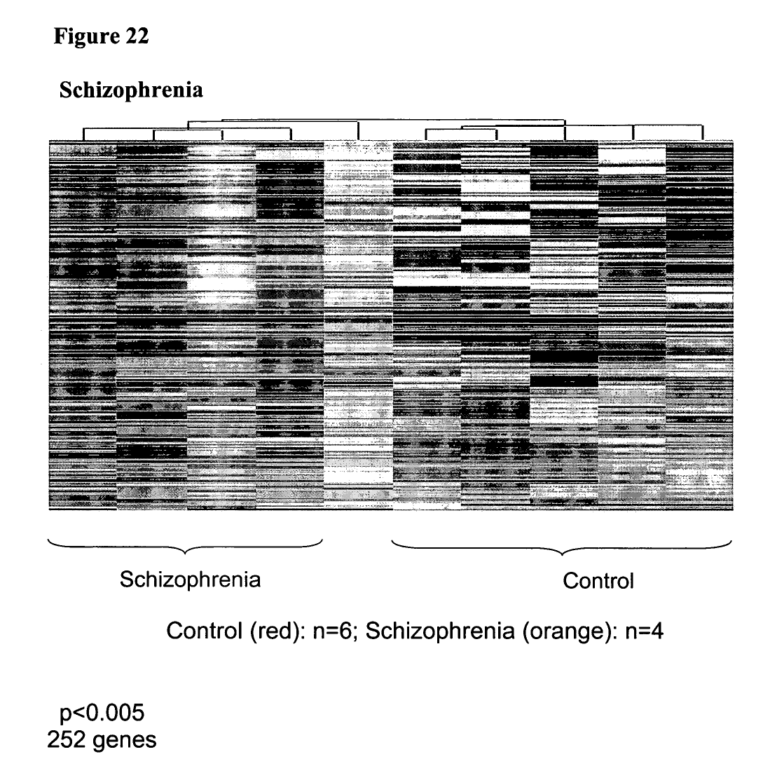

As used herein, “schizophrenia” is defined as a psychotic disorders characterized by distortions of reality and disturbances of thought and language and withdrawal from social contact. Patients diagnosed with “schizophrenia” can include patients having any of the following diagnosis: an acute schizophrenic episode, borderline schizophrenia, catatonia, catatonic schizophrenia, catatonic type schizophrenia, disorganized schizophrenia, disorganized type schizophrenia, hebephrenia, hebephrenic schizophrenia, latent schizophrenia, paranoic type schizophrenia, paranoid schizophrenia, paraphrenia, paraphrenic schizophrenia, psychosis, reactive schizophrenia or the like. Whole blood samples were taken from patients who were diagnosed with schizophrenia as defined herein. In each case, the diagnosis of schizophrenia was corroborated by a skilled Board certified physician. FIG. 22 shows a diagrammatic representation of RNA expression profilesRNA expression profiles of Whole blood samples from individuals who were identified as having schizophrenia as described herein as compared with RNA expression profilesRNA expression profiles from non schizophrenic individuals. Expression profiles were generated using GeneSpring™ software analysis as described herein. Each column represents the hybridization pattern resulting from a single individual. Control samples presented without schizophrenia but may have presented with other medical conditions and may be under various treatment regimes. Hybridizations to create said RNA expression profilesRNA expression profiles were done using the Affymetrix® U133A chip. A dendogram analysis is shown above. Samples are clustered and marked as representing patients who have schizophrenia or control individuals. The number of hybridizations profiles determined for patients with schizophrenia or who are controls are shown. Various experiments were performed as outlined above, and analyzed using either the Wilcox Mann Whitney rank sum test, or other statistical tests as described herein, and those genes identified with a p value of <0.05 as between the patients with schizophrenia as compared with patients without schizophrenia are shown in Table 1AA.

Classification or class prediction of a test sample from an unknown patient in order to diagnose said individual with schizophrenia can be done using the differentially expressed genes as shown in Table 1AA in combination with well known statistical algorithms for class prediction as would be understood by a person skilled in the art and is described herein. Commercially available programs such as those provided by Silicon Genetics (e.g. GeneSpring™) for Class Prediction are also available.

Chagas Disease

This example demonstrates the use of the claimed invention to identify biomarkers of Chagas' disease and use of same.

As used herein, “Chagas' disease” is defined as a condition wherein an individual is infected with the protozoan parasite Trypanosoma cruzi and includes both acute and chronic infection. Acute infection with T. cruzi can be diagnosed by detection of parasites by either microscopic examination of fresh anticoagulated blood or the buffy coat, giemsa-stained thin and thick blood smears and/or mouse inoculation and culturing of the blood of a potentially infected individual. Even in the absence of a positive result from the above, an accurate determination of infection can be made by xenodiagnosis wherein reduviid bugs are allowed to feed on the patient's blood and subsequently the bugs are examined for infection. Chronic infection can be determined by detection of antibodies specific to the T. cruzi antigens and/or immunoprecipitation and electrophoresis of the T. cruzi antigens.

As used herein “Symptomatic Chagas disease” includes symptomatic acute chagas and symptomatic chronic chagas disease. Acute symptomatic chagas disease can be characterized by one or more of the following: area of erythema and swelling (a chagoma); local lymphadenopathy; generalized lymphadenopathy; mild hepatosplenomegaly; unilateral painless edema of the palpebrae and periocular tissues; malaise; fever; anorexia and/or edema of the face and lower extremities. Symptomatic chronic Chagas' disease include one or more of the following symptoms: heart rhythm disturbances, cardiomyopathy, thromboembolism, electrocardiographic abnormalities including right bundle-branch blockage; atrioventricular block; premature ventricular contractions and tachy- and bradyarrhythmias; dysphagia; odynophagia, chest pain; regurgitation; weight loss, cachexia and pulmonary infections.

As used herein “Asymptomatic Chagas disease” is meant to refer to individuals who are infected with T. cruzi but who do not show either acute or chronic symptoms of the disease.

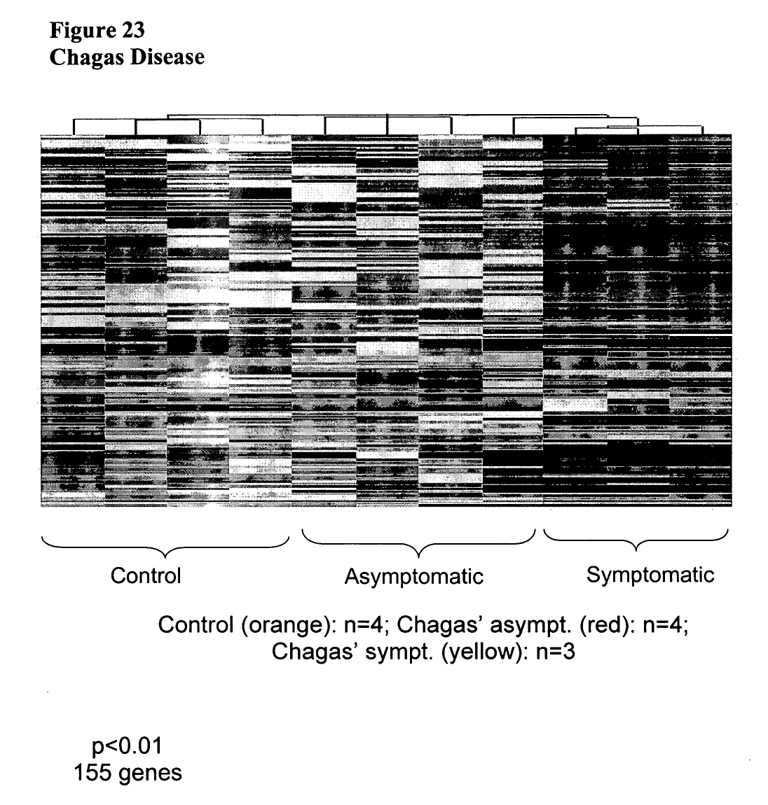

Whole blood samples were taken from patients who were diagnosed symptomatic or asymptomatic Chagas disease as defined herein. In each case, the diagnosis of Chagas disease was corroborated by a qualified physician. FIG. 23 shows a diagrammatic representation of RNA expression profilesRNA expression profiles of Whole blood samples from individuals who were identified as having symptomatic Chagas disease; asymptomatic Chagas disease or who were control individuals as described herein as compared with RNA expression profilesRNA expression profiles from individuals not having Chagas Disease. Expression profiles were generated using GeneSpring™ software analysis as described herein. Each column represents the hybridization pattern resulting from a single individual. Control samples presented without Chagas disease but may have presented with other medical conditions and may be under various treatment regimes. Hybridizations to create said RNA expression profilesRNA expression profiles were done using the Affymetrix® U133A chip. A dendogram analysis is shown above. Samples are clustered and marked as representing patients who have symptomatic chagas disease; asymptomatic chagas disease or control. The number of hybridizations profiles determined for patients with chagas disease; asymptomatic chagas disease or who are controls are shown. Various experiments were performed as outlined above, and analyzed using either the Wilcox Mann Whitney rank sum test, or other statistical tests as described herein. Those genes identified with a p value of <0.05 as between the patients with Chagas disease as compared with patients without Chagas disease are shown in Table 1AB. Those genes identified with a p value of <0.05 as between the patients with Asymptomatic Chagas disease as compared with patients without Chagas disease are shown in Table 5T. Those genes identified with a p value of <0.05 as between the patients with Symptomatic Chagas disease as compared with patients without Chagas disease are shown in Table 5U.

Classification or class prediction of a test sample from an unknown patient in order to diagnose said individual with symptomatic chagas disease can be done using the differentially expressed genes as shown in Table 5U in combination with well known statistical algorithms for class prediction as would be understood by a person skilled in the art and is described herein. Commercially available programs such as those provided by Silicon Genetics (e.g. GeneSpring™) for Class Prediction are also available. Classification or class prediction of a test sample from an unknown patient in order to diagnose said individual with asymptomatic chagas disease can be done using the differentially expressed genes as shown in Table 5T.

Asthma

This example demonstrates the use of the claimed invention to identify biomarkers of asthma disease and use of same.

As used herein, “asthma” indicates a chronic disease of the airways in the lungs characterized by constriction (the tightening of the muscles surrounding the airways) and inflammation (the swelling and irritation of the airways). Together constriction and inflammation cause narrowing of the airways, which results in symptoms such as wheezing, coughing, chest tightness, and shortness of breath. Whole blood samples were taken from patients who were diagnosed with asthma as defined herein. In each case, the diagnosis of asthma was corroborated by a skilled Board certified physician. Expression profiles were generated using GeneSpring™ software analysis as described herein. Each column represents the hybridization pattern resulting from a single individual. Hybridizations to create said RNA expression profilesRNA expression profiles were done using the ChondroChip™ and the Affymetrix Chip. Samples are clustered and marked as representing patients who have asthma or control individuals. The number of hybridizations profiles determined for patients with asthma and controls are shown. Various experiments were performed as outlined above, and analyzed using either the Wilcox Mann Whitney rank sum test, or other statistical tests as described herein. Those genes identified with a p value of <0.05 as between the patients with asthma as compared with patients without asthma using the ChondroChip™ are shown in Table 1AD. Those genes identified with a p value of <0.05 as between the patients with asthma as compared with patients without asthma using the Affymetrix® platform are shown in Table 1AE.

Classification or class prediction of a test sample from an unknown patient in order to diagnose said individual with asthma can be done using the differentially expressed genes as shown in Table 1AD and Table 1AE in combination with well known statistical algorithms for class prediction as would be understood by a person skilled in the art and is described herein. Commercially available programs such as those provided by Silicon Genetics (e.g. GeneSpring™) for Class Prediction are also available.

Hypertension

This example demonstrates the use of the claimed invention to identify biomarkers of hypertension and use of same.

As used herein, “hypertension” is defined as high blood pressure or elevated arterial pressure. Patients identified with hypertension herein include persons who have an increased risk of developing a morbid cardiovascular event and/or persons who benefit from medical therapy designed to treat hypertension. Patients identified with hypertension also can include persons having systolic blood pressure of >130 mm Hg or a diastolic blood pressure of >90 mm Hg or a person takes antihypertensive medication. Whole blood samples were taken from patients who were diagnosed with hypertension as defined herein. In each case, the diagnosis of hypertension was corroborated by a skilled Board certified physician. FIG. 5 shows a diagrammatic representation of RNA expression profilesRNA expression profiles of Whole blood samples from individuals who were identified as having hypertension as described herein as compared with RNA expression profilesRNA expression profiles from non hypertensive individuals and normal individuals. Expression profiles were generated using GeneSpring™ software analysis as described herein. Each column represents the hybridization pattern resulting from a single individual. Non hypertensive individuals presented without hypertension but may have presented with other medical conditions and may be under various treatment regimes. Normal individuals presented without any known conditions. Hybridizations to create said RNA expression profilesRNA expression profiles were done using a 15K Chondrogene Microarray Chip (ChondroChip™) as described herein. Samples are clustered and marked as representing patients who have hypertension or control individuals. The number of hybridizations profiles determined for patients with hypertension, without hypertension or who are controls are shown in FIG. 5. Various experiments were performed as outlined above, and analyzed using either the Wilcox Mann Whitney rank sum test, or other statistical tests as described herein, and those genes identified with a p value of <0.05 as between the patients with hypertension as compared with patients without hypertension are shown in Table 1E. Table 1AG shows those genes identified with a p value of <0.05 as between the patients with hypertension as compared with patients without hypertension from gene expressions profiles generated by analogous experiments using the Affymetrix® GeneChip®.

Classification or class prediction of a test sample from an unknown patient in order to diagnose said individual with hypertension can be done using the differentially expressed genes as shown in Table 1E and 1AG in combination with well known statistical algorithms for class prediction as would be understood by a person skilled in the art and is described herein. Commercially available programs such as those provided by Silicon Genetics (e.g. GeneSpring™) for Class Prediction are also available.

Obesity

This example demonstrates the use of the claimed invention to identify biomarkers of obesity and use of same.

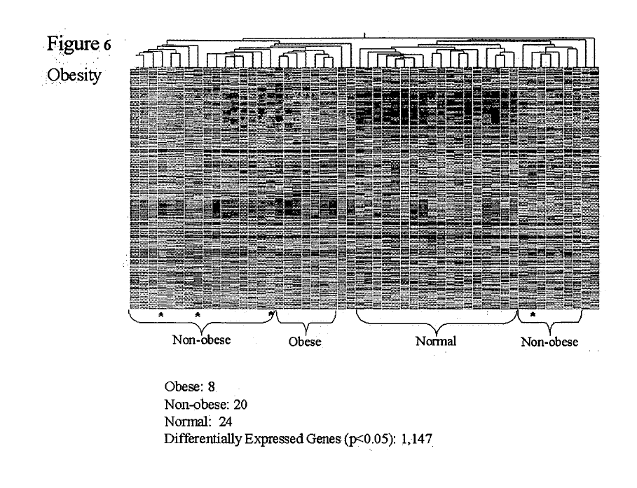

As used herein, “obesity” is defined as an excess of adipose tissue that imparts a health risk. Obesity is assessed in terms of height and weight in the relevance of age. Patients who are considered obese include, but are not limited to, patients having a body mass index or BMI ((defined as body weight in kg divided by (height in meters)²) greater than or equal to 30.0. Patients having obesity as defined herein are those with a BMI of greater than or equal to 30.0. Whole blood samples were taken from patients who were diagnosed with obesity as defined herein. In each case, the diagnosis of obesity was corroborated by a skilled Board certified physician. FIG. 6 shows a diagrammatic representation of RNA expression profilesRNA expression profiles of Whole blood samples from individuals who were identified as having obesity as described herein as compared with RNA expression profilesRNA expression profiles from non obese individuals. RNA expression profilesRNA expression profiles were generated using GeneSpring™ software analysis as described herein. Hybridizations to create said RNA expression profilesRNA expression profiles were done using the 15K Chondrogene Microarray Chip (ChondroChip™) as described herein. Samples are clustered and marked as representing patients who have obesity, those who are not obese, and normal individuals. The number of hybridizations profiles determined for patients with obesity, were not obese, and normal individuals are shown. Various experiments were performed as outlined above, and analyzed using either the Wilcox Mann Whitney rank sum test, or other statistical tests as described herein, and those genes identified with a p value of <0.05 as between the patients with obesity as compared with patients without obesity are shown in Table 1F. Table 1AH shows those genes identified with a p value of <0.05 as between the patients with obesity as compared with patients without obesity from gene expressions profiles generated by analogous experiments using the Affymetrix® GeneChip® platforms (U133A and U133 Plus 2.0).

Classification or class prediction of a test sample from an unknown patient in order to diagnose said individual with obesity can be done using the differentially expressed genes as shown in Table 1F in combination with well known statistical algorithms for class prediction as would be understood by a person skilled in the art and is described herein. Commercially available programs such as those provided by Silicon Genetics (e.g. GeneSpring™) for Class Prediction are also available.

Psoriasis

This example demonstrates the use of the claimed invention to identify biomarkers of psoriasis and use of same.

As used herein, “psoriasis” is defined as a common multifactorial inherited condition characterized by the eruption of circumscribed, discrete and confluent, reddish, silvery-scaled maculopapules; the lesions occur predominantly on the elbows, knees, scalp, and trunk, and microscopically show characteristic parakeratosis and elongation of rete ridges with shortening of epidermal keratinocyte transit time due to decreased cyclic guanosine monophosphate, according to Stedman's Online Medical Dictionary, 27th Edition. Whole blood samples were taken from patients who were diagnosed with psoriasis as defined herein. In each case, the diagnosis of psoriasis was corroborated by a skilled Board certified physician. RNA expression profilesRNA expression profiles of Whole blood samples from individuals who were identified as having psoriasis as opposed to not having psoriasis as described herein were generated using GeneSpring™ software analysis as described herein. Hybridizations to create said RNA expression profilesRNA expression profiles were done using the Affymetrix® GeneChip® platforms (U133A and U133 Plus 2.0) as described herein (data not shown). Various experiments were performed as outlined above, and analyzed using either the Wilcox Mann Whitney rank sum test, or other statistical tests as described herein, and those genes identified with a p value of <0.05 as between the patients with psoriasis as compared with patients without psoriasis are shown in Table 5A.

Classification or class prediction of a test sample from an unknown patient in order to diagnose said individual with psoriasis can be done using the differentially expressed genes as shown in Table 5A in combination with well known statistical algorithms for class prediction as would be understood by a person skilled in the art and is described herein. Commercially available programs such as those provided by Silicon Genetics (e.g. GeneSpring™) for Class Prediction are also available.

Thyroid Disorder

This example demonstrates the use of the claimed invention to identify biomarkers of thyroid disorder and use of same.

As used herein, “thyroid disorder” is defined as an overproduction of thyroid hormone (hyperthyroidism), underproduction of thyroid hormone (hypothyroidism), benign (noncancerous) thyroid disease, and thyroid cancer. Thyroid disorders include Anaplastic carcinoma of the thyroid, Chronis thyroiditis (Hashimoto's disease), colloid nodular goiter, hyperthyroidism, hyperpituitarism, hypothyridism-primary, hypothyridism-secondary, medullary thyroid carcinoma, painless (silent) thyroiditis, papillary carcinoa of the thyroid, subacute thyroiditis, thyroid cancer and congenital goiter, according to MEDLINE plus Illustrated Medical Encyclopedia. Whole blood samples were taken from patients who were diagnosed with a thyroid disorder as defined herein. In each case, the diagnosis of a thyroid disorder was corroborated by a skilled Board certified physician. RNA expression profilesRNA expression profiles of Whole blood samples from individuals who were identified as having a thyroid disorder as described herein were generated using GeneSpring™ software analysis as described herein. Hybridizations to create said RNA expression profilesRNA expression profiles were done using the Affymetrix® GeneChip® platforms (U133A and U133 Plus 2.0) as described herein (data not shown). Various experiments were performed as outlined above, and analyzed using either the Wilcox Mann Whitney rank sum test, or other statistical tests as described herein, and those genes identified with a p value of <0.05 as between the patients with a thyroid disorder as compared with patients without a thyroid disorder are shown in Table 5B.

Classification or class prediction of a test sample from an unknown patient in order to diagnose said individual with thyroid disorder can be done using the differentially expressed genes as shown in Table 5B in combination with well known statistical algorithms for class prediction as would be understood by a person skilled in the art and is described herein. Commercially available programs such as those provided by Silicon Genetics (e.g. GeneSpring™) for Class Prediction are also available.

Irritable Bowel Syndrome

This example demonstrates the use of the claimed invention to identify biomarkers of irritable bowel syndrome and use of same.

As used herein, “irritable bowel syndrome” is defined as a common gastrointestinal disorder involving an abnormal condition of gut contractions (motility) characterized by abdominal pain, bloating, mucous in stools, and irregular bowel habits with alternating diarrhea and constipation, symptoms that tend to be chronic and to wax and wane over the years, according to MedicineNet, Inc., an online, healthcare media publishing company. Whole blood samples were taken from patients who were diagnosed with irratble bowel syndrome as defined herein. In each case, the diagnosis of irratble bowel syndrome was corroborated by a skilled Board certified physician. RNA expression profilesRNA expression profiles of Whole blood samples from individuals who were generated using GeneSpring™ software analysis as described herein. Hybridizations to create said RNA expression profilesRNA expression profiles were done using the Affymetrix® GeneChip® platforms (U133A and U133 Plus 2.0) as described herein (data not shown). Samples are clustered and marked as representing patients who have irratble bowel syndrome or control individuals. Various experiments were performed as outlined above, and analyzed using either the Wilcox Mann Whitney rank sum test, or other statistical tests as described herein, and those genes identified with a p value of <0.05 as between the patients with irritable bowel syndrome as compared with patients without irratble bowel syndrome are shown in Table 5C.

Classification or class prediction of a test sample from an unknown patient in order to diagnose said individual with irritable bowel syndrome can be done using the differentially expressed genes as shown in Table 5C in combination with well known statistical algorithms for class prediction as would be understood by a person skilled in the art and is described herein. Commercially available programs such as those provided by Silicon Genetics (e.g. GeneSpring™) for Class Prediction are also available.

Osteoporosis

This example demonstrates the use of the claimed invention to identify biomarkers of osteoporosis and use of same.

As used herein, “osteoporosis” is defined as a reduction in the quantity of bone or atrophy of skeletal tissue; an age-related disorder characterized by decreased bone mass and increased susceptibility to fractures, according to Stedman's Online Medical Dictionary, 27th Edition. Whole blood samples were taken from patients who were diagnosed with osteoporosis syndrome as defined herein. In each case, the diagnosis of osteoporosis was corroborated by a skilled Board certified physician. RNA expression profilesRNA expression profiles of Whole blood samples from individuals who were identified as having osteoporosis as described herein as compared with RNA expression profilesRNA expression profiles from individuals not having osteoporosis, were generated using GeneSpring™ software analysis as described herein. Hybridizations to create said RNA expression profilesRNA expression profiles were done using the Affymetrix® GeneChip® platforms (U133A and U133 Plus 2.0) as described herein (data not shown). Samples are clustered and marked as representing patients who have osteoporosis or control individuals. Various experiments were performed as outlined above, and analyzed using either the Wilcox Mann Whitney rank sum test or other statistical tests as described herein, and those genes identified with a p value of <0.05 as between the patients with osteoporosis as compared with patients without osteoporosis are shown in Table 5D.

Migraine Headaches

This example demonstrates the use of the claimed invention to identify biomarkers of migraine headaches and use of same.

As used herein, “Migraine Headaches” is defined as a symptom complex occurring periodically and characterized by pain in the head (usually unilateral), vertigo, nausea and vomiting, photophobia, and scintillating appearances of light. Classified as classic migraine, common migraine, cluster headache, hemiplegic migraine, ophthalmoplegic migraine, and ophthalmic migraine, according to Stedman's Online Medical Dictionary, 27th Edition. Whole blood samples were taken from patients who were diagnosed with migraine headaches as defined herein. In each case, the diagnosis of migraine headaches was corroborated by a skilled Board certified physician. RNA expression profiles of Whole blood samples from individuals who were identified as having migraine headaches as described herein as compared with RNA expression profiles from individuals not having migraine headaches, were generated using GeneSpring™ software analysis as described herein. Hybridizations to create said RNA expression profiles were done using the Affymetrix® GeneChip® platforms (U133A and U133 Plus 2.0) as described herein (data not shown). Samples are clustered and marked as representing patients who have migraine headaches or control individuals. Various experiments were performed as outlined above, and analyzed using either the Wilcox Mann Whitney rank sum test or other statistical tests as described herein, and those genes identified with a p value of <0.05 as between the patients with migraine headaches as compared with patients without migraine headaches are shown in Table 5E.

Classification or class prediction of a test sample from an unknown patient in order to diagnose said individual with migraine headaches can be done using the differentially expressed genes as shown in Table 5E in combination with well known statistical algorithms for class prediction as would be understood by a person skilled in the art and is described herein. Commercially available programs such as those provided by Silicon Genetics (e.g. GeneSpring™) for Class Prediction are also available.

Eczema

This example demonstrates the use of the claimed invention to identify biomarkers of eczema and use of same.

As used herein, “Eczema” is defined as inflammatory conditions of the skin, particularly with vesiculation in the acute stage, typically erythematous, edematous, papular, and crusting; followed often by lichenification and scaling and occasionally by duskiness of the erythema and, infrequently, hyperpigmentation; often accompanied by sensations of itching and burning; the vesicles form by intraepidermal spongiosis; often hereditary and associated with allergic rhinitis and asthma, according to Stedman's Online Medical Dictionary, 27th Edition. Whole blood samples were taken from patients who were diagnosed with eczema as defined herein. In each case, the diagnosis of eczema headaches was corroborated by a skilled Board certified physician. RNA expression profiles of Whole blood samples from individuals who were identified as having eczema as described herein as compared with RNA expression profiles from individuals not having eczema, were generated using GeneSpring™ software analysis as described herein. Hybridizations to create said RNA expression profiles were done using Affymetrix® GeneChip® platforms (U133A and U133 Plus 2.0) as described herein (data not shown). Samples are clustered and marked as representing patients who have eczema or control individuals. Various experiments were performed as outlined above, and analyzed using either the Wilcox Mann Whitney rank sum test or other statistical tests as described herein, and those genes identified with a p value of <0.05 as between the patients with eczema as compared with patients without eczema are shown in Table 5F.

Classification or class prediction of a test sample from an unknown patient in order to diagnose said individual with eczema can be done using the differentially expressed genes as shown in Table 5F in combination with well known statistical algorithms for class prediction as would be understood by a person skilled in the art and is described herein. Commercially available programs such as those provided by Silicon Genetics (e.g. GeneSpring™) for Class Prediction are also available.

Manic Depression Syndrome

This example demonstrates the use of the claimed invention to identify biomarkers of manic depression syndrome and use of same.

As used herein, “Manic Depression Syndrome (MDS)” refers to a mood disorder characterized by alternating mania and depression. Whole blood samples were taken from patients who were diagnosed with manic depression as defined herein. In each case, the diagnosis of manic depression was corroborated by a skilled Board certified physician. RNA expression profiles of Whole blood samples from individuals who were identified as having manic depression as described herein as compared with RNA expression profiles from individuals not having manic depression, were generated using GeneSpring™ software analysis as described herein. Hybridizations to create said RNA expression profiles were done using Affymetrix® GeneChip® platforms (U133A and U133 Plus 2.0) platforms (U133A and U133 Plus 2.0) as described herein (data not shown). Samples are clustered and marked as representing patients who have manic depression or control individuals. Various experiments were performed as outlined above, and analyzed using either the Wilcox Mann Whitney rank sum test or other statistical tests as described herein, and those genes identified with a p value of <0.05 as between the patients with manic depression syndrome as compared with patients without manic depression syndrome are shown in Table 5I.

Classification or class prediction of a test sample from an unknown patient in order to diagnose said individual with manic depression syndrome can be done using the differentially expressed genes as shown in Table 5I in combination with well known statistical algorithms for class prediction as would be understood by a person skilled in the art and is described herein. Commercially available programs such as those provided by Silicon Genetics (e.g. GeneSpring™) for Class Prediction are also available.

Crohn's Colitis

This example demonstrates the use of the claimed invention to identify biomarkers of Crohn's Colitis and use of same.

As used herein, “Crohn's Colitis” is defined as a chronic granulomatous inflammatory disease of unknown etiology, involving any part of the gastrointestinal tract from mouth to anus, but commonly involving the terminal ileum with scarring and thickening of the bowel wall; it frequently leads to intestinal obstruction and fistula and abscess formation and has a high rate of recurrence after treatment, according to Dorland's Illustrated Medical Dictionary. In each case, the diagnosis of Crohn's colitis was corroborated by a skilled Board certified physician. RNA expression profiles of Whole blood samples from individuals who were identified as having Crohn's colitis as described herein as compared with RNA expression profiles from individuals not having Crohn's colitis, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have Crohn's colitis or control individuals. Various experiments were performed as outlined above, and analyzed using either the Wilcox Mann Whitney rank sum test or other statistical tests as described herein, and those genes identified with a p value of <0.05 as between the patients with Crohn's colitis as compared with patients without Crohn's colitis are shown in Table 5J.

Classification or class prediction of a test sample from an unknown patient in order to diagnose said individual with Crohn's colitis can be done using the differentially expressed genes as shown in Table 5J in combination with well known statistical algorithms for class prediction as would be understood by a person skilled in the art and is described herein. Commercially available programs such as those provided by Silicon Genetics (e.g. GeneSpring™) for Class Prediction are also available.

Chronic Cholecystitis

This example demonstrates the use of the claimed invention to identify biomarkers of Chronic Cholecystitis and use of same.

As used herein, “Chronic cholecystitis” is defined as chronic inflammation of the gallbladder, usually secondary to lithiasis, with lymphocytic infiltration and fibrosis that may produce marked thickening of the wall, according to Stedman's Online Medical Dictionary, 27th Edition. In each case, the diagnosis of chronic cholecystitis was corroborated by a skilled Board certified physician. RNA expression profiles of Whole blood samples from individuals who were identified as having chronic cholecystitis as described herein as compared with RNA expression profiles from individuals not having chronic cholecystitis, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have chronic cholecystitis or control individuals. Various experiments were performed as outlined above, and analyzed using either the Wilcox Mann Whitney rank sum test or other statistical tests as described herein, and those genes identified with a p value of <0.05 as between the patients with chronic cholecystitis as compared with patients without chronic cholecystitis are shown in Table 5K.

Classification or class prediction of a test sample from an unknown patient in order to diagnose said individual with chronic cholecystitis can be done using the differentially expressed genes as shown in Table 5K in combination with well known statistical algorithms for class prediction as would be understood by a person skilled in the art and is described herein. Commercially available programs such as those provided by Silicon Genetics (e.g. GeneSpring™) for Class Prediction are also available.

Cervical Cancer

This example demonstrates the use of the claimed invention to identify biomarkers of cervical cancer and use of same.

As used herein, “Cervical Cancer” is defined as cancer of the uterine cervix, the portion of the uterus attached to the top of the vagina. Ninety percent of cervical cancers arise from the flattened or “squamous” cells covering the cervix. Most of the remaining 10% arise from the glandular, mucus-secreting cells of the cervical canal leading into the uterus. In each case, the diagnosis of cervical cancer was corroborated by a skilled Board certified physician. RNA expression profiles of Whole blood samples from individuals who were identified as having cervical cancer as described herein as compared with RNA expression profiles from individuals not having cervical cancer, were generated using GeneSpring™ software analysis as described herein. Hybridizations to create said RNA expression profiles were done using Affymetrix® GeneChip® platforms (U133A and U133 Plus 2.0) platforms (U133A and U133 Plus 2.0) as described herein (data not shown). Samples are clustered and marked as representing patients who have cervical cancer or control individuals. Various experiments were performed as outlined above, and analyzed using either the Wilcox Mann Whitney rank sum test or other statistical tests as described herein, and those genes identified with a p value of <0.05 as between the patients with cervical cancer as compared with patients without cervical cancer are shown in Table 5M.

Classification or class prediction of a test sample from an unknown patient in order to diagnose said individual with cervical cancer can be done using the differentially expressed genes as shown in Table 5M in combination with well known statistical algorithms for class prediction as would be understood by a person skilled in the art and is described herein. Commercially available programs such as those provided by Silicon Genetics (e.g. GeneSpring™) for Class Prediction are also available.

Stomach Cancer

This example demonstrates the use of the claimed invention to identify biomarkers of stomach cancer and use of same.

As used herein, “Stomach Cancer” is defined as are malignacies of the stomach, the most common type being adenocarcinoma. Stomach is divided into. Cancer can develop in any of five different layers of the stomach. In each case, the diagnosis of stomach cancer was corroborated by a skilled Board certified physician. RNA expression profiles of Whole blood samples from individuals who were identified as having stomach cancer as described herein as compared with RNA expression profiles from individuals not having stomach cancer, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have stomach cancer or control individuals. Various experiments were performed as outlined above, and analyzed using either the Wilcox Mann Whitney rank sum test or other statistical tests as described herein, and those genes identified with a p value of <0.05 as between the patients with stomach cancer as compared with patients without stomach cancer are shown in Table 5N.

Classification or class prediction of a test sample from an unknown patient in order to diagnose said individual with stomach cancer can be done using the differentially expressed genes as shown in Table 5N in combination with well known statistical algorithms for class prediction as would be understood by a person skilled in the art and is described herein. Commercially available programs such as those provided by Silicon Genetics (e.g. GeneSpring™) for Class Prediction are also available.

Kidney Cancer

This example demonstrates the use of the claimed invention to identify biomarkers of kidney cancer and use of same.

As used herein, “Kidney Cancer” is defined as are malignacies of the kidney, the most common type being renal cell carcinoma In each case, the diagnosis of kidney cancer was corroborated by a skilled Board certified physician. RNA expression profiles of Whole blood samples from individuals who were identified as having kidney cancer as described herein as compared with RNA expression profiles from individuals not having kidney cancer, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have stomach cancer or control individuals. Various experiments were performed as outlined above, and analyzed using either the Wilcox Mann Whitney rank sum test or other statistical tests as described herein, and those genes identified with a p value of <0.05 as between the patients with kidney cancer as compared with patients without kidney cancer are shown in Table 50.

Classification or class prediction of a test sample from an unknown patient in order to diagnose said individual with kidney cancer can be done using the differentially expressed genes as shown in Table 50 in combination with well known statistical algorithms for class prediction as would be understood by a person skilled in the art and is described herein. Commercially available programs such as those provided by Silicon Genetics (e.g. GeneSpring™) for Class Prediction are also available.

Testicular Cancer

This example demonstrates the use of the claimed invention to identify biomarkers of testicular cancer and use of same.

As used herein, “Testicular Cancer” is defined as an abnormal, rapid, and invasive growth of cancerous (malignant) cells in the testicles. In each case, the diagnosis of testicular cancer was corroborated by a skilled Board certified physician. RNA expression profiles of Whole blood samples from individuals who were identified as having testicular cancer as described herein were compared with RNA expression profiles from individuals not having testicular cancer, using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with testicular cancer as compared with patients without testicular cancer are shown in Table 5P

Classification or class prediction of a test sample from an unknown patient in order to diagnose said individual with testicular cancer can be done using the differentially expressed genes as shown in Table 5P in combination with well known statistical algorithms for class prediction as would be understood by a person skilled in the art and is described herein. Commercially available programs such as those provided by Silicon Genetics (e.g. GeneSpring™) for Class Prediction are also available.

Colon Cancer

This example demonstrates the use of the claimed invention to identify biomarkers of colon cancer and use of same.

As used herein, “Colon Cancer” is defined as cancer of the colon and includes carcinoma, which arises from the lining of the large intestine, and lymphoma, melanoma, carcinoid tumors, and sarcomas. In each case, the diagnosis of colon cancer was corroborated by a skilled Board certified physician. RNA expression profiles of Whole blood samples from individuals who were identified as having colon cancer as described herein as compared with RNA expression profiles from individuals not having colon cancer, were generated using GeneSpring™ software analysis as described herein. Hybridizations to create said RNA expression profiles were done using Affymetrix® GeneChip® platforms (U133A and U133 Plus 2.0) platforms (U133A and U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with colon cancer as compared with patients without colon cancer are shown in Table 5Q.

Classification or class prediction of a test sample from an unknown patient in order to diagnose said individual with colon cancer can be done using the differentially expressed genes as shown in Table 5Q in combination with well known statistical algorithms for class prediction as would be understood by a person skilled in the art and is described herein. Commercially available programs such as those provided by Silicon Genetics (e.g. GeneSpring™) for Class Prediction are also available.

Hepatitis B

This example demonstrates the use of the claimed invention to identify biomarkers of hepatitis B and use of same.

As used herein, “Hepatitis B” is a serious disease caused by hepatitis B virus (HBV) that attacks human liver. The virus can cause lifelong infection, cirrhosis (scarring) of the liver, liver cancer, liver failure, and death. HBV is transmitted horizontally by blood and blood products and sexual transmission. It is also transmitted vertically from mother to infant in the perinatal period. In each case, the diagnosis of hepatitis B was corroborated by a skilled Board certified physician. RNA expression profiles of Whole blood samples from individuals who were identified as having hepatitis as described herein as compared with RNA expression profiles from individuals not having hepatitis, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have hepatitis or control individuals. Various experiments were performed as outlined above, and analyzed using either the Wilcox Mann Whitney rank sum test or other statistical tests as described herein, and those genes identified with a p value of <0.05 as between the patients with hepatitis as compared with patients without hepatitis are shown in Table 5R.

Classification or class prediction of a test sample from an unknown patient in order to diagnose said individual with hepatitis B can be done using the differentially expressed genes as shown in Table 5R in combination with well known statistical algorithms for class prediction as would be understood by a person skilled in the art and is described herein. Commercially available programs such as those provided by Silicon Genetics (e.g. GeneSpring™) for Class Prediction are also available.

Pancreatic Cancer

This example demonstrates the use of the claimed invention to identify biomarkers of pancreatic cancer and use of same.

As used herein, “Pancreatic Cancer” is defined as cancer of the colon and includes carcinoma, which arises from the lining of the large intestine, and lymphoma, melanoma, carcinoid tumors, and sarcomas. In each case, the diagnosis of pancreatic cancer was corroborated by a skilled Board certified physician. RNA expression profiles of Whole blood samples from individuals who were identified as having pancreatic cancer as described herein as compared with RNA expression profiles from individuals not having pancreatic cancer, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with pancreatic cancer as compared with patients without pancreatic cancer are shown in Table 5S.

Classification or class prediction of a test sample from an unknown patient in order to diagnose said individual with pancreatic cancer can be done using the differentially expressed genes as shown in Table 5S in combination with well known statistical algorithms for class prediction as would be understood by a person skilled in the art and is described herein. Commercially available programs such as those provided by Silicon Genetics (e.g. GeneSpring™) for Class Prediction are also available.

Nonalcoholic Steatohepatitis (NASH)

This example demonstrates the use of the claimed invention to identify biomarkers of nonalcoholic steatohepatitis and use of same.

As used herein, “nonalcoholic steatohepatitis”, (NASH) is defined as an inflammatory disease of the liver associated with the accumulation of fat in the liver. NASH is of uncertain pathogenesis and histologically resembling alcoholic hepatitis, but occurring in nonalcoholic patients, most often obese women with non-insulin-dependent diabetes mellitus; clinically it is generally asymptomatic or mild, but fibrosis or cirrhosis may result. The diagnosis is confirmed by a liver biopsy. In each case, the diagnosis of nonalcoholic steatohepatitis was corroborated by a skilled Board certified physician. RNA expression profiles of Whole blood samples from individuals who were identified as having nonalcoholic steatohepatitis as described herein as compared with RNA expression profiles from individuals not having nonalcoholic steatohepatitis, were generated using GeneSpring™ software analysis as described herein. Hybridizations to create said RNA expression profiles were done using Affymetrix® GeneChip® platforms (U133A and U133 Plus 2.0) platforms (U133A and U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with nonalcoholic steatohepatitis as compared with patients without nonalcoholic steatohepatitis are shown in Table 5G.

Classification or class prediction of a test sample from an unknown patient in order to diagnose said individual with NASH can be done using the differentially expressed genes as shown in Table 5G in combination with well known statistical algorithms for class prediction as would be understood by a person skilled in the art and is described herein. Commercially available programs such as those provided by Silicon Genetics (e.g. GeneSpring™) for Class Prediction are also available.

Alzheimer's Disease

As used herein, “alzheimer's disease” refers to a degenerative disease of the central nervous system characterized especially by premature senile mental deterioration. In each case, the diagnosis of alzheimer's disease was corroborated by a skilled Board certified physician. RNA expression profiles of Whole blood samples from individuals who were identified as having Alzheimer's Diseaseas described herein as compared with RNA expression profiles from individuals not having alzheimer's disease, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have alzheimer's disease or control individuals. Various experiments were performed as outlined above, and analyzed using either the Wilcox Mann Whitney rank sum test or other statistical tests as described herein, and those genes identified with a p value of <0.05 as between the patients with alzheimer's disease as compared with patients without alzheimer's disease are shown in Table 5H.

Classification or class prediction of a test sample from an unknown patient in order to diagnose said individual with alzheimer's disease can be done using the differentially expressed genes as shown in Table 5H in combination with well known statistical algorithms for class prediction as would be understood by a person skilled in the art and is described herein. Commercially available programs such as those provided by Silicon Genetics (e.g. GeneSpring™) for Class Prediction are also available.

Heart Failure

This example demonstrates the use of the claimed invention to identify biomarkers of heart failure and use of same.

As used herein, “heart failure” is defined as an inadequacy of the heart so that as a pump it fails to maintain the circulation of blood, with the result that congestion and edema develop in the tissues; Heart failure is synonymous with congestive heart failure, myocardial insufficiency, cardiac insufficiency, cardiac failure, and includes right ventricular failure, forward heart failure, backward heart failure and left ventricular failure. Resulting clinical syndromes include shortness of breath or nonpitting edema, enlarged tender liver, engorged neck veins, and pulmonary rales in various combinations. In each case, the diagnosis of heart failure was corroborated by a skilled Board certified physician. RNA expression profiles of Whole blood samples from individuals who were identified as having heart failure described herein as compared with RNA expression profiles from individuals not having hearat failure, were generated using GeneSpring™ software analysis as described herein. Hybridizations to create said RNA expression profiles were done using Affymetrix® GeneChip® platforms (U133A and U133 Plus 2.0) platforms (U133A and U133 Plus 2.0) as described herein (data not shown). Samples are clustered and marked as representing patients who have heart failure or control individuals. Various experiments were performed as outlined above, and analyzed using either the Wilcox Mann Whitney rank sum test or other statistical tests as described herein, and those genes identified with a p value of <0.05 as between the patients with heart failure as compared with patients without heart failure are shown in Table 5L.

Classification or class prediction of a test sample from an unknown patient in order to diagnose said individual with heart failure can be done using the differentially expressed genes as shown in Table 5L in combination with well known statistical algorithms for class prediction as would be understood by a person skilled in the art and is described herein. Commercially available programs such as those provided by Silicon Genetics (e.g. GeneSpring™) for Class Prediction are also available.

Ankylosing Spondylitis

This example demonstrates the use of the claimed invention to identify biomarkers of ankylosing spondylitis and use of same.

As used herein “ankylosing spondylitis” refers to a chronic inflammatory disease that affects the joints between the vertebrae of the spine, and/or the joints between the spine and the pelvis and can eventually cause the affected vertebrae to fuse or grow together. In each case, the diagnosis of heart failure was corroborated by a skilled Board certified physician. RNA expression profiles of Whole blood samples from individuals who were identified as having heart failure described herein as compared with RNA expression profiles from individuals not having hearat failure, were generated using GeneSpring™ software analysis as described herein. Hybridizations to create said RNA expression profiles were done using the Affymetrix® GeneChip® platforms (U133A and U133 Plus 2.0) platforms (U133A and U133 Plus 2.0) as described herein (data not shown). Samples are clustered and marked as representing patients who have ankylosing spondylitis or control individuals. Various experiments were performed as outlined above, and analyzed using either the Wilcox Mann Whitney rank sum test or other statistical tests as described herein, and those genes identified with a p value of <0.05 as between the patients with ankylosing spondylitis as compared with patients without ankylosing spondylitis are shown in Table 1AI.

Classification or class prediction of a test sample from an unknown patient in order to diagnose said individual with ankylosing spondylitis can be done using the differentially expressed genes as shown in Table 1AI in combination with well known statistical algorithms for class prediction as would be understood by a person skilled in the art and is described herein. Commercially available programs such as those provided by Silicon Genetics (e.g. GeneSpring™) for Class Prediction are also available.

### EXAMPLE 3

In addition to methods to identify biomarkers associated with a condition, this invention also includes methods to identify biomarkers that can identify markers for a condition in an individual or group of individuals, despite the presence of one or more second conditions in the same individual or group of individuals. The invention also includes methods to identify biomarkers of a co-morbid condition. The following examples illustrate embodiments of methods comprising individuals presenting with Ostearthritis and various second conditions, but the invention is not limited to these sets of examples.

Osteoarthritis and Hypertension

ChondroChip™ Microarray Data Analysis of RNA expression profiles of Whole blood samples from co-morbid individuals having osteoarthritis and hypertension as compared with RNA expression profiles from normal individuals.

This example demonstrates the use of the claimed invention to detect biomarkers of patients with osteoarthritis and hypertension or use of same.

Whole blood samples were taken from patients who were diagnosed with osteoarthritis and hypertension as defined herein. RNA expression profiles were then analysed and compared to profiles from patients unaffected by any disease. In each case, the diagnosis of osteoarthritis and hypertension was corroborated by a skilled Board certified physician.

Total mRNA from whole blood was isolated from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with disease as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

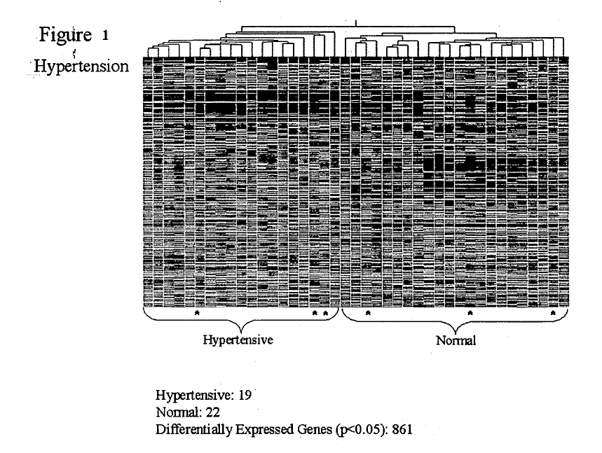

FIG. 1 shows a diagrammatic representation of RNA expression profiles of Whole blood samples from individuals having hypertension and osteoarthritis as compared with RNA expression profiles from normal individuals. Expression profiles were generated using GeneSpring™ software analysis as described herein. Each column represents the hybridization pattern resulting from a single individual. In this example, hypertensive patients also presented with OA, as described herein. Normal individuals have no known medical conditions and were not taking any known medication. Hybridizations to create said RNA expression profiles were done using the ChondroChip™. A dendogram analysis is shown above. Samples are clustered and marked as representing patients who are hypertensive or normal. The “*” indicates those patients who abnormally clustered as either hypertensive, or normal despite presenting with the reverse. The number of hybridizations profiles determined for either hypertensive patients or normal individuals are shown. 861 differentially expressed genes were identified as being differentially expressed with a p value of <0.05 as between the hypertensive patients and normal individuals. The identity of the differentially expressed genes is shown in Table 1A.

Classification or class prediction of a test sample as having hypertension and OA or being normal can be done using the differentially expressed genes as shown in Table 1A in combination with well known statistical algorithms for class prediction as would be understood by a person skilled in the art and described herein. Commercially available programs such as those provided by Silicon Genetics (e.g. GeneSpring™) for Class Predication are also available.

Osteoarthritis and Obesity

ChondroChip™ Microarray Data Analysis of RNA expression profiles of Whole blood samples from co-morbid individuals to identify biomarkers of osteoarthritis and obesityRNA expression profiles.

This example demonstrates the use of the claimed invention to detect differential gene expression in Whole blood samples taken from patients with obesity and OA as compared to Whole blood samples taken from healthy patients.

Whole blood samples were taken from patients who were diagnosed with osteoarthritis and obesity as defined herein. RNA expression profiles were then analysed and compared to profiles from patients unaffected by any disease. In each case, the diagnosis of the disease was corroborated by a skilled Board certified physician. Total mRNA from a blood taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with disease as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

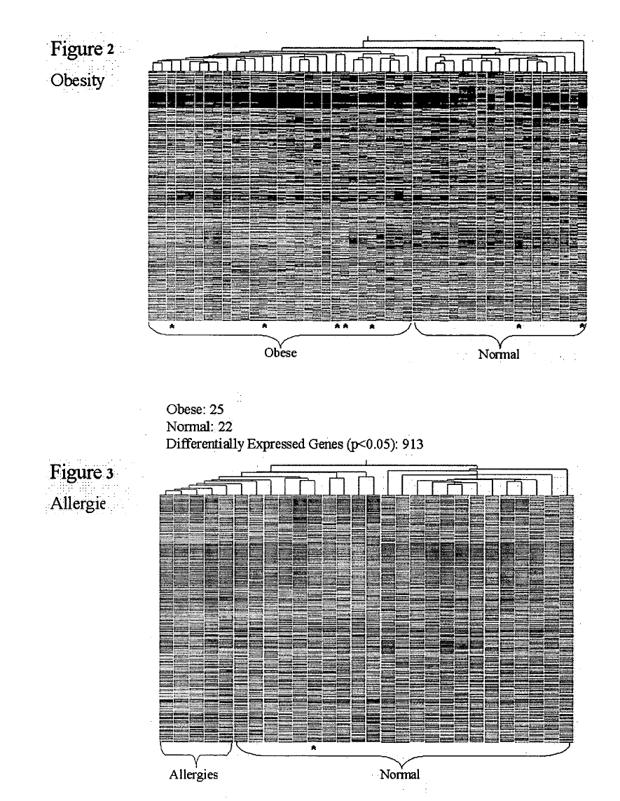

FIG. 2 shows a diagrammatic representation of RNA expression profiles of Whole blood samples from individuals who were identified as obese as described herein as compared with RNA expression profiles from normal individuals. Expression profiles were generated using GeneSpring™ software analysis as described herein. Each column represents the hybridization pattern resulting from a single individual. In this example, obese patients also presented with OA, as described herein. Normal individuals have no known medical conditions and were not taking any known medication. Hybridizations to create said RNA expression profiles were done using the ChondroChip™. A dendogram analysis is shown above. Samples are clustered and marked as representing patients who are obese or normal. The “*” indicates those patients who abnormally clustered as either obese or normal despite presenting with the reverse. The number of hybridization profiles determined for obese patients with OA and normal individuals are shown. 913 genes were identified as being differentially expressed with a p value of <0.05 as between the obese patients with OA and normal individuals is noted. The identity of the differentially expressed genes is shown in Table 1B.

Classification or class prediction of a test sample as either having obesity and OA or being normal can be done using the differentially expressed genes as shown in Table 1B in combination with well known statistical algorithms as would be understood by a person skilled in the art and described herein. Commercially available programs such as those provided by Silicon Genetics (e.g. GeneSpring™) for Class Predication are also available.

Osteoarthritis and Allergies

ChondroChip™ Microarray Data Analysis of RNA expression profiles of Whole blood samples from co-morbid individuals having osteoarthritis and allergies as compared with RNA expression profiles from normal individuals.

This example demonstrates the use of the claimed invention to detect differential biomarkers of osteoarthritis and allergies.

Whole blood samples were taken from patients who were diagnosed with osteoarthritis and allergies as defined herein. These patients are classified as presenting with co-morbidity, or multiple disease states. RNA expression profiles were then analysed and compared to profiles from patients unaffected by any disease. In each case, the diagnosis of osteoarthritis and allergies was corroborated by a skilled Board certified physician.

Total mRNA from blood taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with osteoarthritis and allergies as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

FIG. 3 shows a diagrammatic representation of RNA expression profiles of Whole blood samples from individuals who were identified as having allergies as described herein as compared with RNA expression profiles from normal individuals. Expression profiles were generated using GeneSpring™ software analysis as described herein. Each column represents the hybridization pattern resulting from a single individual. In this example, patients with allergies also presented with OA, as described herein. Normal individuals had no known medical conditions and were not taking any known medication. Hybridizations to create said RNA expression profiles were done using the ChondroChip™. A dendogram analysis is shown above. Samples are clustered and marked as representing patients who are obese or normal. The “*” indicates those patients who abnormally clustered as either having allergies or being normal despite presenting with the reverse. The number of hybridizations profiles determined for patients with allergies and normal individuals are shown. 633 genes were identified as being differentially expressed with a p value of <0.05 as between patients with allergies and normal individuals is noted. The identity of the differentially expressed genes is shown in Table 1C.

Classification or class prediction of a test sample to determine whether said individual has allergies and OA or is normal can be done using the differentially expressed genes as shown in Table 1C in combination with well known statistical algorithms as would be understood by a person skilled in the art and described herein. Commercially available programs such as those provided by Silicon Genetics (e.g. GeneSpring™) for Class Predication are also available.

Osteoarthritis and Systemic Steroids

ChondroChip™ Microarray Data Analysis of RNA expression profiles of Whole blood samples from co-morbid individuals having osteoarthritis and subject to systemic steroids as compared with RNA expression profiles from normal individuals

This example demonstrates the use of the claimed invention to detect biomarkers in blood of patients subject to systemic steroids and having osteoarthritis.

As used herein, “systemic steroids” indicates a person subjected to artificial levels of steroids as a result of medical intervention. Such systemic steroids include birth control pills, prednisone, and hormones as a result of hormone replacement treatment. A person identified as having systemic steroids is one who is on one or more of the following of the above treatment regimes.

Whole blood samples were taken from patients who were diagnosed with osteoarthritis and subject to systemic steroids as defined herein. RNA expression profiles were then analysed and compared to profiles from patients unaffected by any disease. In each case, the diagnosis of osteoarthritis and systemic steroids was corroborated by a skilled Board certified physician.

Total mRNA from blood taken from each patient was isolated using TRIzol® reagent (GIBCO) and fluorescently labeled probes for each blood sample were generated as described above. Each probe was denatured and hybridized to the 15K Chondrogene Microarray Chip (ChondroChip™) as described herein. Identification of genes differentially expressed in Whole blood samples from patients with osteoarthritis and subject to systemic steroids as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

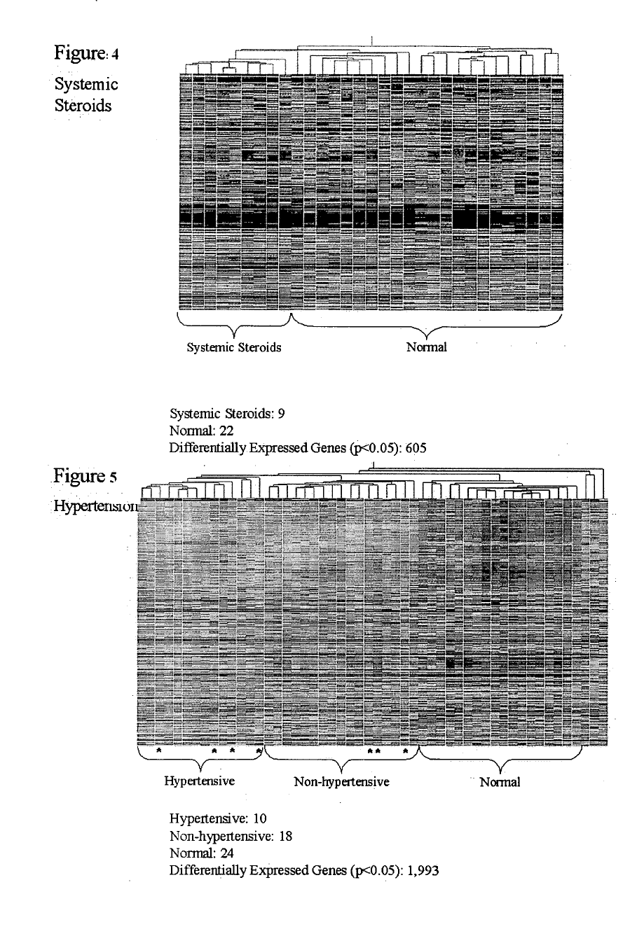

FIG. 4 shows a diagrammatic representation of RNA expression profiles of Whole blood samples from individuals who were subject to systemic steroids as described herein as compared with RNA expression profiles from normal individuals. Expression profiles were generated using GeneSpring™ software analysis as described herein. Each column represents the hybridization pattern resulting from a single individual. In this example, patients taking systemic steroids also presented with OA, as described herein. Normal individuals have no known medical conditions and were not taking any known medication. Hybridizations to create said RNA expression profiles were done using the ChondroChip™ (A dendogram analysis is shown above. Samples are clustered and marked as representing patients who are taking systemic steroids or normal. The “*” indicates those patients who abnormally clustered as either systemic steroids or normal despite presenting with the reverse. The number of hybridizations profiles determined for patients with systemic steroids and normal individuals are shown. 605 genes were identified as being differentially expressed with a p value of <0.05 as between patients with systemic steroids and normal individuals is noted. The identity of the differentially expressed genes is shown in Table 1D.

Classification or class prediction of a test sample from a patient as indicating said patient takes systemic steroids and has OA or as being normal can be done using the differentially expressed genes as shown in Table 1D in combination with well known statistical algorithms for class prediction as would be understood by a person skilled in the art and is described herein. Commercially available programs such as those provided by Silicon Genetics (e.g. GeneSpring™) for Class Predication are also available.

Osteoarthritis and Hypertension Compared with Osteoarthritis Only

ChondroChip™ Microarray Data Analysis of RNA expression profiles of Whole blood samples from individuals having osteoarthritis and hypertension as compared with RNA expression profiles from patients having osteoarthritis only.

This example demonstrates the use of the claimed invention to identify biomarkers in Whole blood samples which are specific to hypertension by comparing gene expression in blood from co-morbid patients with osteoarthritis and hypertension to Whole blood samples taken from OA patients only.

Whole blood samples were taken from patients who were diagnosed with osteoarthritis and hypertension as defined herein. RNA expression profiles were then analysed and compared to profiles from patients having OA only. In each case, the diagnosis of osteoarthritis and/or hypertension was corroborated by a skilled Board certified physician.

Total mRNA from blood taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with disease as compared to OA patients only was determined by statistical analysis using the Wilcox Mann Whitney rank sum test (Glantz S A. Primer of Biostatistics. 5th ed. New York, USA: McGraw-Hill Medical Publishing Division, 2002).

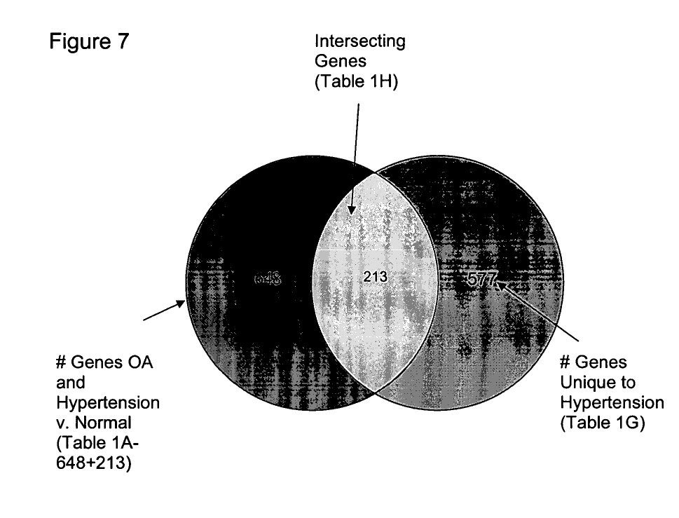

Expression profiles were generated using GeneSpring™ software analysis as described herein (data not shown). The gene list generated from this analysis was identified and those genes previously identified in Table 1A removed so as to identify those genes which are unique to hypertension. 790 differentially expressed genes were identified as being differentially expressed with a p value of <0.05 as between the OA and hypertensive patients when compared with OA individuals. 577 genes were identified as unique to hypertension. The identity of these differentially expressed genes are shown in Table 1G. A gene list is also provided of the 213 genes which were found in common as between those genes identified in Table 1A and genes differentially expressed in Whole blood samples taken from patients with osteoarthritis and hypertension as compared to Whole blood samples taken from OA patients only. The identity of these intersecting differentially expressed genes is shown in Table 1H and a venn diagram showing the relationship between the various groups of gene lists is found in FIG. 7.

Classification or class prediction of a test sample as having hypertension or not having hypertension can be done using the differentially expressed genes as shown in Table 1G as the predictor genes in combination with well known statistical algorithms as would be understood by a person skilled in the art and described herein. Commercially available programs such as those provided by Silicon Genetics (e.g. GeneSpring™) for Class Predication are also available. Classification of individuals as having both OA and hypertension using the genes in Table 1H can also be performed.

Osteoarthritis and Obesity Compared with Osteoarthritis Only

ChondroChip™ Microarray Data Analysis of RNA expression profiles of Whole blood samples from co-morbid individuals having osteoarthritis and obesity as compared with RNA expression profiles from patients having osteoarthritis only.

This example demonstrates the use of the claimed invention to identify biomarkers in Whole blood samples which are specific to obesity by comparing gene expression in blood from co-morbid patients with osteoarthritis and obesity to Whole blood samples taken from OA patients only.

Whole blood samples were taken from patients who were diagnosed with osteoarthritis and obesity as defined herein. RNA expression profiles were then analysed and compared to profiles from patients affected by OA only.

In each case, the diagnosis of the disease was corroborated by a skilled Board certified physician. Total mRNA from blood taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with obesity and OA as compared to OA patients only was determined by statistical analysis using the Wilcox Mann Whitney rank sum test (Glantz S A. Primer of Biostatistics. 5th ed. New York, USA: McGraw-Hill Medical Publishing Division, 2002).

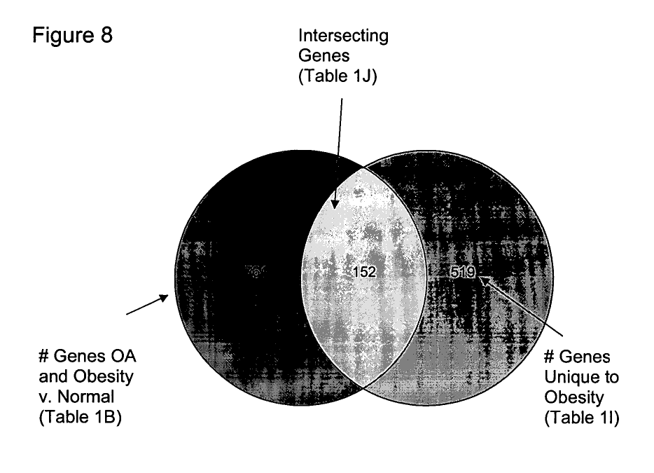

Expression profiles were generated using GeneSpring™ software analysis as described herein (data not shown). 671 genes were identified as being differentially expressed with a p value of <0.05 as between the obese patients with OA and those patients with only OA. Those genes previously identified in Table 1B were removed so as to identify those genes which are unique to obesity. The identity of these 519 genes unique to obesity are shown in Table 1I. A gene list is also provided of those genes which were found in common as between those genes identified in Table 1B and genes differentially expressed in Whole blood samples taken from patients with osteoarthritis and obesity as compared to Whole blood samples taken from OA patients only. 152 genes are shown in Table 1J. A venn diagram showing the relationship between the various groups of gene lists is found in FIG. 8.

Classification or class prediction of a test sample as having obesity or not having obesity can be done using the differentially expressed genes as shown in Table 1I as the predictor genes in combination with well known statistical algorithms as would be understood by a person skilled in the art and described herein. Commercially available programs such as those provided by Silicon Genetics (e.g. GeneSpring™) for Class Predication are also available. Classification of individuals as having both OA and obesity using the genes in Table 1J can also be performed. Osteoarthritis and Allergies Compared with Osteoarthritis Only

ChondroChip™ Microarray Data Analysis of RNA expression profiles of Whole blood samples from individuals having osteoarthritis (OA) and allergies as compared with RNA expression profiles from individuals with OA only.

This example demonstrates the use of the claimed invention to identify biomarkers in Whole blood samples which are specific to allergies by comparing gene expression in blood from co-morbid patients with osteoarthritis and allergies to Whole blood samples taken from OA patients only.

Whole blood samples were taken from patients who were diagnosed with osteoarthritis and allergies as defined herein. RNA expression profiles were then analysed and compared to profiles from patients affected by OA only. In each case, the diagnosis of osteoarthritis and allergies was corroborated by a skilled Board certified physician.

Total mRNA from blood taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with osteoarthritis and allergies as compared to OA patients only was determined by statistical analysis using the Wilcox Mann Whitney rank sum test (Glantz S A. Primer of Biostatistics. 5th ed. New York, USA: McGraw-Hill Medical Publishing Division, 2002).

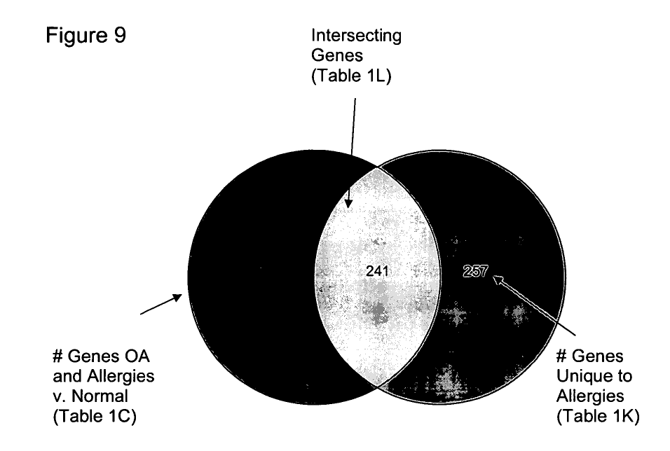

Expression profiles were generated using GeneSpring™ software analysis as described herein (data not shown). 498 genes were identified as being differentially expressed with a p value of <0.05 as between patients with allergies and OA as compared with patients with OA only. Of the 498 genes identified, those genes previously identified in Table 1C were removed so as to identify those genes which are unique to allergies. 257 differentially expressed genes were identified as being as unique to allergies. The identity of these differentially expressed genes are shown in Table 1K. A gene list is also provided of the 241 genes which were found in common as between those genes identified in Table 3C and genes differentially expressed in Whole blood samples taken from patients with osteoarthritis and allergies as compared to Whole blood samples taken from OA patients only. The identity of these intersecting differentially expressed genes is shown in Table 1L and a venn diagram showing the relationship between the various groups of gene lists is found in FIG. 9.

Classification or class prediction of a test sample as having allergies or not having allergies can be done using the differentially expressed genes as shown in Table 1K as the predictor genes in combination with well known statistical algorithms as would be understood by a person skilled in the art and described herein. Commercially available programs such as those provided by Silicon Genetics (e.g. GeneSpring™) for Class Predication are also available. Classification of individuals as having both OA and allergies using the genes in Table 1L can also be performed.

RNA Expression profilesRNA Expression profilesRNA Expression profilesRNA Expression profilesRNA Expression profilesRNA Expression Profiles Osteoarthritis and Systemic Steroids Compared with Osteoarthritis Only

ChondroChip™ Microarray Data Analysis of RNA expression profiles of Whole blood samples from co-morbid individuals having osteoarthritis and subject to systemic steroids as compared with RNA expression profiles from with osteoarthritis only.

This example demonstrates the use of the claimed invention to identify biomarkers in Whole blood samples which are specific to systemic steroids by comparing gene expression in blood from co-morbid patients with osteoarthritis and systemic steroids to Whole blood samples taken from OA patients only.

Whole blood samples were taken from patients who were diagnosed with osteoarthritis and subject to systemic steroids as defined herein. RNA expression profiles were then analysed and compared to profiles from patients having OA only. In each case, the diagnosis of osteoarthritis and systemic steroids was corroborated by a skilled Board certified physician.

Total mRNA from blood was taken from each patient was isolated using TRIzol® reagent (GIBCO) and fluorescently labeled probes for each blood sample were generated as described above. Each probe was denatured and hybridized to the 15K Chondrogene Microarray Chip (ChondroChip™) as described herein. Identification of genes differentially expressed in Whole blood samples from patients with osteoarthritis and subject to systemic steroids as compared patients with OA only was determined by statistical analysis using the Wilcox Mann Whitney rank sum test (Glantz S A. Primer of Biostatistics. 5th ed. New York, USA: McGraw-Hill Medical Publishing Division, 2002).

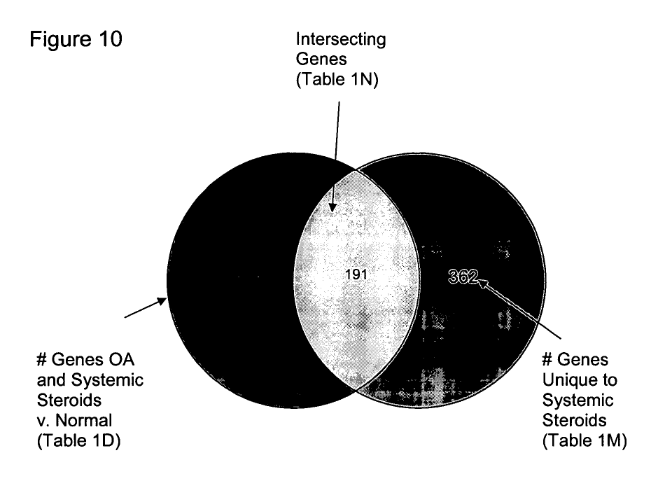

Expression profiles were generated using GeneSpring™ software analysis as described herein (data not shown). 553 genes were identified as being differentially expressed with a p value of <0.05 as between patients taking systemic steroids and OA as compared with patients with OA only. Of the 553 genes identified, those genes previously identified in Table 1D were removed so as to identify those genes which are unique to systemic steroids. 362 differentially expressed genes were identified as being as unique to systemic steroids. The identity of these differentially expressed genes are shown in Table 1M. A gene list is also provided of the 191 genes which were found in common as between those genes identified in Table 3D and genes differentially expressed in Whole blood samples taken from patients with osteoarthritis and systemic steroids as compared to Whole blood samples taken from OA patients only. The identity of these intersecting differentially expressed genes is shown in Table 1N and a venn diagram showing the relationship between the various groups of gene lists is found in FIG. 10.

Classification or class prediction of a test sample of an individual as either taking systemic steroids or not taking systemic steroids can be done using the differentially expressed genes as shown in Table 1M as the predictor genes in combination with well known statistical algorithms as would be understood by a person skilled in the art and described herein. Commercially available programs such as those provided by Silicon Genetics (e.g. GeneSpring™) for Class Predication are also available. Classification of individuals as having both OA and taking systemic steroids using the genes in Table 1N can also be performed.

Osteoarthritis and Systemic Steroids Compared with Normal so as to Differentiate Between Types of Systemic Steroids.

ChondroChip™ Microarray Data Analysis of RNA expression profiles of Whole blood samples from co-morbid individuals having osteoarthritis and subject to systemic steroids as compared with RNA expression profiles from normal individuals.

This example demonstrates the use of the claimed invention to identify biomarkers in Whole blood samples which are specific to individual types of systemic steroids by comparing gene expression in blood from co-morbid patients with osteoarthritis and either on prednisone, birth control pills or taking hormones to Whole blood samples taken from OA patients only.

As used herein, “systemic steroids” indicates a person subjected to artificial levels of steroids as a result of medical intervention. Such systemic steroids include birth control pills, prednisone, and hormones as a result of hormone replacement treatment. A person identified as having systemic steroids is one who is on one or more of the following of the above treatment regimes.

Whole blood samples were taken from patients who were diagnosed with osteoarthritis and subject to systemic steroids as defined herein. RNA expression profiles were then analysed and compared as between the systemic steroids as compared to profiles from patients unaffected by any disease. In each case, the diagnosis of osteoarthritis and systemic steroids was corroborated by a skilled Board certified physician.

Total mRNA from blood taken from each patient was isolated using TRIzol® reagent (GIBCO) and fluorescently labeled probes for each blood sample were generated as described above. Each probe was denatured and hybridized to the 15K Chondrogene Microarray Chip (ChondroChip™) as described herein. Identification of genes differentially expressed in Whole blood samples from patients with osteoarthritis and subject to systemic steroids as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

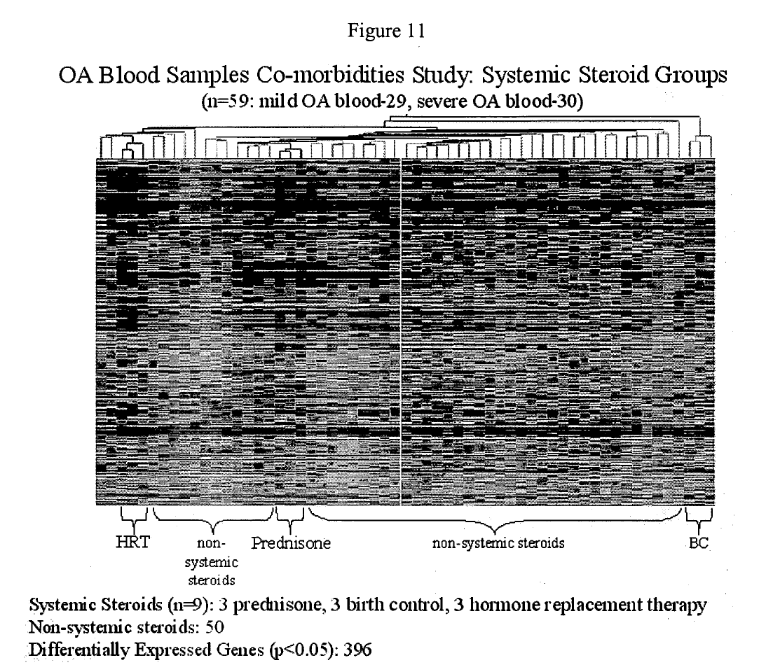

FIG. 11 shows a diagrammatic representation of RNA expression profiles of Whole blood samples from individuals who were subject to either birth control, prednisone, or hormone replacement therapy as described herein as compared with RNA expression profiles from normal individuals. Expression profiles were generated using GeneSpring™ software analysis as described herein. Each column represents the hybridization pattern resulting from a single individual. In this example, patients taking with each of the systemic steroids also presented with OA, as described herein. Normal individuals have no known medical conditions and were not taking any known medication. Hybridizations to create said RNA expression profiles were done using the ChondroChip™. A dendogram analysis is shown above. Samples are clustered and marked as representing patients who are taking birth control, prednisone, hormone replacement therapy or normal. The “*” indicates those patients who abnormally clustered. The number of hybridizations profiles determined for patients with birth control, prednisone, hormone replacement therapy or normal individuals are shown. 396 genes were identified as being differentially expressed with a p value of <0.05 as between patients with systemic steroids and normal individuals is noted. The identity of the differentially expressed genes is shown in Table 1O.

Classification or class prediction of a test sample from a patient as indicating said patient takes systemic steroids and has OA or as being normal can be done using the differentially expressed genes as shown in Table 1O in combination with well known statistical algorithms for class prediction as would be understood by a person skilled in the art and is described herein. Commercially available programs such as those provided by Silicon Genetics (e.g. GeneSpring™) for Class Predication are also available.

Osteoarthritis and Asthma Compared with Osteoarthritis Only

ChondroChip™ Microarray Data Analysis of RNA expression profiles of Whole blood samples from individuals having osteoarthritis (OA) and asthma as compared with RNA expression profiles from individuals with OA only.

This example demonstrates the use of the claimed invention to identify biomarkers in Whole blood samples which are specific to asthma.

Whole blood samples were taken from patients who were diagnosed with osteoarthritis and asthma as defined herein. RNA expression profiles were then analysed and compared to profiles from patients affected by asthma only. In each case, the diagnosis of osteoarthritis and asthma was corroborated by a skilled Board certified physician.

Total mRNA from blood was taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with osteoarthritis and asthma as compared to OA patients only was determined by statistical analysis using the Wilcox Mann Whitney rank sum test (Glantz S A. Primer of Biostatistics. 5th ed. New York, USA: McGraw-Hill Medical Publishing Division, 2002). FIG. 24 shows a diagrammatic representation of RNA expression profiles of Whole blood samples from individuals who had asthma and osteoarthritis as described herein as compared with RNA expression profiles from osteoarthritic individuals. Expression profiles were generated using GeneSpring™ software analysis as described herein. Each column represents the hybridization pattern resulting from a single individual. Hybridizations to create said RNA expression profiles were done using the ChondroChip™ and the Affymetrix Chip. (A dendogram analysis is shown above). Samples are clustered and marked as representing patients who have asthma and OA or those patients who have just OA. The number of hybridizations profiles determined for patients with asthma and patients without asthma are shown. Various experiments were performed using the ChondroChip™as outlined above and analyzed using either the Wilcox Mann Whitney rank sum test, or other statistical tests as described herein, and those genes identified with a p value of <0.05 as between the patients with asthma and OA and patients with just OA are shown in Table 1AC. Additionally experiments were performed using the Affymetrix® GeneChip® platforms (U133A and U133 Plus 2.0) platforms (U133A and U133 Plus 2.0) as described herein (data not shown) using either the Wilcox Mann Whitney rank sum test, or other statistical tests as described herein, and those genes identified with a p value of <0.05 as between the patients with asthma and without asthma are shown in Table 1AD.

### EXAMPLE 4

In addition to methods to identify biomarkers associated with a specific disease or condition, this invention also includes methods to identify biomarkers that distinguish between different stages of the condition. The following examples illustrate embodiments of the application of the instant methods as applied to identifying biomarkers associated with specific stages of bladder cancer and osteoarthritis, however, this aspect of the invention is not limited to these particular conditions.

Bladder Cancer

Affymetrix Chip Microarray Data Analysis of RNA expression profiles of Whole blood samples from individuals having early or advanced bladder cancer as compared with RNA expression profiles from normal individuals.

This example demonstrates the use of the claimed invention to identify biomarkers in Whole blood samples which are specific to a stage of bladder cancer by comparing gene expression in blood from individuals with advanced bladder cancer and those without bladder cancer.

As used herein, “early stage bladder cancer” includes bladder cancer wherein the detection of the anatomic extent of the tumor, both in its primary location and in metastatic sites, as defined by the TNM staging system in accordance with Harrison's Principles of Internal Medicine 14th edition can be considered early stage. More specifically, early stage bladder cancer can include those instances wherein the carcinoma is mainly superficial.

As used herein, “advanced stage bladder cancer” is defined as bladder cancer wherein the detection of the anatomic extent of the tumor, both in its primary location and in metastatic sites, as defined by the TNM staging system in accordance with Harrison's Principles of Internal Medicine 14th edition, can be considered as advanced stage. More specifically, advanced stage carcinomas can involve instances wherein the cancer has infiltrated the muscle and wherein metastasis has occurred.

Whole blood samples were taken from patients who were diagnosed with early or advanced late stage bladder cancer as defined herein. RNA expression profiles were then analysed and compared to profiles from patients unaffected by any disease. In each case, the diagnosis of early or advanced late stage bladder cancer was corroborated by a skilled Board certified physician.

Total mRNA from a blood sample taken from each patient was isolated using TRIzol® reagent (GIBCO) and fluorescently labeled probes for each blood sample were generated as described above. Each probe was denatured and hybridized to a Affymetrix U133A Chip as described herein. Identification of genes differentially expressed in Whole blood samples from patients with early or advanced late stage bladder cancer as compared to healthy patients was determined by statistical analysis using the Wilcox Mann Whitney rank sum test (Glantz S A. Primer of Biostatistics. 5th ed. New York, USA: McGraw-Hill Medical Publishing Division, 2002).

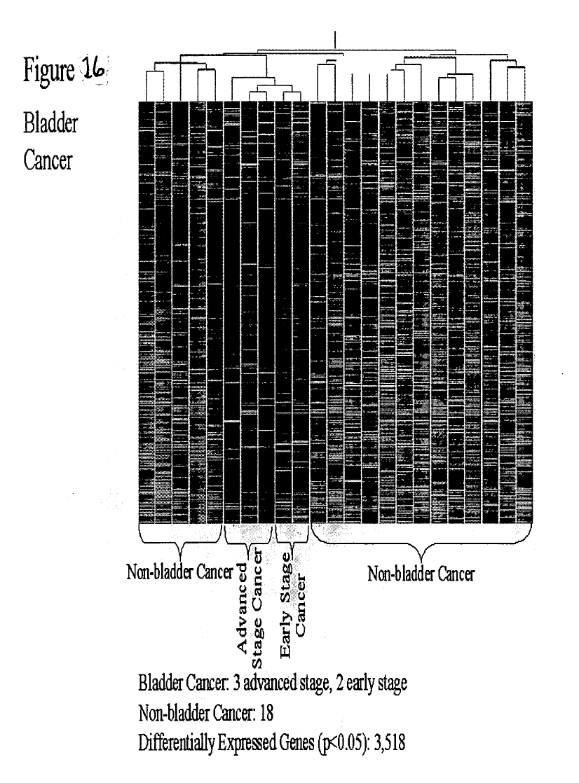

FIG. 16 shows a diagrammatic representation of RNA expression profiles of Whole blood samples from individuals who were identified as having advanced stage bladder cancer or early stage bladder cancer as described herein as compared with RNA expression profiles from non bladder cancer individuals. Expression profiles were generated using GeneSpring™ software analysis as described herein. Each column represents the hybridization pattern resulting from a single individual. Non bladder cancer individuals presented without bladder cancer, but may have presented with other medical conditions and may be under various treatment regimes. Hybridizations to create said RNA expression profiles were done using the Affymetrix U133A chip. A dendogram analysis is shown above. Samples are clustered and marked as representing patients who have early stage bladder cancer, advanced stage bladder cancer, or do not have bladder cancer. The “*” indicates those patients who abnormally clustered despite actual presentation. The number of hybridizations profiles determined for either early stage bladder cancer, advanced bladder cancer or non-bladder cancer are shown. 3,518 genes were identified as being differentially expressed with a p value of <0.05 using an ANOVA analysis. The identity of the differentially expressed genes identified is shown in Table 1T. Various experiments were also performed as outlined above, and analyzed using either the Wilcox Mann Whitney rank sum test or other statistical tests as described herein, and those genes identified with a p value of <0.05 as between the patients with any stage of advanced bladder cancer as compared with patients without bladder cancer are shown in Table 5V.

Classification or class prediction of a test sample of an individual to determine whether said individual has advanced bladder cancer, early stage bladder cancer or does not have bladder cancer can be done using the differentially expressed genes as shown in Table 1T and/or 5V as the predictor genes in combination with well known statistical algorithms as would be understood by a person skilled in the art and described herein. Commercially available programs such as those provided by Silicon Genetics (e.g. GeneSpring™) for Class Predication are also available.

Osteoarthritis Staging

This example demonstrates the use of the claimed invention to identify biomarkers in Whole blood samples which are specific various stages of osteoarthritis so as to allow the monitoring (progression or regression) of disease.

Osteoarthritis (OA), as used herein also known as “degenerative joint disease”, represents failure of a diarthrodial (movable, synovial-lined) joint. It is a condition, which affects joint cartilage, and or subsequently underlying bone and supporting tissues leading to pain, stiffness, movement problems and activity limitations. It most often affects the hip, knee, foot, and hand, but can affect other joints as well.

OA severity can be graded according to the system described by Marshall (Marshall K W. J Rheumatol, 1996:23(4) 582:85). Briefly, each of the six knee articular surfaces was assigned a cartilage grade with points based on the worst lesion seen on each particular surface. Grade 0 is normal (0 points), Grade I cartilage is soft or swollen but the articular surface is intact (1 point). In Grade II lesions, the cartilage surface is not intact but the lesion does not extend down to subchondral bone (2 points). Grade III damage extends to subchondral bone but the bone is neither eroded nor eburnated (3 points). In Grade IV lesions, there is eburnation of or erosion into bone (4 points). A global OA score is calculated by summing the points from all six cartilage surfaces. If there is any associated pathology, such as meniscus tear, an extra point will be added to the global score. Based on the total score, each patient is then categorized into one of four OA groups: mild (1-6), moderate (7-12), marked (13-18), and severe (>18). As used herein, patients identified with OA may be categorized in any of the four OA groupings as described above.

Whole blood samples were taken from patients who were diagnosed with osteoarthritis and a specific stage of osteoarthritis as defined herein. RNA expression profiles were then analysed and compared to profiles from patients unaffected by any disease. In each case, the diagnosis of osteoarthritis and the stage of osteoarthritis was corroborated by a skilled Board certified physician.

Total mRNA from a blood sample taken from each patient was isolated using TRIzol® reagent (GIBCO) and fluorescently labeled probes for each blood sample were generated as described above. Each probe was denatured and hybridized to a 15K Chondrogene Microarray Chip (ChondroChip™) as described herein. Identification of genes differentially expressed in Whole blood samples from patients with disease as compared to healthy patients was determined by statistical analysis using the Wilcox Mann Whitney rank sum test (Glantz S A. Primer of Biostatistics., 5th ed. New York, USA: McGraw-Hill Medical Publishing Division, 2002).

FIG. 20 shows a diagrammatic representation of RNA expression profiles of Whole blood samples from individuals having osteoarthritis as compared with RNA expression profiles from normal individuals. Expression profiles were generated using GeneSpring™ software analysis as described herein. Each column represents the hybridization pattern resulting from a single individual. Normal individuals have no known medical conditions and were not taking any known medication. Hybridizations to create said RNA expression profiles were done using the ChondroChip™ and the Affymetrix™ Chip. A dendogram analysis is shown above. Samples are clustered and marked as representing patients who presented with different stages of osteoarthritis or normal. The “*” indicates those patients who abnormally clustered despite actual presentation. The number of hybridizations profiles determined for either osteoarthritis patients or normal individuals are shown. Differentially expressed genes were identified as being differentially expressed using ANOVA analysis and those genes with a p value of <0.05 identified. The identity of the differentially expressed genes is shown in Tables 1Y. In addition, various experiments were also performed as outlined above, and analyzed using either the Wilcox Mann Whitney rank sum test or other statistical tests as described herein, and using a pairwise comparison, those genes identified with a p value of <0.05 as between the patients with any stage of osteoarthritis as compared with patients without osteoarthritis are shown in Table 4A and 4B.

Classification or class prediction of a test sample from an unknown patient in order to diagnose said individual with osteoarthritis can be done using the differentially expressed genes as shown in Table 4A and 4B in combination with well known statistical algorithms for class prediction as would be understood by a person skilled in the art and is described herein. Commercially available programs such as those provided by Silicon Genetics (e.g. GeneSpring™) for Class Prediction are also available.

Differentially expressed genes were also identified as being differentially expressed with a p value of <0.05 as between patients with mild osteoarthritis and normal individuals. The identity of the differentially expressed genes is shown in Tables 4C and 4D. Differentially expressed genes were also identified as being differentially expressed with a p value of <0.05 as between patients with moderate osteoarthritis and normal individuals. Classification or class prediction of a test sample of an individual to determine whether said individual has mild osteoarthritis can be done using the differentially expressed genes as shown in Table 4C and/or 4D as the predictor genes in combination with well known statistical algorithms as would be understood by a person skilled in the art and described herein. Commercially available programs such as those provided by Silicon Genetics (e.g. GeneSpring™) for Class Predication are also

Differentially expressed genes were also identified as being differentially expressed with a p value of <0.05 as between patients with moderate osteoarthritis and normal individuals. The identity of the differentially expressed genes is shown in Tables 4E and 4F. Classification or class prediction of a test sample of an individual to determine whether said individual has moderate osteoarthritis can be done using the differentially expressed genes as shown in Table 4E and/or 4F as the predictor genes in combination with well known statistical algorithms as would be understood by a person skilled in the art and described herein. Commercially available programs such as those provided by Silicon Genetics (e.g. GeneSpring™) for Class Prediction are also

Differentially expressed genes were also identified as being differentially expressed with a p value of <0.05 as between patients with marked osteoarthritis and normal individuals. The identity of the differentially expressed genes is shown in Tables 4G and 4H.

Classification or class prediction of a test sample of an individual to determine whether said individual has marked osteoarthritis can be done using the differentially expressed genes as shown in Table 4G and/or 4H as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with severe osteoarthritis and patients without osteoarthritis. The identity of the differentially expressed genes is shown in Tables 41 and 4J.

Classification or class prediction of a test sample of an individual to differentiate as to whether said individual has severe osteoarthritis can be done using the differentially expressed genes as shown in Table 4I and/or 4J as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with mild osteoarthritis and patients with moderate osteoarthritis. The identity of the differentially expressed genes is shown in Tables 4K and 4L. Classification or class prediction of a test sample of an individual to differentiate as to whether said individual has mild or moderate osteoarthritis can be done using the differentially expressed genes as shown in Table 4K and/or 4L as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with mild osteoarthritis and patients with marked osteoarthritis. The identity of the differentially expressed genes is shown in Tables 4M and 4N.

Classification or class prediction of a test sample of an individual to differentiate as to whether said individual has mild or marked osteoarthritis can be done using the differentially expressed genes as shown in Table 4M and/or 4N as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with mild osteoarthritis and patients with severeosteoarthritis. The identity of the differentially expressed genes is shown in Tables 4O and 4P.

Classification or class prediction of a test sample of an individual to differentiate as to whether said individual has mild or severe osteoarthritis can be done using the differentially expressed genes as shown in Table 4O and/or 4P as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with moderate osteoarthritis and patients with marked osteoarthritis. The identity of the differentially expressed genes is shown in Tables 4Q and 4R.

Classification or class prediction of a test sample of an individual to differentiate as to whether said individual has moderate or marked osteoarthritis can be done using the differentially expressed genes as shown in Table 4Q and/or 4R as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with moderate osteoarthritis and patients with severe osteoarthritis. The identity of the differentially expressed genes is shown in Tables 4S and 4T.

Classification or class prediction of a test sample of an individual to differentiate as to whether said individual has moderate or severe osteoarthritis can be done using the differentially expressed genes as shown in Table 4S and/or 4T as the predictor genes in combination with well known statistical algorithms as would be understood by a person skilled in the art and described herein. Commercially available programs such as those provided by Silicon Genetics (e.g. GeneSpring™) for Class Prediction are also available.

Differentially expressed genes were also identified as being differentially expressed with a p value of <0.05 as between patients with marked osteoarthritis and patients with severe osteoarthritis. The identity of the differentially expressed genes is shown in Tables 4U and 4V.

Classification or class prediction of a test sample of an individual to differentiate as to whether said individual has marked or severe osteoarthritis can be done using the differentially expressed genes as shown in Table 4U and/or 4V as the predictor genes in combination with well known statistical algorithms as would be understood by a person skilled in the art and described herein. Commercially available programs such as those provided by Silicon Genetics (e.g. GeneSpring™) for Class Prediction are also available.

### EXAMPLE 5

In addition to methods to identify biomarkers associated with a specific disease or condition, or stage thereof, this invention also includes methods to identify biomarkers that distinguish between two conditions. The pair of conditions can be closely related, can have unrelated etiology but display similar overt symptoms, or can be unrelated. The following examples illustrate embodiments of methods of this aspect of the invention, but the invention is not limited to these embodiments.

Manic Depression Syndrome as Compared with Schizophrenia RNA Expression Profiles

This example demonstrates the use of the claimed invention to identify biomarker which are capable of differentiating between manic depression syndrome and schizophrenia and use of same.

Whole blood samples were taken from patients diagnosed with MDS and Whole blood samples were taken from patients diagnosed with schizophrenia as defined herein. RNA expression profiles were then analyzed and the profiles generated for individuals having MDS compared with the profiles generated for individuals having schizophrenia. In each case, the diagnosis of MDS and schizophrenia is corroborated by a skilled Board certified physician. RNA expression profiles of Whole blood samples from individuals who were identified as having MDS as described herein as compared with RNA expression profiles from individuals identified as having schizophrenia were generated using GeneSpring™ software analysis as described herein. Hybridizations to create said RNA expression profiles were done using the Affymetrix® GeneChip® platforms (U133A and U133 Plus 2.0) platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with MDS as compared with patients schizophrenia are shown in Table 3A.

Classification or class prediction of a test sample of an individual to differentiate as to whether said individual has schizophrenia or MDS can be done using the differentially expressed genes as shown in Table 3A as the predictor genes in combination with well known statistical algorithms as would be understood by a person skilled in the art and described herein. Commercially available programs such as those provided by Silicon Genetics (e.g. GeneSpring™) for Class Prediction are also available.

Hepatitis as Compared with Liver Cancer RNA Expression Profiles

This example demonstrates the use of the claimed invention to identify biomarker which are capable of differentiating between hepatitis B and liver cancer and use of same.

Whole blood samples were taken from patients diagnosed with hepatitis B and Whole blood samples were taken from patients diagnosed with liver cancer as defined herein. RNA expression profiles from were then analyzed and the profiles generated for individuals having hepatitis B compared with the profiles generated for individuals having liver cancer. In each case, the diagnosis of hepatitis B or liver cancer is corroborated by a skilled Board certified physician. RNA expression profiles of Whole blood samples from individuals who were identified as having hepatitis B as described herein as compared with RNA expression profiles from individuals identified as having schizophrenia were generated using GeneSpring™ software analysis as described herein. Hybridizations to create said RNA expression profiles were done using the Affymetrix® GeneChip® platforms (U133A and U133 Plus 2.0) platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with MDS as compared with patients schizophrenia are shown in Table 3B.

Classification or class prediction of a test sample of an individual to differentiate as to whether said individual has hepatitis or liver cancer can be done using the differentially expressed genes as shown in Table 3B as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Bladder Cancer as Compared with Kidney Cancer RNA Expression Profiles

This example demonstrates the use of the claimed invention to identify biomarker which are capable of differentiating between bladder cancer and kidney cancer and use of same.

Whole blood samples were taken from patients diagnosed with bladder cancer and Whole blood samples were taken from patients diagnosed with kidney cancer as defined herein. RNA expression profiles were then analyzed and the profiles generated. In each case, the diagnosis of bladder cancer and kidney cancer was corroborated by a skilled Board certified physician. RNA expression profiles of Whole blood samples from individuals who were identified as having bladder cancer as described herein as compared with RNA expression profiles from individuals identified as having kidney cancer were generated using GeneSpring™ software analysis as described herein. Hybridizations to create said RNA expression profiles were done using the Affymetrix® GeneChip® platforms (U133A and U133 Plus 2.0) platforms (Ul33A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with bladder cancer as compared with patients with kidney cancer are shown in Table 3C.

Classification or class prediction of a test sample of an individual to differentiate as to whether said individual has bladder cancer or kidney cancer can be done using the differentially expressed genes as shown in Table 3C as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Bladder Cancer as Compared with Testicular Cancer RNA Expression Profiles

This example demonstrates the use of the claimed invention to identify biomarker which are capable of differentiating between bladder cancer and testicular cancer and use of same.

Whole blood samples were taken from patients diagnosed with bladder cancer and Whole blood samples were taken from patients diagnosed with testicular cancer as defined herein. RNA expression profiles were then analyzed and the profiles generated for individuals having bladder cancer as compared with the profiles generated for individuals having testicular cancer. In each case, the diagnosis of bladder cancer and testicular cancer is corroborated by a skilled Board certified physician. RNA expression profiles of Whole blood samples from individuals who were identified as having bladder cancer as described herein as compared with RNA expression profiles from individuals identified as having testicular cancer were generated using GeneSpring™ software analysis as described herein. Hybridizations to create said RNA expression profiles were done using the Affymetrix® GeneChip® platforms (U133A and U133 Plus 2.0) platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with bladder cancer as compared with patients testicular cancer are shown in Table 3D.

Classification or class prediction of a test sample of an individual to differentiate as to whether said individual has bladder cancer or testicular cancer can be done using the differentially expressed genes as shown in Table 3D as the predictor genes in combination with well known statistical algorithms as would be understood by a person skilled in the art and described herein. Commercially available programs such as those provided by Silicon Genetics (e.g. GeneSpring™) for Class Prediction are also available.

RNA expression profiles Kidney Cancer as compared with Testicular Cancer RNA Expression Profiles This example demonstrates the use of the claimed invention to identify biomarker which are capable of differentiating between kidney cancer and testicular cancer and use of same.

Whole blood samples were taken from patients diagnosed with kidney cancer and Whole blood samples were taken from patients diagnosed with testicular cancer as defined herein. RNA expression profiles were then analyzed and the profiles generated for individuals having kidney cancer as compared with the profiles generated for individuals having testicular cancer. In each case, the diagnosis of kidney cancer and testicular cancer is corroborated by a skilled Board certified physician. RNA expression profiles of Whole blood samples from individuals who were identified as having kidney cancer as described herein as compared with RNA expression profiles from individuals identified as having testicular cancer were generated using GeneSpring™ software analysis as described herein. Hybridizations to create said RNA expression profiles were done using the Affymetrix® GeneChip® platforms (U133A and U133 Plus 2.0) platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with bladder cancer as compared with patients with testicular cancer are shown in Table 3E.

Classification or class prediction of a test sample of an individual to differentiate as to whether said individual has bladder cancer or testicular cancer can be done using the differentially expressed genes as shown in Table 3E as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Liver Cancer as Compared with Stomach Cancer RNA Expression Profiles

This example demonstrates the use of the claimed invention to identify biomarker which are capable of differentiating between liver cancer and stomach cancer and use of same.

Whole blood samples were taken from patients diagnosed with liver cancer and Whole blood samples were taken from patients diagnosed with stomach cancer as defined herein. RNA expression profiles were then analyzed and the profiles generated for individuals having liver cancer as compared with the profiles generated for individuals having stomach cancer. In each case, the diagnosis of liver cancer and stomach cancer is corroborated by a skilled Board certified physician. RNA expression profiles of Whole blood samples from individuals who were identified as having liver cancer as described herein as compared with RNA expression profiles from individuals identified as having stomach cancer were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with bladder cancer as compared with patients testicular cancer are shown in Table 3F.

Classification or class prediction of a test sample of an individual to differentiate as to whether said individual has bladder cancer or testicular cancer can be done using the differentially expressed genes as shown in Table 3F as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Liver Cancer as Compared with Colon Cancer

This example demonstrates the use of the claimed invention to identify biomarker which are capable of differentiating between liver cancer and colon cancer and use of same.

Whole blood samples were taken from patients diagnosed with liver cancer and Whole blood samples were taken from patients diagnosed with colon cancer as defined herein. RNA expression profiles were then analyzed and the profiles generated for individuals having liver cancer as compared with the profiles generated for individuals having colon cancer. In each case, the diagnosis of liver cancer and colon cancer is corroborated by a skilled Board certified physician. RNA expression profiles of Whole blood samples from individuals who were identified as having liver cancer as described herein as compared with RNA expression profiles from individuals identified as having colon cancer were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with liver cancer as compared with patients with colon cancer are shown in Table 3G.

Classification or class prediction of a test sample of an individual to differentiate as to whether said individual has liver cancer or colon cancer can be done using the differentially expressed genes as shown in Table 3G as the predictor genes in combination with well known statistical algorithms as would be understood by a person skilled in the art and described herein. Commercially available programs such as those provided by Silicon Genetics (e.g. GeneSpring™) for Class Prediction are also available.

3G.

Stomach Cancer as Compared with Colon Cancer

This example demonstrates the use of the claimed invention to identify biomarkers which are capable of differentiating between stomach cancer and colon cancer and use of same.

Whole blood samples were taken from patients diagnosed with stomach cancer and Whole blood samples were taken from patients diagnosed with colon cancer as defined herein. RNA expression profiles were then analyzed and the profiles generated for individuals having stomach cancer as compared with the profiles generated for individuals having colon cancer. In each case, the diagnosis of stomach cancer and colon cancer is corroborated by a skilled Board certified physician. RNA expression profiles of Whole blood samples from individuals who were identified as having stomach cancer as described herein as compared with RNA expression profiles from individuals identified as having colon cancer were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with stomach cancer as compared with patients colon cancer are shown in Table 3H.

Classification or class prediction of a test sample of an individual to differentiate as to whether said individual has stomach cancer or colon cancer can be done using the differentially expressed genes as shown in Table 3H as the predictor genes in combination with well known statistical algorithms as would be understood by a person skilled in the art and described herein. Commercially available programs such as those provided by Silicon Genetics (e.g. GeneSpring™) for Class Prediction are also available.

Osteoarthritis as Compared with Rheumatoid Arthritis

This example demonstrates the use of the claimed invention to identify biomarkers which are capable of differentiating between OA and RA and use of same.

Whole blood samples were taken from patients diagnosed with OA and Whole blood samples were taken from patients diagnosed with RA as defined herein. RNA expression profiles were then analyzed and the profiles generated for individuals having OA as compared with the profiles generated for individuals having RA. In each case, the diagnosis of OA and RA is corroborated by a skilled Board certified physician. RNA expression profiles of Whole blood samples from individuals who were identified as having OA as described herein as compared with RNA expression profiles from individuals identified as having RA were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with OA as compared with patients with RA are shown in Table 3I.

Classification or class prediction of a test sample of an individual to differentiate as to whether said individual has OA or RA can be done using the differentially expressed genes as shown in Table 3I as the predictor genes in combination with well known statistical algorithms as would be understood by a person skilled in the art and described herein. Commercially available programs such as those provided by Silicon Genetics (e.g. GeneSpring™) for Class Prediction are also available.

Chagas Disease as compared with Heart Failure This example demonstrates the use of the claimed invention to identify biomarkers which are capable of differentiating between Chagas' disease and heart failure and use of same.

Whole blood samples were taken from patients diagnosed with Chagas' disease and Whole blood samples were taken from patients diagnosed with heart failure as defined herein. RNA expression profiles were then analyzed and the profiles generated for individuals having Chagas' disease as compared with the profiles generated for individuals having heart failure. In each case, the diagnosis of Chagas' disease and heart failure is corroborated by a skilled Board certified physician. RNA expression profiles of Whole blood samples from individuals who were identified as having Chagas' disease as described herein as compared with RNA expression profiles from individuals identified as having heart failure were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with Chagas' disease as compared with patients with heart failure are shown in Table 3I.

Classification or class prediction of a test sample of an individual to differentiate as to whether said individual has Chagas' disease or heart failure can be done using the differentially expressed genes as shown in Table 3I as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Chagas Disease as compared with Coronary Artery Disease This example demonstrates the use of the claimed invention to identify biomarkers which are capable of differentiating between Chagas' disease and coronary artery disease and use of same.

Whole blood samples were taken from patients diagnosed with Chagas' disease and Whole blood samples were taken from patients diagnosed with coronary artery disease as defined herein. RNA expression profiles were then analyzed and the profiles generated for individuals having stomach cancer as compared with the profiles generated for individuals having coronary artery disease. In each case, the diagnosis of Chagas' disease and coronary artery disease is corroborated by a skilled Board certified physician. RNA expression profiles of Whole blood samples from individuals who were identified as having Chagas' disease as described herein as compared with RNA expression profiles from individuals identified as having coronary artery disease were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with Chagas' disease as compared with patients coronary artery disease are shown in Table 3L.

Classification or class prediction of a test sample of an individual to differentiate as to whether said individual has Chagas' disease or coronary artery disease can be done using the differentially expressed genes as shown in Table 3L as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

RNA expression profilesRNA expression profilesRNA expression profilesRNA expression profiles.

Coronary Artery Disease (CAD) as Compared with Heart Failure

This example demonstrates the use of the claimed invention to identify biomarkers which are capable of differentiating between Coronary Artery Disease (CAD) and Heart Failure and use of same.

Whole blood samples were taken from patients diagnosed with having Coronary Artery Disease (CAD) and Whole blood samples were taken from patients diagnosed with having Heart Failure as defined herein. RNA expression profiles were then analyzed and the profiles generated for individuals having CAD as compared with the profiles generated for individuals heart failure. In each case, the diagnosis of heart failure and coronary artery disease is corroborated by a skilled Board certified physician. RNA expression profiles of Whole blood samples from individuals who were identified as having coronary artery disease as described herein as compared with RNA expression profiles from individuals identified as having heart failure were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with coronary artery disease as compared with patients with heart failure are shown in Table 3N.

Classification or class prediction of a test sample of an individual to differentiate as to whether said individual has coronary artery disease or heart failure can be done using the differentially expressed genes as shown in Table 3N as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

Asymptomatic Chagas Disease as compared with Symptomatic Chagas Disease

This example demonstrates the use of the claimed invention to identify biomarkers which are capable of differentiating between Asymptomatic Chagas Disease and Symptomatic Chagas Disease and use of same.

Whole blood samples were taken from patients diagnosed with having Asymptomatic Chagas Disease and Whole blood samples were taken from patients diagnosed with Symptomatic Chagas Disease as defined herein. RNA expression profiles were then analyzed and the profiles generated for individuals having Asymptomatic Chagas Disease as compared with the profiles generated for individuals with Symptomatic Chagas Disease. In each case, the diagnosis of Asymptomatic Chagas Disease and and Symptomatic Chagas Disease is corroborated by a skilled Board certified physician. RNA expression profiles of Whole blood samples from individuals who were identified as having Asymptomatic Chagas Disease as described herein as compared with RNA expression profiles from individuals identified as having Symptomatic Chagas Disease were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with Asymptomatic Chagas Disease as compared with patients with Symptomatic Chagas Disease are shown in Table 3P.

Classification or class prediction of a test sample of an individual to differentiate as to whether said individual has Asymptomatic Chagas Disease or Symptomatic Chagas Disease can be done using the differentially expressed genes as shown in Table 3P as the predictor genes in combination with well known statistical algorithms as would be understood by a person skilled in the art and described herein. Commercially available programs such as those provided by Silicon Genetics (e.g. GeneSpring™) for Class Prediction are also available.

Alzheimer's Disease as Compared with Schizophrenia

This example demonstrates the use of the claimed invention to identify biomarkers which are capable of differentiating between Alzheimer's Disease and Schizophrenia and use of same.

Whole blood samples were taken from patients diagnosed with having Alzheimer's Disease Disease and Whole blood samples were taken from patients diagnosed with Schizophrenia as defined herein. RNA expression profiles were then analyzed and the profiles generated for individuals having Alzheimer's Disease as compared with the profiles generated for individuals with Schizophrenia. In each case, the diagnosis of Alzheimer's Disease and Schizophrenia is corroborated by a skilled Board certified physician. RNA expression profiles of Whole blood samples from individuals who were identified as having Alzheimer's Disease as described herein as compared with RNA expression profiles from individuals identified as Schizophrenia were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with Alzheimer's Disease as compared with patients Schizophrenia are shown in Table 3Q.

Classification or class prediction of a test sample of an individual to differentiate as to whether said individual has Alzheimer's Disease or Schizophrenia can be done using the differentially expressed genes as shown in Table 3Q as the predictor genes in combination with well known statistical algorithms as would be understood by a person skilled in the art and described herein. Commercially available programs such as those provided by Silicon Genetics (e.g. GeneSpring™) for Class Prediction are also available.

Alzheimer's Disease as Compared with Manic Depression

This example demonstrates the use of the claimed invention to identify biomarkers which are capable of differentiating between Alzheimer's Disease and Manic Depression and use of same.

Whole blood samples were taken from patients diagnosed with having Alzheimer's Disease Disease and Whole blood samples were taken from patients diagnosed with Manic Depression as defined herein. RNA expression profiles were then analyzed and the profiles generated for individuals having Alzheimer's Disease as compared with the profiles generated for individuals with Manic Depression. In each case, the diagnosis of Alzheimer's Disease and Manic Depression is corroborated by a skilled Board certified physician. RNA expression profiles of Whole blood samples from individuals who were identified as having Alzheimer's Disease as described herein as compared with RNA expression profiles from individuals identified as Manic Depression were generated using GeneSpring™ software analysis as described herein. Hybridizations to create said RNA expression profiles were done using the Affymetrix® GeneChip® platforms (U133A and/or U133 Plus 2.0) as described herein (data not shown). Various experiments were performed as outlined above, and analyzed using either the Wilcox Mann Whitney rank sum test or other statistical tests as described herein, and those genes identified with a p value of <0.05 as between the patients with Alzheimer's Disease as compared with patients Manic Depression are shown in Table 3R.

Classification or class prediction of a test sample of an individual to differentiate as to whether said individual has Alzheimer's Disease or Manic Depression can be done using the differentially expressed genes as shown in Table 3R as the predictor genes in combination with well known statistical algorithms as would be understood by a person skilled in the art and described herein. Commercially available programs such as those provided by Silicon Genetics (e.g. GeneSpring™) for Class Prediction are also available.

RNA Expression Profiles

### EXAMPLE 5

In addition to methods to identify markers that distinguish between two diseases or conditions, this invention also includes methods to identify biomarkers specific for a group of three or more related diseases or conditions. The following three examples present methods to identify biomarkers for the following groups of diseases or conditions: cancer, cardiovascular disease and neurological disease, and the identified markers thereof. However the invention is not limited to these three groups of diseases or conditions.

Cancer

This example demonstrates the use of the claimed invention to identify biomarkers of cancer and use of same.

As used herein “Cancer” is defined as any of the various types of malignant neoplasms, most of which invade surrounding tissues, may metastasize to several sites, and are likely to recur after attempted removal and to cause death of the patient unless adequately treated; especially, any such carcinoma or sarcoma, but, in ordinary usage, especially the former. In each case, the diagnosis of Cancer was corroborated by a skilled Board certified physician. RNA expression profilesRNA expression profiles of Whole blood samples from individuals who were identified as having cancer as described herein as compared with RNA expression profiles from individuals not having cancer, were generated using GeneSpring™ software analysis as described herein. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein, and those genes identified with a p value of <0.05 as between the patients with cancer as compared with patients without cancer are shown in Table 6A.

Classification or class prediction of a test sample from an unknown patient in order to diagnose said individual with cancer can be done using the differentially expressed genes as shown in Table 6A in combination with well known statistical algorithms for class prediction as would be understood by a person skilled in the art and is described herein. Commercially available programs such as those provided by Silicon Genetics (e.g. GeneSpring™) for Class Prediction are also available.

Cardiovascular Disease

This example demonstrates the use of the claimed invention to identify biomarkers of cardiovascular disease and use of same.

As used herein in this example “Cardiovascular Disease” is defined as a disease affecting the heart or blood vessels. Cardiovascular diseases include coronary artery disease, hearart failure, and hypertension. In each case, the diagnosis of Cardiovascular Disease was corroborated by a skilled Board certified physician. RNA expression profiles of Whole blood samples from individuals who were identified as having Cardiovascular Disease as described herein as compared with RNA expression profiles from individuals not having Cardiovascular Disease, were generated using GeneSpring™ software analysis as described herein. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein, and those genes identified with a p value of <0.05 as between the patients with Cardiovascular Disease as compared with patients without Cardiovascular Disease are shown in Table 6B.

Classification or class prediction of a test sample from an unknown patient in order to diagnose said individual with Cardiovascular Disease can be done using the differentially expressed genes as shown in Table 6B in combination with well known statistical algorithms for class prediction as would be understood by a person skilled in the art and is described herein

Neurological Diseases

This example demonstrates the use of the claimed invention to identify biomarkers of Neurological Disease and use of same.

As used herein “Neurological Disease” is defined as a disorder of the nervous system, and include disorders that involve the central nervous system (brain, brainstem and cerebellum), the peripheral nervous system (including cranial nerves), and the autonomic nervous system (parts of which are located in both central and peripheral nervous system). In particular neurological disease includes alzheimers', schizophrenia, and manic depression syndrome. In each case, the diagnosis of Neurological Disease was corroborated by a skilled Board certified physician. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein, and those genes identified with a p value of <0.05 as between the patients with neurological Disease as compared with patients without neurological Disease are shown in Table 6C.

Classification or class prediction of a test sample from an unknown patient in order to diagnose said individual with Neurological Disease can be done using the differentially expressed genes as shown in Table 6C in combination with well known statistical algorithms for class prediction as would be understood by a person skilled in the art and is described herein

### EXAMPLE 6

In addition to methods to identify biomarkers that are associated with a specific group of diseases or conditions, another aspect of this invention includes methods to identify biomarkers that are associated with the administration of a specific drug or exogenous substance, or a specific grouping of drugs or exogenous substances thereof. In essence this aspect of the invention provides a method of providing an individuals drug signature. The administration of the exogenous substance(s) or drug(s) can be via any route and the instant methods of identifying these markers can be applied at any specifies time point(s) after said administration. The following examples illustrate embodiments of this drug signature aspect of the invention, but the invention is not limited to the methods comprising the drug(s) and exogenous substance(s), or groups of drugs amd exogenous substances illustrated below.

Celebrex^{R}

Celebrex Versus Other COX Inhibitors:

This example demonstrates the use of the claimed invention to identify biomarkers associated with Celebrex^{R} and use of same.

This example demonstrates the use of the claimed invention to detect differential gene expression in Whole blood samples taken from individuals who have been adminstered with Celebrex^{R} as compared to Whole blood samples taken from individuals who have been adminstered with any Cox inhibitor except Celebrex^{R}.

As used herein “Cox Inhibitor” is defined as anti-inflammatory drug that covalently modifies cyclooxygenases (Cox). RNA expression profiles from individuals who have been adminstered with Celebrex^{R} were analyzed and compared to profiles from individuals who have been adminstered with any Cox inhibitor except Celebrex^{R}. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Celebrex^{R} as compared to individuals who have been adminstered with any Cox inhibitor except Celebrex^{R} is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Celebrex^{R} as compared to individuals who have been adminstered with any Cox inhibitor except Celebrex^{R,} are shown in Table 7A.

Celebrex Versus no Celebrex:

This example demonstrates the use of the claimed invention to detect differential gene expression in Whole blood samples taken from individuals who have been adminstered with Celebrex^{R} as compared to Whole blood samples taken from individuals who have been not been adminstered with Celebrex^{R}. RNA expression profiles from individuals who have been adminstered with Celebrex^{R} were analyzed and compared to profiles from individuals who have not been adminstered with Celebrex^{R}. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Celebrex^{R} as compared to individuals who have been not been adminstered with Celebrex^{R} is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Celebrex^{R} as compared to individuals who have not been adminstered with Celebrex^{R,} are shown in Table 7B.

Vioxx^{R}

Vioxx^{R} Versus no Vioxx^{R}:

This example demonstrates the use of the claimed invention to detect differential gene expression in Whole blood samples taken from individuals who have been adminstered with Vioxx^{R} as compared to Whole blood samples taken from individuals who have been not been adminstered with Vioxx^{R}. RNA expression profiles from individuals who have been adminstered with Vioxx^{R} were analyzed and compared to profiles from individuals who have not been adminstered with Vioxx^{R}. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Vioxx^{R} as compared to individuals who have been not been adminstered with Vioxx^{R} is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Vioxx^{R} as compared to individuals who have not been adminstered with Vioxx^{R} are shown in Table 7C.

Vioxx^{R} Versus other COX Inhibitors

This example demonstrates the use of the claimed invention to detect differential gene expression in Whole blood samples taken from individuals who have been adminstered with Vioxx^{R} as compared to Whole blood samples taken from individuals who have been adminstered with any Cox inhibitor except Vioxx^{R}.

RNA expression profiles from individuals who have been adminstered with Vioxx^{R} were analyzed and compared to profiles from individuals who have been adminstered with any Cox inhibitor except Vioxx^{R}. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Vioxx^{R} as compared to individuals who have been adminstered with any Cox inhibitor except Vioxx^{R} is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Vioxx^{R} as compared to individuals who have been adminstered with any Cox inhibitor except Vioxx^{R} are shown in Table 7D.

Non-Steroidal Anti-Inflammatory Agents (NSAIDs)

This example demonstrates the use of the claimed invention to detect differential gene expression in Whole blood samples taken from individuals who have been adminstered with non-steroidal anti-inflammatory agents as compared to Whole blood samples taken from individuals who have been not been adminstered with non-steroidal anti-inflammatory agents. As defined herein, non-steroidal anti-inflammatory agents are defined as a large group of anti-inflammatory agents that work by inhibiting the production of prostaglandins. They exert anti-inflammatory, analgesic and antipyretic actions and include: ibuprofen, ketoprofen, piroxicam, naproxen, sulindac, aspirin, choline subsalicylate, diflunisal, fenoprofen, indomethacin, meclofenamate, salsalate, tolmetin and magnesium salicylate. Not icluded are steroidal compounds (such as hydrocortisone or prednisone) exerting anti-inflammatory activity. RNA expression profiles from individuals who have been adminstered with non-steroidal anti-inflammatory agents were analyzed and compared to profiles from individuals who have not been adminstered with non-steroidal anti-inflammatory agents. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with non-steroidal anti-inflammatory agents as compared to individuals who have been not been adminstered with non-steroidal anti-inflammatory agents is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with non-steroidal anti-inflammatory agents as compared to individuals who have not been adminstered with non-steroidal anti-inflammatory agents are shown in Table 7E.

Cortisone

This example demonstrates the use of the claimed invention to detect differential gene expression in Whole blood samples taken from individuals who have been adminstered with Cortisone as compared to Whole blood samples taken from individuals who have been not been adminstered with Cortisone. RNA expression profiles from individuals who have been adminstered with Cortisone were analyzed and compared to profiles from individuals who have not been adminstered with Cortisone. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms (U133A and U133 Plus 2.0) as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Cortisone as compared to individuals who have been not been adminstered with Cortisone is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Cortisone as compared to individuals who have not been adminstered with Cortisone are shown in Table 7F.

Visco Supplement

This example demonstrates the use of the claimed invention to detect differential gene expression in Whole blood samples taken from individuals who have been adminstered with Visco Supplement as compared to Whole blood samples taken from individuals who have been not been adminstered with Visco Supplement. RNA expression profiles from individuals who have been adminstered with Visco Supplement were analyzed and compared to profiles from individuals who have not been adminstered with Visco Supplement. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms platforms (U133A and U133 Plus 2.0) as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Visco Supplement as compared to individuals who have been not been adminstered with Visco Supplement is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Visco Supplement as compared to individuals who have not been adminstered with Visco Supplement are shown in Table 7G.

Lipitor

This example demonstrates the use of the claimed invention to detect differential gene expression in Whole blood samples taken from individuals who have been adminstered with Lipitor as compared to Whole blood samples taken from individuals who have been not been adminstered with Lipitor. RNA expression profiles from individuals who have been adminstered with Lipitor were analyzed and compared to profiles from individuals who have not been adminstered with Lipitor. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have been adminstered with Lipitor as compared to individuals who have been not been adminstered with Lipitor is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have been adminstered with Lipitor as compared to individuals who have not been adminstered with Lipitor are shown in Table 7H.

Smoking

This example demonstrates the use of the claimed invention to detect differential gene expression in Whole blood samples taken from individuals who have smoked cigarettes and cigars as compared to Whole blood samples taken from individuals who have not smoked cigarettes and cigars. RNA expression profiles from individuals who have smoked were analyzed and compared to profiles from individuals who have not smoked. Preferably healthy individuals are chosen who are age and sex matched to said individuals being compared. Total mRNA from a blood sample is taken from each individual and isolated using TRIzol® reagent (GIBCO) and fluorescently labelled probes for each blood sample is generated as described above. Hybridizations to create said RNA expression profiles were done using the Affymetrix® GeneChip® platforms (U133A and U133 Plus 2.0) platforms as described herein (data not shown). Various experiments were performed as outlined above, and analyzed using either the Wilcox Mann Whitney rank sum test or other statistical tests as described herein. Identification of genes differentially expressed in Whole blood samples from individuals who have smoked as compared to individuals who have not smoked is determined by statistical analysis using the Wilcox Mann Whitney rank sum test using either the Wilcox Mann Whitney rank sum test or other statistical tests as described herein. Those differentially expressed genes identified with a p value of <0.05 as between the individuals who have smoked as compared to individuals who have not smoked are shown in Table 7I.

### EXAMPLE 7

Identification of Genes Specific for OA Only by Removing Genes Relevant to Co-Morbidities and Other Disease States.

This example demonstrates the use of the claimed invention to detect differential gene expression in blood unique to Osteoarthritis as compared with other disease states.

Whole blood samples were taken from patients who were diagnosed with mild OA or severe OA and compared with individuals who were identified as normal individuals as defined herein. RNA expression profiles were then analysed to identify genes which are differentially expressed in OA as compared with normal. In each case, the diagnosis of OA was corroborated by a qualified physician.

Total mRNA from a blood sample taken from each patient was isolated using TRIzol® reagent (GIBCO) and fluorescently labeled probes for each blood sample were generated as described above. Each probe was denatured and hybridized to a ChondroChip™ as described herein. Identification of genes differentially expressed in Whole blood samples from patients with mild or severe OA as compared to healthy patients was determined by statistical analysis using the Weltch ANOVA test (Michelson and Schofield, 1996). (Dendogram analysis not shown).

In order to identify genes differentially expressed in blood unique to OA but not differentially expressed as a result of possible co-morbidities including hypertension, obesity, asthma, taking systemic steroids, or allergies, genes identified as differentially expressed in both OA and any of the genes identified as differentially expressed as a result of co-morbidity, e.g., Table 1A (co-morbidity of OA and hypertension v. normal), Table 1B (co-morbidity of OA and obesity v. normal), Table 3C (co-morbidity of OA and allergy v. normal), Table 3D (co-morbidity of OA and taking systemic steroids v. normal), and genes in common with people identified as having asthma and OA (Table 3AA) were removed. Similarly any genes and unique to obesity (Table 3R), hypertension (Table 3P), allergies (Table 3T), systemic steroids (Table 3V) were also removed. As a result of these comparisons, a list of genes unique to individuals with OA were identified. The identity of the differentially expressed genes is shown in Table 3AB.

It would be clear to a person skilled in the art that rather than simply remove those genes which are relevant to other disease states, one could use a more refined analysis and remove those genes which show the same trend in gene expression, e.g. remove those genes which show up regulation in a co-morbid state and also show up-regulation in the single disease state, but retain those genes which show a different trend in gene expression e.g. retain those genes which show up regulation in a co-morbid state as compared to down regulation in a single disease state.

Classification or class prediction of a test sample of an individual to determine whether said individual has OA or does not have OA can be done using the differentially expressed genes as shown in Table 3AB, irrespective of whether the individual presents with co-morbidity using well known statistical algorithms as would be understood by a person skilled in the art and described herein. Commercially available programs such as those provided by Silicon Genetics (e.g. GeneSpring™) for Class Predication are also available.

Brain Cancer

Analysis of RNA expression profiles of Whole blood samples from individuals having brain cancer as compared with RNA expression profiles from normal individuals.

This example demonstrates the use of the claimed invention to detect differential gene expression in Whole blood samples taken from patients diagnosed with brain cancer as compared to Whole blood samples taken from healthy patients.

As used herein “brain cancer” refers to all forms of primary brain tumors, both intracranial and extracranial and includes one or more of the following: Glioblastoma, Ependymoma, Gliomas, Astrocytoma, Medulloblastoma, Neuroglioma, Oligodendroglioma, Meningioma, Retinoblastoma, and Craniopharyngioma.

Whole blood samples are taken from patients diagnosed with brain cancer as defined herein. RNA expression profiles are then analysed and compared to profiles from patients unaffected by any disease. Preferably healthy patients are chosen who are age and sex matched to said patients diagnosed with disease. In each case, the diagnosis of brain cancer is corroborated by a skilled Board certified physician.

Total mRNA from a blood sample is taken from each patient and isolated using TRIzol® reagent (GIBCO) and fluorescently labeled probes for each blood sample are generated as described above. Each probe is denatured and hybridized to a Affymetrix U133A Chip and/or ChondroChip™ as described herein. Identification of genes differentially expressed in Whole blood samples from patients with brain cancer as compared to healthy patients is determined by statistical analysis using the Wilcox Mann Whitney rank sum test (Glantz S A. Primer of Biostatistics, 5th ed., New York, USA: McGraw-Hill Medical Publishing Division, 2002).

Classification or class prediction of a test sample of an individual to determine whether said individuals has brain cancer or does not having brain cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Prostate Cancer

Analysis of RNA expression profiles of Whole blood samples from individuals having prostate cancer as compared with RNA expression profiles from normal individuals.

This example demonstrates the use of the claimed invention to detect differential gene expression in Whole blood samples taken from patients diagnosed with prostate cancer as compared to Whole blood samples taken from healthy patients

As used herein “prostate cancer” refers to a malignant cancer originating within the prostate gland. Patients identified as having prostate cancer can have any stage of prostate cancer, as determined clinically (by digital rectal exam or PSA testing) and or pathologically. Staging of prostate cancer can done in accordance with TNM or the Staging System of the American Joint Committee on Cancer (AJCC). In addition to the TNM system, other systems may be used to stage prostate cancer, for example, the Whitmore-Jewett system.

Whole blood samples are taken from patients diagnosed with prostate cancer as defined herein. RNA expression profiles are then analysed and compared to profiles from patients unaffected by any disease to identify genes which differentiate as between the two groups. Similarly RNA expression profiles can be analysed so as to differentiate as between the severity of the prostate cancer. Preferably healthy patients are chosen who are age and sex matched to said patients diagnosed with disease or with a specific stage of said disease. In each case, the diagnosis of prostate cancer is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with prostate cancer as compared to healthy patients is determined by statistical analysis using the Wilcox Mann Whitney rank sum test (Glantz S A. Primer of Biostatistics. 5th ed. New York, USA: McGraw-Hill Medical Publishing Division, 2002).

Classification or class prediction of a test sample of an individual to determine whether said individuals has prostate cancer, has a specific stage of prostate cancer, or does not having prostate cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Ovarian Cancer

Analysis of RNA expression profiles of Whole blood samples from individuals having ovarian cancer as compared with RNA expression profiles from normal individuals.

This example demonstrates the use of the claimed invention to detect differential gene expression in Whole blood samples taken from patients diagnosed with ovarian cancer as compared to Whole blood samples taken from healthy patients.

As used herein “ovarian cancer” refers to a malignant cancerous growth originating within the ovaries. Patients identified as having ovarian cancer can have any stage of ovarian cancer. Staging is done by combining information from imaging tests with the results of a surgical examination done during a laprotomy. Numbered stages I to IV are used to describe the extent of the cancer and whether it has spread (metastasized) to more distant organs.

Whole blood samples are taken from patients diagnosed with ovarian cancer, or with a specific stage of ovarian cancer as defined herein. RNA expression profiles are then analysed and compared to profiles from patients unaffected by any disease. Preferably healthy patients are chosen who are age and sex matched to said patients diagnosed with disease or with a specific stage of said disease. In each case, the diagnosis of ovarian cancer is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with ovarian cancer and or a specific stage of ovarian cancer as compared to healthy patients is determined by statistical analysis using the Wilcox Mann Whitney rank sum test (Glantz S A. Primer of Biostatistics. 5th ed. New York, USA: McGraw-Hill Medical Publishing Division, 2002).

Classification or class prediction of a test sample of an individual to determine whether said individuals has ovarian cancer, has a specific stage of ovarian cancer or does not having ovarian cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Gastric Cancer

Analysis of RNA expression profiles of Whole blood samples from individuals having gastric cancer as compared with RNA expression profiles from normal individuals.

This example demonstrates the use of the claimed invention to detect differential gene expression in Whole blood samples taken from patients diagnosed with gastric cancer as compared to Whole blood samples taken from healthy patients.

As used herein “gastric or stomach cancer” refers to a cancerous growth originating within the stomach and includes gastric adenocarcinoma, primary gastric lymphoma and gastric nonlymphoid sarcoma. Patients identified as having stomac can also be categorized by stage of said cancer as determined by the System of the American Joint Committee on Cancer (AJCC).

Whole blood samples are taken from patients diagnosed with stomach cancer, or with a specific stage of stomach cancer as defined herein. RNA expression profiles are then analysed and compared to profiles from patients unaffected by any disease. Preferably healthy patients are chosen who are age and sex matched to said patients diagnosed with disease or with a specific stage of said disease. In each case, the diagnosis of stomach cancer is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with stomach cancer and or a specific stage of stomach cancer as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has stomach cancer, has a specific stage of stomach cancer or does not having stomach cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Breast Cancer

Analysis of RNA expression profiles of Whole blood samples from individuals having breast cancer as compared with RNA expression profiles from normal individuals.

This example demonstrates the use of the claimed invention to detect differential gene expression in Whole blood samples taken from patients diagnosed with breast cancer as compared to Whole blood samples taken from healthy patients.

As used herein “breast cancer” refers to a cancerous growth originating within the breast and includes invasive and non invasive breast cancer such as ductal carcinoma in situ (DCIS), lobular carcinoma in situ (LCIS), infiltrating ductal carcinoma, and infiltrating lobular carcinoma. Patients identified as having breast cancer can also be categorized by stage of said cancer as determined by the System of the American Joint Committee on Cancer (AJCC) or TNM classification.

Whole blood samples are taken from patients diagnosed with breast cancer, or with a specific stage of breast cancer as defined herein. RNA expression profiles are then analysed and compared to profiles from patients unaffected by any disease. Preferably healthy patients are chosen who are age and sex matched to said patients diagnosed with disease or with a specific stage of said disease. In each case, the diagnosis of breast cancer is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with breast cancer and or a specific stage of breast cancer as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has breast cancer, has a specific stage of breast cancer or does not have breast cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Nasopharyngeal Cancer

Analysis of RNA expression profiles of Whole blood samples from individuals having nasopharyngeal cancer as compared with RNA expression profiles from normal individuals.

This example demonstrates the use of the claimed invention to detect differential gene expression in Whole blood samples taken from patients diagnosed with nasopharyngeal cancer as compared to Whole blood samples taken from healthy patients.

As used herein “nasopharyngeal cancer” refers to a cancerous growth arising from the epithelial cells that cover the surface and line the nasopharynx. Patients identified as having nasopharyngeal cancer can also be categorized by stage of said cancer as determined by the System of the American Joint Committee on Cancer (AJCC) or TNM classification.

Whole blood samples are taken from patients diagnosed with nasopharyngeal cancer, or with a specific stage of nasopharyngeal cancer as defined herein. RNA expression profiles are then analysed and compared to profiles from patients unaffected by any disease. Preferably healthy patients are chosen who are age and sex matched to said patients diagnosed with disease or with a specific stage of said disease. In each case, the diagnosis of nasopharyngeal cancer is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with nasopharyngeal cancer and or a specific stage of breast cancer as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has nasopharyngeal cancer, has a specific stage of nasopharyngeal cancer or does not have nasopharyngeal cancer can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Guillain Barre Syndrome

Analysis of RNA expression profiles of Whole blood samples from individuals having Guillain Barre syndrome as compared with RNA expression profiles from normal individuals.

This example demonstrates the use of the claimed invention to detect differential gene expression in Whole blood samples taken from patients diagnosed with Guillain Barre syndrome as compared to Whole blood samples taken from healthy patients.

As used herein “Guillain Barre syndrome” refers to an acute, usually rapidly progressive form of inflammatory polyneuropathy characterized by muscular weakness and mild distal sensory loss.

Whole blood samples are taken from patients diagnosed with Guillain Barre syndrome as defined herein. RNA expression profiles are then analysed and compared to profiles from patients unaffected by any disease. Preferably healthy patients are chosen who are age and sex matched to said patients diagnosed with disease. In each case, the diagnosis of Guillain Barre syndrome is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with Guillain Barre syndrome as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has Guillain Barre syndrome, or does not have Guillain Barre syndrome can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Fibromyalgia

Analysis of RNA expression profiles of Whole blood samples from individuals having Fibromyalgia as compared with RNA expression profiles from normal individuals.

This example demonstrates the use of the claimed invention to detect differential gene expression in Whole blood samples taken from patients diagnosed with Fibromyalgia as compared to Whole blood samples taken from healthy patients.

As used herein “Fibromyalgia” refers to widespread chronic musculoskeletal pain and fatigue. The pain comes from the connective tissues, such as the muscles, tendons, and ligaments and does not involve the joints. Whole blood samples are taken from patients diagnosed with Fibromyalgia as defined herein. RNA expression profiles are then analysed and compared to profiles from patients unaffected by any disease. Preferably healthy patients are chosen who are age and sex matched to said patients diagnosed with disease. In each case, the diagnosis of Fibromyalgia is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with Fibromyalgia as compared to healthy patients is determined by statistical analysis using the Wilcox Mann Whitney rank sum test (Glantz S A. Primer of Biostatistics. 5th ed. New York, USA: McGraw-Hill Medical Publishing Division, 2002).

Classification or class prediction of a test sample of an individual to determine whether said individuals has Fibromyalgia, or does not have Fibromyalgia can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Multiple Sclerosis

Analysis of RNA expression profiles of Whole blood samples from individuals having Multiple Sclerosis as compared with RNA expression profiles from normal individuals.

This example demonstrates the use of the claimed invention to detect differential gene expression in Whole blood samples taken from patients diagnosed with Multiple Sclerosis as compared to Whole blood samples taken from healthy patients.

As used herein “Multiple Sclerosis” refers to chronic progressive nervous disorder involving the loss of myelin sheath surrounding certain nerve fibres. Whole blood samples are taken from patients diagnosed with Multiple Sclerosis as defined herein. RNA expression profiles are then analysed and compared to profiles from patients unaffected by any disease. Preferably healthy patients are chosen who are age and sex matched to said patients diagnosed with disease. In each case, the diagnosis of Multiple Sclerosis is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with Multiple Sclerosis as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has Multiple Sclerosis, or does not have Multiple Sclerosis can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Muscular Dystrophy

Analysis of RNA expression profiles of Whole blood samples from individuals having Muscular Dystrophy as compared with RNA expression profiles from normal individuals.

This example demonstrates the use of the claimed invention to detect differential gene expression in Whole blood samples taken from patients diagnosed with Muscular Dystrophy as compared to Whole blood samples taken from healthy patients.

As used herein “Muscular Dystrophy” refers to a hereditary disease of the muscular system characterized by weakness and wasting of the skeletal muscles. Muscular Dystrophy includes Duchennes' Muscular Dystrophy, limb-girdle muscular dystrophy, myotonia atrophica, myotonic muscular dystrophy, pseudohypertrophic muscular dystrophy, and Steinhardt's disease.

Whole blood samples are taken from patients diagnosed with Muscular Dystrophy as defined herein. RNA expression profiles are then analysed and compared to profiles from patients unaffected by any disease. Preferably healthy patients are chosen who are age and sex matched to said patients diagnosed with disease. In each case, the diagnosis of Muscular Dystrophy is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with Muscular Dystrophy as compared to healthy patients is determined by statistical analysis using the Wilcox Mann Whitney rank sum test (Glantz S A,. Primer of Biostatistics, 5th ed., New York, USA: McGraw-Hill Medical Publishing Division, 2002).

Classification or class prediction of a test sample of an individual to determine whether said individuals has Muscular Dystrophy, or does not have Muscular Dystrophy can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Septic Joint Arthroplasty

Analysis of RNA expression profiles of Whole blood samples from individuals having septic joint arthroplasty as compared with RNA expression profiles from normal individuals.

This example demonstrates the use of the claimed invention to detect differential gene expression in Whole blood samples taken from patients diagnosed with septic joint arthroplasty as compared to Whole blood samples taken from healthy patients.

As used herein “septic joint arthroplasty” refers to an inflammation of the joint caused by a bacterial infection.

Whole blood samples are taken from patients diagnosed with septic joint arthroplasty as defined herein. RNA expression profiles are then analysed and compared to profiles from patients unaffected by any disease. Preferably healthy patients are chosen who are age and sex matched to said patients diagnosed with disease. In each case, the diagnosis of septic joint arthroplasty is corroborated by a skilled Board certified physician.

Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with septic joint arthroplasty as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has septic joint arthroplasty, or does not have septic joint arthroplasty can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Hepatitis

Analysis of RNA expression profiles of Whole blood samples from individuals having hepatitis as compared with RNA expression profiles from normal individuals.

This example demonstrates the use of the claimed invention to detect gene expression in Whole blood samples taken from patients diagnosed with hepatitis as compared to Whole blood samples taken from healthy patients. As used herein “hepatitis” refers to an inflammation of the liver caused by a virus or toxin and can include hepatitis A, hepatitis B, hepatitis C, hepatitis D, hepatitis E, and hepatitis F. Whole blood samples are taken from patients diagnosed with hepatitis as defined herein. RNA expression profiles are then analysed and compared to profiles from patients unaffected by any disease. Preferably healthy patients are chosen who are age and sex matched to said patients diagnosed with disease. In each case, the diagnosis of hepatitis is corroborated by a skilled Board certified physician. Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with hepatitis as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has hepatitis, or does not have hepatitis can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Malignant Hyperthermia Susceptibility

Analysis of RNA expression profiles of Whole blood samples from individuals having Malignant Hyperthermia Susceptibility as compared with RNA expression profiles from normal individuals.

This example demonstrates the use of the claimed invention to detect differential gene expression in Whole blood samples taken from patients diagnosed with Malignant Hyperthermia Susceptibility as compared to Whole blood samples taken from healthy patients. As used herein “Malignant Hyperthermia Susceptibility” refers to a pharmacogenetic disorder of skeletal muscle calcium regulation often developing during or after a general anaesthesia.

Whole blood samples are taken from patients diagnosed with Malignant Hyperthermia Susceptibility as defined herein. RNA expression profiles are then analysed and compared to profiles from patients unaffected by any disease. Preferably healthy patients are chosen who are age and sex matched to said patients diagnosed with disease. In each case, the diagnosis of Malignant Hyperthermia Susceptibility is corroborated by a skilled Board certified physician. Total mRNA from a blood sample is taken from each patient and isolated using TRIzol® reagent (GIBCO) and fluorescently labeled probes for each blood sample is generated as described above. Each probe is denatured and hybridized to a Affymetrix U133A Chip and/or a ChondroChip™ as described herein. Identification of genes differentially expressed in Whole blood samples from patients with Malignant Hyperthermia Susceptibility as compared to healthy patients is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of an individual to determine whether said individuals has Malignant Hyperthermia Susceptibility, or does not have Malignant Hyperthermia Susceptibility can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Osteoarthritic Horses

Analysis of RNA expression profiles of Whole blood samples from horses having osteoarthritis as compared with RNA expression profiles from normal or non-osteoarthritic horses.

This example demonstrates the use of the claimed invention to detect differential gene expression in Whole blood samples taken from horses so as to diagnose equine arthritis as compared to Whole blood samples taken from healthy horses.

As used herein “arthritis” in reference to horses refers to a degenerative joint disease that affects horses by causing lameness. Although it can appear in any joint, most common areas are the upper knee joint, front fetlocks, hocks, or coffin joints in the front feet. The condition can be caused by trauma, mineral or dietary deficiency, old age, poor conformation, over exertion or infection. The different structures that can be damaged in arthritis are the cartilage inside joints, the bone in the joints, the joint capsule, the synovial membranes, the ligaments around the joints and lastly the fluid that lubricates the insides of ‘synovial joints’. In severe cases all of these structures are affected. In for example osteochondrosis only the cartilage may be affected.

Regardless of the cause, the disease begins when the synovial fluid that lubricates healthy joints begins to thin. The decrease in lubrication causes the cartilage cushion to break down, and eventually the bones begin to grind painfully against each other. Diagnostic tests used to confirm arthritis include X-rays, joint fluid analysis, and ultrasound.

Whole blood samples are taken from horses diagnosed with arthritis as defined herein. RNA expression profiles are then analysed and compared to profiles from horses unaffected by any disease. Preferably healthy horses are chosen who are age and sex matched to said horses diagnosed with disease. In each case, the diagnosis of arthritis is corroborated by a certified veterinarian.

Total mRNA from a blood sample is taken from each horse and isolated using TRIzol® reagent (GIBCO) and fluorescently labeled probes for each blood sample is generated as described above. Each probe is denatured and hybridized to a Affymetrix U133A Chip and/or a ChondroChip™ as described herein. An equine specific microarray representing the equine genome can also be used. Identification of genes differentially expressed in Whole blood samples from horses with arthritis as compared to healthy horses is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of a horse to determine whether said horse has arthritis or does not have arthritis can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Osteoarthritic Dogs

Analysis of RNA expression profiles of Whole blood samples from dogs having osteoarthritis as compared with RNA expression profiles from normal or non-osteoarthritic dogs.

This example demonstrates the use of the claimed invention to detect differential gene expression in Whole blood samples taken from dogs so as to diagnose equine arthritis as compared to Whole blood samples taken from healthy horses.

As used herein “osteoarthritis” in reference to dogs is a form of degenerative joint disease which involves the deterioration of and changes to the cartilage and bone. In response to inflammation in and about the joint, the body responds with bony remodeling around the joint structure. This process can be slow and gradual with minimal outward symptoms, or more rapidly progressive with significant pain and discomfort. Osteoarthritic changes can occur in response to infection and injury of the joint as well.

Whole blood samples are taken from dogs diagnosed with osteoarthritis as defined herein. RNA expression profiles are then analysed and compared to profiles from dogs unaffected by any disease. Preferably healthy dogs are chosen who are age, sex and breed matched to said dogs diagnosed with disease. In each case, the diagnosis of osteoarthritis is corroborated by a certified veterinarian.

Total mRNA from a blood sample is taken from each dog and isolated using TRIzol® reagent (GIBCO) and fluorescently labeled probes for each blood sample is generated as described above. Each probe is denatured and hybridized to a Affymetrix U133A Chip and/or a ChondroChip™ as described herein. A canine specific microarray representing the canine genome can also be used. Identification of genes differentially expressed in Whole blood samples from dogs with osteoarthritis as compared to healthy horses is determined by statistical analysis using the Wilcox Mann Whitney rank sum test (Glantz S A, Primer of Biostatistics, 5th ed., New York, USA: McGraw-Hill Medical Publishing Division, 2002).

Classification or class prediction of a test sample of a dog to determine whether said dog has osteoarthritis or does not have osteoarthritis can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Manic Depression Syndrome (MDS) as Compared with Schizophrenia RNA Expression Profiles

Analysis of RNA expression profiles of Whole blood samples from individuals having Manic Depression Syndrome (MDS) as compared with RNA expression profiles from individuals having Schizophrenia.

This example demonstrates the use of the claimed invention to detect differential gene expression in Whole blood samples taken from patients diagnosed with MDS as compared to Whole blood samples taken from schizophrenic patients.

As used herein “Manic Depression Syndrome (MDS)” refers to a mood disorder characterized by alternating mania and depression. As used herein, “schizophrenia” is defined as a psychotic disorders characterized by distortions of reality and disturbances of thought and language and withdrawal from social contact. Patients diagnosed with “schizophrenia” can include patients having any of the following diagnosis: an acute schizophrenic episode, borderline schizophrenia, catatonia, catatonic schizophrenia, catatonic type schizophrenia, disorganized schizophrenia, disorganized type schizophrenia, hebephrenia, hebephrenic schizophrenia, latent schizophrenia, paranoic type schizophrenia, paranoid schizophrenia, paraphrenia, paraphrenic schizophrenia, psychosis, reactive schizophrenia or the like.

Whole blood samples are taken from patients diagnosed with MDS or Schizophrenia as defined herein. RNA expression profiles are then analyzed and compared to profiles from patients unaffected by any disease. Preferably healthy patients are chosen who are age and sex matched to said patients diagnosed with disease. In each case, the diagnosis of MDS and Schizophrenia is corroborated by a skilled Board certified physician. Total mRNA from a blood sample is taken from each patient and isolated using TRIzol* reagent (GIBCO) and fluorescently labeled probes for each blood sample is generated as described above.

Each probe is denatured and hybridized to a Affymetrix U133A Chip and/or a ChondroChip™ as described herein. Identification of genes differentially expressed in Whole blood samples from patients with MDS as compared to Schizophrenic patients as compared to normal individuals is determined by statistical analysis using the Wilcox Mann Whitney rank sum test (Glantz S A, Primer of Biostatistics, 5th ed., New York, USA: McGraw-Hill Medical Publishing Division, 2002) (data not shown). 294 genes were identified as being differentially expressed with a p value of <0.05 as between the schizophrenic patients, the MDS patients and those control individuals. The identity of the differentially expressed genes is shown in Table 3AC.

Classification or class prediction of a test sample of an individual to determine whether said individuals has MDS, has Schizophrenia or is normal can be done using the differentially expressed genes identified as described above as the predictor genes in combination with well known statistical algorithms as would be understood by a person skilled in the art and described herein. Commercially available programs such as those provided by Silicon Genetics (e.g. GeneSpring™) for Class Predication are also available.

Prediction of Progression of Osteoarthritis.

Analysis of RNA expression profiles of Whole blood samples of Individuals So as to Predict Progression of Osteoarthritis.

This example demonstrates the use of the claimed invention to predict the progression of Osteoarthritis.

As used herein “osteoarthritis” is a form of degenerative joint disease which involves the deterioration of and changes to the cartilage and bone. In response to inflammation in and about the joint, the body responds with bony remodeling around the joint structure. This process can be slow and gradual with minimal outward symptoms, or more rapidly progressive with significant pain and discomfort. Osteoarthritic changes can occur in response to infection and injury of the joint as well.

Whole blood samples are taken from test individuals not having any symptoms of osteoarthritis and RNA expression profiles are then analyzed and compared to profiles from individuals having mild osteoarthritis.

Classification or class prediction of a test sample of said individual to determine whether said individual has mild osteoarthritis or does not have osteoarthritis can be done using the differentially expressed genes identified as described herein as the predictor genes in combination with well known statistical algorithms as would be understood by a person skilled in the art and described herein. Commercially available programs such as those provided by Silicon Genetics (e.g. GeneSpring™) for Class Predication are also available.

Individuals identified with mild osteoarthritis as a result of this classification have a significantly greater chance of developing moderate, marked and/or severe osteoarthris than those individuals not diagnosed with mild osteoarthritis.

Therapy

Microarray Data Analysis of RNA expression profiles of Whole blood samples from individuals having a condition as compared with RNA expression profiles from individuals not having said condition, and wherein said individual is undergoing therapeutic treatment in light of said condition.

This example demonstrates the use of the claimed invention to detect differential gene expression in Whole blood samples taken from individuals undergoing therapeutic treatment of a condition as compared with RNA expression profiles from individuals not undergoing treatment.

Whole blood samples are taken from patients who are undergoing therapeutic treatment. RNA expression profiles are then analysed and compared to profiles from patients not undergoing treatment.

Total mRNA from a blood sample taken from each patient is isolated using TRIzol® reagent (GIBCO) and fluorescently labeled probes for each blood sample are generated as described above. Each probe is denatured and hybridized to a microarray for example the 15K Chondrogene Microarray Chip (ChondroChip™), Affymetrix Genechip or Blood chip as described herein.

Identification of genes differentially expressed in Whole blood samples from patients undergoing therapeutic treatment as compared to patients not undergoing treatment is determined by statistical analysis using the Wilcox Mann Whitney rank sum test (Glantz S A. Primer of Biostatistics., differentially expressed genes are then identified as being differentially expressed with a p value of <0.05.

One skilled in the art will appreciate readily that the present invention is well adapted to carry out the objects and obtain the ends and advantages mentioned, as well as those objects, ends and advantages inherent herein.

The present examples, along with the methods, procedures, treatments, molecules, and specific compounds described herein are presently representative of preferred embodiments, are exemplary, and are not intended as limitations on the scope of the invention.

Changes therein and other uses will occur to those skilled in the art which are encompassed within the spirit of the invention as defined by the scope of the claims.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying one or more biomarkers for a disease of interest, wherein each of said one or more biomarkers corresponds to an RNA transcript, comprising the method steps of:
a) determining the level of one or more RNA transcripts expressed in blood obtained from one or more individuals having said disease of interest, wherein each of said one or more RNA transcripts is a candidate biomarker for said disease of interest; and
b) comparing the level of each of said one or more RNA transcripts from said step a) with the level of each of said one or more RNA transcripts in blood obtained from one or more individuals not having said disease of interest,
wherein the RNA transcripts which display differing levels in the comparison of step b), are identified as being biomarkers for said disease of interest, and/or
c) determining the level of one or more RNA transcripts expressed in blood obtained from one or more individuals having said disease of interest, wherein each of said one or more transcripts is a candidate biomarker for said disease of interest; and
d) comparing the level of each of said one or more RNA transcripts from said step c) with the level of each of said one or more transcripts in blood obtained from one or more individuals having said disease of interest,
wherein the RNA transcripts which display the same levels in the comparison of step d), are identified as being biomarkers for said disease of interest.

2. The method of claim 1, wherein the disease is selected from the group consisting of: liver cancer, bladder cancer, brain cancer, prostate cancer, ovarian cancer, kidney cancer, gastric cancer, lung cancer, breast cancer, nasopharyngeal cancer, pancreatic cancer, osteoarthritis, depression, hypertension, heart failure, obesity, rheumatoid arthritis, hyperlipidemia, lung disease, chagas disease, allergies, schizophrenia, asthma, manic depression syndrome, ankylosing spondylitis, guillain barre syndrome, fibromyalgia, multiple sclerosis, muscular dystrophy, septic joint arthroplasty, hepatitis, crohn's disease or colitis, or malignant hyperthermia susceptibility, psoriasis, thyroid disorder, irritable bowel syndrome, osteoporosis, migraines, eczema, or a heart murmer.

3. A method of identifying one or more biomarkers of a stage of progression or regression of a disease of interest, wherein each of said one or more biomarkers corresponds to an RNA transcript, comprising the steps of:
a) determining the level of one or more RNA transcripts expressed in blood obtained from one or more individuals having a stage of said disease of interest, wherein said one or more individuals are at the same progressive or regressive stage of said disease of interest, and wherein each of said one or more transcripts is a candidate biomarker for determining the stage of progression or regression of said disease of interest, and;
b) comparing the level of each of said one or more RNA transcripts from said step a) with the level of each of said one or more transcripts in blood obtained from one or more individuals who are at a progressive or regressive stage of said disease of interest distinct from that of said one or more individuals of step a),
wherein those compared transcripts which display differing levels in the comparison of step b) are identified as being biomarkers for the stage of progression or regression of said disease of interest, and/or
c) determining the level of one or more RNA transcripts expressed in blood obtained from one or more individuals having a stage of said disease of interest, wherein said one or more individuals are at the same progressive or regressive stage of said disease of interest, and wherein each of said one or more transcripts is a candidate biomarker for determining the stage of progression or regression of said disease of interest, and;
d) comparing the level of each of said one or more RNA transcripts from said step c) with the level of each of said one or more RNA transcripts in blood obtained from one or more individuals who are at a progressive or regressive stage of said disease of interest as that of said one or more individuals of step c),
wherein those compared transcripts which display the same levels in the comparison of step d) are identified as being biomarkers for the stage of progression or regression of said disease of interest.

4. The method of claim 3, wherein said disease of interest is selected from the group consisting of: liver cancer, bladder cancer, brain cancer, prostate cancer, ovarian cancer, kidney cancer, gastric cancer, lung cancer, breast cancer, nasopharyngeal cancer, pancreatic cancer, osteoarthritis, depression, hypertension, heart failure, obesity, rheumatoid arthritis, hyperlipidemia, lung disease, chagas disease, allergies, schizophrenia and asthma, manic depression syndrome, ankylosing spondylitis, guillain barre syndrome, fibromyalgia, multiple sclerosis, muscular dystrophy, septic joint arthroplasty, hepatitis, crohn's disease or colitis, malignant hyperthermia susceptibility, psoriasis, thyroid disorder, irritable bowel syndrome, osteoporosis, migraines, eczema, or a heart murmer, alzheimer's, CAD, Diabetes, or colorectal cancer.

5. A method of identifying one or more biomarkers for a condition of interest, wherein each of said one or more biomarkers corresponds to an RNA transcript, comprising the steps of:
a) determining the level of one or more RNA transcripts expressed in blood obtained from one or more individuals having said condition of interest, wherein each of said one or more transcripts is a candidate biomarker for said condition of interest; and
b) comparing the level of each of said one or more RNA transcripts from said step a) with the level of each of said one or more RNA transcripts in blood obtained from one or more individuals not having said condition of interest,
wherein those compared transcripts which display differing levels in the comparison of step b) are identified as being biomarkers for said condition of interest, and/or
c) determining the level of one or more RNA transcripts expressed in blood obtained from one or more individuals having said condition of interest, wherein each of said one or more transcripts is a candidate biomarker for said condition of interest; and
d) comparing the level of each of said one or more RNA transcripts from said step c) with the level of each of said one or more RNA transcripts in blood obtained from one or more individuals having said condition of interest,
wherein those compared transcripts which display the same levels in the comparison of step d) are identified as being biomarkers for said condition of interest.

6. The method of claim 5, wherein said condition of interest is the condition resulting from the administration of medication wherein said medication is selected from the group consisting of: Celebrex, Vioxx, NSAIDS, Cortozone, Hyaluronic Acid, Systemic Steroids, hormone replacement therapy, pregnazone and birth control pills.

7. The method of claim 5, wherein said condition of interest is the condition resulting from exposure to an environmental condition, wherein said environmental condition is cigarette smoke.

8. A method of identifying one or more biomarkers to differentiate between a pair of disease and/or conditions, wherein said pair consists of a first and a second disease or condition of interest, and wherein each of said one or more biomarkers corresponds to an RNA transcript, comprising the steps of:
a) determining the level of one or more RNA transcripts expressed in blood obtained from one or more individuals having said first disease or condition of interest while not having said second disease or condition of interest, wherein each of said one or more transcripts is a candidate biomarker for differentiating said first disease or condition of interest from said second disease or condition of interest; and
b) comparing the level of each of said one or more RNA transcripts from said step a) with the level of each of said one or more genes transcripts in blood obtained from one or more individuals having said second disease or condition of interest while not having said first disease or condition of interest,
wherein those compared transcripts which display differing levels in the comparison of step b) are identified as being biomarkers for differentiating said first disease or condition of interest from said second disease or condition of interest.

9. The method of claim 8, wherein said first and second disease or condition of interest of said pair of disease and/or conditions, are selected from the group consisting of rheumatoid arthritis, osteoarthritis, schizophrenia, manic depression syndrome, liver cancer, hepatitis, bladder cancer, kidney cancer, bladder cancer, testicular cancer, pancreatic cancer, kidney cancer, liver cancer, stomach cancer, colon cancer, Chagas disease, heart failure, coronary artery disease, asymptomatic Chagas Disease, symptomatic Chagas Disease, alzheimer's Disease, allergies, systemic steroids, allergy, Type II Diabetes, obesity, hypertension, hyperliidemia, lung disease, bladder cancer, asthma, psoriasis, thyroid disorder, irritable bowel syndrome, osteoporosis, migraine headaches, excema, NASH, Crohn's colitis, chronic cholecystitis, cervical cancer, cardiovascular disease, and neurological disease.

10. The method of any one of claims 1-2, wherein said disease of interest is hypertension and wherein said one or more RNA transcripts are transcribed from one or more genes selected from the group consisting of the genes listed in Tables 1A, 1E, 1P and 1Q, or wherein said disease of interest is obesity and wherein said one or more RNA transcripts are transcribed from one or more genes selected from the group consisting of the genes listed in Tables 1B, 1F, 1R, and 1S, or wherein said disease of interest is allergies and wherein said one or more RNA transcripts are transcribed from one or more genes selected from the group consisting of the genes listed in Tables 1C, 1T, and 1U, or wherein said disease of interest is type II diabetes, and wherein said one or more RNA transcripts are transcribed from one or more genes selected from the group consisting of the genes listed in Table 1G, and wherein said marker does not identify the insulin gene, or wherein said disease of interest is hyperlipidemia, and wherein said one or more RNA transcripts are transcribed from one or more genes selected from the group consisting of the genes listed in Table 1H, or wherein said disease of interest is lung disease, and wherein said one or more RNA transcripts are transcribed from one or more genes selected from the group consisting of the genes listed in Table 1I, or wherein said disease of interest is bladder cancer, and wherein said one or more RNA transcripts are transcribed from one or more genes selected from the group consisting of the genes listed in Tables 1J and 1K, or wherein said disease of interest is coronary artery disease, and wherein said one or more RNA transcripts are transcribed from one or more genes selected from the group consisting of the genes listed in Table 1L wherein said marker does not identify a gene selected from the group consisting of ANF, ZFP and □MyHC, or wherein said disease of interest is rheumatoid arthritis, and wherein said one or more RNA transcripts are transcribed from one or more genes selected from the group consisting of the genes listed in Table 1M, or wherein said disease of interest is osteoarthritis, and wherein said one or more RNA transcripts are transcribed from one or more genes selected from the group consisting of the genes listed in 1AB, or wherein said disease of interest is depression, and wherein said one or more RNA transcripts are transcribed from one or more genes selected from the group consisting of the genes listed in Table 1N, or wherein said disease of interest is liver cancer, and wherein said one or more RNA transcripts are transcribed from one or more genes selected from the group consisting of the genes listed in Table 1X, or wherein said disease of interest is chagas disease, and wherein said one or more RNA transcripts are transcribed from one or more genes selected from the group consisting of the genes listed in Table 1Z, or wherein said disease of interest is asthma, and wherein said one or more RNA transcripts are transcribed from one or more genes selected from the group consisting of the genes listed in Table 1AA, or wherein said disease of interest is ankylosing spondylitis, and wherein said one or more RNA transcripts are transcribed from one or more genes selected from the group consisting of the genes listed in Table 1AM, wherein said disease of interest is manic depression, and wherein said one or more RNA transcripts are transcribed from one or more genes selected from the group consisting of the genes listed in Table 1AN, or wherein said disease of interest is alzheimers disease, and wherein said one or more RNA transcripts are transcribed from one or more genes selected from the group consisting of the genes listed in Table 1AO and wherein said marker does not identify the APP gene, or wherein said disease of interest is cervical cancer, and wherein said one or more RNA transcripts are transcribed from one or more genes selected from the group consisting of the genes listed in Table 1AQ, or wherein said disease of interest is gastric cancer, and wherein said one or more RNA transcripts are transcribed from one or more genes selected from the group consisting of the genes listed in Table 1AR, or wherein said disease of interest is kidney cancer, and wherein said one or more RNA transcripts are transcribed from one or more genes selected from the group consisting of the genes listed in Table 1AS, or wherein said disease of interest is testicular cancer, and wherein said one or more RNA transcripts are transcribed from one or more genes selected from the group consisting of the genes listed in Table 1AT, or wherein said disease of interest is colon cancer, and wherein said one or more RNA transcripts are transcribed from one or more genes selected from the group consisting of the genes listed in Table 1AU, or wherein said disease of interest is heart failure, and wherein said one or more RNA transcripts are transcribed from one or more genes selected from the group consisting of the genes listed in Table 1AV, or wherein said disease of interest is hepatitis, and wherein said one or more RNA transcripts are transcribed from one or more genes selected from the group consisting of the genes listed in Table 1AW, or wherein said disease of interest is either Crohn's disease or colitis, and wherein said one or more RNA transcripts are transcribed from one or more genes selected from the group consisting of the genes listed in Table 1AX, or wherein said disease of interest is osteoporosis, and wherein said one or more RNA transcripts are transcribed from one or more genes selected from the group consisting of the genes listed in Table 1AY, or wherein said condition of interest is the taking of systemic steroids, and wherein said one or more RNA transcripts are transcribed from one or more genes selected from the group consisting of the genes listed in Tables 1D, 1V, 1W, and 1AD.

11. The method of any one of claims 5, 6 or 7, wherein said condition of interest is the administration of systemic steroids and said one or more RNA transcripts are transcribed from one or more genes selected from the group consisting of the genes listed in Tables 1D, 1V, 1W and AD, wherein said condition of interest is the administration of Celebrex, and said one or more RNA transcripts are transcribed from one or more genes selected from the group consisting of the genes listed in Table 7B, wherein said condition of interest is the administration of Vioxx, and said one or more RNA transcripts are transcribed from one or more genes selected from the group consisting of the genes listed in Table 7C, wherein said condition of interest is the administration of NSAIDs, and said one or more RNA transcripts are transcribed from one or more genes selected from the group consisting of the genes listed in Table 7E, wherein said condition of interest is the administration of Cortisone, and said one or more RNA transcripts are transcribed from one or more genes selected from the group consisting of the genes listed in Table 7F, wherein said condition of interest is the administration of Lipitor, and said one or more RNA transcripts are transcribed from one or more genes selected from the group consisting of the genes listed in Table 7H, and wherein said condition of interest is one of smoking, and said one or more RNA transcripts are transcribed from one or more genes selected from the group consisting of the genes listed in Table 7I.

12. A method of identifying one or more biomarkers specific for a group of related diseases and/or conditions of interest, wherein said group of related diseases and/or conditions comprise those diseases and/or conditions which display a similar phenotype and/or which originate from and/or effect the same physiological system, or which have the same or similar etiology, or which are medically classified together, and wherein each of said one or more biomarkers corresponds to a gene transcript, comprising the steps of:
a) determining the level of one or more RNA transcripts expressed in blood obtained from one or more individuals having a disease and/or condition that is encompassed by said group of related diseases and/or conditions of interest, wherein each of said one or more transcripts is transcribed from a gene that is a candidate biomarker for said group of related diseases and/or conditions of interest,
b) comparing the level of each of said one or more RNA transcripts from said step a) with the level of each of said one or more genes transcripts in blood obtained from one or more individuals not having a disease and/or condition that is encompassed by said group of related diseases and/or conditions of interest,
wherein those compared transcripts which display differing levels in the comparison of step b) are identified as being biomarkers for identifying one or more biomarkers specific for said group of related diseases and/or conditions of interest.

13. The method of claim 12, wherein said group of related diseases and/or conditions of interest is selected from the following groups of related diseases and/or conditions: cancer, cardiovascular disease and neurological disease.

14. The method of claim 13 wherein the disease of cancer is consists of: cervical cancer, stomach cancer, kidney cancer, testicular cancer, bladder cancer, liver cancer, lung cancer and colon cancer

15. The method of claim 13 wherein the cardiovascular disease and/or condition consists of coronary artery disease, heart failure and hypertension.

16. The method of claim 13, wherein said neurological disease and/or condition consists of alzheimer's disease, Manic Depression and Schizophrenia.

17. A method of diagnosing or prognosing a condition of interest and/or a stage of progression or regression thereof, in an individual suspected as having said condition of interest, comprising the steps of:
a) determining the level of one or more gene transcripts expressed in blood corresponding to the biomarkers identified in any one of claims 1 through 16 and claim 22, obtained from said individual, and
b) comparing the level of each of said one or more gene transcripts in said blood according to step a) with the level of each of said one or more gene transcripts in blood from one or more individuals having said condition of interest,
c) comparing the level of each of said one or more gene transcripts in said blood according to step a) with the level of each of said one or more gene transcripts in blood from one or more individuals not having said condition,
d) determining whether the level of said one or more gene transcripts of step a) classify with the levels of said transcripts in step b) as compared with levels of said transcripts in step c), wherein said determination is indicative of said individual of step a) having said disease of interest.

18. A kit comprising one or more biomarkers identified in any one of claims 1 through 16 and claim 22.

19. A kit for diagnosing or prognosing a condition of interest comprising: a) two gene-specific priming means designed to produce double stranded DNA complementary to a transcript which correlates to one or more biomarkers identified in any one of claims 1 through 16 and claim 22, wherein said first priming means contains a sequence which can hybridize to said RNA transcript to create an extension product and said second priming means capable of hybridizing to said extension product; b) an enzyme with reverse transcriptase activity c) an enzyme with thermostable DNA polymerase activity and d) a labeling means; wherein said primers are used to detect the quantitative expression levels of said RNA transcript(s) in a test subject.

20. A kit for monitoring a course of therapeutic treatment of a condition of interest, comprising a) two gene-specific priming means designed to produce double stranded DNA complementary to a transcript which correlates to one or more biomarkers identified in any one of claims 1 through 16 and claim 22, wherein said first priming means contains a sequence which can hybridize to said RNA transcript to create an extension product and said second priming means capable of hybridizing to said extension product; b) an enzyme with reverse transcriptase activity c) an enzyme with thermostable DNA polymerase activity and d) a labeling means; wherein said primers are used to detect the quantitative expression levels of said RNA transcript(s) in a test subject.

21. A kit for monitoring progression or regression of coronary artery disease, comprising: a) two gene-specific priming means designed to produce double stranded DNA complementary to a transcript which correlates to one or more biomarkers identified in any one of claims 1 through 16 and claim 22, wherein said first priming means contains a sequence which can hybridize to said RNA transcript to create an extension product and said second priming means capable of hybridizing to said extension product; b) an enzyme with reverse transcriptase activity c) an enzyme with thermostable DNA polymerase activity and d) a labeling means; wherein said primers are used to detect the quantitative expression levels of said RNA transcript(s) in a test subject.

22. The method of claim 9, wherein said first and second disease or condition of interest of said pair of disease and/or conditions, are selected from the group consisting of rheumatoid arthritis and osteoarthritis, respectively, schizophrenia and manic depression syndrome, respectively, liver cancer and hepatitis, respectively, bladder cancer and kidney cancer, respectively, bladder cancer and testicular cancer, respectively, testicular cancer and kidney cancer, respectively, liver cancer and stomach cancer, respectively, liver cancer and colon cancer, respectively, stomach cancer and colon cancer, respectively, Chagas disease and heart failure, respectively, Chagas disease and coronary artery disease, respectively, coronary artery disease and heart failure, respectively, asymptomatic Chagas Disease and symptomatic Chagas Disease, respectively, Alzheimer's Disease and Schizophrenia, respectivley, and Alzheimer's Disease and Manic Depression, respectively.


SEQUENCE LISTING (233 sequences)

SEQ ID NO: 1 - DNA, 155 bp, Human
cagcgtggcgagcaggcttgccgccgagtgcatcctgagcaagcggcatcgaaggcaagctgagtaccttggtcaagtggcgcggcttgtcctccaaggagggtgttttgcattcccggaaggccttctcttgcacaactgcttccgcggccccg

SEQ ID NO: 2 - DNA, 145 bp, Human
ttaagaagggcccctattccacttggcagcagctttatttctcagtagccatgatgatgacgatgatatttaatccccttaaactttgcttttttaggggaggagctcccccccatctaacattttcctcctgttctttcagggg

SEQ ID NO: 3 - DNA, 129 bp, Human
gtttctttttcctaaaacggttttatttaactcaatgtgtcaaagtttttttttaataatcccaagagggatgaagccgtgtccacagggatatatacatcattatggttcccatctttcatacatgaa

SEQ ID NO: 4 - DNA, 105 bp, Human
ctccaggagaggcaggtcgacctgcctgccaggcccgatgggctgccgggcttctggtggaacctgccggcctgccttgggagcttcgggcctatgcctctgccc

SEQ ID NO: 5 - DNA, 102 bp, Human
agaaacacttaagatacaaggttcttttgaattcaacagcaagatgcttgcgatgcagtgcggtaaggtaattctcacctcctgtggaatgggcttcaaccc

SEQ ID NO: 6 - DNA, 105 bp, Human
caaatcctgaagcatccttggccaaccgcaacagcattggtgagcagaggcatgacaaggaacatagggaggccagttttggcacttggaattcaattcctcaga

SEQ ID NO: 7 - DNA, 361 bp, Human
  misc_feature (13)..(14): n is a, c, g, or t
  misc_feature (16)..(16): n is a, c, g, or t
  misc_feature (20)..(21): n is a, c, g, or t
  misc_feature (25)..(28): n is a, c, g, or t
  misc_feature (42)..(42): n is a, c, g, or t
  misc_feature (52)..(52): n is a, c, g, or t
  misc_feature (55)..(55): n is a, c, g, or t
  misc_feature (57)..(57): n is a, c, g, or t
  misc_feature (59)..(59): n is a, c, g, or t
  misc_feature (63)..(63): n is a, c, g, or t
  misc_feature (65)..(65): n is a, c, g, or t
  misc_feature (68)..(68): n is a, c, g, or t
  misc_feature (72)..(72): n is a, c, g, or t
  misc_feature (74)..(74): n is a, c, g, or t
  misc_feature (77)..(77): n is a, c, g, or t
  misc_feature (91)..(91): n is a, c, g, or t
  misc_feature (102)..(102): n is a, c, g, or t
  misc_feature (117)..(117): n is a, c, g, or t
  misc_feature (127)..(128): n is a, c, g, or t
  misc_feature (169)..(169): n is a, c, g, or t
  misc_feature (172)..(172): n is a, c, g, or t
  misc_feature (192)..(192): n is a, c, g, or t
  misc_feature (203)..(203): n is a, c, g, or t
  misc_feature (243)..(244): n is a, c, g, or t
  misc_feature (248)..(249): n is a, c, g, or t
  misc_feature (253)..(253): n is a, c, g, or t
  misc_feature (283)..(283): n is a, c, g, or t
  misc_feature (288)..(288): n is a, c, g, or t
  misc_feature (299)..(299): n is a, c, g, or t
  misc_feature (318)..(318): n is a, c, g, or t
  misc_feature (327)..(327): n is a, c, g, or t
  misc_feature (334)..(334): n is a, c, g, or t
  misc_feature (348)..(348): n is a, c, g, or t
  misc_feature (359)..(360): n is a, c, g, or t
gggggcttttttnnancggnnccgnnnncccttcctgggaanttttgggccnttntntnaaangnggncttncnggnaaatgggttttttnagggggctggncaaaggttttttctntaatgggatnnggccggcattttaaaaaaacccgctttggcctttttgctanatnggaaaaaatttttttaaaangcctaagacanggttttcccttcatatgccaaactttccctaacatttggnntttnngggngggcagggggggatttttaaaccggatttngggtnaaaaaaaatcnggggggaatttttgggganaaaaccttnggggggnccccctttgaaaanaaagggtgggnng

SEQ ID NO: 8 - DNA, 214 bp, Human
catcctccagagaaggtggcccctggccccgcccctggttacagaggcaactaatatcctgccctgaaccgggaaccgaaaaacaattatatgtctaattcccctaagaaatataagaagagcgagccccctaattgaaggaaaaagacaggacaagaggcctctcttagacaaccaactctagtggccccattcaggacacacttgtagcaca

SEQ ID NO: 9 - DNA, 143 bp, Human
cggcagagggaaggtgctggaacgtactggaaagtgacgcgcatgacggggcccagctaggcgacaggacttgactacatacagaggacgagggacaaaatgatattgacacagggacattacatacataaaccaaaaacaaa

SEQ ID NO: 10 - DNA, 127 bp, Human
tctaaacttctgggaacaaacaggacacattggagcttgagaacccctcgctgacattcaccccagttttcaggcaggaggttgttgtcaacacacacaattcaacctggacaagacaaccctgtca

SEQ ID NO: 11 - DNA, 235 bp, Human
ctccagggaccaagtgttgagatttcaaggccggggtgagaccaaaggtacggttatgtcacagtcctaatgtgtggccttctctgattggtacatctccaacccaagagagtgtacacatacaaggcacccgaggacatgtcgcggggagacgcgagatcgtcgcactagagggctctgtgggccctctgtgggcgccctgttagagtgttacactacggggggccactacttc

SEQ ID NO: 12 - DNA, 192 bp, Human
gattagttatgttaaacgctacttgcaagtcttgcttcttttggatatcaaaatgtatttgtgatgtactaagatacttggtcctgaaggctacccaaatattatagggtcaatttaggccaattcaatatctcttatatagtataaaacgatggccttggaaatgggctgaggaatttatgccattgtgat

SEQ ID NO: 13 - DNA, 145 bp, Human
ggaactcttctgtatctaaaacaatacatctcaatcttgggccagggaaaatgggcttcttttctgggttggcacctagccaccttagggaatttcggttgagattttcaacaagagtaagacacacacattccaggtaaagggg

SEQ ID NO: 14 - DNA, 91 bp, Human
gggatccctcatctgttcctccgggaccctggtgctgcctctaactgacaattcccttggggcttcctgtaacctctttgagagaggcctt

SEQ ID NO: 15 - DNA, 310 bp, Human
ctcgtgccgaattcggcacgagcttggttgggaggaccagagcctgtctcagggaatttgcctgcttgggtgatgcagggaggaaggtctagtgcagggaagagggggcctggctcagctgttcaccagcactttttgaccacagtctcgctactgcgggcccctgccctagaggtttagagcgatagacctccctctccctcgggggacatatctgggacacaggctctctcgaaatcgtctctcctgtcccagacacaggtttgagtctcagacgtctcgatctactgtagacgacgaagacgacata

SEQ ID NO: 16 - DNA, 250 bp, Human
gagaccaaggccgccccgctctggtctcagaccagttgtgctgctcttgctctggctcagctggtgtggggcgcaggcgggaaacgagacctctagcatctggctgaaggctctgccaagctcctcttcagggctgcagtctgcctgcctgcatataccgacttggccagacactgctgctaaattccagggactctttctcccctcctctgctctccagccaatccttgaggatttaataactggaagg

SEQ ID NO: 17 - DNA, 108 bp, Human
ggcaggatggcagtcaatatcagagagagttgtcgctgaggcatctcaagctgactgatgagtctctcctcggagctggctgaaggatggctactctggagtggaggt

SEQ ID NO: 18 - DNA, 254 bp, Human
gtggttgaggtaagtcaagtatttagtagagagtgagatgagtaagcagcatgaatatagggaggcatcaagcacatggtattgagtgatgtcgaggagtgataatttagagaagggttgttagtgaaaagaagtttaagtggcgacgagatacgcgattgtaagatatcgatacgagctacagcactcacgatttacagagtatgatgagataagtttgcaatgtctatactttcgcttttggagactacgac

SEQ ID NO: 19 - DNA, 245 bp, Human
ctcgtgccgaattcggcacgaggaattttcatgtaagatcctatggccggtgcagtcgctcacggctgtaattccaagcaacttttgggggccaaggaggttggatcacttaagtcaagagagtttagagacacagtttggaaacacatgtgtgaaccccctctgtgtatctaaaatatacaaaatttgcgctgggaaatgggtgcgcggatatctgataattcctagacactggggtgcgctga

SEQ ID NO: 20 - DNA, 220 bp, Human
ggagcagctctgtgcttggacatcagtgggccaaggttcttgtccctggttcactgtgatttggctttccggttctttcctgggatgcctttttggggttccttgggtttgggttggaagaggccatttccctaatttaacctctaggttttccgaggcccttgggcctggttttttttagctttggacagtgtgcccctttttccttctggtttgcctc

SEQ ID NO: 21 - DNA, 200 bp, Human
cgacgacgcgcgtgttccgggagcttggagctggagctggaccggctgcgcgccgaggaacctccagcttccttacccaggaccggacttcaccggcagccaggagccagccccggtttccaaggtttgggggcttggcttgaactttttttgggggaagggggccaaggggactttttacaagttatggaatttaacat

SEQ ID NO: 22 - DNA, 318 bp, Human
cctgcagagtactccatggaaacaattgccgagcacgtgctcgcaatttgccgagcacggtccggtttgaactcctagactaagactaggtaggtgatacataccttcttcccaccaagtactcacgatccaaactatgaattttagattcggatcaaacgaggattgatccgagggaccaacgttgtgataaatcttacgtcgtcttatatattaagtttttgtggaggatcggataagtctatagtgtttgtcacagatagtcccgtaccacaccccagaccataggagtcgctctccggaccgcggtctaatggg

SEQ ID NO: 23 - DNA, 111 bp, Human
cgcgctgtgcctgctcgtgttgcctacaatgttccttgctgttagaggcgcttcttcagcttgcaaccctttccttgtcttataaggagctctccttttgacccctctcta

SEQ ID NO: 24 - DNA, 135 bp, Human
ctggagttggcaccctgcagcagttatgatccagatctatgttctgtgacggtcgtagacaagccaatttatttgcttaatctgtgaaaccacggatcaatccctccaaaacccgttgaaaaaaggataaattct

SEQ ID NO: 25 - DNA, 155 bp, Human
tctcacattggacatactcaaaattcacttataatcttcacaccaccaaaaacttacccatatcaaattataaacccacccacattacttaaaattttttacatttcccaataaaaaacccaaataaacaaaaacttccaatctccatttaaaat

SEQ ID NO: 26 - DNA, 112 bp, Human
tctcaatcctaatttctcctccctttctttttcccttgcttcaggaaactccacatctgcctaaaaaccaaaggagggcttcctcttggaggccaaggggaaaggggggtgc

SEQ ID NO: 27 - DNA, 178 bp, Human
cttaatatttgcatgataagctagtttattgggttagtattcttgttggttacggaatggatcaactaattcctgggttatctaaccaccgagggctagaagcgcgcgcgaaagaggtcctgggtacacagagccatgagccacccattttataagacactctgtatttctaaaagtt

SEQ ID NO: 28 - DNA, 135 bp, Human
ttggctgccttgtgaaatgattccctgcagtaaacggacttttcattttattttagatcattacaaactttccatttcacattcttccattgatttaccagaacaaccactgggggatattgtaggacttaggtt

SEQ ID NO: 29 - DNA, 228 bp, Human
ttggtcattatgaccaggtgagcatgctactataccgcagaggatccaaccatgagctcttcccagcctaggggaagctggatttgccctcatgtaggagctggctaggactgttgactctctcagctgagtcagggacactcaggcactggacagttgggcatttgaggccgtgtgtaattctgctctcatgctgagtctccatttcttccctttctctgtcatcca

SEQ ID NO: 30 - DNA, 143 bp, Human
gtgaattatagaggaaatacagagagaacctctctcactcttacttttcgtccaaataaaattgataggtgtaccagcaagttgaaggatccggtttaagaatttggggcttactctcattacaaattaggccccccacacay

SEQ ID NO: 31 - DNA, 114 bp, Human
  misc_feature (14)..(14): n is a, c, g, or t
  misc_feature (27)..(27): n is a, c, g, or t
  misc_feature (37)..(37): n is a, c, g, or t
  misc_feature (39)..(39): n is a, c, g, or t
  misc_feature (43)..(43): n is a, c, g, or t
  misc_feature (50)..(50): n is a, c, g, or t
  misc_feature (52)..(52): n is a, c, g, or t
  misc_feature (60)..(60): n is a, c, g, or t
  misc_feature (65)..(65): n is a, c, g, or t
  misc_feature (95)..(95): n is a, c, g, or t
  misc_feature (106)..(106): n is a, c, g, or t
gcgcttgcacgccnacactagtggatncaaagaattntncacnacagtgncngagtgagnttctngggggcccagcttcctccataggtggcagnaatggcccggntactaggg

SEQ ID NO: 32 - DNA, 128 bp, Human
  misc_feature (15)..(15): n is a, c, g, or t
  misc_feature (19)..(19): n is a, c, g, or t
  misc_feature (38)..(38): n is a, c, g, or t
  misc_feature (46)..(46): n is a, c, g, or t
  misc_feature (48)..(48): n is a, c, g, or t
  misc_feature (51)..(51): n is a, c, g, or t
  misc_feature (55)..(55): n is a, c, g, or t
  misc_feature (57)..(57): n is a, c, g, or t
  misc_feature (75)..(75): n is a, c, g, or t
  misc_feature (84)..(84): n is a, c, g, or t
  misc_feature (86)..(86): n is a, c, g, or t
  misc_feature (114)..(114): n is a, c, g, or t
  misc_feature (118)..(118): n is a, c, g, or t
  misc_feature (121)..(121): n is a, c, g, or t
cgacactagtggatncaangaattcggcacgaggccantgtgcagngngantttntngatcttcagctacatttncggctttgngngaaaccttaccatctaacacgatggccngcancgntaccaac

SEQ ID NO: 33 - DNA, 100 bp, Human
  misc_feature (3)..(3): n is a, c, g, or t
  misc_feature (15)..(15): n is a, c, g, or t
  misc_feature (38)..(38): n is a, c, g, or t
  misc_feature (41)..(41): n is a, c, g, or t
  misc_feature (53)..(53): n is a, c, g, or t
  misc_feature (93)..(93): n is a, c, g, or t
ggngcttgcgggtcnacactagtggattcaaagaattnggnacgagctgaacnctggaggcctacccatcaccccatcccgcaattccgccanagccaag

SEQ ID NO: 34 - DNA, 107 bp, Human
  misc_feature (12)..(12): n is a, c, g, or t
  misc_feature (18)..(18): n is a, c, g, or t
  misc_feature (25)..(25): n is a, c, g, or t
  misc_feature (29)..(29): n is a, c, g, or t
  misc_feature (41)..(41): n is a, c, g, or t
  misc_feature (55)..(56): n is a, c, g, or t
  misc_feature (65)..(67): n is a, c, g, or t
  misc_feature (71)..(71): n is a, c, g, or t
  misc_feature (76)..(76): n is a, c, g, or t
  misc_feature (79)..(79): n is a, c, g, or t
  misc_feature (86)..(86): n is a, c, g, or t
gggcgcttgcangacccncactagnggantcaaagaatttnttcgagggcaaatnnagattatgnnnctcnaattntgntacttgnttggctgttcatgtggtcacg

SEQ ID NO: 35 - DNA, 118 bp, Human
  misc_feature (6)..(6): n is a, c, g, or t
  misc_feature (12)..(12): n is a, c, g, or t
  misc_feature (17)..(17): n is a, c, g, or t
  misc_feature (21)..(21): n is a, c, g, or t
  misc_feature (30)..(30): n is a, c, g, or t
  misc_feature (34)..(34): n is a, c, g, or t
  misc_feature (42)..(42): n is a, c, g, or t
  misc_feature (49)..(49): n is a, c, g, or t
  misc_feature (56)..(56): n is a, c, g, or t
  misc_feature (66)..(69): n is a, c, g, or t
  misc_feature (74)..(74): n is a, c, g, or t
  misc_feature (99)..(99): n is a, c, g, or t
  misc_feature (105)..(105): n is a, c, g, or t
  misc_feature (110)..(110): n is a, c, g, or t
  misc_feature (114)..(114): n is a, c, g, or t
  misc_feature (116)..(116): n is a, c, g, or t
tacagngacaancagancatnctgccttanaggngctagatncccgaanttagaanaccctttctnnnncagtnatgaagttataaatatcagcttgtncatccnagccnctgncnga

SEQ ID NO: 36 - DNA, 102 bp, Human
  misc_feature (2)..(2): n is a, c, g, or t
  misc_feature (4)..(4): n is a, c, g, or t
  misc_feature (62)..(62): n is a, c, g, or t
gncnacactagtggattcaaagaattcttcactacaagccaagacgggaactgaaggtgtgngtgtcgagccctctggcccagggttaacactgggtcaaat

SEQ ID NO: 37 - DNA, 100 bp, Human
  misc_feature (2)..(2): n is a, c, g, or t
  misc_feature (40)..(40): n is a, c, g, or t
  misc_feature (45)..(45): n is a, c, g, or t
  misc_feature (55)..(55): n is a, c, g, or t
  misc_feature (59)..(59): n is a, c, g, or t
  misc_feature (62)..(62): n is a, c, g, or t
cnggagcttgcaaggcgacactagtggattcaaagaattntttangagtgacctncacntcnccgccctgcgtgcaagtgaagcggaatgactacgtgcc

SEQ ID NO: 38 - DNA, 101 bp, Human
  misc_feature (2)..(2): n is a, c, g, or t
  misc_feature (8)..(8): n is a, c, g, or t
  misc_feature (18)..(18): n is a, c, g, or t
  misc_feature (44)..(44): n is a, c, g, or t
  misc_feature (47)..(48): n is a, c, g, or t
  misc_feature (55)..(55): n is a, c, g, or t
  misc_feature (59)..(59): n is a, c, g, or t
  misc_feature (64)..(64): n is a, c, g, or t
  misc_feature (67)..(67): n is a, c, g, or t
  misc_feature (70)..(70): n is a, c, g, or t
  misc_feature (76)..(76): n is a, c, g, or t
cnggcgcntgcaggcccnacactagtggattcaaagaatttttncannagggaangagngaggnacnggngacgtnaagctcctgaagcacaaggagaaag

SEQ ID NO: 39 - DNA, 131 bp, Human
  misc_feature (12)..(12): n is a, c, g, or t
  misc_feature (14)..(14): n is a, c, g, or t
  misc_feature (38)..(38): n is a, c, g, or t
  misc_feature (53)..(53): n is a, c, g, or t
  misc_feature (59)..(60): n is a, c, g, or t
  misc_feature (71)..(71): n is a, c, g, or t
  misc_feature (75)..(75): n is a, c, g, or t
  misc_feature (90)..(90): n is a, c, g, or t
  misc_feature (96)..(96): n is a, c, g, or t
  misc_feature (100)..(100): n is a, c, g, or t
  misc_feature (127)..(127): n is a, c, g, or t
gcgcttgcacgncnacactagtggattaaaagaattcngcactagcagggagngaaggnnataacacgganccanctccacccttcctcncttgangcanaaaggactcaagattgccaaaggcctnttgt

SEQ ID NO: 40 - DNA, 168 bp, Human
  misc_feature (6)..(6): n is a, c, g, or t
  misc_feature (15)..(15): n is a, c, g, or t
  misc_feature (37)..(38): n is a, c, g, or t
  misc_feature (47)..(47): n is a, c, g, or t
  misc_feature (56)..(57): n is a, c, g, or t
  misc_feature (94)..(94): n is a, c, g, or t
  misc_feature (127)..(127): n is a, c, g, or t
  misc_feature (155)..(155): n is a, c, g, or t
  misc_feature (157)..(157): n is a, c, g, or t
ggcccntgggggggnagggccttttcggggccggggnngggcccccntttggcccnnggggggtttcccggggaacccaaccctttaaggggtnggggggaatttccccccaaaaaaagggaaaaanttttccggggggcccacccgggaagggntnccggggaaggg

SEQ ID NO: 41 - DNA, 235 bp, Human
  misc_feature (5)..(5): n is a, c, g, or t
  misc_feature (15)..(15): n is a, c, g, or t
  misc_feature (39)..(39): n is a, c, g, or t
  misc_feature (47)..(47): n is a, c, g, or t
  misc_feature (52)..(52): n is a, c, g, or t
  misc_feature (69)..(69): n is a, c, g, or t
  misc_feature (154)..(154): n is a, c, g, or t
  misc_feature (207)..(207): n is a, c, g, or t
  misc_feature (217)..(217): n is a, c, g, or t
aaggncttttccggnccggcccggccccccttggcccangggggttnccggnaaaccaccctttaaggnttgggggaattcccccaaaaaaggaaaaaattttcccgggggcccacccggaaagggggaaggcccccaaaaccggggggggggnaaaaaggtgggtttccccctttttccaattcccaaaaccaatttccaaaaggnaaaccaaccnttcccaaaatgggaaagg

SEQ ID NO: 42 - DNA, 446 bp, Human
gccaagagcaagagtgtgggtgtgaacgtagagaatcctcctttttgccccaaaggggtgaagtgtttgatgcaggtcatggaggagaaagcatggtgtgggtaagacacggaaggaatgaaggagaggtgagatgaggccacagaaacagggtgtagaggtgttggcaccttggaaacattgaggaccgtgtgtcaataaagggcatggcgagacgatggaaggccagaggacacaacagagagaggaaaccactgttccttagaggcagaactgagaatacaggacggttaggggtgaactgagacagcagatggactcagtacagcaggttgaggacatggaagctggcagtggtgtcatcagtggggggcagggcaggaaggggtcagagttcaggaaagattcctgagtctgtggattgacttggaggtggcagggcatgc

SEQ ID NO: 43 - DNA, 227 bp, Human
  misc_feature (2)..(2): n is a, c, g, or t
  misc_feature (8)..(8): n is a, c, g, or t
  misc_feature (27)..(27): n is a, c, g, or t
  misc_feature (31)..(31): n is a, c, g, or t
  misc_feature (36)..(37): n is a, c, g, or t
  misc_feature (64)..(64): n is a, c, g, or t
  misc_feature (76)..(76): n is a, c, g, or t
  misc_feature (126)..(126): n is a, c, g, or t
  misc_feature (140)..(140): n is a, c, g, or t
  misc_feature (158)..(158): n is a, c, g, or t
  misc_feature (171)..(171): n is a, c, g, or t
  misc_feature (183)..(183): n is a, c, g, or t
  misc_feature (198)..(198): n is a, c, g, or t
  misc_feature (220)..(220): n is a, c, g, or t
cntggggnaggctttcggcgggcccgncccntggcnnggggtcccggaaccaccttaggtgggnatccccaaaagnaatttccgggcacggaagggcttacctggggaggtcattttccggttttnccacttcatttcanccccaaaccttcaggggnttctcccccattnttgaagcccagncctgctgggggggantttactcatcctccatttcccntgggaat

SEQ ID NO: 44 - DNA, 447 bp, Human
  misc_feature (272)..(272): n is a, c, g, or t
  misc_feature (334)..(334): n is a, c, g, or t
  misc_feature (391)..(391): n is a, c, g, or t
  misc_feature (396)..(396): n is a, c, g, or t
ggggcacaggcaccaaagtccgccaaggcacccttgaaagaaaggcgcgctacaattgcttcagtgccaggagaacattccgcgtcaccaagcccttgcacccccaagaaccaaaagcaaaaggccaaaagcccaagaaaagggaagggaaaaggacttagaacgccaagcctggatgccaagggagcccctgggtgtcacattggggcccttggcccaccgcccttccctttttcccagggccccgagaatgtgaccccacccaggtgccnttcttgtcttgcttcgtttagctttttaattcaattcattgcccctggcctttgttcccttnttcacttccccagcccccaccccctaaggtggcccaaaaggtggggagggacaaaanggganttcttgggaaagctttgagccctccccccaaaagcaatgtgagtcccagag

SEQ ID NO: 45 - DNA, 294 bp, Human
  misc_feature (18)..(19): n is a, c, g, or t
  misc_feature (27)..(27): n is a, c, g, or t
  misc_feature (47)..(47): n is a, c, g, or t
  misc_feature (66)..(66): n is a, c, g, or t
  misc_feature (76)..(76): n is a, c, g, or t
  misc_feature (82)..(82): n is a, c, g, or t
  misc_feature (108)..(108): n is a, c, g, or t
  misc_feature (133)..(133): n is a, c, g, or t
  misc_feature (162)..(162): n is a, c, g, or t
  misc_feature (210)..(210): n is a, c, g, or t
  misc_feature (260)..(260): n is a, c, g, or t
  misc_feature (276)..(276): n is a, c, g, or t
aaaccaaccctttaaggnntggggggnaattccccccaaaaaaaggnaaaaattttttccgggggnccaaaccggnaaaggntttgggaaaaccaaattttttttggncccaaccccccccaaattggggggnaaaccaaatttaaggggggaagggggggncccccccgggaaaggcccaaggggggaaaatttttccgggggtgggtngggggaaccaatttaaggggggggcccccgggggggttccccttgggccntttttccttttgggtnaaaaaaaaaaaccccttg

SEQ ID NO: 46 - DNA, 242 bp, Human
  misc_feature (17)..(17): n is a, c, g, or t
  misc_feature (40)..(40): n is a, c, g, or t
  misc_feature (59)..(59): n is a, c, g, or t
  misc_feature (76)..(76): n is a, c, g, or t
  misc_feature (107)..(107): n is a, c, g, or t
  misc_feature (165)..(165): n is a, c, g, or t
  misc_feature (189)..(189): n is a, c, g, or t
  misc_feature (216)..(216): n is a, c, g, or t
  misc_feature (221)..(221): n is a, c, g, or t
  misc_feature (230)..(230): n is a, c, g, or t
gagtcagactgtaaggnacgaaccctcggggtccccacgntgttccccccggggtaacntcggcccgggcccgggnagcccttcccgggcttttcccccgggggggncccgggggggacctttaggcggcaccccaacaacaccaggccctactttttccaaggncggggaagcccatgggttctgggnaacgggcaatgcgggcttgcaacgggnggaanaaaaacagncccaaaagaatg

SEQ ID NO: 47 - DNA, 100 bp, Human
  misc_feature (2)..(2): n is a, c, g, or t
  misc_feature (6)..(6): n is a, c, g, or t
  misc_feature (12)..(12): n is a, c, g, or t
  misc_feature (14)..(14): n is a, c, g, or t
  misc_feature (19)..(19): n is a, c, g, or t
  misc_feature (21)..(21): n is a, c, g, or t
  misc_feature (26)..(26): n is a, c, g, or t
  misc_feature (28)..(28): n is a, c, g, or t
  misc_feature (31)..(32): n is a, c, g, or t
  misc_feature (37)..(37): n is a, c, g, or t
  misc_feature (42)..(42): n is a, c, g, or t
  misc_feature (44)..(44): n is a, c, g, or t
  misc_feature (46)..(46): n is a, c, g, or t
  misc_feature (49)..(50): n is a, c, g, or t
  misc_feature (55)..(56): n is a, c, g, or t
  misc_feature (58)..(59): n is a, c, g, or t
  misc_feature (62)..(65): n is a, c, g, or t
  misc_feature (68)..(68): n is a, c, g, or t
  misc_feature (73)..(75): n is a, c, g, or t
  misc_feature (79)..(79): n is a, c, g, or t
  misc_feature (83)..(84): n is a, c, g, or t
  misc_feature (86)..(86): n is a, c, g, or t
  misc_feature (89)..(89): n is a, c, g, or t
  misc_feature (97)..(97): n is a, c, g, or t
anaccncgaagngnggggncncaggntncannctaancccanancnccnnccgcnnanngannnngancttcnnnggcncccnngnccnttgggggnggg

SEQ ID NO: 48 - DNA, 100 bp, Human
  misc_feature (17)..(17): n is a, c, g, or t
  misc_feature (19)..(19): n is a, c, g, or t
  misc_feature (27)..(29): n is a, c, g, or t
  misc_feature (37)..(38): n is a, c, g, or t
  misc_feature (46)..(46): n is a, c, g, or t
  misc_feature (49)..(49): n is a, c, g, or t
  misc_feature (65)..(65): n is a, c, g, or t
  misc_feature (72)..(72): n is a, c, g, or t
acaccttcccacttgcngnaaaggggnnnggcccccnncttgggcngancattaagcctttttgnggctgcngcccctgtgcctggtgccacaacaaatg

SEQ ID NO: 49 - DNA, 100 bp, Human
  misc_feature (7)..(7): n is a, c, g, or t
  misc_feature (9)..(9): n is a, c, g, or t
  misc_feature (28)..(28): n is a, c, g, or t
  misc_feature (31)..(33): n is a, c, g, or t
  misc_feature (35)..(35): n is a, c, g, or t
  misc_feature (39)..(40): n is a, c, g, or t
  misc_feature (42)..(42): n is a, c, g, or t
  misc_feature (44)..(44): n is a, c, g, or t
  misc_feature (55)..(56): n is a, c, g, or t
  misc_feature (58)..(58): n is a, c, g, or t
  misc_feature (62)..(62): n is a, c, g, or t
  misc_feature (68)..(68): n is a, c, g, or t
  misc_feature (71)..(71): n is a, c, g, or t
  misc_feature (74)..(74): n is a, c, g, or t
  misc_feature (80)..(80): n is a, c, g, or t
atgccancntaacaggtggcaactcggnagnnntnttgnnangnacttgcctgcnnantggngggtgncgnccnaccttntcccctccccccccccgccc

SEQ ID NO: 50 - DNA, 227 bp, Human
  misc_feature (5)..(5): n is a, c, g, or t
  misc_feature (13)..(13): n is a, c, g, or t
  misc_feature (71)..(71): n is a, c, g, or t
  misc_feature (88)..(88): n is a, c, g, or t
  misc_feature (91)..(91): n is a, c, g, or t
  misc_feature (109)..(109): n is a, c, g, or t
  misc_feature (157)..(157): n is a, c, g, or t
  misc_feature (177)..(177): n is a, c, g, or t
  misc_feature (199)..(199): n is a, c, g, or t
  misc_feature (207)..(207): n is a, c, g, or t
  misc_feature (211)..(211): n is a, c, g, or t
ccggncaccaccnttaaggttgggggatttccccaaaaaaggaaaattttcggcggccaacgggaaggccnttggggaaaaaaccaanggncaaacccccccaaccacncggccccccccaaggggggtggggaagagccaaatttctttgggaaanaacgcccccttggggaaaanaaggccaaccacctttcaacancccccaangcgnggaagccatttcttgg

SEQ ID NO: 51 - DNA, 269 bp, Human
  misc_feature (7)..(8): n is a, c, g, or t
  misc_feature (40)..(40): n is a, c, g, or t
  misc_feature (76)..(76): n is a, c, g, or t
  misc_feature (89)..(89): n is a, c, g, or t
  misc_feature (135)..(135): n is a, c, g, or t
  misc_feature (154)..(154): n is a, c, g, or t
  misc_feature (210)..(210): n is a, c, g, or t
  misc_feature (224)..(224): n is a, c, g, or t
  misc_feature (240)..(240): n is a, c, g, or t
  misc_feature (251)..(251): n is a, c, g, or t
gtcccanngggggttcggggaaaccaaccctttaagggtnggggggaaattcccccaaaaaaagggaaaaattttnccggggggcccanccgggaaggggggggaaaaaaaagaaaaggggggaacccttccccncccaggggtttttaagggnccccccaaggccccttggggaacccccaatggggaccccttgggaaaaggtttttnccccccagggcccntttttttaaaaccctnttttttttttnaaaaaaaaaacccttggg

SEQ ID NO: 52 - DNA, 100 bp, Human
  misc_feature (9)..(9): n is a, c, g, or t
  misc_feature (23)..(23): n is a, c, g, or t
  misc_feature (35)..(36): n is a, c, g, or t
  misc_feature (47)..(47): n is a, c, g, or t
  misc_feature (51)..(53): n is a, c, g, or t
  misc_feature (63)..(63): n is a, c, g, or t
  misc_feature (69)..(69): n is a, c, g, or t
  misc_feature (74)..(74): n is a, c, g, or t
  misc_feature (77)..(77): n is a, c, g, or t
  misc_feature (79)..(79): n is a, c, g, or t
  misc_feature (85)..(85): n is a, c, g, or t
  misc_feature (88)..(88): n is a, c, g, or t
  misc_feature (90)..(92): n is a, c, g, or t
  misc_feature (97)..(97): n is a, c, g, or t
  misc_feature (99)..(99): n is a, c, g, or t
gaattcttncacgagcggtagcngtagaatagatnntgtaccccccnaggnnnccccccgcangggagncccanttnanttgtanagnannnccggnang

SEQ ID NO: 53 - DNA, 492 bp, Human
  misc_feature (299)..(299): n is a, c, g, or t
  misc_feature (333)..(333): n is a, c, g, or t
  misc_feature (353)..(353): n is a, c, g, or t
  misc_feature (410)..(411): n is a, c, g, or t
  misc_feature (460)..(460): n is a, c, g, or t
  misc_feature (474)..(474): n is a, c, g, or t
  misc_feature (479)..(479): n is a, c, g, or t
cggcttcgggccaagcgtttccagagtttgccgaactgctgagcaagttcgctattctccagatcgcctagccctttgcgggcgaccaccacgatgtcccagcctgtcaggttgtcctgattgaggcgaaaggactcgcggatttgacgcttgatgcggttgcgctcgacggcgagcttgacgctctttttgccgatcaccaaacctaggcggggatgatcaagctggttatcgcgcgctagcagcaggacacttttgcccgggagctttaccgcttggggagtcgaagactgccttgnattgccggggagtcagcagtcgctttttcccggncgaagcctcgaactcaccancctgtctggattaattagacagcaagacgcttgcggcccctttggcgcgaacgaacnncgaaaaggacttgcgcggcccgtttctttggggggccaataccggggcncggggaaaacccgnggggnggccaaacccccc

SEQ ID NO: 54 - DNA, 231 bp, Human
  misc_feature (18)..(18): n is a, c, g, or t
  misc_feature (47)..(47): n is a, c, g, or t
  misc_feature (59)..(59): n is a, c, g, or t
  misc_feature (74)..(74): n is a, c, g, or t
  misc_feature (138)..(138): n is a, c, g, or t
  misc_feature (174)..(174): n is a, c, g, or t
  misc_feature (176)..(176): n is a, c, g, or t
  misc_feature (211)..(211): n is a, c, g, or t
aagaaattccgggcacgnaggcacgcccctggtaattccccaggcgnacttctgggganggctggaaggcttgnagggcagaaaagggatccgcctttgggaggaacccaggtaaggtttaagaaggaacccaccctnggggccaaacaaaaacttaaaaacccccccatttcntnccccccaaaaaaaaaatttttaaaaaaaatttttngcccccgggggcattggggg

SEQ ID NO: 55 - DNA, 104 bp, Human
gacaataagctggagctccgcgcgcttgcggtcgacactagtggatccaaagaattcggcacgagctcagaaattggctttaaaaaaaacaaccaccaaaaaaa

SEQ ID NO: 56 - DNA, 109 bp, Human
  misc_feature (1)..(2): n is a, c, g, or t
  misc_feature (12)..(12): n is a, c, g, or t
  misc_feature (29)..(29): n is a, c, g, or t
  misc_feature (34)..(34): n is a, c, g, or t
nncgaacaatangtctggagctcgtgcgncctgnaggtgcgacactagtggatccaaagaattcggcacgagggattacagtcgtgagccactgcacctggctgcaatt

SEQ ID NO: 57 - DNA, 100 bp, Human
  misc_feature (4)..(5): n is a, c, g, or t
  misc_feature (91)..(91): n is a, c, g, or t
cgtnnagtggatccaaagaattcggcacgaggccagtatgctgggggaggagaaataccgccaggacctgactgtgcctccaggctactgncagtacttc

SEQ ID NO: 58 - DNA, 100 bp, Human
  misc_feature (3)..(6): n is a, c, g, or t
  misc_feature (8)..(8): n is a, c, g, or t
  misc_feature (13)..(13): n is a, c, g, or t
  misc_feature (15)..(15): n is a, c, g, or t
  misc_feature (20)..(20): n is a, c, g, or t
  misc_feature (27)..(27): n is a, c, g, or t
  misc_feature (32)..(33): n is a, c, g, or t
  misc_feature (40)..(40): n is a, c, g, or t
  misc_feature (49)..(50): n is a, c, g, or t
  misc_feature (57)..(57): n is a, c, g, or t
  misc_feature (63)..(63): n is a, c, g, or t
  misc_feature (67)..(67): n is a, c, g, or t
  misc_feature (69)..(69): n is a, c, g, or t
  misc_feature (76)..(76): n is a, c, g, or t
  misc_feature (78)..(78): n is a, c, g, or t
  misc_feature (87)..(87): n is a, c, g, or t
  misc_feature (89)..(89): n is a, c, g, or t
tcnnnntntggtntnggctntccgagnggcanngagtgantgcccgttnntattgancaccantcantngttgccntntgatacccnanacaaaattgaa

SEQ ID NO: 59 - DNA, 123 bp, Human
  misc_feature (45)..(45): n is a, c, g, or t
  misc_feature (62)..(62): n is a, c, g, or t
  misc_feature (121)..(121): n is a, c, g, or t
ctggagcctcgcgcgcctgcaggtcgacactagtggatccaaagnaattcggcacgaggccngctgacggacaactgagtctccggcccacctaccaccgccgcccgggtcccccaggtgngc

SEQ ID NO: 60 - DNA, 129 bp, Human
  misc_feature (19)..(19): n is a, c, g, or t
  misc_feature (22)..(22): n is a, c, g, or t
  misc_feature (62)..(62): n is a, c, g, or t
acaataagctggagctccnccnctcgaaggtcgacactagtggatccaaagaattcggcacnagattatttgagtgttgttggaccatgtgtgatcacgactgctatctgaataaaataaggatttgtc

SEQ ID NO: 61 - DNA, 131 bp, Human
  misc_feature (13)..(13): n is a, c, g, or t
  misc_feature (15)..(15): n is a, c, g, or t
  misc_feature (25)..(25): n is a, c, g, or t
  misc_feature (42)..(42): n is a, c, g, or t
  misc_feature (58)..(58): n is a, c, g, or t
  misc_feature (62)..(62): n is a, c, g, or t
  misc_feature (64)..(64): n is a, c, g, or t
  misc_feature (68)..(68): n is a, c, g, or t
  misc_feature (73)..(73): n is a, c, g, or t
  misc_feature (84)..(84): n is a, c, g, or t
  misc_feature (92)..(92): n is a, c, g, or t
  misc_feature (94)..(95): n is a, c, g, or t
  misc_feature (110)..(111): n is a, c, g, or t
acaacgaatttcncnctgtgttggngaatataccatggagcnagtgaacgcgcgatcnttcnanaacntgaanacagcccgtcncatcactnannctcctaaacaaagtnngagaatatgtctatacgaga

SEQ ID NO: 62 - DNA, 100 bp, Human
  misc_feature (14)..(14): n is a, c, g, or t
  misc_feature (16)..(16): n is a, c, g, or t
  misc_feature (34)..(34): n is a, c, g, or t
  misc_feature (53)..(53): n is a, c, g, or t
  misc_feature (58)..(58): n is a, c, g, or t
  misc_feature (73)..(73): n is a, c, g, or t
  misc_feature (75)..(75): n is a, c, g, or t
  misc_feature (88)..(88): n is a, c, g, or t
  misc_feature (90)..(90): n is a, c, g, or t
  misc_feature (93)..(94): n is a, c, g, or t
ttttgccgctggtnanaggacgacgcatttcacnatttgtgtggcgaatatangcatnggcagacgagtgaangngcgatcatttcgntnacnngcataa

SEQ ID NO: 63 - DNA, 100 bp, Human
  misc_feature (5)..(5): n is a, c, g, or t
  misc_feature (7)..(7): n is a, c, g, or t
  misc_feature (27)..(27): n is a, c, g, or t
  misc_feature (41)..(41): n is a, c, g, or t
  misc_feature (44)..(44): n is a, c, g, or t
  misc_feature (55)..(55): n is a, c, g, or t
  misc_feature (60)..(60): n is a, c, g, or t
  misc_feature (69)..(69): n is a, c, g, or t
  misc_feature (72)..(73): n is a, c, g, or t
  misc_feature (78)..(79): n is a, c, g, or t
  misc_feature (82)..(82): n is a, c, g, or t
  misc_feature (92)..(92): n is a, c, g, or t
  misc_feature (96)..(96): n is a, c, g, or t
cggcncnagggacccctttacctgtcnttacgatgcgcaantantaccggatttngtccngatggtcgnaannttagnnttncagcctgtgncacngcca

SEQ ID NO: 64 - DNA, 100 bp, Human
  misc_feature (12)..(12): n is a, c, g, or t
  misc_feature (26)..(26): n is a, c, g, or t
  misc_feature (46)..(46): n is a, c, g, or t
  misc_feature (49)..(49): n is a, c, g, or t
  misc_feature (55)..(55): n is a, c, g, or t
  misc_feature (60)..(60): n is a, c, g, or t
  misc_feature (63)..(63): n is a, c, g, or t
  misc_feature (69)..(70): n is a, c, g, or t
  misc_feature (73)..(73): n is a, c, g, or t
  misc_feature (79)..(79): n is a, c, g, or t
  misc_feature (92)..(92): n is a, c, g, or t
  misc_feature (96)..(96): n is a, c, g, or t
tcgggcgggganccctttacctgtcnttacgatgcgcaagtagatnccngatttngtccnganggtcgnnaanttaggnttccagcctgcgncacngcca

SEQ ID NO: 65 - DNA, 104 bp, Human
  misc_feature (2)..(2): n is a, c, g, or t
  misc_feature (7)..(7): n is a, c, g, or t
  misc_feature (27)..(27): n is a, c, g, or t
  misc_feature (34)..(34): n is a, c, g, or t
  misc_feature (57)..(57): n is a, c, g, or t
  misc_feature (63)..(64): n is a, c, g, or t
  misc_feature (67)..(67): n is a, c, g, or t
  misc_feature (73)..(73): n is a, c, g, or t
  misc_feature (75)..(78): n is a, c, g, or t
  misc_feature (80)..(80): n is a, c, g, or t
  misc_feature (90)..(90): n is a, c, g, or t
  misc_feature (96)..(98): n is a, c, g, or t
cntgctnttacgatgcgcaaggtagtnccgtgantttagtccgtgatgtgtcgaaanattagnnttncagccngnnnnantgccattttngctctnnngagaaa

SEQ ID NO: 66 - DNA, 102 bp, Human
  misc_feature (5)..(5): n is a, c, g, or t
  misc_feature (12)..(12): n is a, c, g, or t
  misc_feature (28)..(28): n is a, c, g, or t
  misc_feature (32)..(32): n is a, c, g, or t
  misc_feature (38)..(38): n is a, c, g, or t
  misc_feature (44)..(44): n is a, c, g, or t
  misc_feature (57)..(57): n is a, c, g, or t
  misc_feature (68)..(68): n is a, c, g, or t
  misc_feature (76)..(76): n is a, c, g, or t
  misc_feature (82)..(82): n is a, c, g, or t
  misc_feature (92)..(92): n is a, c, g, or t
tgggntggcccngcttaacttttgcccncganctcggngttcgnacaggggcgaagnaaaccgccaanttttttcnaacccnacttgttttnggttttagtt

SEQ ID NO: 67 - DNA, 100 bp, Human
  misc_feature (3)..(3): n is a, c, g, or t
  misc_feature (21)..(21): n is a, c, g, or t
  misc_feature (27)..(28): n is a, c, g, or t
  misc_feature (38)..(38): n is a, c, g, or t
  misc_feature (47)..(47): n is a, c, g, or t
  misc_feature (50)..(50): n is a, c, g, or t
  misc_feature (53)..(53): n is a, c, g, or t
  misc_feature (61)..(61): n is a, c, g, or t
  misc_feature (73)..(73): n is a, c, g, or t
  misc_feature (78)..(78): n is a, c, g, or t
  misc_feature (83)..(83): n is a, c, g, or t
  misc_feature (85)..(85): n is a, c, g, or t
  misc_feature (97)..(97): n is a, c, g, or t
agnacgcctttacagctttangatgcnngagagagtancggatttgnccntgntggtgganaaattagggttncagcntgtgnantgccattttcgntaa

SEQ ID NO: 68 - DNA, 100 bp, Human
  misc_feature (21)..(22): n is a, c, g, or t
  misc_feature (28)..(28): n is a, c, g, or t
  misc_feature (32)..(32): n is a, c, g, or t
  misc_feature (65)..(65): n is a, c, g, or t
  misc_feature (67)..(68): n is a, c, g, or t
  misc_feature (70)..(70): n is a, c, g, or t
  misc_feature (77)..(77): n is a, c, g, or t
  misc_feature (83)..(83): n is a, c, g, or t
  misc_feature (92)..(92): n is a, c, g, or t
cacgatagcatcagacggcgnncttggngccnttttgcccgctggtcacaggacaacgcatttcncnntntggtgtncggctntcacgcatnggcgcgag

SEQ ID NO: 69 - DNA, 153 bp, Human
  misc_feature (4)..(4): n is a, c, g, or t
  misc_feature (8)..(8): n is a, c, g, or t
  misc_feature (29)..(29): n is a, c, g, or t
  misc_feature (43)..(44): n is a, c, g, or t
  misc_feature (46)..(46): n is a, c, g, or t
  misc_feature (57)..(57): n is a, c, g, or t
  misc_feature (59)..(59): n is a, c, g, or t
  misc_feature (84)..(84): n is a, c, g, or t
  misc_feature (98)..(98): n is a, c, g, or t
  misc_feature (115)..(115): n is a, c, g, or t
  misc_feature (117)..(118): n is a, c, g, or t
tggngccnttttgcccgctggtcacaggnaaacgcatttcacnntntggtgttcggntntcacgcacggcagcgagtgcaatgnccgattcattcttnaacgacgcacacacccngnngccctgtgaaacccataaacagtgggaaatggtgc

SEQ ID NO: 70 - DNA, 100 bp, Human
  misc_feature (3)..(3): n is a, c, g, or t
  misc_feature (26)..(26): n is a, c, g, or t
ganaataagctggagcctcgcgcgcntgcaggtcgacactagtggatccaaagaattcggcacgagacacagtgaagcaaattaaaaaaaaaaaaaaaaa

SEQ ID NO: 71 - DNA, 151 bp, Human
  misc_feature (7)..(7): n is a, c, g, or t
  misc_feature (10)..(10): n is a, c, g, or t
  misc_feature (53)..(53): n is a, c, g, or t
  misc_feature (61)..(61): n is a, c, g, or t
  misc_feature (65)..(66): n is a, c, g, or t
  misc_feature (72)..(72): n is a, c, g, or t
  misc_feature (74)..(75): n is a, c, g, or t
  misc_feature (89)..(89): n is a, c, g, or t
  misc_feature (94)..(94): n is a, c, g, or t
  misc_feature (100)..(100): n is a, c, g, or t
  misc_feature (107)..(107): n is a, c, g, or t
  misc_feature (126)..(126): n is a, c, g, or t
  misc_feature (141)..(141): n is a, c, g, or t
gcgcgcntgnaggccccgacactagtggatccaaagtattttggcacgagctnagttcgangatnnagaccncnnatcacctaatacanccatnactcanatgactntttgtgcgccttttatcanatgcatagcctatcnaaaacatcac

SEQ ID NO: 72 - DNA, 117 bp, Human
  misc_feature (14)..(14): n is a, c, g, or t
  misc_feature (18)..(18): n is a, c, g, or t
  misc_feature (40)..(40): n is a, c, g, or t
  misc_feature (56)..(56): n is a, c, g, or t
  misc_feature (72)..(74): n is a, c, g, or t
  misc_feature (76)..(76): n is a, c, g, or t
  misc_feature (78)..(78): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (90)..(92): n is a, c, g, or t
  misc_feature (98)..(99): n is a, c, g, or t
  misc_feature (107)..(107): n is a, c, g, or t
  misc_feature (115)..(115): n is a, c, g, or t
tttcgcgcgcctgnaggnccgacactagtggatccaaagnaatttggcacgagganctcggacgcttgtgannngngnganaangantgnnntctttnnttaataanagaaatgntt

SEQ ID NO: 73 - DNA, 101 bp, Human
  misc_feature (21)..(21): n is a, c, g, or t
  misc_feature (45)..(45): n is a, c, g, or t
  misc_feature (61)..(61): n is a, c, g, or t
  misc_feature (74)..(74): n is a, c, g, or t
actagtggatccaaagaattnggcacgagctattgaaatgacaanttttcatcttactgcncaatcaaaatganattgataggaatgaactcagaggctgg

SEQ ID NO: 74 - DNA, 122 bp, Human
  misc_feature (3)..(3): n is a, c, g, or t
  misc_feature (13)..(13): n is a, c, g, or t
  misc_feature (40)..(40): n is a, c, g, or t
  misc_feature (45)..(45): n is a, c, g, or t
  misc_feature (50)..(51): n is a, c, g, or t
  misc_feature (61)..(61): n is a, c, g, or t
  misc_feature (78)..(78): n is a, c, g, or t
  misc_feature (84)..(84): n is a, c, g, or t
  misc_feature (97)..(97): n is a, c, g, or t
  misc_feature (99)..(99): n is a, c, g, or t
  misc_feature (105)..(105): n is a, c, g, or t
  misc_feature (110)..(110): n is a, c, g, or t
cgnctagtggttnccaaagcaattcggcacgagcgctggncgaantggcnngcaaggaganagtaggataagctggcngcacanggccccatgggancntcaggnccagntggagcccgggg

SEQ ID NO: 75 - DNA, 100 bp, Human
  misc_feature (4)..(4): n is a, c, g, or t
  misc_feature (13)..(13): n is a, c, g, or t
  misc_feature (27)..(27): n is a, c, g, or t
  misc_feature (53)..(53): n is a, c, g, or t
  misc_feature (58)..(58): n is a, c, g, or t
  misc_feature (60)..(61): n is a, c, g, or t
  misc_feature (65)..(65): n is a, c, g, or t
  misc_feature (72)..(72): n is a, c, g, or t
gcgngactagtgnatctaaagaattcngcacagaggccagctaagccaggctncctgngnnctgngaggaanggtacccatcccccatgccccttatggg

SEQ ID NO: 76 - DNA, 100 bp, Human
  misc_feature (3)..(3): n is a, c, g, or t
  misc_feature (11)..(11): n is a, c, g, or t
  misc_feature (40)..(40): n is a, c, g, or t
  misc_feature (53)..(53): n is a, c, g, or t
  misc_feature (67)..(67): n is a, c, g, or t
  misc_feature (96)..(97): n is a, c, g, or t
tcnaggcgcgncacatagtggatctaaagaattcggcacnagccagtgctggngagctctctctttntgcaactaatctcatttcaccaggagctnncag

SEQ ID NO: 77 - DNA, 100 bp, Human
  misc_feature (2)..(2): n is a, c, g, or t
  misc_feature (10)..(10): n is a, c, g, or t
  misc_feature (61)..(61): n is a, c, g, or t
  misc_feature (63)..(63): n is a, c, g, or t
  misc_feature (72)..(72): n is a, c, g, or t
  misc_feature (79)..(79): n is a, c, g, or t
gngcgcttgnaggccgacactaggggatccaaagaattcggcacgagctcgtgccgaattngncacgagttnggctgcntctttatacaacttttcttca

SEQ ID NO: 78 - DNA, 102 bp, Human
  misc_feature (3)..(3): n is a, c, g, or t
  misc_feature (21)..(21): n is a, c, g, or t
  misc_feature (41)..(41): n is a, c, g, or t
  misc_feature (62)..(62): n is a, c, g, or t
  misc_feature (65)..(65): n is a, c, g, or t
  misc_feature (69)..(69): n is a, c, g, or t
cgngcgcttgaaggtcgacantagtggatccaaaaaatttngcacgagcacgatctggactnganctcntttgaactggacttacagttttccgaagataag

SEQ ID NO: 79 - DNA, 100 bp, Human
  misc_feature (5)..(5): n is a, c, g, or t
  misc_feature (7)..(8): n is a, c, g, or t
  misc_feature (10)..(10): n is a, c, g, or t
  misc_feature (15)..(16): n is a, c, g, or t
  misc_feature (20)..(20): n is a, c, g, or t
  misc_feature (24)..(24): n is a, c, g, or t
  misc_feature (71)..(71): n is a, c, g, or t
aaagngnntnctggnnttangcanttaacccaggcactggggcgctgaacagctactcagctgcttaagtngtcccactggtccagaccagcgacccagc

SEQ ID NO: 80 - DNA, 100 bp, Human
ctggagcgcccactattgactctaaagaattccaattcaaatactccccagactcccgagggagctgaaggatgtggctgatagtgctaacagtgcaaac

SEQ ID NO: 81 - DNA, 102 bp, Human
  misc_feature (80)..(80): n is a, c, g, or t
  misc_feature (91)..(91): n is a, c, g, or t
ttcccccaggatctttcttatatctatcagatctaggtgaaaggattactgtcttgtaggtgtcctgaaggacaagccgnttcgtttgaanctgtgaaatac

SEQ ID NO: 82 - DNA, 325 bp, Human
ttcggcacgaggagaagagaggagccgtcagaacatatgggggatgtgttcaagaagcagatttgtggtcggaagctttgcaaagaggggacctgggtctgagtgacatgcgtggccactggtgctcctgcgtttggactgtgcaggcctctcctatgctgatgcgtctccccactcctgagctaatttctgctctgctccttctgtgacatgtggcagcgtgggaaatagccactgtcccctgtccctgctgttcctggtgtcacccagcaccaggccactctgggagccagggcagatggtcctccctgtggtcctggcctct

SEQ ID NO: 83 - DNA, 102 bp, Human
  misc_feature (3)..(3): n is a, c, g, or t
gancaaacatatccatgatgcatgaataatgcgctttgaagcaagatttcaagctatcaggaagcatactatggatgctaatcatgtatagaattccatgat

SEQ ID NO: 84 - DNA, 100 bp, Human
  misc_feature (7)..(7): n is a, c, g, or t
  misc_feature (17)..(17): n is a, c, g, or t
  misc_feature (32)..(32): n is a, c, g, or t
  misc_feature (36)..(36): n is a, c, g, or t
  misc_feature (49)..(49): n is a, c, g, or t
  misc_feature (59)..(59): n is a, c, g, or t
  misc_feature (92)..(92): n is a, c, g, or t
  misc_feature (96)..(96): n is a, c, g, or t
  misc_feature (99)..(99): n is a, c, g, or t
aaaaagnccaaatttcnggagcttgcgcgccngcantagggcactaaangaattcaaanaatagggctcgaggtcttcgcctgcgggcacgnaggncanc

SEQ ID NO: 85 - DNA, 103 bp, Human
  misc_feature (14)..(14): n is a, c, g, or t
  misc_feature (31)..(31): n is a, c, g, or t
  misc_feature (36)..(36): n is a, c, g, or t
  misc_feature (63)..(63): n is a, c, g, or t
  misc_feature (73)..(73): n is a, c, g, or t
  misc_feature (93)..(93): n is a, c, g, or t
  misc_feature (98)..(98): n is a, c, g, or t
gtggcccaaggggnactgaaggggccctccntaagnggaggggttggggagtaaggcctgggnaggaccctgntgactcggggggcgggagcnggganccagg

SEQ ID NO: 86 - DNA, 154 bp, Human
  misc_feature (4)..(5): n is a, c, g, or t
  misc_feature (10)..(10): n is a, c, g, or t
  misc_feature (13)..(13): n is a, c, g, or t
  misc_feature (32)..(32): n is a, c, g, or t
  misc_feature (34)..(34): n is a, c, g, or t
  misc_feature (68)..(68): n is a, c, g, or t
  misc_feature (75)..(76): n is a, c, g, or t
  misc_feature (83)..(83): n is a, c, g, or t
  misc_feature (88)..(88): n is a, c, g, or t
  misc_feature (94)..(94): n is a, c, g, or t
  misc_feature (110)..(110): n is a, c, g, or t
  misc_feature (116)..(116): n is a, c, g, or t
  misc_feature (133)..(133): n is a, c, g, or t
ccgnnagggnacntgcggggccaaaaccaagngnatccggggcagggggccttgaacttggaaaaagnagtttgnngccgatngcaantacatntttaaaaactggggtncttggnaggggggcaaagccccnaaagtccgccaaggacccttg

SEQ ID NO: 87 - DNA, 100 bp, Human
  misc_feature (53)..(53): n is a, c, g, or t
ctggcgcttgcgcgcccgcactaggggactcaaggaattcggttcgaggagcntactaggttatgggggcatgcttgcatgctcctatgaacccctttcg

SEQ ID NO: 88 - DNA, 100 bp, Human
  misc_feature (2)..(2): n is a, c, g, or t
  misc_feature (11)..(11): n is a, c, g, or t
  misc_feature (27)..(28): n is a, c, g, or t
  misc_feature (34)..(34): n is a, c, g, or t
  misc_feature (38)..(39): n is a, c, g, or t
  misc_feature (41)..(41): n is a, c, g, or t
  misc_feature (49)..(50): n is a, c, g, or t
  misc_feature (61)..(62): n is a, c, g, or t
  misc_feature (67)..(68): n is a, c, g, or t
  misc_feature (76)..(76): n is a, c, g, or t
  misc_feature (79)..(80): n is a, c, g, or t
  misc_feature (85)..(85): n is a, c, g, or t
  misc_feature (89)..(89): n is a, c, g, or t
  misc_feature (93)..(93): n is a, c, g, or t
anagagatcantgatttattgctgggnncctgtntganngntctaaggnntgaagattatnncattnngcaagcgnacnngcgcngccnagcngaccagg

SEQ ID NO: 89 - DNA, 106 bp, Human
  misc_feature (8)..(8): n is a, c, g, or t
  misc_feature (24)..(24): n is a, c, g, or t
  misc_feature (33)..(34): n is a, c, g, or t
  misc_feature (47)..(48): n is a, c, g, or t
  misc_feature (58)..(58): n is a, c, g, or t
  misc_feature (63)..(63): n is a, c, g, or t
  misc_feature (68)..(68): n is a, c, g, or t
  misc_feature (71)..(71): n is a, c, g, or t
  misc_feature (73)..(73): n is a, c, g, or t
  misc_feature (77)..(77): n is a, c, g, or t
  misc_feature (88)..(88): n is a, c, g, or t
  misc_feature (95)..(96): n is a, c, g, or t
atatttcnggagcttgcagcggcnacactaggnnactaaaagaattnnagaaagaggnctatnggacnagnanacangaaacctgcanacttggnngcttggaagt

SEQ ID NO: 90 - DNA, 100 bp, Human
  misc_feature (74)..(74): n is a, c, g, or t
gatgtggagatgcttgataggttactgggcggcaatccaggagttgatgaagcgcatatgcgaacatttcacgngcatattgcggtgcaagggcttactg

SEQ ID NO: 91 - DNA, 101 bp, Human
  misc_feature (7)..(8): n is a, c, g, or t
  misc_feature (17)..(17): n is a, c, g, or t
  misc_feature (23)..(23): n is a, c, g, or t
  misc_feature (33)..(33): n is a, c, g, or t
  misc_feature (42)..(42): n is a, c, g, or t
  misc_feature (47)..(48): n is a, c, g, or t
  misc_feature (52)..(52): n is a, c, g, or t
  misc_feature (55)..(55): n is a, c, g, or t
  misc_feature (60)..(60): n is a, c, g, or t
  misc_feature (62)..(62): n is a, c, g, or t
  misc_feature (66)..(66): n is a, c, g, or t
  misc_feature (70)..(71): n is a, c, g, or t
  misc_feature (74)..(74): n is a, c, g, or t
  misc_feature (84)..(84): n is a, c, g, or t
  misc_feature (86)..(87): n is a, c, g, or t
  misc_feature (96)..(96): n is a, c, g, or t
  misc_feature (99)..(100): n is a, c, g, or t
ccccccnncccttcttntccccnaaagaataanataagaatngctanngagnaancgacnanggtnttannagntatatgtatntnncaaaccaantanna

SEQ ID NO: 92 - DNA, 100 bp, Human
  misc_feature (5)..(6): n is a, c, g, or t
  misc_feature (33)..(33): n is a, c, g, or t
  misc_feature (35)..(36): n is a, c, g, or t
  misc_feature (42)..(42): n is a, c, g, or t
  misc_feature (46)..(46): n is a, c, g, or t
  misc_feature (48)..(48): n is a, c, g, or t
  misc_feature (55)..(55): n is a, c, g, or t
  misc_feature (59)..(59): n is a, c, g, or t
  misc_feature (69)..(69): n is a, c, g, or t
  misc_feature (81)..(81): n is a, c, g, or t
  misc_feature (87)..(87): n is a, c, g, or t
  misc_feature (91)..(91): n is a, c, g, or t
  misc_feature (94)..(94): n is a, c, g, or t
  misc_feature (97)..(97): n is a, c, g, or t
aaggnnaggctcgttgggggaaaaaacccgccntnncgggcncccngnaaacccncacnaggggacccnaaaaaccggaanaaaccncccnagnaancca

SEQ ID NO: 93 - DNA, 183 bp, Human
  misc_feature (46)..(46): n is a, c, g, or t
  misc_feature (105)..(105): n is a, c, g, or t
tcctcctcaaccacaccatcttggctggagttcacagcaatgaatnactctggtggtttagaacggaaactgaggaatgaggaaaccagcccgtcctctctttgnaagggataaacaaaccctcccccctacccaaatttccaagggcaaagggtgggggttgtaaaaaagggtgagatgggb

SEQ ID NO: 94 - DNA, 121 bp, Human
ggaaaaataaaagtggaacctttgaattttggggtaattagaaaaaaaaaaaatttttaattgggggagaaggttgtttatggggagaatatgtggaatggaaataaacctaaattcaagg

SEQ ID NO: 95 - DNA, 417 bp, Human
tatgacaataaattagtattctgatatttgctagattattttcatgataaaccccgacactaagctattaatacggcttggtcttaaggatatcttggagggcatgctctgaaattcccatataagggaggttaaacctgaaccttaaggggccctcaattatacaaggggtatggaactcaggaaaataaaatctctggtagctcgacccctcttcgcctgtgtggagagaatatattctctatatacagggggaaactgtgagctgtgatacacgtcaaagagagtctctcactgggcgcgactctcgagagggagagaatgagagagaaagagataatgcgatatcatcaaacgtggcgcggtgtggggctcctcctctgtgagagagagtaatacaggagtacactgctccta

SEQ ID NO: 96 - DNA, 142 bp, Human
tccactcttcagttctgagaaaaaggactgctcttgcacttggggggccttcgggtttccgaggctcattgggacggcttggctcttatgaaagccccaaagggttccttaggacggcttggatacccctgtaaggtttaaa

SEQ ID NO: 97 - DNA, 188 bp, Human
aaaataatgaggattgactgcttcatctggggaagtactgtacgtctgcgttttgtggcataggatgccttccctgtggcatcttcgactcctcttacctcaagttctggggcacaactggggaatgtcattattcgctcttcgagatgttatcaataaattacacatgggggctttccaagtaatgg

SEQ ID NO: 98 - DNA, 250 bp, Human
tggagaggctggagacactgtacagtttggcagaagtatattcagaaaaacggtgcaactttataaggatgcgaaatgggatatcaatttgttctccccacggaaaacagctaaactttcaacagggcggaaacctgggctgacttttcttcggagagcgggcccccatatacatgtggagaccccccccctcggctgggctatatattagacataaaaggggccacacccttttatttacaaaggactt

SEQ ID NO: 99 - DNA, 160 bp, Human
cagtattaagctataaatactcaaaagtggcctcgagttaaatagtcattgttatttcatctaagtcatgtaggttgatattttcaacatatcattggagaattaatcttctttattaaaatagggcattttcttctttctggcgaaagttgggagtaac

SEQ ID NO: 100 - DNA, 142 bp, Human
tgtgacacccaagagaggtgccaccacgcacaccctcccgcactcacacgcgagggaaccattacctctcacagacaaagaggcctgggatatgaggactcggggggggtgaaagcatcatggggcagacagatggggatgg

SEQ ID NO: 101 - DNA, 230 bp, Human
acccccccgacaaccgggggcggagcagcacgatgaggcacggcaagcccatgagccacgcctcccggcggattaccgaacaagatgaggccacactagacgatctgcgcagaccggatcaaaggactgggacagctgtgcatagacagaggagccaaggaggagcctgctaagcgaagaaatgaactacaagggagcgacagtgccacacaaacaactaataaaggaag

SEQ ID NO: 102 - DNA, 102 bp, Human
ttacctacgtcagcagtgggaactgcaacttggggctttgcgaataaaatttagctgccttgttgaaaaaaaaaaaaagaaaagaaagaaaaacaaaaagaa

SEQ ID NO: 103 - DNA, 112 bp, Human
gccttctcttgatggggccgagattgcactgggccgcggcggacgagatgctgtcggtttgattgataggagagtaaggcggctacttaaaacatatagtcaataggctact

SEQ ID NO: 104 - DNA, 200 bp, Human
  misc_feature (19)..(19): n is a, c, g, or t
  misc_feature (39)..(39): n is a, c, g, or t
  misc_feature (48)..(48): n is a, c, g, or t
  misc_feature (62)..(62): n is a, c, g, or t
  misc_feature (97)..(97): n is a, c, g, or t
  misc_feature (105)..(105): n is a, c, g, or t
  misc_feature (107)..(107): n is a, c, g, or t
  misc_feature (164)..(164): n is a, c, g, or t
  misc_feature (174)..(174): n is a, c, g, or t
  misc_feature (189)..(189): n is a, c, g, or t
cgctgatcgaaaacttgcnccagggagagaacccttgcntatgttganttccactgcctctnctcatacagaagcgatgttggaaccgttcttttgntggctgantnatggttttttaaggataaacaaaagtttttattacatctgaaagaaggaaagtaaanggacaagttnaataaaaagggcctnccctttagaat

SEQ ID NO: 105 - DNA, 124 bp, Human
  misc_feature (34)..(34): n is a, c, g, or t
tggtcggaggctagaaggccgagtggactctgcngaccgccagcaacacgttcgtgaagccccattttcagttcgggacatcgaaccgaggccaaagcgggaagggttgcgccgagcctcatac

SEQ ID NO: 106 - DNA, 116 bp, Human
  misc_feature (48)..(48): n is a, c, g, or t
gtcttaagttttaagggaacttggctccttgaacctccctgaaagaanctctaccatttaattaagaaaagcagttgcctgttcggaaagcatctttgagaggaaacaggaaaagt

SEQ ID NO: 107 - DNA, 163 bp, Human
  misc_feature (8)..(8): n is a, c, g, or t
  misc_feature (10)..(10): n is a, c, g, or t
  misc_feature (16)..(16): n is a, c, g, or t
  misc_feature (64)..(64): n is a, c, g, or t
  misc_feature (100)..(100): n is a, c, g, or t
  misc_feature (122)..(122): n is a, c, g, or t
  misc_feature (151)..(151): n is a, c, g, or t
  misc_feature (156)..(156): n is a, c, g, or t
aaaagccnangggcgngagaaaccggccatgatgactcatgcatgactcagttctctgcggggnggggataaaaggccatgaccaaaggctcgcagattnttgagaagagttttgggaaagnattactcttgtcttctgctgctcctgggnaaagngatttgg

SEQ ID NO: 108 - DNA, 140 bp, Human
  misc_feature (26)..(26): n is a, c, g, or t
  misc_feature (89)..(89): n is a, c, g, or t
  misc_feature (126)..(126): n is a, c, g, or t
ggcttcccctacgcaaaaggacccangggcggatacgccagcaggtctctgcccagccgcttgtggaaaatcggcctcggaagaggaangaaattcccggggttatattcaaggcgggcttctttncagaatccatcgga

SEQ ID NO: 109 - DNA, 118 bp, Human
  misc_feature (21)..(21): n is a, c, g, or t
taaagcgggcccaagaggagnataaagccaaagaatttccttggcagcctgaagcgccagtaagcggctggtggggaaccgagacttcgcccccgagggggaagcaggggaacccagc

SEQ ID NO: 110 - DNA, 160 bp, Human
  misc_feature (73)..(73): n is a, c, g, or t
  misc_feature (94)..(94): n is a, c, g, or t
  misc_feature (134)..(134): n is a, c, g, or t
  misc_feature (139)..(139): n is a, c, g, or t
aggattagtgacctaagattatttttgctgtcccgttttttgtaaatcaaaatgaaaattataaaagaagganttctgacagtaggaattttgnacatattgattatatgggggtccaaataaaaaatataaantgatnaaagactggaaataaaaataa

SEQ ID NO: 111 - DNA, 130 bp, Human
  misc_feature (44)..(44): n is a, c, g, or t
  misc_feature (82)..(82): n is a, c, g, or t
  misc_feature (107)..(107): n is a, c, g, or t
taattctgagaaggcacgcatagaaatatcttaagaaacacagnaggaaagttactcagaactaaactttaagagcttaagngactactttcttggaaaatgacatnctgagctcaaaaggaatgggtaa

SEQ ID NO: 112 - DNA, 146 bp, Human
  misc_feature (12)..(12): n is a, c, g, or t
  misc_feature (41)..(41): n is a, c, g, or t
  misc_feature (62)..(62): n is a, c, g, or t
  misc_feature (100)..(100): n is a, c, g, or t
  misc_feature (115)..(115): n is a, c, g, or t
  misc_feature (134)..(134): n is a, c, g, or t
acgagttcgagnccgggaaccttccaaggctctttctgggngctggaagaagaacctcgggncctcaaggctcagctcaggggagcggaaaaattacgtnagctaaggaaatagnaagttgggggggggggggnaagctatcataa

SEQ ID NO: 113 - DNA, 210 bp, Human
  misc_feature (38)..(38): n is a, c, g, or t
  misc_feature (43)..(43): n is a, c, g, or t
  misc_feature (77)..(77): n is a, c, g, or t
  misc_feature (82)..(82): n is a, c, g, or t
  misc_feature (119)..(119): n is a, c, g, or t
  misc_feature (140)..(140): n is a, c, g, or t
  misc_feature (151)..(151): n is a, c, g, or t
  misc_feature (153)..(153): n is a, c, g, or t
  misc_feature (202)..(202): n is a, c, g, or t
aatgatgcttaaaatcataccgtgagggctttgggagngaggnggaacatgggattagcctatctcatctagaaaanagatntaaaaccaaggggggggctcaatccagggctcttccntacctttctccccctcttaantcatttggccngnctttagttcatgctgggtaagggaataaaacgagcaatcacaaaacaangcctggaa

SEQ ID NO: 114 - DNA, 104 bp, Human
taagcagagccatttgtgactagattgttcagtaagtataaatttatgatgggagtgcggggctcatgcctggcagagccagcacttggggctaggctttgcca

SEQ ID NO: 115 - DNA, 100 bp, Human
  misc_feature (14)..(14): n is a, c, g, or t
  misc_feature (22)..(22): n is a, c, g, or t
  misc_feature (34)..(34): n is a, c, g, or t
  misc_feature (41)..(41): n is a, c, g, or t
  misc_feature (43)..(43): n is a, c, g, or t
  misc_feature (45)..(46): n is a, c, g, or t
  misc_feature (49)..(49): n is a, c, g, or t
  misc_feature (57)..(57): n is a, c, g, or t
  misc_feature (65)..(65): n is a, c, g, or t
  misc_feature (77)..(77): n is a, c, g, or t
  misc_feature (80)..(80): n is a, c, g, or t
  misc_feature (89)..(89): n is a, c, g, or t
aaaacaggtgtgtnttttcttnttttaattggcncaccttntncnntanatctgtgncttgcacnaaacaaattggnggnagccctganattttctggtc

SEQ ID NO: 116 - DNA, 116 bp, Human
  misc_feature (11)..(11): n is a, c, g, or t
  misc_feature (19)..(19): n is a, c, g, or t
  misc_feature (29)..(29): n is a, c, g, or t
  misc_feature (31)..(31): n is a, c, g, or t
  misc_feature (34)..(34): n is a, c, g, or t
  misc_feature (52)..(52): n is a, c, g, or t
  misc_feature (60)..(60): n is a, c, g, or t
  misc_feature (68)..(68): n is a, c, g, or t
  misc_feature (70)..(70): n is a, c, g, or t
  misc_feature (74)..(74): n is a, c, g, or t
  misc_feature (88)..(88): n is a, c, g, or t
  misc_feature (109)..(109): n is a, c, g, or t
ttttcattttnaagggggnccccgggggntnagntcccaaaaaggattaaanattggggncccccccntntttntcttttggcaaaanttggcctaaccttaggtggtntcctccg

SEQ ID NO: 117 - DNA, 100 bp, Human
  misc_feature (2)..(2): n is a, c, g, or t
  misc_feature (8)..(8): n is a, c, g, or t
  misc_feature (30)..(30): n is a, c, g, or t
  misc_feature (39)..(39): n is a, c, g, or t
  misc_feature (48)..(49): n is a, c, g, or t
  misc_feature (52)..(52): n is a, c, g, or t
  misc_feature (81)..(81): n is a, c, g, or t
cncagaangaatgcttactcagttttgtgngcagccagnataataaannccnggggagggacagaagattcaataagagantatgaagatgggatggagg

SEQ ID NO: 118 - DNA, 101 bp, Human
  misc_feature (2)..(2): n is a, c, g, or t
  misc_feature (11)..(11): n is a, c, g, or t
  misc_feature (15)..(15): n is a, c, g, or t
  misc_feature (43)..(43): n is a, c, g, or t
  misc_feature (48)..(48): n is a, c, g, or t
  misc_feature (69)..(74): n is a, c, g, or t
anggcgggagnaaanccaaaaaaaaaaaaaaaaaaaaattttntgggnaaaaaaaggagaaaaaaaaannnnnnaaaaaaaaaaaagggggggggcggggg

SEQ ID NO: 119 - DNA, 453 bp, Human
  misc_feature (5)..(6): n is a, c, g, or t
  misc_feature (13)..(13): n is a, c, g, or t
  misc_feature (18)..(18): n is a, c, g, or t
  misc_feature (22)..(22): n is a, c, g, or t
  misc_feature (39)..(39): n is a, c, g, or t
  misc_feature (45)..(45): n is a, c, g, or t
  misc_feature (48)..(48): n is a, c, g, or t
  misc_feature (54)..(54): n is a, c, g, or t
  misc_feature (65)..(65): n is a, c, g, or t
  misc_feature (70)..(70): n is a, c, g, or t
  misc_feature (72)..(72): n is a, c, g, or t
  misc_feature (80)..(80): n is a, c, g, or t
  misc_feature (83)..(83): n is a, c, g, or t
  misc_feature (90)..(90): n is a, c, g, or t
  misc_feature (94)..(94): n is a, c, g, or t
  misc_feature (96)..(96): n is a, c, g, or t
  misc_feature (98)..(98): n is a, c, g, or t
  misc_feature (102)..(102): n is a, c, g, or t
  misc_feature (104)..(104): n is a, c, g, or t
  misc_feature (113)..(113): n is a, c, g, or t
  misc_feature (115)..(115): n is a, c, g, or t
  misc_feature (117)..(117): n is a, c, g, or t
  misc_feature (121)..(121): n is a, c, g, or t
  misc_feature (123)..(123): n is a, c, g, or t
  misc_feature (125)..(125): n is a, c, g, or t
  misc_feature (128)..(128): n is a, c, g, or t
  misc_feature (142)..(142): n is a, c, g, or t
  misc_feature (144)..(144): n is a, c, g, or t
  misc_feature (160)..(160): n is a, c, g, or t
  misc_feature (174)..(175): n is a, c, g, or t
  misc_feature (180)..(181): n is a, c, g, or t
  misc_feature (186)..(186): n is a, c, g, or t
  misc_feature (192)..(192): n is a, c, g, or t
  misc_feature (198)..(198): n is a, c, g, or t
  misc_feature (205)..(205): n is a, c, g, or t
  misc_feature (215)..(215): n is a, c, g, or t
  misc_feature (223)..(224): n is a, c, g, or t
  misc_feature (233)..(234): n is a, c, g, or t
  misc_feature (238)..(238): n is a, c, g, or t
  misc_feature (243)..(243): n is a, c, g, or t
  misc_feature (253)..(253): n is a, c, g, or t
  misc_feature (257)..(257): n is a, c, g, or t
  misc_feature (268)..(268): n is a, c, g, or t
  misc_feature (280)..(280): n is a, c, g, or t
  misc_feature (291)..(291): n is a, c, g, or t
  misc_feature (296)..(296): n is a, c, g, or t
  misc_feature (300)..(300): n is a, c, g, or t
  misc_feature (305)..(305): n is a, c, g, or t
  misc_feature (308)..(308): n is a, c, g, or t
  misc_feature (311)..(311): n is a, c, g, or t
  misc_feature (319)..(319): n is a, c, g, or t
  misc_feature (322)..(322): n is a, c, g, or t
  misc_feature (326)..(326): n is a, c, g, or t
  misc_feature (328)..(328): n is a, c, g, or t
  misc_feature (334)..(335): n is a, c, g, or t
  misc_feature (338)..(339): n is a, c, g, or t
  misc_feature (347)..(347): n is a, c, g, or t
  misc_feature (352)..(352): n is a, c, g, or t
  misc_feature (358)..(358): n is a, c, g, or t
  misc_feature (361)..(361): n is a, c, g, or t
  misc_feature (367)..(367): n is a, c, g, or t
  misc_feature (382)..(382): n is a, c, g, or t
  misc_feature (389)..(389): n is a, c, g, or t
  misc_feature (395)..(395): n is a, c, g, or t
  misc_feature (410)..(410): n is a, c, g, or t
  misc_feature (413)..(414): n is a, c, g, or t
  misc_feature (418)..(418): n is a, c, g, or t
  misc_feature (425)..(425): n is a, c, g, or t
  misc_feature (436)..(436): n is a, c, g, or t
  misc_feature (443)..(443): n is a, c, g, or t
ttccnnagctgtnacganacantcttgaattgaaattgnacacanctngtgtgnagccctgatanggccngnaagcaatntanaggatanccgnangntatngnaacacattncncnagcntntncancagctgatgcaggncncctatgatgcgattanggactacgactatnnctcanngtctnaacagncgcganggctgantactaaaagnacacaaanntgtgcaccnncatnactcncgttgactgnacantgtagacctgnaatacctggctnaaaggggtctnactgncatnagagntgnagntgcccctncantagngngagctnnaannggcctgtntttgntttacntcntcgganaggcgatgccattanagacccnagaacncattggtgatatacnctnnaccnggagggnttacattgggnaatgatnattatggggg

SEQ ID NO: 120 - DNA, 100 bp, Human
  misc_feature (12)..(12): n is a, c, g, or t
  misc_feature (18)..(18): n is a, c, g, or t
  misc_feature (48)..(48): n is a, c, g, or t
  misc_feature (56)..(56): n is a, c, g, or t
acccagggaaantcggtnttatggccgggggactttccactgtacagnatttcagncatcattcactatgactcttttttcttgactgttgcttgttctt

SEQ ID NO: 121 - DNA, 301 bp, Human
  misc_feature (2)..(2): n is a, c, g, or t
  misc_feature (7)..(7): n is a, c, g, or t
  misc_feature (10)..(10): n is a, c, g, or t
  misc_feature (18)..(19): n is a, c, g, or t
  misc_feature (27)..(27): n is a, c, g, or t
  misc_feature (36)..(36): n is a, c, g, or t
  misc_feature (38)..(39): n is a, c, g, or t
  misc_feature (41)..(41): n is a, c, g, or t
  misc_feature (48)..(48): n is a, c, g, or t
  misc_feature (60)..(60): n is a, c, g, or t
  misc_feature (68)..(68): n is a, c, g, or t
  misc_feature (72)..(72): n is a, c, g, or t
  misc_feature (82)..(82): n is a, c, g, or t
  misc_feature (84)..(84): n is a, c, g, or t
  misc_feature (86)..(88): n is a, c, g, or t
  misc_feature (112)..(112): n is a, c, g, or t
  misc_feature (125)..(125): n is a, c, g, or t
  misc_feature (129)..(129): n is a, c, g, or t
  misc_feature (142)..(144): n is a, c, g, or t
  misc_feature (146)..(146): n is a, c, g, or t
  misc_feature (152)..(152): n is a, c, g, or t
  misc_feature (154)..(154): n is a, c, g, or t
  misc_feature (157)..(157): n is a, c, g, or t
  misc_feature (160)..(163): n is a, c, g, or t
  misc_feature (165)..(165): n is a, c, g, or t
  misc_feature (169)..(169): n is a, c, g, or t
  misc_feature (175)..(176): n is a, c, g, or t
  misc_feature (181)..(181): n is a, c, g, or t
  misc_feature (186)..(186): n is a, c, g, or t
  misc_feature (192)..(192): n is a, c, g, or t
  misc_feature (194)..(194): n is a, c, g, or t
  misc_feature (196)..(196): n is a, c, g, or t
  misc_feature (199)..(199): n is a, c, g, or t
  misc_feature (212)..(212): n is a, c, g, or t
  misc_feature (216)..(216): n is a, c, g, or t
  misc_feature (231)..(231): n is a, c, g, or t
  misc_feature (238)..(238): n is a, c, g, or t
  misc_feature (243)..(243): n is a, c, g, or t
  misc_feature (259)..(262): n is a, c, g, or t
  misc_feature (269)..(269): n is a, c, g, or t
  misc_feature (278)..(278): n is a, c, g, or t
  misc_feature (285)..(285): n is a, c, g, or t
  misc_feature (287)..(287): n is a, c, g, or t
  misc_feature (296)..(296): n is a, c, g, or t
  misc_feature (299)..(299): n is a, c, g, or t
gntctgnctnaatagccnnttctctcntattcattncnntncaaatgnggctactgaggngtaggccnatantgcccccttnancnnnctcgacctgcacctcggtggtgcnacacctcctgctncacnatgcatatataannntnacaccntntcngannnnancatnggcacnnattgntgatntgagcngncncantgatgatctgaangacntgtcgcgggcctcangacacangctgngcgtgctactaactgnnnngagcaanccagatacntgccgcncntatcgagangangg

SEQ ID NO: 122 - DNA, 109 bp, Human
  misc_feature (7)..(7): n is a, c, g, or t
  misc_feature (20)..(20): n is a, c, g, or t
  misc_feature (30)..(30): n is a, c, g, or t
  misc_feature (34)..(34): n is a, c, g, or t
  misc_feature (37)..(37): n is a, c, g, or t
  misc_feature (44)..(44): n is a, c, g, or t
  misc_feature (46)..(46): n is a, c, g, or t
  misc_feature (49)..(49): n is a, c, g, or t
  misc_feature (55)..(55): n is a, c, g, or t
  misc_feature (67)..(67): n is a, c, g, or t
  misc_feature (75)..(75): n is a, c, g, or t
  misc_feature (78)..(78): n is a, c, g, or t
  misc_feature (81)..(83): n is a, c, g, or t
  misc_feature (86)..(87): n is a, c, g, or t
  misc_feature (89)..(89): n is a, c, g, or t
  misc_feature (101)..(102): n is a, c, g, or t
cagaagnaaactatgccaantaacaaaagnaaanaanaaaaatntncanaaagcngaaaaggaaaanaaggaaanaangannnaannanaaaaaaaaaatnngctgctg

SEQ ID NO: 123 - DNA, 352 bp, Human
ggccctggtccacaatcttcttccttataacaagttagaagctattatttaattcaagttggcaattttctcagtccaccagttgtgccacatctaacaacatagtaggggttgcatgtctgtgatctgagaagacactacgatgtacaacatggaaccttgtcctgctgccccaggcctttttataggggaagcgtatctttgcctattagcaattccagggggaggtcttaaagtgcaagaatctttctgcacaagtctgccttagttggatccaaacttaaattccaaagacacaagttacttcttcccctgcatgaaattgccctgaagtaattttttatatataatc

SEQ ID NO: 124 - DNA, 100 bp, Human
  misc_feature (9)..(9): n is a, c, g, or t
  misc_feature (20)..(20): n is a, c, g, or t
  misc_feature (28)..(28): n is a, c, g, or t
  misc_feature (38)..(38): n is a, c, g, or t
  misc_feature (40)..(40): n is a, c, g, or t
  misc_feature (44)..(44): n is a, c, g, or t
  misc_feature (46)..(46): n is a, c, g, or t
  misc_feature (48)..(49): n is a, c, g, or t
  misc_feature (52)..(52): n is a, c, g, or t
  misc_feature (54)..(54): n is a, c, g, or t
  misc_feature (62)..(62): n is a, c, g, or t
  misc_feature (65)..(65): n is a, c, g, or t
  misc_feature (76)..(77): n is a, c, g, or t
  misc_feature (97)..(97): n is a, c, g, or t
gcaggaacntgtatttattngggttatnaaggtgcgtntnggcncngnnttntnctggtttnacngcactcggtannctattcatgattaatcaggnaga

SEQ ID NO: 125 - DNA, 733 bp, Human
  misc_feature (13)..(14): n is a, c, g, or t
  misc_feature (17)..(17): n is a, c, g, or t
  misc_feature (20)..(20): n is a, c, g, or t
  misc_feature (22)..(22): n is a, c, g, or t
  misc_feature (27)..(27): n is a, c, g, or t
  misc_feature (29)..(29): n is a, c, g, or t
  misc_feature (31)..(31): n is a, c, g, or t
  misc_feature (36)..(36): n is a, c, g, or t
  misc_feature (43)..(43): n is a, c, g, or t
  misc_feature (46)..(46): n is a, c, g, or t
  misc_feature (58)..(59): n is a, c, g, or t
  misc_feature (74)..(74): n is a, c, g, or t
  misc_feature (78)..(78): n is a, c, g, or t
  misc_feature (81)..(81): n is a, c, g, or t
  misc_feature (85)..(85): n is a, c, g, or t
  misc_feature (87)..(88): n is a, c, g, or t
  misc_feature (90)..(92): n is a, c, g, or t
  misc_feature (95)..(96): n is a, c, g, or t
  misc_feature (105)..(105): n is a, c, g, or t
  misc_feature (107)..(107): n is a, c, g, or t
  misc_feature (109)..(109): n is a, c, g, or t
  misc_feature (115)..(117): n is a, c, g, or t
  misc_feature (120)..(120): n is a, c, g, or t
  misc_feature (123)..(123): n is a, c, g, or t
  misc_feature (148)..(148): n is a, c, g, or t
  misc_feature (154)..(156): n is a, c, g, or t
  misc_feature (167)..(167): n is a, c, g, or t
  misc_feature (169)..(172): n is a, c, g, or t
  misc_feature (179)..(179): n is a, c, g, or t
  misc_feature (183)..(183): n is a, c, g, or t
  misc_feature (185)..(187): n is a, c, g, or t
  misc_feature (194)..(194): n is a, c, g, or t
  misc_feature (200)..(203): n is a, c, g, or t
  misc_feature (205)..(205): n is a, c, g, or t
  misc_feature (235)..(236): n is a, c, g, or t
  misc_feature (242)..(242): n is a, c, g, or t
  misc_feature (244)..(244): n is a, c, g, or t
  misc_feature (246)..(247): n is a, c, g, or t
  misc_feature (250)..(250): n is a, c, g, or t
  misc_feature (303)..(303): n is a, c, g, or t
  misc_feature (305)..(305): n is a, c, g, or t
  misc_feature (318)..(318): n is a, c, g, or t
  misc_feature (327)..(327): n is a, c, g, or t
  misc_feature (331)..(331): n is a, c, g, or t
  misc_feature (335)..(336): n is a, c, g, or t
  misc_feature (347)..(347): n is a, c, g, or t
  misc_feature (349)..(349): n is a, c, g, or t
  misc_feature (353)..(353): n is a, c, g, or t
  misc_feature (362)..(362): n is a, c, g, or t
  misc_feature (365)..(365): n is a, c, g, or t
  misc_feature (388)..(388): n is a, c, g, or t
  misc_feature (395)..(395): n is a, c, g, or t
  misc_feature (405)..(405): n is a, c, g, or t
  misc_feature (407)..(407): n is a, c, g, or t
  misc_feature (412)..(412): n is a, c, g, or t
  misc_feature (416)..(416): n is a, c, g, or t
  misc_feature (429)..(429): n is a, c, g, or t
  misc_feature (435)..(435): n is a, c, g, or t
  misc_feature (444)..(444): n is a, c, g, or t
  misc_feature (475)..(475): n is a, c, g, or t
  misc_feature (481)..(481): n is a, c, g, or t
  misc_feature (485)..(485): n is a, c, g, or t
  misc_feature (488)..(488): n is a, c, g, or t
  misc_feature (505)..(507): n is a, c, g, or t
  misc_feature (525)..(525): n is a, c, g, or t
  misc_feature (528)..(528): n is a, c, g, or t
  misc_feature (532)..(532): n is a, c, g, or t
  misc_feature (534)..(535): n is a, c, g, or t
  misc_feature (543)..(543): n is a, c, g, or t
  misc_feature (551)..(553): n is a, c, g, or t
  misc_feature (555)..(555): n is a, c, g, or t
  misc_feature (561)..(561): n is a, c, g, or t
  misc_feature (568)..(568): n is a, c, g, or t
  misc_feature (573)..(573): n is a, c, g, or t
  misc_feature (584)..(584): n is a, c, g, or t
  misc_feature (589)..(589): n is a, c, g, or t
  misc_feature (598)..(598): n is a, c, g, or t
  misc_feature (605)..(605): n is a, c, g, or t
  misc_feature (612)..(612): n is a, c, g, or t
  misc_feature (618)..(618): n is a, c, g, or t
  misc_feature (633)..(633): n is a, c, g, or t
  misc_feature (647)..(647): n is a, c, g, or t
  misc_feature (653)..(654): n is a, c, g, or t
  misc_feature (664)..(664): n is a, c, g, or t
  misc_feature (679)..(679): n is a, c, g, or t
  misc_feature (689)..(689): n is a, c, g, or t
  misc_feature (695)..(695): n is a, c, g, or t
  misc_feature (698)..(698): n is a, c, g, or t
  misc_feature (711)..(711): n is a, c, g, or t
  misc_feature (720)..(720): n is a, c, g, or t
atatgacctgcgnncanacncnctaanangngactngttaaanacnttccgtggaatnnactcagactgcaaantgtnatnctgncnnannntgnngactgtccngncngatttnnngcntgnaatactattgcctcttatatacacnaccaannntgcgaagggcnannnnacctttnccantnnnctggggncccacnnnngngaactgagagtggatcttgtgtacctgacnnaccagntntnnagnagggcgctcactctgattggtgcaccatggttacacagtgtgtgcaaagaccngnctatctcactgangatgattgncagngccnntgggtggcacnangggnactgatgancancactgaccctgccgacgccagangccgcanatccggagantncatgngacnatataggttaccnccttcnaccgggcancaatctgcttctatggtgaatgcagaccatntagaantctntcnctataggcatgattttnnncagtgcgtcagccttganaangaancnnacttttgntagatgannngntgctcncccttgnggctnacaaattccancaccnttggtggcngcagccnttaagancacttnttttgggttgcgctnttggatgaattacnaatagnntgttttgttncaaggcccttctgcnaaatatgaanaaaagngcncttagctttttgngggaactgnactggaaattttg

SEQ ID NO: 126 - DNA, 119 bp, Human
  misc_feature (17)..(17): n is a, c, g, or t
  misc_feature (21)..(23): n is a, c, g, or t
  misc_feature (28)..(29): n is a, c, g, or t
  misc_feature (31)..(31): n is a, c, g, or t
  misc_feature (35)..(35): n is a, c, g, or t
  misc_feature (39)..(41): n is a, c, g, or t
  misc_feature (45)..(45): n is a, c, g, or t
  misc_feature (50)..(50): n is a, c, g, or t
  misc_feature (52)..(52): n is a, c, g, or t
  misc_feature (54)..(57): n is a, c, g, or t
  misc_feature (61)..(61): n is a, c, g, or t
  misc_feature (63)..(63): n is a, c, g, or t
  misc_feature (71)..(71): n is a, c, g, or t
  misc_feature (79)..(80): n is a, c, g, or t
  misc_feature (87)..(93): n is a, c, g, or t
  misc_feature (110)..(112): n is a, c, g, or t
cagacgattttaaagangggnnnaacannancccngggnnngggnttggngncnnnngggncnaacccccnaaaagggnngggaaannnnnnnaaagggggggccccccnnnaaaaaaa

SEQ ID NO: 127 - DNA, 100 bp, Human
  misc_feature (13)..(13): n is a, c, g, or t
  misc_feature (15)..(15): n is a, c, g, or t
  misc_feature (37)..(37): n is a, c, g, or t
  misc_feature (42)..(42): n is a, c, g, or t
  misc_feature (44)..(45): n is a, c, g, or t
  misc_feature (50)..(50): n is a, c, g, or t
  misc_feature (57)..(59): n is a, c, g, or t
  misc_feature (70)..(70): n is a, c, g, or t
  misc_feature (74)..(74): n is a, c, g, or t
  misc_feature (80)..(80): n is a, c, g, or t
gatcagacaagancntggtccacagcgggacgagagntctcnannctgcnggggagnnnccaagtacgcnagcnctgaanctaaagcaagcaagaaaaag

SEQ ID NO: 128 - DNA, 393 bp, Human
atttccatcgtttgagagactctgctgtctctcttttgatgagccctgtcgtggatgattccttgagttcaattattgttgggaacctggtactgtcccgggtcggcgacataagcctcatgtggatatatattttatctatattcactctttttaagaaagtgatcattctgttcctgttgtctcggacagccacattttaagccaagtgacaacgggagcgggctgatgttacaacacttgcgaagtgaaatggaagatgacatctgaatgatgactttgaggggtcctatgactaaatgttttacaagctatcacttgttattgaatttagcttgcctacttttagatgacaaaagatcaatttaaacctgaaaagtaggaagcagaaga

SEQ ID NO: 129 - DNA, 355 bp, Human
  misc_feature (258)..(258): n is a, c, g, or t
  misc_feature (263)..(263): n is a, c, g, or t
  misc_feature (273)..(273): n is a, c, g, or t
  misc_feature (301)..(301): n is a, c, g, or t
ctgtgtttgctataaattgggagcattgtgttaagaatgctgaaaaataagaaaaaagaactgctataaggcagcatttgagtacattattcaatttttaagaagtagaaaggattttcagtagcttaaacatttttaaaaagttcatatctgatcagtagtgtatcaggtttttgttgtcttgttgatatcattttaagcttcctctgtatttcatcaatgctgccttaatttaattcatgtattaaatacagatgntttgntttcctcagngaaagggagatttttctttgcaagcagngataaaatgtaaaataagataagtgacaactgttttatagaacccattggggat

SEQ ID NO: 130 - DNA, 161 bp, Human
  misc_feature (7)..(7): n is a, c, g, or t
  misc_feature (19)..(19): n is a, c, g, or t
  misc_feature (38)..(38): n is a, c, g, or t
  misc_feature (81)..(81): n is a, c, g, or t
  misc_feature (86)..(87): n is a, c, g, or t
  misc_feature (103)..(103): n is a, c, g, or t
  misc_feature (116)..(116): n is a, c, g, or t
  misc_feature (122)..(122): n is a, c, g, or t
  misc_feature (153)..(153): n is a, c, g, or t
gaatatngtagtacccccnaaaacctttacctggactngggggttggattttaataaaaaaagccttggttttctggcctnccttnnctggaaaaggcccttngggaattttgganaaggtncccccccccggaaaaagttttttttaaaaanttttcccc

SEQ ID NO: 131 - DNA, 103 bp, Human
  misc_feature (26)..(26): n is a, c, g, or t
  misc_feature (44)..(44): n is a, c, g, or t
  misc_feature (49)..(49): n is a, c, g, or t
  misc_feature (59)..(60): n is a, c, g, or t
  misc_feature (63)..(63): n is a, c, g, or t
  misc_feature (89)..(89): n is a, c, g, or t
  misc_feature (97)..(97): n is a, c, g, or t
aagtagctggtataatcccagtatcngtcatgattaagaagtgnattgnagttctgatnnacnaacaatacatagaacgcatccaggcnctggttgntgaata

SEQ ID NO: 132 - DNA, 130 bp, Human
  misc_feature (5)..(5): n is a, c, g, or t
  misc_feature (30)..(30): n is a, c, g, or t
  misc_feature (102)..(102): n is a, c, g, or t
gtaancctaagacccttactatccaacaanatctgggtcagatttcactttttgatcccagggaggaatagctctccccttgccagcttatatacccttagnaatatctatgatcccaggactgacgtgg

SEQ ID NO: 133 - DNA, 100 bp, Human
  misc_feature (12)..(12): n is a, c, g, or t
  misc_feature (85)..(85): n is a, c, g, or t
  misc_feature (91)..(91): n is a, c, g, or t
  misc_feature (99)..(99): n is a, c, g, or t
attgtggctggnatggaacaacataccttggggacccctggctggcacaccttgcctctgaatttctgggcttggtcttccaggnggcatntgcctgang

SEQ ID NO: 134 - DNA, 269 bp, Human
tgtagaagttatgtgtaatttttaaaaacatttcttgtcataggttctattagtaaaaatatatacagatatatatgcaaagtcttaggagatattttccatgcgaattaaacttttaactgctttaattgttttttcaaatagagaacagcatagaaaatatgacattttctgtgtgccctgttcagtttagtttacattaattaccacataaaattccaagatctatgatcaaagtttaataactgacaagttactagtaatttagt

SEQ ID NO: 135 - DNA, 108 bp, Human
  misc_feature (89)..(89): n is a, c, g, or t
  misc_feature (95)..(95): n is a, c, g, or t
tacgactatagagactagttcaggcctcccagatactgacaaacgtggggaagtgacccagctggctggcaacctggcagaggaccatnatgccngctgtcaaaagag

SEQ ID NO: 136 - DNA, 209 bp, Human
ctcatacacctgtggctactgttttctacagagtgccaaaactattcgagagaataggctctggactggacactgtatacccacatgcaagatgaagttggccccttacatcctatacgcaggagaattgcgtcatttaaagcctgttgacgcttttctcccgcagacgaatggaaagattaattgggagtgggggctgaaacaattcg

SEQ ID NO: 137 - DNA, 321 bp, Human
  misc_feature (5)..(5): n is a, c, g, or t
  misc_feature (19)..(19): n is a, c, g, or t
  misc_feature (27)..(27): n is a, c, g, or t
  misc_feature (31)..(31): n is a, c, g, or t
  misc_feature (34)..(34): n is a, c, g, or t
  misc_feature (52)..(52): n is a, c, g, or t
  misc_feature (97)..(97): n is a, c, g, or t
  misc_feature (122)..(122): n is a, c, g, or t
  misc_feature (130)..(130): n is a, c, g, or t
  misc_feature (144)..(144): n is a, c, g, or t
  misc_feature (165)..(165): n is a, c, g, or t
  misc_feature (171)..(171): n is a, c, g, or t
  misc_feature (202)..(202): n is a, c, g, or t
  misc_feature (226)..(226): n is a, c, g, or t
  misc_feature (256)..(256): n is a, c, g, or t
  misc_feature (267)..(267): n is a, c, g, or t
  misc_feature (300)..(300): n is a, c, g, or t
agggnaaaagattttttanccccccantggnaanaaagtggggttggggggnaattttttttttggtttttggtttttggtttttttggtttttttnggtttttggtttggtttttggtttnggtttttnggtttttttttttnggcccaccttaaaattttttnaaggtnattttccattttcctggccatttgcctaagnataaaaaaaagcctggaaaggttnaccttttaatggtttttgggcccttttttnaaatggccttncaattttccaaataattgggacaatttttggtnaagttttgaacccgggggggg

SEQ ID NO: 138 - DNA, 262 bp, Human
aattttgctgttacatggtggctcaactgagtcccatactttgaaggccgggagttaatcacctggtcaccgagttgcgaaccagcctccaatatgtggaaccctgtactctctaaaaatcaaatcaccggcatggagattgcgcctgtggtcccaaaatactcgggctgggacacgatgagttgcttggcccaaggaaggagggttgtatggctgatcacactggtccgcctgggtgacagagcgagactccatctctaat

SEQ ID NO: 139 - DNA, 350 bp, Human
aaacctctctaactatatatcacaataacctgcgcataagatttacgctccgatcttttcatcctactagcttggaggatttgaaccgattatgaatacgcaatactcccggtcctcatgtatcatgtgtaagcccatctcctgggagggctaacatactaccatctccaaggagaggcatgattccgaatcacccacagacagctcgatcaccatacgtatcacccaacatatataccttctaagacttgctagaaacaaccaccacatttgatgcttaatcaccactctgacgcgcattaaagtgaggggactctcctaatttctgtaagttgatttttgcattctga

SEQ ID NO: 140 - DNA, 258 bp, Human
taacatggtaagagggatacatgactgctcatgctgactataagaatgctgccactgatgagctgcagtcaccactagcggttctagcggatgatgaacacccagcgtacggtgtgcccatggccaagacccatatctctaatcaggatactatagtgattctcatgaacaactcactgaagaacaacgacgtaagcccattgtctttgaaaaagaagagatgctttgtcttattgctcatcaatagagaaaaacttg

SEQ ID NO: 141 - DNA, 341 bp, Human
taatcccagatggaagcgtggagatggaaagcatggaacgggcgggaaattaaaggaaataatcccagatggaagcgtggaggtggaaagcatggaacgggcgggaaattaaaggaaataatcccagatggaagcgtggaggtggaaagcatggaacgggcgggaaattaaaggaaataatcccagatggaagcgtggagatggaaagcatggaacgggcgggaaattaaaggaaataatcccagatggaagcgtggagatggaaagcatggaacgggcgggaaattaaaggaaataatcccagatggaagcgtggagatggaaagcatggaacgggcggg

SEQ ID NO: 142 - DNA, 309 bp, Human
gccagatgccgtgtttcctcgatgaactctttacatcattggctattcagtggagtgtttcattatcacctctcactctcgcgtgttacctaactctccctcgcaggggaaatcactccatatatttcaaatgtcttgctaacagtggttactttgctctatccttagctatacgtctcgaggcacattgttcctctatgccccgctacgctttgccctagagctcggcggtatctatatcttaactgccctcttgatccttacgtgccggagaaggtggaggcagaaattttgtcaaatctgattaga

SEQ ID NO: 143 - DNA, 245 bp, Human
  misc_feature (173)..(175): n is a, c, g, or t
ctccatttgcttgttcttaaaaacatttgtaagtagcttgtaatattaccagtaccaattattgttctttgcaattgcttcagcccaagaaagcttgtgtatttgttttaaaaattctgtaaaaatttatttggtgattgattcattttagcattaaagaagaaggtggacgnnngaagggtttttccttattgtattcaaaacttttgtcttattaatttattgtcattcttattgtaccttag

SEQ ID NO: 144 - DNA, 414 bp, Human
gtaccataggtagattatagattatagttatagataatgttttaaatgttcctttatttttgtctgagtcatgtaagtttgagcacaggatggtcttaaaaagtcttaaaatgtcagtttcaaaaaacaaattttacgtcttaaaagtgttaattttcaataaaagtggtcatacactcaaaaagggttttattataaataaaagtctattgtgaaaaagaataaaataattttagcatgcaatatttttgataaaccattttattcccaaacttgcatagaatattgtacattttaagaaaaaaaaactggggataatataaaagacaaacattttctcatgaatgtgttaaaggcttatgccatttaattattagcaaattcatctgtcattatgtaatgtgtttcacatag

SEQ ID NO: 145 - DNA, 279 bp, Human
ggagctgttattccttcttgacgtttggacttcagagatgaagcgtactcaagccgctctgctgtgcttctgaatgggaacactgtgtgtgtgtgtgtgtgttaaaggctgactccagatcagttcctcactcagacgttcactcctctgctgtggttcatctgtcggcatgcttcacctttactgcagttcagtttcctcttgttcggtgtcattttgacagacatgtacagaaccgtttgcaaacttgcatcaagtttatgaataaagaattttaag

SEQ ID NO: 146 - DNA, 100 bp, Human
  misc_feature (19)..(19): n is a, c, g, or t
  misc_feature (30)..(30): n is a, c, g, or t
  misc_feature (50)..(50): n is a, c, g, or t
  misc_feature (53)..(53): n is a, c, g, or t
  misc_feature (57)..(57): n is a, c, g, or t
  misc_feature (67)..(68): n is a, c, g, or t
gactcgagcaagcttatgnctgcggccganatttcgagctcacttggccnatntcgncctatagagnnttacgaatacttcttagatcggtgcgcgaaga

SEQ ID NO: 147 - DNA, 100 bp, Human
  misc_feature (3)..(3): n is a, c, g, or t
  misc_feature (5)..(5): n is a, c, g, or t
  misc_feature (14)..(14): n is a, c, g, or t
  misc_feature (25)..(25): n is a, c, g, or t
  misc_feature (30)..(30): n is a, c, g, or t
  misc_feature (42)..(42): n is a, c, g, or t
  misc_feature (59)..(59): n is a, c, g, or t
aangngggaaagangagaaaattanagggnttcttaatcggngcgcgaagaatggtaanagagacggcggaagttcctgcgccggatggagaagcaagca

SEQ ID NO: 148 - DNA, 100 bp, Human
  misc_feature (18)..(19): n is a, c, g, or t
  misc_feature (47)..(47): n is a, c, g, or t
  misc_feature (54)..(54): n is a, c, g, or t
  misc_feature (63)..(63): n is a, c, g, or t
  misc_feature (68)..(68): n is a, c, g, or t
  misc_feature (70)..(70): n is a, c, g, or t
  misc_feature (81)..(81): n is a, c, g, or t
actcgagcaagcttatgnntgcggccgacattcgagctcacttggcnaattcgncctataganagtangnatacttcttanctcagcgcgagaagatatt

SEQ ID NO: 149 - DNA, 100 bp, Human
  misc_feature (2)..(2): n is a, c, g, or t
  misc_feature (15)..(15): n is a, c, g, or t
  misc_feature (29)..(29): n is a, c, g, or t
  misc_feature (37)..(38): n is a, c, g, or t
  misc_feature (44)..(44): n is a, c, g, or t
  misc_feature (65)..(65): n is a, c, g, or t
  misc_feature (74)..(75): n is a, c, g, or t
  misc_feature (82)..(85): n is a, c, g, or t
  misc_feature (88)..(88): n is a, c, g, or t
tntggcccggggccnaaggttagactgantaactttnngtgtantttttaatttgaatgttgggnctttttaanngacccannnntanaggggaaccttt

SEQ ID NO: 150 - DNA, 100 bp, Human
  misc_feature (4)..(4): n is a, c, g, or t
  misc_feature (10)..(10): n is a, c, g, or t
  misc_feature (32)..(32): n is a, c, g, or t
  misc_feature (87)..(87): n is a, c, g, or t
cggngatctnacaggaatgtgcctaggaaccngattatcatttaatactgaaacagctgaggaagggacagagaaggtacaagggcnaggcggcacagca

SEQ ID NO: 151 - DNA, 222 bp, Human
  misc_feature (34)..(34): n is a, c, g, or t
  misc_feature (37)..(37): n is a, c, g, or t
  misc_feature (43)..(43): n is a, c, g, or t
  misc_feature (47)..(47): n is a, c, g, or t
  misc_feature (56)..(56): n is a, c, g, or t
  misc_feature (80)..(80): n is a, c, g, or t
  misc_feature (94)..(94): n is a, c, g, or t
  misc_feature (104)..(104): n is a, c, g, or t
  misc_feature (127)..(127): n is a, c, g, or t
  misc_feature (129)..(129): n is a, c, g, or t
  misc_feature (177)..(177): n is a, c, g, or t
  misc_feature (198)..(198): n is a, c, g, or t
  misc_feature (206)..(206): n is a, c, g, or t
  misc_feature (214)..(214): n is a, c, g, or t
ctttttggttgatacaatgattctattaatctanatnatcttnggtncttatgggnagcccataagcgtaaataaggggnccattaaattgggnaagggagagnccaccaccaccttttggttaaangnttccaaggaaagtgggggtttccctttttaccccaggggggggggggnaggaccagggaaaaaaaaaantggggggngagtttcncccccccc

SEQ ID NO: 152 - DNA, 104 bp, Human
  misc_feature (1)..(1): n is a, c, g, or t
  misc_feature (7)..(7): n is a, c, g, or t
  misc_feature (29)..(30): n is a, c, g, or t
  misc_feature (42)..(43): n is a, c, g, or t
  misc_feature (52)..(52): n is a, c, g, or t
  misc_feature (72)..(72): n is a, c, g, or t
naaagcnttttaaataaaagtcttgggannacctactaaacnnaaagagtantaaaacattttctgtaggcnagcaattagccagccaggataaaaaaccaaac

SEQ ID NO: 153 - DNA, 122 bp, Human
  misc_feature (11)..(11): n is a, c, g, or t
  misc_feature (16)..(16): n is a, c, g, or t
  misc_feature (27)..(27): n is a, c, g, or t
  misc_feature (35)..(35): n is a, c, g, or t
  misc_feature (39)..(39): n is a, c, g, or t
  misc_feature (54)..(54): n is a, c, g, or t
  misc_feature (59)..(59): n is a, c, g, or t
  misc_feature (67)..(67): n is a, c, g, or t
  misc_feature (97)..(97): n is a, c, g, or t
ggccgaaaagnggccnatcctcttacntaaaaaantgcnaaaaaatttagcccnagggnggtgggtngggccattaccgcccctggtaagttccccnagccttaccttccagggaaggcctg

SEQ ID NO: 154 - DNA, 125 bp, Human
  misc_feature (5)..(5): n is a, c, g, or t
  misc_feature (19)..(19): n is a, c, g, or t
  misc_feature (29)..(29): n is a, c, g, or t
  misc_feature (38)..(38): n is a, c, g, or t
  misc_feature (42)..(42): n is a, c, g, or t
  misc_feature (45)..(45): n is a, c, g, or t
  misc_feature (49)..(49): n is a, c, g, or t
  misc_feature (61)..(61): n is a, c, g, or t
  misc_feature (77)..(77): n is a, c, g, or t
  misc_feature (79)..(79): n is a, c, g, or t
  misc_feature (88)..(88): n is a, c, g, or t
  misc_feature (90)..(90): n is a, c, g, or t
  misc_feature (100)..(100): n is a, c, g, or t
  misc_feature (110)..(110): n is a, c, g, or t
cggtngcaattgggggccncatacgcgcngacgagtantggncangctncttgactacacngacgcgccgtacaggntnaattatggnancttacatggnaaaggggcanctcaatgtcccacag

SEQ ID NO: 155 - DNA, 104 bp, Human
cacccgggaattcggcattatggccggggaacaaggcaggccgccagccaatcctagaagctttctggtttgggaaggctgcaaattgtaagctgggtgttggg

SEQ ID NO: 156 - DNA, 241 bp, Human
  misc_feature (15)..(15): n is a, c, g, or t
  misc_feature (103)..(103): n is a, c, g, or t
  misc_feature (116)..(116): n is a, c, g, or t
  misc_feature (167)..(167): n is a, c, g, or t
tgtaaatcccccaancactttttggaaagggcttgaagggcaggccaagaattggctttgaagggtcaagggaaattccgaagaacccagcccctggcccaancattggccagaanccccttttcttcccacttaaaaaaataccaaaaaattagtcaagggtgttngggtgggcaccgccaagcttggtaattcttcagcttacctcttggaaggcttgaaacttgggagaaatcacttt

SEQ ID NO: 157 - DNA, 367 bp, Human
  misc_feature (11)..(11): n is a, c, g, or t
  misc_feature (35)..(35): n is a, c, g, or t
  misc_feature (37)..(38): n is a, c, g, or t
  misc_feature (43)..(43): n is a, c, g, or t
  misc_feature (56)..(56): n is a, c, g, or t
  misc_feature (67)..(67): n is a, c, g, or t
  misc_feature (71)..(71): n is a, c, g, or t
  misc_feature (75)..(75): n is a, c, g, or t
atagcaaaagngggtaaaacccctgagtttgcganannagtantcttgtaggggcnaactctacttnagangaantcctcgcaaaatccttgaatcaccgcttcagtgcagtgatatcaccgccatgaaatttctgctcgattagcttacgttgtttggatagaggccaaacaaggctgttatcggtacgaggaatggatgttcgatttcgtagaatacgcctgagagacggcgaatactctcacgagaggcagcaggcgcgtaaattacccaattacaacaagtagaggtagcgaaggaaaatatgaggggtggcaaggttttgcctgttacattctcaaatggaagcaaattagatatgtcattg

SEQ ID NO: 158 - DNA, 178 bp, Human
  misc_feature (1)..(1): n is a, c, g, or t
  misc_feature (12)..(12): n is a, c, g, or t
  misc_feature (26)..(26): n is a, c, g, or t
  misc_feature (30)..(30): n is a, c, g, or t
  misc_feature (40)..(40): n is a, c, g, or t
  misc_feature (45)..(45): n is a, c, g, or t
  misc_feature (53)..(53): n is a, c, g, or t
  misc_feature (78)..(78): n is a, c, g, or t
  misc_feature (86)..(86): n is a, c, g, or t
  misc_feature (88)..(88): n is a, c, g, or t
  misc_feature (93)..(93): n is a, c, g, or t
  misc_feature (96)..(96): n is a, c, g, or t
  misc_feature (109)..(109): n is a, c, g, or t
  misc_feature (143)..(143): n is a, c, g, or t
  misc_feature (166)..(166): n is a, c, g, or t
ntggcccggganagtgccaccttttntgangttctgaaanttcantggttccncttgacctttttgcgtcaccttaantcccaaantnaaccnaanttcagggttgaantcttgaaattggctttctcaggcctcaaggtaancagtgttctttgtggtttgaccnaattgtttttct

SEQ ID NO: 159 - DNA, 407 bp, Human
  misc_feature (8)..(8): n is a, c, g, or t
  misc_feature (23)..(23): n is a, c, g, or t
  misc_feature (30)..(30): n is a, c, g, or t
  misc_feature (83)..(83): n is a, c, g, or t
  misc_feature (97)..(97): n is a, c, g, or t
  misc_feature (106)..(106): n is a, c, g, or t
  misc_feature (110)..(110): n is a, c, g, or t
  misc_feature (251)..(251): n is a, c, g, or t
ttaagccntccagccttgtcccntaagaangccagtttggtccaccactaaaagggaagtctttaaggggacctttggaaaantgtattccattgantaaacctantaanttttattttggatggtttttggatcaaaagaaataacccagatgcccattattttttccctgaaagggaaattgcctggaccatttaccaccttgtttttaggggtgtcattcattttcaccagaggtttaaatacctggngggagtgaccacccagaaccacagcccgaagaggcctagaaagccaagaaaaggatctgcatgataacctttgcagcttgagaatagttccctaattcattcaacgtaacaaacaaagcttttggggtgtcccatgatatacccaggcactatgct

SEQ ID NO: 160 - DNA, 109 bp, Human
  misc_feature (3)..(3): n is a, c, g, or t
  misc_feature (11)..(11): n is a, c, g, or t
  misc_feature (20)..(20): n is a, c, g, or t
  misc_feature (22)..(22): n is a, c, g, or t
  misc_feature (29)..(29): n is a, c, g, or t
  misc_feature (33)..(33): n is a, c, g, or t
  misc_feature (42)..(42): n is a, c, g, or t
  misc_feature (46)..(46): n is a, c, g, or t
  misc_feature (57)..(57): n is a, c, g, or t
  misc_feature (61)..(61): n is a, c, g, or t
  misc_feature (78)..(78): n is a, c, g, or t
  misc_feature (96)..(97): n is a, c, g, or t
  misc_feature (100)..(100): n is a, c, g, or t
cangtgggtgnaggggggangnagggggnaggnggggggggnaggnagaggcggtgnggangggggagggggccagangcagcggagaacaaaggnnggnctgggacag

SEQ ID NO: 161 - DNA, 236 bp, Human
atacctagagtgggataatgctttatagcgcccccacaaggaaggggttgctgacaactctcgattggtgctgagtatagcagtctgctgtaaacgacagtgatgctgaacgataacttgaagcagctttccaaactcgttagtggtgtggactgattcttcgctgtgtgtgtgctcacatcagcgccttcattgttatcgggattgctgtcctgatttgtaaatcgaggagctct

SEQ ID NO: 162 - DNA, 373 bp, Human
  misc_feature (11)..(11): n is a, c, g, or t
  misc_feature (20)..(20): n is a, c, g, or t
  misc_feature (24)..(24): n is a, c, g, or t
  misc_feature (31)..(31): n is a, c, g, or t
  misc_feature (38)..(38): n is a, c, g, or t
  misc_feature (53)..(53): n is a, c, g, or t
  misc_feature (61)..(62): n is a, c, g, or t
  misc_feature (66)..(66): n is a, c, g, or t
  misc_feature (83)..(84): n is a, c, g, or t
  misc_feature (92)..(92): n is a, c, g, or t
  misc_feature (109)..(109): n is a, c, g, or t
  misc_feature (115)..(115): n is a, c, g, or t
  misc_feature (146)..(146): n is a, c, g, or t
  misc_feature (153)..(153): n is a, c, g, or t
  misc_feature (166)..(166): n is a, c, g, or t
ctttaaaaacntgttagacnaacnttaaaanttacccnttttcctgaactgantcctgggnntaantaaaaagggtgaagaannttacttcncttggtcctaaaaaacnttttcntcagttattaccaaaatatttggaccattantaaagantagggccaacccnaatttttcttgaaatttccgttaaatagccgttaaatgtttttacccatttcatattggataccttaaattataataatggattttattgttaaattgtgtgtgtgtggtgtgtatgccctgtcttttctcctctaccattattgtcactttatgtttggaaccccctttacccttccttaaaggaaaaaaagggcccggggttttt

SEQ ID NO: 163 - DNA, 128 bp, Human
  misc_feature (10)..(10): n is a, c, g, or t
  misc_feature (20)..(20): n is a, c, g, or t
  misc_feature (29)..(30): n is a, c, g, or t
  misc_feature (35)..(35): n is a, c, g, or t
  misc_feature (49)..(49): n is a, c, g, or t
  misc_feature (92)..(92): n is a, c, g, or t
tcctagtaanctggtttacnctgaaagannaagangcctcccctgttcnctgaaataccaccttgatgttcaagtatttaagaccctatgcnaatattttttaccttttctaataaaccatgtttgtt

SEQ ID NO: 164 - DNA, 254 bp, Human
  misc_feature (30)..(30): n is a, c, g, or t
  misc_feature (55)..(55): n is a, c, g, or t
  misc_feature (70)..(71): n is a, c, g, or t
  misc_feature (95)..(95): n is a, c, g, or t
  misc_feature (159)..(159): n is a, c, g, or t
  misc_feature (178)..(178): n is a, c, g, or t
cgggaaatctttgggagggaagccaagaanccagccaaaaggggtggatgcctgnctccagccagggtcnnccccttcaaggggccccaaagaangttattttttccccattccccccgtgggaagcaccttggagggaagggaagattcccgttatcnagattcccgcccgaccganctgcaagccagtttcggggagggagttcaaacctcatttgcccatctggacacatacaaggaaccttttgaactgg

SEQ ID NO: 165 - DNA, 213 bp, Human
  misc_feature (9)..(9): n is a, c, g, or t
  misc_feature (88)..(88): n is a, c, g, or t
  misc_feature (98)..(98): n is a, c, g, or t
  misc_feature (105)..(105): n is a, c, g, or t
  misc_feature (107)..(107): n is a, c, g, or t
  misc_feature (127)..(127): n is a, c, g, or t
  misc_feature (142)..(142): n is a, c, g, or t
  misc_feature (147)..(147): n is a, c, g, or t
  misc_feature (161)..(161): n is a, c, g, or t
  misc_feature (166)..(167): n is a, c, g, or t
  misc_feature (171)..(171): n is a, c, g, or t
  misc_feature (180)..(180): n is a, c, g, or t
  misc_feature (196)..(196): n is a, c, g, or t
  misc_feature (206)..(206): n is a, c, g, or t
cccattggnacagacccccaaaatgggtacattttttaggaaaccaggacctttccaaggggccaggccttccctttaaaaaaaaatnaccgtttttngggggangnaacctttaaaaggggaaaanaaatcctttttaaanggaantccaagggaaggancctgnncaanacttccccnccaataaaaaaaaccnttttggaaanggggaaa

SEQ ID NO: 166 - DNA, 102 bp, Human
  misc_feature (12)..(12): n is a, c, g, or t
  misc_feature (14)..(14): n is a, c, g, or t
  misc_feature (17)..(17): n is a, c, g, or t
  misc_feature (28)..(29): n is a, c, g, or t
  misc_feature (36)..(36): n is a, c, g, or t
  misc_feature (51)..(51): n is a, c, g, or t
  misc_feature (55)..(56): n is a, c, g, or t
tcgttaagtaantngantgttaccaacnnggggtantttactcctagttcnaganntcaggtaatgccttcctgcaggaagtgaagtttcctagatttgagc

SEQ ID NO: 167 - DNA, 369 bp, Human
  misc_feature (2)..(3): n is a, c, g, or t
  misc_feature (5)..(5): n is a, c, g, or t
  misc_feature (8)..(8): n is a, c, g, or t
  misc_feature (11)..(11): n is a, c, g, or t
  misc_feature (14)..(15): n is a, c, g, or t
  misc_feature (20)..(20): n is a, c, g, or t
  misc_feature (26)..(26): n is a, c, g, or t
  misc_feature (39)..(40): n is a, c, g, or t
  misc_feature (44)..(44): n is a, c, g, or t
  misc_feature (50)..(51): n is a, c, g, or t
  misc_feature (57)..(57): n is a, c, g, or t
  misc_feature (60)..(60): n is a, c, g, or t
  misc_feature (66)..(66): n is a, c, g, or t
  misc_feature (73)..(73): n is a, c, g, or t
  misc_feature (80)..(80): n is a, c, g, or t
  misc_feature (86)..(87): n is a, c, g, or t
  misc_feature (90)..(90): n is a, c, g, or t
  misc_feature (93)..(93): n is a, c, g, or t
  misc_feature (99)..(99): n is a, c, g, or t
  misc_feature (102)..(102): n is a, c, g, or t
  misc_feature (106)..(106): n is a, c, g, or t
  misc_feature (133)..(133): n is a, c, g, or t
  misc_feature (137)..(137): n is a, c, g, or t
  misc_feature (156)..(156): n is a, c, g, or t
anncnttntcnttnngggcnaacccnacctttttttacnnggtncgcccnntaagtntcnttcccntttttcntaagaanaggtcnntanttngggaantcnctantaagttattggtaagccccttttttcnaganctgggcctttcctttttcnacctttaaataaactattgccaaatttaaagggtttcccctcccattgttccatttttcatgggccctaaatagtgccattttatttttaagcacctgaataatacctcccattgtctagatgaattaggtttatcccattcaccctatttgaaagacttcttgggggggtttccaaggttttggcaattatgaataaagcctggtggtaaac

SEQ ID NO: 168 - DNA, 103 bp, Human
  misc_feature (1)..(1): n is a, c, g, or t
  misc_feature (12)..(12): n is a, c, g, or t
  misc_feature (17)..(17): n is a, c, g, or t
  misc_feature (22)..(23): n is a, c, g, or t
  misc_feature (47)..(47): n is a, c, g, or t
  misc_feature (65)..(65): n is a, c, g, or t
  misc_feature (78)..(78): n is a, c, g, or t
natttagaagcntgccnagaannagacctggtgaaaaagtaaacccntttcacctacaaaatttnaccctgcaaaccnttaaaaccctgcaaaattttccttt

SEQ ID NO: 169 - DNA, 411 bp, Human
cgctcgcgccgagtacgctctccaccagctggccaagcgtgacaagaactgggctcagcgtgagcccggtgtcatcgtcgtctttgtcattgttttcctcgtcgccgtcctggtcattgccatgttcatcaacaagaagaggcaagccgccaaaggcgtcgcataagatacccccttgattcgagttggagcgcagccgagcagtacgatatgaaggaggacaagacatgacccccgatacgaagtcataaagctgtatgctggacgggcacgagcttgtgtataatgtgtttcaatatttgcgtataggataacgcgggctttccttgatgggactgcactgtggtgcgaacgcctatattcctaggcttcgtagtaataatgaacaagtcaaatacccattggaaaaaa

SEQ ID NO: 170 - DNA, 132 bp, Human
ccaaaccatttacccaatagggtataggcgatagagattgaaacctcggcgcaatagatatagtaccacaaaggaaaagatgaaaaattataccccacgcataatatagcaaaggcctacccccctatacct

SEQ ID NO: 171 - DNA, 239 bp, Human
tcagtggcccatttctgctttccgtcatcccactcccaagccctggtggcctttttccaaagatatagtggtaggggcttcccaaaggcgatccaaaggcatactcctaccgtttgcccggcccctgctggtggctgcccctcgtcgctgcgggtccccaaagtttgtgaggccctttgtgcccgcgctcgcttccagaaatacttccgcttaagaccctcgtggctctcaaactccct

SEQ ID NO: 172 - DNA, 100 bp, Human
acaaaaagcccctttaaacttgggcccgctcgaggtcgtttcgactgggccgagacttccgaaaagaaaatggttttttttgccgaaatcaaccgggtaa

SEQ ID NO: 173 - DNA, 183 bp, Human
actgccaagactctgcgagtcgaactgtaaatccgccgaaagcagggctgacatacccgagaaaggtgaaatggaaaaccaaagagggacagctctgtggaaaataggaaaaaaaccttggagagagaggaaaaaataagacccgccatgagcaggcgaagcagccgccaccaatgaaaaaga

SEQ ID NO: 174 - DNA, 223 bp, Human
tattctgcacaaaagggtattagtcactgtaccactggctttactacaggaaagggtgccattcagtttaattcaccctttgtcagaaaaggagttggaggggtattaacagcttagggggcaggcattaattaaaaggggtactctaaatgtacccctattctcagacacaggaaggggatttatgtgcacatgtataaaatgtttaaactctttgtatata

SEQ ID NO: 175 - DNA, 176 bp, Human
cgagcgcctgatacgcagctaggaatattgataggacccggttcttttttttggtttcggactaggcccgattaagagggacgcccggggcattcgtttttcgccgtagaggggaaattttggaccggcgaagagggcccaaaacgaaagtttttccaagattttttttatttccc

SEQ ID NO: 176 - DNA, 531 bp, Human
tatagcgggcgttataaacataccacttcccggtacaacggatttcaaggttaggggtgcaacccagaacgaacgcgttaagtgcgcgttatcttcctaggatagagtcggtgacgggaatcttttaccccggcactcgggtccaccctcgcggcaccagaggtattctccggcgagtcgttaaccatcgcaatcgccgaccgagtttaaggaccactccccacctttctcattagttaaggagaacgctactttaccccatagacggagaaatcgctactcaactaccaggcgcgcgccgtcgagtccctcttcctctctttatgcatttagagcgctttcgtaagagttttccctagattcttctaagcgtagcgcgtctactccaatgttttcgttaatccagcccgaactaacgccgcggaggagtcgatccgtctactcctatcccgtcggctcggatttactacaggagctaagaaaacaaaaagtaccagccctaaaggaaagtcaaaggacgcccgtaaaaaa

SEQ ID NO: 177 - DNA, 530 bp, Human
aatctcgatcgcaaacatacggcactctccctcttgccgcggttttcgtccagcgctttccattcggtccagtgcctcgccctattagcccttaagcccaccgtttctaaaactcccagaacagccaaaccggtccgcccaaggcctccgtcgttttataatatattccgtttacgtataaggaacgaaccccccttcattaccacggtcccgcgtccgcctccttctccattcgcaacagttctattcctttcagcctcccgtacctgcttccagaacatcgcaccgccatagtcgaaagatagcaaagattacccagcttctattcctcgccccagagccgagtaaatcgaagtttatagaggcggaatccaaccattcaagagttataacaagttatcggcactcgggggatcagaatataaacttaatgtcccctttattctcccggacgccccttttaaccacttcttcctatctttcgctaacaagccattgacggcgctttgccgcgcgggcccatctcgcgt

SEQ ID NO: 178 - DNA, 274 bp, Human
tggttggggacgccaaatctcgcaaggaagcctttaatcacaaatccacaattttagtcgcggctctcgagagatcgaagacctgaggtctttcggttagggtagtttcctaggagtcttccagcaaaggatccactaaatcggccctaaagtatagagtctaagcaacccatattagaaacattaagggcggagacgtcttaagtcgtcggcgaagacgtacgaacaggcttaattaagtcgcatacccggaaaaaaaaaaaaaaaaaaaaaa

SEQ ID NO: 179 - DNA, 560 bp, Human
ttgggggaacaaataaacctagccctctttatcccaactctataatatttcctcgatcttagacgcgcgcggaggggggccttttatgtaccaagtaaacaagtaaccccgtagtagatattagtcgggcgcttctcaacctaacctatcctccatatcattcggagacagtactagacattacaggccgaaggtctagcgttataagacggtctttatcctagacgataactagaagttttagacggcgattaagggcttcaattaatcagtcacaagccagggcctttacaattttcctccgagtcttaccagcccgtcagagagttctatagaatctggaaatatcacatagctacgtcgcatacaacgataccctaacgctagttcgtacagataacggcgactttacaagaccacaacatcgcacccccatcgcattcccctcacgcccaggcacgcccagacagataaacagctaacttccgaataatccgggcccgaatcgcgcgggcttatccacatacggacccccccctaacaaacgcaccggaaaacca

SEQ ID NO: 180 - DNA, 151 bp, Human
  misc_feature (29)..(29): n is a, c, g, or t
  misc_feature (52)..(52): n is a, c, g, or t
tgttcagtttcattctgtattctcagtantatttcttattcatatacatttngtgtacaactggtttaataagcggcaaaccatatgctataggctatgcaggtcatccattctgtaaggtctctgtggcaagtctttcaatagggatact

SEQ ID NO: 181 - DNA, 332 bp, Human
  misc_feature (37)..(37): n is a, c, g, or t
  misc_feature (47)..(47): n is a, c, g, or t
  misc_feature (108)..(108): n is a, c, g, or t
  misc_feature (112)..(112): n is a, c, g, or t
  misc_feature (129)..(129): n is a, c, g, or t
  misc_feature (134)..(134): n is a, c, g, or t
  misc_feature (152)..(152): n is a, c, g, or t
  misc_feature (168)..(168): n is a, c, g, or t
  misc_feature (196)..(196): n is a, c, g, or t
  misc_feature (239)..(239): n is a, c, g, or t
ccatttatgggccggggatatacccacatggtacagnacattacatntttatggcaccatttccaccggcctggttttggtttttccataattaattaaccagggggnccanttaaaaaaaattaaggnaaggnttaaaaaatttaaccaanggggggtttaaagggntttttttttttaaaaaaaaaggttaaanccccccctttttttttgggttggggtgggaaaattttgggaanccttaacccccgggtttttgggtttttttggccaaaaccccccggaaaaaaattaaaaaaaggaccggtttccattttaatgggtattgggaa

SEQ ID NO: 182 - DNA, 165 bp, Human
tctctacagagtgaaggtttaaatccaaggtcatggcaaacatctgaaggtcatcgccaaggctgtggttggacaaggaggggacggtggaaggcgattacaaggcccctaaacagggattcctaaatatggatgggcaaaattggagcccataaggattggggg

SEQ ID NO: 183 - DNA, 212 bp, Human
tagatgatctgcctttcaggttatctcaagggcagtttcacctttccataatataaatgtgacccttagatattaagggtattattctttccttcttcctggttaggcccttttcctggggtttgccttaccgggcttcccccatatatggacaggatgatatcaggtattttttaggcctattttagtagagtaccggggggggacccact

SEQ ID NO: 184 - DNA, 117 bp, Human
attccttaaccctggaggcagaggtttcagctagccaagatcacaccgcatcaccgtcagccctggcgacagagacagacactgtctataaaaaaaaaaaaaaaaaaaaaaaaaaaa

SEQ ID NO: 185 - DNA, 154 bp, Human
atcccatagtgaaatgctcaaatactgagtgccttatagtattcaacaaggacaacatgagacgtgggcggtattgcaatgatgaattaaatccacctctaaaaaaaaaaacaaaaagtttagaaaagacttgtccacccctttgcccaggaaa

SEQ ID NO: 186 - DNA, 126 bp, Human
agaactaagccggaccaagcttagacgacagtggaacgaaaacaaagcaacggggaaggcccgcggcggggtgttgaccgcgatgagaactctgcccagtgctctgaatgtcaaaaaaaaaaaaaa

SEQ ID NO: 187 - DNA, 100 bp, Human
  misc_feature (25)..(26): n is a, c, g, or t
  misc_feature (28)..(28): n is a, c, g, or t
  misc_feature (32)..(32): n is a, c, g, or t
  misc_feature (34)..(34): n is a, c, g, or t
  misc_feature (44)..(45): n is a, c, g, or t
  misc_feature (49)..(49): n is a, c, g, or t
  misc_feature (56)..(56): n is a, c, g, or t
  misc_feature (62)..(63): n is a, c, g, or t
  misc_feature (66)..(66): n is a, c, g, or t
  misc_feature (74)..(74): n is a, c, g, or t
  misc_feature (79)..(79): n is a, c, g, or t
  misc_feature (97)..(97): n is a, c, g, or t
  misc_feature (99)..(100): n is a, c, g, or t
ggcctcgctgatgtctgataagcanngncagngngcaaaaggcnntacntcagaanggccanntanagcaaacnaaggngaaaattttagaaacagncnn

SEQ ID NO: 188 - DNA, 250 bp, Human
  misc_feature (21)..(21): n is a, c, g, or t
  misc_feature (27)..(27): n is a, c, g, or t
  misc_feature (43)..(43): n is a, c, g, or t
  misc_feature (47)..(47): n is a, c, g, or t
  misc_feature (68)..(68): n is a, c, g, or t
  misc_feature (85)..(85): n is a, c, g, or t
  misc_feature (89)..(89): n is a, c, g, or t
  misc_feature (93)..(93): n is a, c, g, or t
  misc_feature (104)..(104): n is a, c, g, or t
  misc_feature (107)..(108): n is a, c, g, or t
  misc_feature (112)..(112): n is a, c, g, or t
  misc_feature (116)..(118): n is a, c, g, or t
  misc_feature (135)..(135): n is a, c, g, or t
  misc_feature (143)..(143): n is a, c, g, or t
  misc_feature (154)..(155): n is a, c, g, or t
  misc_feature (158)..(158): n is a, c, g, or t
  misc_feature (173)..(174): n is a, c, g, or t
  misc_feature (178)..(178): n is a, c, g, or t
  misc_feature (196)..(196): n is a, c, g, or t
  misc_feature (198)..(198): n is a, c, g, or t
  misc_feature (209)..(209): n is a, c, g, or t
  misc_feature (215)..(216): n is a, c, g, or t
  misc_feature (219)..(219): n is a, c, g, or t
caattttttgttaaaaccctnagaaangaacaaaaccgccttngagnacctcaatttcaggaaagaancggcaatgaaaccaggncccncagnccaccaggganggnngaangggnnngggtaaaaatggcctcnaccaaatntgggggccatnnccntaaagggggtttttnncccnattccccccgggggggcncnggccaaaaggntggggnnccnacccttaaaaaaaccaaattcccttaaaaaa

SEQ ID NO: 189 - DNA, 310 bp, Human
  misc_feature (164)..(164): n is a, c, g, or t
  misc_feature (212)..(212): n is a, c, g, or t
aaagatccgcgagacccgggaggtttggtaataactaataacggggggaagccacaggacccaaagcagccccttttacaggccaggtaggagagagaaaaaaaatgccaaaaaccacctagcgggcaagaaccaatttttaataacaaaaagggttccgcccnatggggggggcaaaatggaaccggttaggaaaaggggctttcccaaanccaaccaaggaaggggccccaaggggccaaaccggaaaaccaaattccaggtccattaccaggggcataaaaccaataaaggcaaaatttttccccca

SEQ ID NO: 190 - DNA, 143 bp, Human
cgagtgatctaaaacgttagatagagtgtggccttgtgggttcctgttgctggagacttcctaggatggagccgccctaaaccgccgggcccggtgcccttgcgcgggagttggggtccaaataaagttcttgggatgtaaaa

SEQ ID NO: 191 - DNA, 152 bp, Human
  misc_feature (121)..(121): n is a, c, g, or t
  misc_feature (136)..(136): n is a, c, g, or t
aatttaggcttttggtgggttggacccggaatcctaacaagtccagcaattagccaggcagcccagctttatcagggaaaaccaaacggagattttgagaaaagaggtttcggccattggnggaacaaagctggantggattcaaaaaaaga

SEQ ID NO: 192 - DNA, 160 bp, Human
  misc_feature (79)..(79): n is a, c, g, or t
  misc_feature (94)..(94): n is a, c, g, or t
  misc_feature (112)..(112): n is a, c, g, or t
aacgcaaaggaacaaggacattctttagctggaacctaactaatgaaaggggcctttaccacacgccatttaaataagncccaattttttcttntttgccatttgggaagcnttgcccattggggcttaaatggcacatgggggagggacaccgggatct

SEQ ID NO: 193 - DNA, 137 bp, Human
ttatgccacaacgaactggctgacgagtggaatacctggaaatgctttcaatgagacatggctgggtgagctggggctgcctatcacgagcttaagaacataccaactgggaaatggaaaaccccgaaactattacc

SEQ ID NO: 194 - DNA, 506 bp, Human
  misc_feature (5)..(5): n is a, c, g, or t
  misc_feature (25)..(25): n is a, c, g, or t
  misc_feature (38)..(39): n is a, c, g, or t
  misc_feature (44)..(44): n is a, c, g, or t
  misc_feature (46)..(46): n is a, c, g, or t
  misc_feature (59)..(59): n is a, c, g, or t
  misc_feature (66)..(66): n is a, c, g, or t
  misc_feature (70)..(70): n is a, c, g, or t
  misc_feature (74)..(74): n is a, c, g, or t
  misc_feature (80)..(80): n is a, c, g, or t
  misc_feature (82)..(84): n is a, c, g, or t
  misc_feature (86)..(86): n is a, c, g, or t
  misc_feature (90)..(92): n is a, c, g, or t
  misc_feature (112)..(113): n is a, c, g, or t
  misc_feature (117)..(117): n is a, c, g, or t
  misc_feature (126)..(126): n is a, c, g, or t
  misc_feature (129)..(129): n is a, c, g, or t
  misc_feature (133)..(136): n is a, c, g, or t
  misc_feature (152)..(152): n is a, c, g, or t
  misc_feature (156)..(157): n is a, c, g, or t
  misc_feature (162)..(162): n is a, c, g, or t
  misc_feature (164)..(165): n is a, c, g, or t
  misc_feature (174)..(174): n is a, c, g, or t
  misc_feature (178)..(178): n is a, c, g, or t
  misc_feature (181)..(181): n is a, c, g, or t
  misc_feature (185)..(187): n is a, c, g, or t
  misc_feature (190)..(191): n is a, c, g, or t
  misc_feature (194)..(194): n is a, c, g, or t
  misc_feature (206)..(206): n is a, c, g, or t
  misc_feature (227)..(227): n is a, c, g, or t
  misc_feature (231)..(231): n is a, c, g, or t
  misc_feature (235)..(235): n is a, c, g, or t
  misc_feature (244)..(244): n is a, c, g, or t
  misc_feature (247)..(247): n is a, c, g, or t
  misc_feature (251)..(251): n is a, c, g, or t
  misc_feature (255)..(255): n is a, c, g, or t
  misc_feature (258)..(258): n is a, c, g, or t
  misc_feature (260)..(261): n is a, c, g, or t
  misc_feature (263)..(263): n is a, c, g, or t
  misc_feature (270)..(270): n is a, c, g, or t
  misc_feature (273)..(276): n is a, c, g, or t
  misc_feature (285)..(285): n is a, c, g, or t
  misc_feature (287)..(287): n is a, c, g, or t
  misc_feature (291)..(292): n is a, c, g, or t
  misc_feature (302)..(302): n is a, c, g, or t
  misc_feature (305)..(306): n is a, c, g, or t
  misc_feature (311)..(311): n is a, c, g, or t
  misc_feature (318)..(319): n is a, c, g, or t
  misc_feature (321)..(321): n is a, c, g, or t
  misc_feature (327)..(329): n is a, c, g, or t
  misc_feature (333)..(333): n is a, c, g, or t
  misc_feature (348)..(349): n is a, c, g, or t
  misc_feature (352)..(352): n is a, c, g, or t
  misc_feature (357)..(357): n is a, c, g, or t
  misc_feature (363)..(363): n is a, c, g, or t
  misc_feature (366)..(367): n is a, c, g, or t
  misc_feature (369)..(369): n is a, c, g, or t
  misc_feature (371)..(371): n is a, c, g, or t
  misc_feature (376)..(377): n is a, c, g, or t
  misc_feature (385)..(385): n is a, c, g, or t
  misc_feature (390)..(390): n is a, c, g, or t
  misc_feature (398)..(398): n is a, c, g, or t
  misc_feature (401)..(401): n is a, c, g, or t
  misc_feature (405)..(405): n is a, c, g, or t
  misc_feature (420)..(420): n is a, c, g, or t
  misc_feature (429)..(429): n is a, c, g, or t
  misc_feature (437)..(437): n is a, c, g, or t
  misc_feature (440)..(440): n is a, c, g, or t
  misc_feature (442)..(443): n is a, c, g, or t
  misc_feature (453)..(453): n is a, c, g, or t
  misc_feature (455)..(456): n is a, c, g, or t
  misc_feature (473)..(474): n is a, c, g, or t
  misc_feature (477)..(477): n is a, c, g, or t
  misc_feature (481)..(481): n is a, c, g, or t
  misc_feature (487)..(488): n is a, c, g, or t
  misc_feature (495)..(497): n is a, c, g, or t
  misc_feature (500)..(500): n is a, c, g, or t
  misc_feature (502)..(502): n is a, c, g, or t
  misc_feature (506)..(506): n is a, c, g, or t
ttggngggggggcgagatcctactngagacccttgatnntgggnanggaccgaagatcnattaganaccnatgngatggncnnncnaaannnttaaagtgagagtccatctnngaanaaaatgggnaantttnnnnggggggggggaaaaancccnngggtnanngggggcccngggnttnaaannnggnnctnggggggggaaanttttggcccccccccgggggntttncctnaaaaaaaanccntttnaaanacngnnanaattttnccnnnncggggaggngngganntttttttttnaannagccntttttgnnanaaaaannntggncccccccctattccnnggnttttnggaccnttnnancntgggnntttttagnccttnaaaaaaangcnaatnttaaggtaaaaattnggggggggngggggggnggngnntttttttttntnnggaggggtttttttccnncgngggngaaagnntggggcnnnctncngccn

SEQ ID NO: 195 - DNA, 312 bp, Human
agagcatagaacttatcccaatcagatttgatggatgaatgaaaggaagaaaactgactgttcttagtgtttgctggaaagaggaaaaccatgagcaatgggataaattaaacatttcagttgaataaatgtttaccaaattgctaataaagaataggctctgtacttggccttgggcaaacaatagtgtaaaatatacagtctgccctctagttgcttattggagtgagcatagtacattctgataaatgtacaagagctagaatccagaacatagaattttccttgtggatttgtttaaaaaaaaaaaaa

SEQ ID NO: 196 - DNA, 100 bp, Human
  misc_feature (34)..(34): n is a, c, g, or t
  misc_feature (45)..(45): n is a, c, g, or t
  misc_feature (58)..(58): n is a, c, g, or t
  misc_feature (61)..(61): n is a, c, g, or t
  misc_feature (73)..(73): n is a, c, g, or t
  misc_feature (76)..(76): n is a, c, g, or t
  misc_feature (84)..(85): n is a, c, g, or t
  misc_feature (87)..(89): n is a, c, g, or t
  misc_feature (92)..(92): n is a, c, g, or t
  misc_feature (95)..(95): n is a, c, g, or t
  misc_feature (98)..(99): n is a, c, g, or t
gtatataaaagagtaaaatgatacagccagtaanaaaaaaaaaantttaaaaaaaganccnggggggcccccnggncccggggnncnnnaanccnggnna

SEQ ID NO: 197 - DNA, 289 bp, Human
tttgcttgagcttcacctttaagggtctgatgataaacctgtgacttgcctgcctgcctgcctgatttttgtcaaaaggcaaaggtggtacaaaaatgtgttgtaggccaccttcgtaattctctgcacgaaatctctcaatcagatttaagcagacttcattatattacatttcattatatcagaatatcttctattttttacagtcatagctgtttggaaagggacaagtaatcgttaacatttaaaaataaaagatttggaaaaaaaaaaaaaaaaaaaaaaaaaa

SEQ ID NO: 198 - DNA, 400 bp, Human
  misc_feature (11)..(11): n is a, c, g, or t
  misc_feature (24)..(24): n is a, c, g, or t
  misc_feature (40)..(40): n is a, c, g, or t
  misc_feature (48)..(48): n is a, c, g, or t
  misc_feature (51)..(51): n is a, c, g, or t
  misc_feature (71)..(71): n is a, c, g, or t
  misc_feature (80)..(80): n is a, c, g, or t
  misc_feature (83)..(83): n is a, c, g, or t
  misc_feature (91)..(91): n is a, c, g, or t
  misc_feature (95)..(95): n is a, c, g, or t
  misc_feature (99)..(99): n is a, c, g, or t
  misc_feature (111)..(111): n is a, c, g, or t
  misc_feature (115)..(116): n is a, c, g, or t
  misc_feature (118)..(118): n is a, c, g, or t
  misc_feature (121)..(121): n is a, c, g, or t
  misc_feature (123)..(124): n is a, c, g, or t
  misc_feature (126)..(126): n is a, c, g, or t
  misc_feature (129)..(129): n is a, c, g, or t
  misc_feature (131)..(131): n is a, c, g, or t
  misc_feature (133)..(133): n is a, c, g, or t
  misc_feature (135)..(135): n is a, c, g, or t
  misc_feature (148)..(148): n is a, c, g, or t
  misc_feature (153)..(153): n is a, c, g, or t
  misc_feature (170)..(170): n is a, c, g, or t
  misc_feature (173)..(173): n is a, c, g, or t
  misc_feature (175)..(175): n is a, c, g, or t
  misc_feature (177)..(177): n is a, c, g, or t
  misc_feature (192)..(192): n is a, c, g, or t
  misc_feature (195)..(196): n is a, c, g, or t
  misc_feature (198)..(198): n is a, c, g, or t
  misc_feature (200)..(201): n is a, c, g, or t
  misc_feature (211)..(212): n is a, c, g, or t
  misc_feature (219)..(219): n is a, c, g, or t
  misc_feature (222)..(223): n is a, c, g, or t
  misc_feature (226)..(226): n is a, c, g, or t
  misc_feature (231)..(231): n is a, c, g, or t
  misc_feature (240)..(240): n is a, c, g, or t
  misc_feature (243)..(244): n is a, c, g, or t
  misc_feature (249)..(249): n is a, c, g, or t
  misc_feature (255)..(255): n is a, c, g, or t
  misc_feature (269)..(269): n is a, c, g, or t
  misc_feature (280)..(280): n is a, c, g, or t
  misc_feature (283)..(283): n is a, c, g, or t
  misc_feature (289)..(289): n is a, c, g, or t
  misc_feature (293)..(293): n is a, c, g, or t
  misc_feature (311)..(311): n is a, c, g, or t
  misc_feature (324)..(324): n is a, c, g, or t
  misc_feature (326)..(326): n is a, c, g, or t
  misc_feature (341)..(341): n is a, c, g, or t
  misc_feature (348)..(348): n is a, c, g, or t
  misc_feature (350)..(350): n is a, c, g, or t
  misc_feature (354)..(355): n is a, c, g, or t
  misc_feature (367)..(367): n is a, c, g, or t
  misc_feature (383)..(384): n is a, c, g, or t
  misc_feature (391)..(391): n is a, c, g, or t
  misc_feature (398)..(398): n is a, c, g, or t
  misc_feature (400)..(400): n is a, c, g, or t
catgaaggaanaagcctgtactanctgccggtatccatgntaatctgnggngatgtcagcagacccagctnagcagatanctncatttctntctnaagncctttggtctgnaggnngncantnnanctncngntnaacatcacagctnctccnagcatcaccctgctagntancngngggttttctcttatntgnngncnnaacatctgcnngctctgntannaanaattncataccgcncanngtctntgacgntgtgatgcatacgnttgggcagagngancaatangtgngcatatgcgtgccttacncaaggatacggangngcttgaaattgatgngaccaanantttnngtacggtaagtnacccaaccacttctgnnttcactntaagagncn

SEQ ID NO: 199 - DNA, 367 bp, Human
  misc_feature (41)..(41): n is a, c, g, or t
ctgtaataatgataatgaagtattttcataatggtgtaacnagttggcaattcttcaaaaagaaacaaaataatcacacggtggtaatttcctctcttgggtatactacagacccgtgtatcccagcagcctgagtgaccaggcagggtgggttctggttctccttgtactgaggcctttcctatgcatgcagatagagttgaatctacacatgagcagagtaagtaacaatatgcagtaataaaagtgacatgaactgaagtcaggtaaggcggctcatgcattcagccagcacttacaaggcagaggcaggtacatctctgtgatctttaaggccacctcagtctacagagtgagaccttctctc

SEQ ID NO: 200 - DNA, 215 bp, Human
  misc_feature (9)..(9): n is a, c, g, or t
taaaaatcntaattagagcgagaagaggagacccatccctaacttgccagatgatcagtcaggctcatgaaccattgatatcttcctgctattgatgaatgtgacttcagccattctagcgccattactgtggaacgtgattggcagaaagccgcgcgtccgcaccaaaacacctttttatacaaatgacagatgcgtgaattaaagagattaaa

SEQ ID NO: 201 - DNA, 260 bp, Human
  misc_feature (58)..(58): n is a, c, g, or t
  misc_feature (78)..(78): n is a, c, g, or t
cgtggtattgcctttttgccttgaaatttagttcctgataaaggtggaaatgttttantcttctcttttgtggaaaancagataatgggagttggggcaggaaagccctattggcccattccttccaaagaccagaccagaatcaccctggaggccgttcaaaagatataacccaaataaaccaagtcatcccacaatcaatacaacatttcaatactttcccaggtgtgtcagacttgggatgggacgctgatataata

SEQ ID NO: 202 - DNA, 120 bp, Human
ctcacggtgatgtaaaggactgccgatgtgtactggcttcggctgtgctgtcttctgctttactattaaaacgccacaatcagatgcggatagaacactcttgtgctcagcaccaacacg

SEQ ID NO: 203 - DNA, 158 bp, Human
  misc_feature (27)..(27): n is a, c, g, or t
  misc_feature (33)..(33): n is a, c, g, or t
  misc_feature (48)..(48): n is a, c, g, or t
  misc_feature (57)..(57): n is a, c, g, or t
  misc_feature (73)..(73): n is a, c, g, or t
  misc_feature (85)..(85): n is a, c, g, or t
tcctgcggaaagtgtctttgtctcccnttgganaaaaggaaccaaccnacaaaacantgccctcacttggaantttcccaccgcntttgtgaagccgtgtcgtatgaacctaagtaaaacttttgtacaaaaaaaaaaaaaaaaaaaaaaaaaaaaaa

SEQ ID NO: 204 - DNA, 142 bp, Human
  misc_feature (8)..(9): n is a, c, g, or t
  misc_feature (38)..(38): n is a, c, g, or t
  misc_feature (43)..(44): n is a, c, g, or t
  misc_feature (47)..(47): n is a, c, g, or t
  misc_feature (130)..(130): n is a, c, g, or t
gagtagcnnaagtctgcttatgagatgctcggtaacantttcnncantctgacaaaggtcgacaagactgtacagtcaagatcttggctgcttggggggaagcggaaagatttgctaatattgagaatcngttgtatcaaac

SEQ ID NO: 205 - DNA, 100 bp, Human
  misc_feature (4)..(5): n is a, c, g, or t
  misc_feature (30)..(31): n is a, c, g, or t
  misc_feature (58)..(58): n is a, c, g, or t
  misc_feature (65)..(65): n is a, c, g, or t
  misc_feature (78)..(78): n is a, c, g, or t
  misc_feature (95)..(96): n is a, c, g, or t
  misc_feature (98)..(99): n is a, c, g, or t
agcnngtatgaagaggctcgtccgagagcnncatcggaaagctgcgagagaggcaccntgactcncttatgtttgganctccgtaaacacagttnngnnc

SEQ ID NO: 206 - DNA, 100 bp, Human
  misc_feature (17)..(17): n is a, c, g, or t
ttcaggccgtctgcttntacatatactatcgagaatggtgctgtgcactcataacaccgttgcttggtagacgcttttgaacccttcagcgctgaaagta

SEQ ID NO: 207 - DNA, 128 bp, Human
  misc_feature (3)..(3): n is a, c, g, or t
  misc_feature (16)..(16): n is a, c, g, or t
  misc_feature (29)..(29): n is a, c, g, or t
  misc_feature (40)..(40): n is a, c, g, or t
  misc_feature (44)..(44): n is a, c, g, or t
  misc_feature (47)..(47): n is a, c, g, or t
  misc_feature (57)..(57): n is a, c, g, or t
  misc_feature (69)..(69): n is a, c, g, or t
  misc_feature (79)..(79): n is a, c, g, or t
  misc_feature (84)..(84): n is a, c, g, or t
  misc_feature (105)..(105): n is a, c, g, or t
  misc_feature (125)..(125): n is a, c, g, or t
acngttgtgttggganaaaatgaagattncatcaaggtgngaantgnaagaaattgnaaaagcgggttngggcgggcantactngcagacgctcttagtccgagngcgaggattatattcatcgngga

SEQ ID NO: 208 - DNA, 120 bp, Human
  misc_feature (2)..(2): n is a, c, g, or t
  misc_feature (8)..(8): n is a, c, g, or t
  misc_feature (49)..(49): n is a, c, g, or t
  misc_feature (96)..(96): n is a, c, g, or t
gnagttcntgagcatgatgattgggtatttcgtgcacatgtgtgaagangtgccaccctcgaacctttgttaatgaacatcggcacatttgctcantctaacagaaaaaaaaaaaaaaaa

SEQ ID NO: 209 - DNA, 500 bp, Human
aaggtgaacaaatgatgacaggattgacgtttttgtttgaatgttccattttatttaattttttttctcattttctttgttttattatctctgatcttttatttggcttcatttataaatcaaagagataggaaatttgtgcgtcccgctttgccatgtttgtcagacctcacgagtatgttttggtacatttgtgttctgtttgttattgtttttaaagtctaagcctgactgaaggcagagttcagttgtgcaggtcagttcttcagattaagtctgagagaaagtataaaaaaaggaaaatttctatgattgtgtccatgtgtacatactgatgctttgatttatcttcttttcttttcctgcagttgattgaaatgtcctttttcctctggtgggaaaaagagaacgatagcaggcaaatcatttgagcattgcttcgggtttgtatttatctctctttttttttgctttgaatagagctattgcaataaaaaatg

SEQ ID NO: 210 - DNA, 135 bp, Human
  misc_feature (20)..(20): n is a, c, g, or t
tgtaagctgttcctctgganctcaccctctgttgagttgtaaatgtgagagaaaaaagttactatgtgaaatctactactcaagccagcattttgtatttttgcatcattaaaaaaaaaaaaaaaaaaaaaaaaa

SEQ ID NO: 211 - DNA, 286 bp, Human
ctattaaaacgaaaaacagattcagaagaaaaaaataagacatgttgtatatgtttttgtatgtttatttgtttgttgttattaaaaaaaaaattgacactgaaaaaagtggactaagaataaaaaatatattttttattctgtatatatggaaatcaagataccaatatgttgcattgctttgttgtacaaatcaaaaatgtgcttgtggctattttttttaatctaatttattctacagtattttgtcctttgcttcaccattaaagttaaaaggttgtcccct

SEQ ID NO: 212 - DNA, 106 bp, Human
  misc_feature (5)..(5): n is a, c, g, or t
  misc_feature (8)..(8): n is a, c, g, or t
  misc_feature (21)..(21): n is a, c, g, or t
  misc_feature (25)..(25): n is a, c, g, or t
  misc_feature (31)..(31): n is a, c, g, or t
  misc_feature (33)..(33): n is a, c, g, or t
  misc_feature (35)..(35): n is a, c, g, or t
  misc_feature (44)..(44): n is a, c, g, or t
  misc_feature (51)..(51): n is a, c, g, or t
  misc_feature (60)..(60): n is a, c, g, or t
  misc_feature (78)..(78): n is a, c, g, or t
  misc_feature (87)..(87): n is a, c, g, or t
  misc_feature (89)..(89): n is a, c, g, or t
  misc_feature (94)..(94): n is a, c, g, or t
  misc_feature (97)..(97): n is a, c, g, or t
ctctnatntcattcttcaacntttnttcctntntntacaatttntactttntataaacanctttatcatttttataanctttccatnanttttnctntaactacta

SEQ ID NO: 213 - DNA, 132 bp, Human
  misc_feature (121)..(121): n is a, c, g, or t
gttacctaatgttttactctcattttctttttctttatttttcatttgtaaaataggaacattaattgtactactttcaaaagaattaattgaagaaagagagatacagggtatctaggcngaggaagaccc

SEQ ID NO: 214 - DNA, 246 bp, Human
gggggctttagttataactgggctaagcataattgcgctaccaattccatattatctcatggcacttaattttataattgatatatataataaaaaattcaatgcagatattgatataataaaaatagataatggtaatccaagcacgatggtagccatcactctaattgctttggggttaacctataacttattaagtaaagtgccagaatggttctttgacagtattaaaattaaagaaaacag

SEQ ID NO: 215 - DNA, 109 bp, Human
  misc_feature (6)..(6): n is a, c, g, or t
  misc_feature (14)..(14): n is a, c, g, or t
  misc_feature (27)..(27): n is a, c, g, or t
  misc_feature (36)..(37): n is a, c, g, or t
  misc_feature (39)..(39): n is a, c, g, or t
  misc_feature (54)..(54): n is a, c, g, or t
  misc_feature (57)..(57): n is a, c, g, or t
cataangagaggcncaaatggacacantaacaacannanccttaaaggtgaaangantagaggccccacttaaaagacacagactggcaaattggatagagtgacaaga

SEQ ID NO: 216 - DNA, 100 bp, Human
  misc_feature (10)..(10): n is a, c, g, or t
  misc_feature (23)..(23): n is a, c, g, or t
  misc_feature (38)..(39): n is a, c, g, or t
  misc_feature (44)..(44): n is a, c, g, or t
  misc_feature (61)..(61): n is a, c, g, or t
tgtttaaggnggtataagcaatntaacaattttgatgnngaatnaataattcccctttggnaaaaaagagctgaagttatctaagatcagcatactgttg

SEQ ID NO: 217 - DNA, 191 bp, Human
gaaaaacatttagtagtagtatgttttagacttttggagctcattgtttgtctaaataaatatgcaactcattcccagatggtacaagacagattcactgaatgtgttatttttatcaaaagctacattttataatcgtgttgtgttgagtgattgaaacactaattcagaaaataaaaacacatttgatg

SEQ ID NO: 218 - DNA, 396 bp, Human
tctgaagtatggcataatgtttacacagaaaaatctatcaatagagcttaatttaaatattcaaagctgcacataaataaatcagcagcaaacagaatctgagcacgttaaacatgagtggatgtgtccataaagtaaagccaatacaacactgctatctggacttaataagccaactttaaaatatatttttccagatgaacatgaaatagcgctcttatcactgtgtattggtttacatgttttaggatgtatatgtatcatctcgaactctggcaaagaacccaagagtctaaacagatctgcctgtgccatctttagtggagagctacacgcataaataaaacttcagccctgaccggaaaggacaataaaacactgtatcaacatttacag

SEQ ID NO: 219 - DNA, 305 bp, Human
ggagtggagccctgtgagatttcccctatttatgttgggatgtcaattgtgattggaattattcaggtttcgttttctgttttaatcttacagttgctctctcagatcaaagttgtatgggaaaaatatagacaaatcatgctgagtatcatatttctgtcctgtattattaaataagttcatttctagaagtgcttaacctaatttattcctaattatggatgattttttttccaaatagtgtttatggtagtcttcccaggatgattcttttaagataatggatccaacaataaatattatat

SEQ ID NO: 220 - DNA, 290 bp, Human
cagcttagattaggtgctgagtacaggttcctgggtgtactctggacacgagaactactccacacttgtctagcatctcgtgtgcttggctgagccccataggagggagatccatggctgttctgggaggagaccagttcaccaagcaggcattgctccttgtcttgcagactatgaaacttttttctggagaccgagtctcactatgtagaccaggctggctttgaactcacagagatccacccgccctgcctcctgaatgctaagtaagattaaaggtgtgaaccacc

SEQ ID NO: 221 - DNA, 314 bp, Human
  misc_feature (267)..(267): n is a, c, g, or t
tcagtgatttgtgctgggctaagcgaaaagtgctccattcatataggtgatcggctcaatggattcaaaaaatggtaggactcggctgttgagctctatgtgacttgtaaaatgagccagtttacaaaaaaaagtagagtgttccttcaagaccttgacgtgaactatgctgtgtcattctgcaactttacttaatttatccacctaaatacatagatgatatatttttgttggaactctaaatacttaagtgagtattatgtaccnttttttggggtggggggagatttaggttagaatttagtagaaaatcg

SEQ ID NO: 222 - DNA, 427 bp, Human
gaaatttagacccctagtgcacaatcaatttgtcattattgctgagcctggtttctctcaagctttttcttccttgatgaagtttattccttccttgatttgagacttgttgagctgtacatggactagctcttcagtatttagactttcagcagtgaaaattaaactaaattgaaaatcgactctgactgacacaatctacattgtaaaagtgctacgttaataaagataaattgaattaaattaattagaatcttccattataacaattattaaatgtttacctttgtctgccttctagctgagagaagctgaaggagtgacacatttaatggaagaaaagaaaaaagaaagaaacaaaggacaattcacaaacaacgatcagttttaattatgtaaaaataaataaacaaatacatgcaagata

SEQ ID NO: 223 - DNA, 108 bp, Human
ccattatatgttgatgtattaaaagacctgagaacctggtgtaagtaacttagttcaattcagtatttcccaaatttactagactatagcatcctttttttttttaag

SEQ ID NO: 224 - DNA, 18 bp, Artificial Sequence
  forward primer of exon 1 of insulin gene used for quantitative RT-PCR analysis
  primer_bind (1)..(18)
gccctctggggacctgac

SEQ ID NO: 225 - DNA, 18 bp, Artificial Sequence
  reverse primer of exons 1 and 2 of insulin gene used for quantitative RT-PCR analysis
  primer_bind (1)..(18)
cccacctgcaggtcctct

SEQ ID NO: 226 - DNA, 24 bp, Artificial Sequence
  forward primer of MyHC gene used for quantitative RT-PCR analysis
  primer_bind (1)..(24)
gctggaacgtagagactccctgct

SEQ ID NO: 227 - DNA, 24 bp, Artificial Sequence
  reverse primer of MyHC gene used for quantitative RT-PCR analysis
  primer_bind (1)..(24)
ggatccttccagatcatccacttg

SEQ ID NO: 228 - DNA, 20 bp, Artificial Sequence
  forward primer of ANF used for quantitative RT-PCR analysis
  primer_bind (1)..(20)
ggatttcaagaatttgctgg

SEQ ID NO: 229 - DNA, 20 bp, Artificial Sequence
  reverse primer of ANF used for quantitative RT-PCR analysis
  primer_bind (1)..(20)
gcagatcgatcagaggagtc

SEQ ID NO: 230 - DNA, 20 bp, Artificial Sequence
  forward primer of APP used for quantitative RT-PCR analysis
  primer_bind (1)..(20)
ggatgcttcatgtgaacgtg

SEQ ID NO: 231 - DNA, 19 bp, Artificial Sequence
  reverse primer of APP used for quantitative RT-PCR analysis
  primer_bind (1)..(19)
tcattcacaccagcacatg

SEQ ID NO: 232 - DNA, 21 bp, Artificial Sequence
  forward primer of ZFP used for quantitative RT-PCR analysis
  primer_bind (1)..(21)
cacargagrcarggtcaacga

SEQ ID NO: 233 - DNA, 22 bp, Artificial Sequence
  reverse primer of ZFP used for quantitative RT-PCR analysis
  primer_bind (1)..(22)
ggattaaaatgaagcacccaga